(19) United States
(12) Patent Application Publication
(10) Pub. No.: US 2026/0000761 A1
(43) Pub. Date: Jan. 1, 2026

(54) ENGINEERED IMMUNE CELLS WITH ENHANCED POTENCY AND USES OF SAME IN IMMUNOTHERAPY

(71) Applicant: NKARTA, INC., South San Francisco, CA (US)

(72) Inventors: James Barnaby Trager, Albany, CA (US); Ivan Chan, Millbrae, CA (US); Don-Hong Wang, Palo Alto, CA (US); Guangnan Li, Foster City, CA (US); Alexandra Leida Liana Lazetic, Portland, OR (US); Chao Guo, San Francisco, CA (US)

(21) Appl. No.: 18/729,489

(22) PCT Filed: Jan. 18, 2023
(86) PCT No.: PCT/US2023/060850

§ 371 Date: Jul. 16, 2024

Related U.S. Application Data
(60) Provisional application No. 63/300,978, filed on Jan. 19, 2022.
(60) Provisional application No. 63/366,586, filed on Jun. 17, 2022.

(51) Int. Cl. A61K 40/31; A61K 40/11; A61K 40/15; A61K 40/42; C07K 14/725; C07K 16/30

(52) U.S. Cl. CPC A61K 40/31; A61K 40/11; A61K 40/15; A61K 40/42; C07K 14/7051; C07K 16/30

(57) ABSTRACT

Several embodiments of the methods and compositions disclosed herein relate to immune cells that are engineered to express chimeric antigen receptors as well as genetically edited or otherwise engineered enhance the persistence the cells in immunotherapy. In several embodiments, the cells are edited to knock out a target gene that encodes a protein involved in antigen processing and presentation by major histocompatibility complex class I molecules. In several embodiments, a mixture of immune cell types is used, optionally in allogeneic therapy. The engineering and editing of the cells, such as NK cells and/or T cells exhibit enhanced cytotoxicity and/or persistence, as well as reduced risk of reduced graft versus host, host versus graft, and graft versus graft effects.

### CROSS-REFERENCE TO RELATED APPLICATIONS

This application claims priority to U.S. Provisional Patent Application No. 63/300,978, filed Jan. 19, 2022 and U.S. Provisional Patent Application No. 63/366,586, filed Jun. 17, 2022, the entire contents of each of which is incorporated by reference herein.

### FIELD

Several embodiments disclosed herein relate to methods and compositions comprising genetically engineered cells for cancer immunotherapy, in particular combinations of engineered immune cell types. In several embodiments, the present disclosure relates to cells engineered to express chimeric antigen receptors. In several embodiments, further engineering is performed to enhance the efficacy and/or reduce potential side effects when the cells are used in cancer immunotherapy.

### BACKGROUND

As further knowledge is gained about various cancers and what characteristics a cancerous cell has that can be used to specifically distinguish that cell from a healthy cell, therapeutics are under development that leverage the distinct features of a cancerous cell. Immunotherapies that employ engineered immune cells are one approach to treating cancers.

### INCORPORATION BY REFERENCE OF MATERIAL IN SEQUENCE LISTING FILE

This application incorporates by reference the material in the Sequence Listing contained in the following XML file being submitted concurrently herewith: File name: NKT083WO_ST26.XML; created Jan. 18, 2023, which is 1,617,920 bytes in size.

### SUMMARY

Immunotherapy presents a new technological advancement in the treatment of disease, wherein immune cells are engineered to express certain targeting and/or effector molecules that specifically identify and react to diseased or damaged cells. This represents a promising advance due, at least in part, to the potential for specifically targeting diseased or damaged cells, as opposed to more traditional approaches, such as chemotherapy, where all cells are impacted, and the desired outcome is that sufficient healthy cells survive to allow the patient to live. One immunotherapy approach is the recombinant expression of chimeric receptors in immune cells and further engineering or genetically editing the cells to avoid adverse immune responses against the therapeutic cells in order to achieve the efficient and persistent targeted recognition and destruction of aberrant cells of interest.

In several embodiments, there is provided a population of genetically engineered immune cells, comprising genetically engineered immune cells that express a chimeric receptor comprising an extracellular ligand binding domain, a transmembrane domain, and a cytotoxic signaling complex, wherein the immune cells are genetically edited at one or more target sites in the genome of the immune cell to yield reduced levels of expression of a protein which is encoded by a gene which comprises an edited target site as compared to a non-edited immune cell, the genetic edit results in a decrease in the frequency of cell surface expression of major histocompatibility complex class I (MHC I) molecules, and the genetically engineered immune cells are further engineered to express at least one immunosuppressive effector that exerts suppressive effects on the cytotoxic activity of Natural Killer (NK) cells and/or T cells.

In several embodiments, the at least one immunosuppressive effector exerts suppressive effects on the cytotoxic activity of one or more of non-engineered NK cells, non-engineered T cells, genetically engineered NK cells, and genetically engineered T cells. In several embodiments, the population of genetically engineered immune cells comprises genetically engineered NK cells. In several embodiments, the population of genetically engineered immune cells comprises genetically engineered T cells.

In several embodiments, the genetic edit is to TAPBP. In several embodiments, the genetic edit is to TAP-2. In several embodiments, the genetic edit is made to a gene encoding one or more of: TAPBP (Tapasin), TAP-2; UGT-1 (UGTA-1), TAPBPL (TAPBPR), TAP-1, ERp57; Calreticulin (CRT); Endoplasmic reticulum aminopeptidases ERAP1 and/or ERAP2, and/or one or more immunoproteasome components selected from the standard proteasome catalytic subunits β1, β2, and/or β5, and the inducible proteasome catalytic subunits LMP2, MECL-1, and LMP7. In several embodiments, the genetic edit is to a gene involved in antigen processing and/or MHC I complex assembly. In several embodiments, the immune cells are also genetically edited to reduce expression of TCR alpha (TRAC).

In several embodiments, the genetically engineered immune cells comprise one or both of genetically engineered NK cells and genetically engineered T cells. In several embodiments, the population of genetically engineered cells is allogeneic to the NK cells and/or T cells whose cytotoxic activity is suppressed by the at least one immunosuppressive effector. In several embodiments, the at least one immunosuppressive effector exerts suppressive effects on the cytotoxic activity of one or more of non-engineered natural killer cells, non-engineered T cells, and suppressive engineered cells. In several embodiments, the at least one immunosuppressive effector exerts suppressive effects on the cytotoxic activity of one or both of genetically engineered NK cells and genetically engineered T cells. In several embodiments, the at least one immunosuppressive effector exerts suppressive effects on the cytotoxic activity of cells that do not comprise the immunosuppressive effector, and wherein the cells that do not comprise the immunosuppressive effector are either non-engineered or engineered cells.

In several embodiments, a plurality of the genetically engineered immune cells comprises one or more additional genetic edit to a gene encoding one or more of CISH, CBLB, B2M, CD70, adenosine receptor gene, NKG2A, CIITA, TGFBR, and any combination thereof. In several embodiments, the wherein the genetic edit and/or the additional genetic edit is made using a CRISPR/Cas system. In some embodiments, wherein the genetic edit and/or the additional genetic edit is made using a RNA-guided endonuclease. In several embodiments, the genetic edit (and/or the additional edit or edits) reduces host versus graft rejection as compared to immune cells without the genetic edit (and/or the additional edit or edits). In several embodiments, the population of genetically engineered immune cells exhibits one or more of enhanced expansion capability, enhanced cytotoxicity against target cells, and enhanced persistence, as compared to immune cells that do not express the immunosuppressive effector and do not comprise the edited target site.

In several embodiments, there is provided a population of genetically engineered immune cells comprising a plurality of T cells expressing a chimeric receptor, wherein the chimeric receptor comprises an extracellular ligand binding domain, a transmembrane domain, and a cytotoxic signaling complex and the plurality of T cells comprise a genetic edit to TAPBP and/or TAP-2.

In several embodiments, the plurality of T cells comprises a genetic edit to TAPBP, optionally wherein the genetic edit to TAPBP decreases the frequency of T cells in the population that exhibit cell surface expression of MHC I molecules. In several embodiments, the plurality of T cells comprises a genetic edit to TAP-2, optionally wherein the genetic edit to TAP-2 decreases the frequency of T cells in the population that exhibit cell surface expression of MHC I molecules. In several embodiments, the the immune cells are genetically edited to reduce expression of TCR alpha (TRAC). In several embodiments, the population further comprises natural killer (NK cells), optionally wherein a plurality of the NK cells comprises a genetic edit to TAPBP and/or TAP-2. In several embodiments, the T cells and/or the NK cells are genetically engineered to express a chimeric receptor comprising an extracellular ligand binding domain, a transmembrane domain, and a cytotoxic signaling complex.

In several embodiments, the population of genetically engineered immune cells comprises engineered NK cells and engineered T cells. In several embodiments, the chimeric receptor expressed by the engineered T cells binds to one or more of a NKG2D ligand, CD19, CD70, BCMA, CD20, and CD38. In several embodiments, the chimeric receptor expressed by the engineered NK cells binds to one or more of a NKG2D ligand, CD19, CD70, BCMA, CD20, and CD38. In several embodiments, the chimeric receptor expressed by the engineered T cells and the chimeric receptor expressed by the engineered NK cells bind to different targets. In several embodiments, the the cytotoxic signaling complex of the chimeric receptor expressed by the engineered T cells and/or the engineered NK cells comprises a CD3 zeta subdomain. In several embodiments, the cytotoxic signaling complex of the chimeric receptor expressed by the engineered T cells cand/or the engineered NK cells omprises an OX40 subdomain or a 4-1BB subdomain.

In several embodiments, the at least one immunosuppressive effector comprises a virally-derived peptide. In several embodiments, the at least one immunosuppressive effector comprises a peptide derived from a retrovirus. In several embodiments, the at least one immunosuppressive effector comprises a peptide derived from an envelope protein of a retrovirus. In additional embodiments, the at least one immunosuppressive effector comprises at least a portion of human CD47 and/or at least a portion of HLA-E. In several embodiments, the at least one immunosuppressive effector comprises at least a portion of human CD47. In several embodiments, the immunosuppressive effector comprises a peptide having at least 95% sequence identity to SEQ ID NO: 829, 997, 999, 689, or 1014-1017. In several embodiments, the at least one immunosuppressive effector comprises a chimeric construct comprising at least one virally-derived peptide and at least a portion of a human protein and/or at least a portion of a human protein complex.

In several embodiments, the at least one immunosuppressive effector is integrated into the chimeric receptor. In several embodiments, the at least one immunosuppressive effector is integrated into the chimeric receptor between the transmembrane domain and the extracellular ligand-binding domain. In several embodiments, the at least one immunosuppressive effector is integrated into the chimeric receptor within the extracellular ligand-binding domain. In several embodiments, the extracellular ligand-binding domain comprises an scFv and the at least one immunosuppressive effector is integrated into a linker region of the scFv. In several embodiments, the at least one immunosuppressive effector is integrated into the chimeric receptor within an N-terminal region of the chimeric receptor distally positioned from the extracellular ligand-binding domain in relation to the cell membrane. In additional embodiments, the at least one immunosuppressive effector is integrated into the chimeric receptor at a plurality of locations within an extracellular region of the chimeric receptor. In several embodiments, the at least one immunosuppressive effector is bound to an extracellular membrane of the immune cells. In some embodiments, the at least one immunosuppressive effector comprises a transmembrane protein. In some such embodiments, the transmembrane protein is selected from CD8a, CD4, CD3ε, CD3γ, CD3δ, CD3ζ, CD28, CD137, glycophorin A, glycophorin D, nicotinic acetylcholine receptor, a GABA receptor, FcεRIγ, and a T-cell receptor. In several embodiments, the transmembrane protein comprises a CD8α transmembrane protein. In several embodiments, the immunosuppressive effector is expressed on the immune cells by a disulfide trap single chain trimer (dtSCT).

In several embodiments, the immunosuppressive effector is encoded by a nucleic acid or comprises a peptide having at least 85% sequence identity to one or more of the nucleotide or amino acid sequences of SEQ ID NOs: 683-894, 997-1000, 1014-1017, 1020-1023, 1027, 1029, 1031, 1033, 1035, 1037, 1039, 1041, 1046, 1048, 1050, 1052, 1054, 1056, or 1058-1093. In some embodiments, the immunosuppressive effector comprises a peptide having at least 95% sequence identity to SEQ ID NO: 829, 997, 999, 689, or 1014-1017. In several embodiments, the immunosuppressive effector is encoded by a nucleic acid having at least 95% sequence identity to SEQ ID NO: 830, 998, 1000, or 690.

According to several embodiments, the genetically engineered immune cells provided for herein reduce the risk of graft versus host disease as compared to genetically engineered immune cells not having the genetic edit. In several embodiments, the genetically engineered immune cells provided for herein reduce the risk of fratricide among the genetically engineered immune cells. In several embodiments, at least a portion of the genetically engineered immune cells are engineered to express membrane bound IL-15.

Also provided for herein, in several embodiments, is a method for the treatment of cancer in a subject comprising administering to the subject at least a portion of a population of genetically engineered immune cells provided for herein. Additionally, there is provided the use of a population of genetically engineered immune cells provided for herein for the treatment of cancer in a subject or for the preparation of a medicament for the treatment of cancer in a subject.

In several embodiments, there is also provided a method for the treatment of cancer in a subject comprising administering to the subject a population of genetically engineered immune cells, wherein the population of genetically engineered immune cells comprises a plurality of T cells that (i) express a chimeric receptor comprising an extracellular ligand binding domain, a transmembrane domain, and a cytotoxic signaling complex; and (ii) comprise an edit to TAPBP and/or TAP-2. Additonally provided for is the use of a population of genetically engineered immune cells for the treatment of cancer in a subject (or for the preparation of a medicament for the treatment of cancer in a subject), wherein the population of genetically engineered immune cells comprises a plurality of T cells that (i) express a chimeric receptor comprising an extracellular ligand binding domain, a transmembrane domain, and a cytotoxic signaling complex; and (ii) comprise an edit to TAPBP and/or TAP-2.

In several embodiments, the cancer is a hematologic cancer, optionally a B cell cancer. In several embodiments, the cancer comprises a solid tumor. In several embodiments, the population of engineered immune cells is allogeneic to the subject. In several embodiments, treatment of the subject with the population of engineered immune cells produces a decrease immunologic response as compared to treatment of the subject with a population of engineered immune cells not comprising the genetic edit. In several embodiments, the population of engineered immune cells persists for a longer period of time in the subject as compared to a population of engineered immune cells not comprising the genetic edit.

Provided for herein, in several embodiments, is a method of manufacturing a population of genetically engineered immune cells comprising (a) contacting a population of immune cells with an RNA-guided endonuclease to genetically edit one or more target sites in the genome of the immune cell, wherein the genetic editing reduces expression of a protein which is encoded by a gene which comprises an edited target site as compared to a non-edited immune cell, wherein the edit decreases the frequency of cell surface expression of major MHC I molecules within the population of genetically engineered immune cells, (b) contacting the population of immune cells with a polynucleotide encoding a chimeric receptor comprising an extracellular ligand binding domain, a transmembrane domain, and a cytotoxic signaling complex, and (c) contacting the population of immune cells with a polynucleotide encoding at least one immunosuppressive effector that exerts suppressive effects on the cytotoxic activity of suppressive cells. In several embodiments, the immunosuppressive effector is encoded by a nucleic acid or comprises a peptide having at least 85% sequence identity to one or more of the nucleotide or amino acid sequences of SEQ ID NOs: 683-894, 997-1000, 1014-1017, 1020-1023, 1027, 1029, 1031, 1033, 1035, 1037, 1039, 1041, 1046, 1048, 1050, 1052, 1054, 1056, or 1058-1093.

In several embodiments, there is provided a method of enhancing the in vivo persistence of genetically engineered immune cells comprising (a) contacting a population of immune cells with an RNA-guided endonuclease to genetically edit one or more target sites in the genome of the immune cell, wherein the genetic editing reduces expression of a protein which is encoded by a gene which comprises an edited target site as compared to a non-edited immune cell, wherein the edit decreases the frequency of cell surface expression of major MHC I molecules within the population of genetically engineered immune cells, (b) contacting the population of immune cells with a polynucleotide encoding a chimeric receptor comprising an extracellular ligand binding domain, a transmembrane domain, and a cytotoxic signaling complex; and (c) contacting the population of immune cells with a polynucleotide encoding at least one immunosuppressive effector that exerts suppressive effects on the cytotoxic activity of suppressive cells. In several embodiments, the immunosuppressive effector is encoded by a nucleic acid or comprises a peptide having at least 85% sequence identity to one or more of the nucleotide or amino acid sequences of SEQ ID NOs: 683-894, 997-1000, 1014-1017, 1020-1023, 1027, 1029, 1031, 1033, 1035, 1037, 1039, 1041, 1046, 1048, 1050, 1052, 1054, 1056, or 1058-1093. In several embodiments, the genetic edit is made to a gene encoding one or more of TAPBP (Tapasin), TAP-2, UGT-1 (UGTA-1), TAPBPL (TAPBPR), TAP-1, ERp57; Calreticulin (CRT); Endoplasmic reticulum aminopeptidases ERAP1 and/or ERAP2, and/or one or more immunoproteasome components selected from the standard proteasome catalytic subunits β1, β2, and/or β5, and the inducible proteasome catalytic subunits LMP2, MECL-1, and/or LMP7.

In several embodiments, there is provided a method of manufacturing a population of genetically engineered immune cells comprising (a) contacting a population of immune cells with an RNA-guided endonuclease targeting TAPBP or TAP-2, wherein the immune cells comprise T cells and the edit decreases the frequency of cell surface expression of beta-2-microglobulin (B2M) within the population of genetically engineered immune cells, (b) contacting the population of immune cells with a polynucleotide encoding a chimeric receptor comprising an extracellular ligand binding domain, a transmembrane domain, and a cytotoxic signaling complex, and (c) contacting the population of immune cells with a polynucleotide encoding at least one immunosuppressive effector that exerts suppressive effects on the cytotoxic activity of suppressive cells.

Methods are also provided for enhancing the in vivo persistence of genetically engineered immune cells comprising (a) contacting a population of immune cells with an RNA-guided endonuclease targeting TAPBP or TAP-2, wherein the immune cells comprise T cells and the edit decreases the frequency of cell surface expression of beta-2-microglobulin (B2M) within the population of genetically engineered immune cells, (b) contacting the population of immune cells with a polynucleotide encoding a chimeric receptor comprising an extracellular ligand binding domain, a transmembrane domain, and a cytotoxic signaling complex, and (c) contacting the population of immune cells with a polynucleotide encoding at least one immunosuppressive effector that exerts suppressive effects on the cytotoxic activity of suppressive cells. In several embodiments, the immunosuppressive effector is encoded by a nucleic acid or comprises a peptide having at least 85% sequence identity to one or more of the nucleotide or amino acid sequences of SEQ ID NOs: 683-894, 997-1000, 1014-1017, 1020-1023, 1027, 1029, 1031, 1033, 1035, 1037, 1039, 1041, 1046, 1048, 1050, 1052, 1054, 1056, or 1058-1093. In several embodiments, the genetically engineered immune cells exhibit one or more of enhanced expansion capability, enhanced cytotoxicity against target cells, and enhanced persistence, as compared to cells that have not been contacted with a polynucleotide encoding at least one immunosuppressive effector. In several embodiments, the genetic edit is to TAPBP. In several embodiments, the genetic edit is to TAP-2. In several embodiments, a plurality of the genetically engineered immune cells comprises an additional genetic edit to a gene encoding one or more of CISH, CBLB, B2M, CD70, adenosine receptor gene, NKG2A, CIITA, TGFBR, and any combination thereof.

Also provided for herein is a population of genetically engineered immune cells comprising genetically engineered immune cells that express a chimeric receptor comprising an extracellular ligand binding domain, a transmembrane domain, and a cytotoxic signaling complex, wherein the immune cells are genetically edited at one or more target sites in the genome of the immune cell to yield reduced levels of expression of a protein which is encoded by a gene which comprises an edited target site as compared to a non-edited immune cell, the genetic edit results in a decrease in the frequency of cell surface expression of major histocompatibility complex class I (MHC I) molecules, and the genetically engineered immune cells are further engineered to express at least one immunosuppressive effector that exerts suppressive effects on the cytotoxic activity of natural killer cells and/or T cells. In several embodiments, the the genetically engineered immune cells comprise one or both of genetically engineered natural killer (NK) cells and genetically engineered T cells.

Also provided for herein is a population of genetically engineered immune cells one or both of genetically engineered natural killer (NK) cells and genetically engineered T cells wherein a plurality of the genetically engineered immune cells are engineered to express a chimeric receptor comprising an extracellular ligand binding domain, a transmembrane domain, and a cytotoxic signaling complex, the immune cells are genetically edited at one or more target sites in the genome of the immune cell to yield reduced levels of expression of a protein which is encoded by a gene which comprises an edited target site as compared to a non-edited immune cell, the edits result in a decrease in the frequency of cell surface expression of MHC I molecules within the population of genetically engineered immune cells, and the genetically engineered immune cells are further genetically engineered to express at least one immunosuppressive effector that exerts suppressive effects on the cytotoxic activity of one or more of non-engineered natural killer cells, non-engineered T cells, and engineered cells.

Also provided is a population of genetically engineered immune cells, comprising one or both of genetically engineered natural killer (NK) cells and genetically engineered T cells wherein a plurality of the genetically engineered immune cells are engineered to express a chimeric receptor comprising an extracellular ligand binding domain, a transmembrane domain, and a cytotoxic signaling complex, the immune cells are genetically edited at one or more target sites in the genome of the immune cell to yield reduced levels of expression of a protein which is encoded by a gene which comprises an edited target site as compared to a non-edited immune cell, the edits result in a decrease in the frequency of cell surface expression of MHC I molecules within the population of genetically engineered immune cells, and the genetically engineered immune cells are further genetically engineered to express at least one immunosuppressive effector that exerts suppressive effects on cytotoxic activity of suppressive cells.

In several embodiments, the population of genetically engineered immune cells exhibits one or more of enhanced expansion capability, enhanced cytotoxicity against target cells, and enhanced persistence, as compared to immune cells that do not express the immunosuppressive effector and do not comprise the edited target site. In several embodiments, the population of genetically engineered immune cells comprises genetically engineered NK cells. In several embodiments, the population of genetically engineered immune cells comprises genetically engineered T cells. In several embodiments, the genetic edit is made to a gene encoding one or more of: TAPBP (Tapasin), TAP-2; UGT-1 (UGTA-1), TAPBPL (TAPBPR), TAP-1, ERp57; Calreticulin (CRT); Endoplasmic reticulum aminopeptidases ERAP1 and/or ERAP2, and/or one or more immunoproteasome components selected from the standard proteasome catalytic subunits β1, β2, and/or β5, and the inducible proteasome catalytic subunits LMP2, MECL-1, and LMP7.

Additionally, in several embodiments, there is provided a population of genetically engineered immune cells for cancer immunotherapy, comprising genetically engineered immune cells that express a cytotoxic receptor comprising an extracellular ligand binding domain, a transmembrane domain, and a cytotoxic signaling complex, wherein the immune cells are genetically edited at one or more target sites in the genome of the immune cell to yield reduced levels of expression of a protein which is encoded by a gene which comprises an edited target site as compared to a non-edited immune cell, wherein the edits result in a decrease in the frequency of cell surface expression of major histocompatibility complex class I (MHC I) molecules within the population of genetically engineered immune cells, and wherein the genetically engineered immune cells are further engineered to express at least one immunosuppressive effector, wherein the at least one immunosuppressive effector exerts suppressive effects on the cytotoxic activity of natural killer cells and/or T cells, and wherein the genetically engineered and edited immune cells exhibit one or more of enhanced expansion capability, enhanced cytotoxicity against target cells, and enhanced persistence, as compared to cells that do not comprise said immunosuppressive effector and said edited gene.

In several embodiments, there is provided a population of genetically engineered immune cells for cancer immunotherapy, comprising genetically engineered immune cells that express a cytotoxic receptor comprising an extracellular ligand binding domain, a transmembrane domain, and a cytotoxic signaling complex, wherein the immune cells are genetically edited at one or more target sites in the genome of the immune cell to yield reduced levels of expression of a protein which is encoded by a gene which comprises an edited target site as compared to a non-edited immune cell, and wherein the edits result in a decrease in the frequency of cell surface expression of MHC I molecules within the population of genetically engineered immune cells, and wherein the genetically engineered and edited immune cells exhibit one or more of enhanced expansion capability, enhanced cytotoxicity against target cells, and enhanced persistence, as compared to cells that do not comprise said edited gene.

Also provided herein is a T cell expressing a cytotoxic receptor (e.g., a chimeric antigen receptor) and comprising a genetic edit to a gene encoding antigen peptide transporter 1 (TAP-1), antigen peptide transporter 2 (TAP-2), or TAP binding protein (TAPBP).

Also provided herein is a combination of a natural killer (NK) cell and a T cell, wherein the T cell expresses a cytotoxic receptor (e.g., a chimeric antigen receptor) and comprises a genetic edit to a gene encoding TAP-1, TAP-2, or TAPBP. Also provided herein is a composition comprising a combination of NK and T cells as described herein. Also provided herein is a composition comprising a natural killer (NK) cell and a T cell, wherein the T cell expresses a cytotoxic receptor (e.g., a chimeric antigen receptor) and comprises a genetic edit to a gene encoding TAP-1, TAP-2, or TAPBP. In some embodiments, the composition comprises a pharmaceutically acceptable excipient.

In some embodiments, the NK cell expresses a cytotoxic receptor (e.g., a chimeric antigen receptor). In some embodiments, the T cell comprises a genetic edit to a gene encoding TAP-1. In some embodiments, the T cell comprises a genetic edit to the TAP1 gene. In some embodiments, the T cell comprises a genetic edit to a gene encoding TAP-2. In some embodiments, the T cell comprises a genetic edit to the TAP2 gene. In some embodiments, the T cell comprises a genetic edit to a gene encoding TAPBP. In some embodiments, the T cell comprises a genetic edit to the TAPBP gene. In some embodiments, the genetic edit reduces expression (e.g., transcription) of the gene. In some embodiments, the genetic edit eliminates (e.g., knocks out) expression of the gene.

In some embodiments, the T cell expresses a chimeric antigen receptor (CAR). In some embodiments, the NK cell expresses a chimeric antigen receptor (CAR). In some embodiments, the T cell and the NK cell express the same CAR. In some embodiments, the T cell and the NK cell express different CARs. In some embodiments, the CAR expressed by the T cell and the CAR expressed by the NK cell target the same antigen (e.g., a tumor antigen). In some embodiments, the CAR expressed by the T cell and the CAR expressed by the NK cell target different antigens (e.g., tumor antigens).

Also provided herein is use of a T cell, a combination, or a composition as described herein for use in treating a disease or disorder. In some embodiments, the disease or disorder is a cancer.

Additionally, in several embodiments, there is provided a population of genetically engineered immune cells for cancer immunotherapy, comprising a subpopulation of genetically engineered NK cells and a subpopulation of genetically engineered T cells, wherein each of the subpopulations comprise an engineered cytotoxic receptor, wherein each of the subpopulations comprises a genetic edit to a gene involved in antigen processing and/or MHC I complex assembly, resulting in a decrease in the frequency of MHC I molecules within each subpopulation, resulting in a decrease in the frequency of MHC I molecules within each subpopulation, and wherein the genetically engineered and edited immune cells exhibit one or more of enhanced expansion capability, enhanced cytotoxicity against target cells, and enhanced persistence, as compared to cells that do not comprise said edited genetic edits. In several embodiments, one or both of the subpopulations comprises a genetic edit to a gene encoding TAP-2 and/or TAPBP. In several embodiments, the genetically engineered NK cells comprise a genetic edit to a gene encoding TAP-2. In several embodiments, the genetically engineered NK cells comprise a genetic edit to a gene encoding TAPBP. In several embodiments, the genetically engineered T cells comprise a genetic edit to a gene encoding TAP-2. In several embodiments, the genetically engineered T cells comprise a genetic edit to a gene encoding TAPBP.

In several embodiments, the edits are made using an RNA-guided endonuclease. In some embodiments, the edits are made using a Crispr/Cas system. In several embodiments, the genetic edit is to a gene involved in antigen processing and/or MHC I complex assembly. In several embodiments, the genetic edit is to a gene involved in antigen processing. In several embodiments, the genetic edit is to a gene involved in MHC I complex assembly. In several embodiments, the genetic edit is to a gene involved in antigen processing and MHC I complex assembly.

In several embodiments, the suppressive cells comprise host cells. In several embodiments, the suppressive cells comprise one or more of non-engineered natural killer cells, non-engineered T cells, or suppressive engineered cells. In several embodiments, the suppressive cells comprise non-engineered natural killer cells. In several embodiments, the suppressive cells comprise non-engineered T cells. In several embodiments, the suppressive cells comprise suppressive engineered cells. In several embodiments, suppressive engineered cells comprise the genetically engineered immune cells. In several embodiments, the cells that do not comprise said immunosuppressive effector are either non-engineered or engineered cells.

In several embodiments, the genetic edit (or edits) is to one or more of UGT-1 (UGTA-1), TAPBPL (TAPBPR), TAPBP (Tapasin), TAP-1, TAP-2; ERp57; Calreticulin (CRT); Endoplasmic reticulum aminopeptidases ERAP1 and/or ERAP2, and/or one or more immunoproteasome components selected from the standard proteasome catalytic subunits β1, β2, and/or β5, and the inducible proteasome catalytic subunits LMP2, MECL-1, and/or LMP7. In several embodiments, the genetic edit is to UGT-1 (UGTA-1). In several embodiments, the genetic edit is to TAPBPL (TAPBPR). In several embodiments, the genetic edit is to TAPBP (Tapasin). In several embodiments, the genetic edit is to TAP-1. In several embodiments, the genetic edit is to TAP-2. In several embodiments, the genetic edit is to ERp57. In several embodiments, the genetic edit is to Calreticulin (CRT). In several embodiments, the genetic edit is to Endoplasmic reticulum aminopeptidases ERAP1 and/or ERAP2. In several embodiments, the genetic edit is to an immunoproteasome component selected from the group consisting of standard proteasome catalytic subunits β1, β2, and β5, and the inducible proteasome catalytic subunits LMP2, MECL-1, and LMP7.

In some embodiments, the genetic edit is to TAPBP and/or TAP2 and reduced expression of TAPBP and/or TAP2 enables the immune cells to be used in allogeneic cancer immunotherapy with reduced host versus graft rejection as compared to immune cells expressing endogenous levels of TAPBP and/or TAP2. In some embodiments, the genetic edit to TAPBP reduces expression (e.g., protein expression) of TAPBP. In some embodiments, the genetic edit to TAP2 reduces expression (e.g., protein expression) of TAP2. In some embodiments, the genetic edit reduces host versus graft rejection as compared to the absence of the genetic edit.

In several embodiments, the cytotoxic receptor targets one or more of NKG2D ligands, CD19, BCMA, CD70, and CD38 expressed by target tumor cells. In several embodiments, the cytotoxic receptor binds to a NKG2D ligand. In several embodiments, the cytotoxic receptor binds to CD19. In several embodiments, the cytotoxic receptor binds to BCMA. In several embodiments, the cytotoxic receptor binds to CD70. In several embodiments, the cytotoxic receptor binds to CD38. In several embodiments, the cytotoxic signaling complex comprises an OX40 subdomain or a 4-1BB domain, and a CD3zeta subdomain. In several embodiments, the cytotoxic signaling complex comprises an OX40 domain. In several embodiments, the cytotoxic signaling complex comprises 4-1BB domain. In several embodiments, the cytotoxic signaling complex comprises CD3zeta domain. In some embodiments, the cytotoxic signaling complex comprises an OX40 domain and a CD3zeta domain. In some embodiments, the cytotoxic signaling complex comprises a 4-1BB domain and a CD3zeta domain.

In several embodiments, the at least one immunosuppressive effector, when present, comprises a virally-derived peptide. In some embodiments, the least one immunosuppressive effector comprises a peptide derived from a retrovirus or other type of virus. In some embodiments, the least one immunosuppressive effector comprises a peptide derived from a retrovirus. In several embodiments, the at least one immunosuppressive effector comprises a peptide derived from an envelope protein of a retrovirus.

Additionally in some embodiments, the at least one immunosuppressive effector comprises at least a portion of a human protein and/or at least a portion of a human protein complex or at least a portion of human protein. In some embodiments, the at least one immunosuppressive effector comprises a human protein or portion thereof. In several embodiments, the at least one immunosuppressive effector comprises a chimeric construct comprising at least one virally-derived peptide and at least a portion of a human protein and/or at least a portion of a human protein complex.

In some embodiments, the cytotoxic receptor comprises an immunosuppressive effector. In several embodiments, the at least one immunosuppressive effector is integrated into the cytotoxic receptor. In several embodiments, the cytotoxic receptor comprises an immunosuppressive effector between the transmembrane domain and the extracellular ligand-binding domain. In several embodiments, the at least one immunosuppressive effector is integrated into the cytotoxic receptor between the transmembrane domain and the extracellular ligand-binding domain. In several embodiments, the at least one immunosuppressive effector is integrated into the cytotoxic receptor within the extracellular ligand-binding domain. In several embodiments, the cytotoxic receptor comprises an immunosuppressive effector within the extracellular ligand-binding domain. In several embodiments, the extracellular ligand-binding domain comprises an scFv. In some embodiments, the scFv comprises a heavy chain variable region (VH), a light chain variable region (VL). In some embodiments, the scFv comprises a linker between the VH and the VL. In some embodiments, the cytotoxic receptor comprises an immunosuppressive effector within the linker of the scFv. In several embodiments, the extracellular ligand-binding domain comprises an scFv and the at least one immunosuppressive effector is integrated into a linker region of the scFv. In several embodiments, the at least one immunosuppressive effector is integrated into the cytotoxic receptor within an N-terminal region of the cytotoxic receptor distally positioned from the extracellular ligand-binding domain. In several embodiments, the distal position is in relation to the membrane of the cell. In several embodiments, the at least one immunosuppressive effector is integrated into the cytotoxic receptor at a plurality of locations within an extracellular region of the cytotoxic receptor.

In several embodiments, the at least one immunosuppressive effector is bound to an extracellular membrane of the immune cells. In several embodiments, the at least one immunosuppressive effector comprises a transmembrane protein. Depending on the embodiment, the transmembrane protein is selected from CD8α, CD4, CD3ε, CD3γ, CD3δ, CD3ζ, CD28, CD137, glycophorin A, glycophorin D, nicotinic acetylcholine receptor, a GABA receptor, FcεRIγ, and a T-cell receptor. In several embodiments, the transmembrane protein comprises a CD8α transmembrane protein. In several embodiments, the at least one immunosuppressive effector is expressed on the immune cells by a disulfide trap single chain trimer (dtSCT).

In several embodiments, the immunosuppressive effector is encoded by a nucleic acid or comprises a peptide having at least 85% sequence identity to one or more of the nucleotide or amino acid sequences of SEQ ID NOs: 683-894 or 997-1000.

In several embodiments, the immunosuppressive effector comprises a peptide having at least 95% sequence identity to SEQ ID NO: 829, 997, 999, or 689. In several embodiments, the immunosuppressive effector comprises a peptide having at least 95% sequence identity to SEQ ID NO: 829. In several embodiments, the immunosuppressive effector comprises the amino acid sequence set forth in SEQ ID NO: 829. In several embodiments, the immunosuppressive effector comprises a peptide having at least 95% sequence identity to SEQ ID NO: 997. In several embodiments, the immunosuppressive effector comprises the amino acid sequence set forth in SEQ ID NO: 997. In several embodiments, the immunosuppressive effector comprises a peptide having at least 95% sequence identity to SEQ ID NO: 999. In several embodiments, the immunosuppressive effector comprises the amino acid sequence set forth in SEQ ID NO: 999. In several embodiments, the immunosuppressive effector comprises a peptide having at least 95% sequence identity to SEQ ID NO: 689. In several embodiments, the immunosuppressive effector comprises the amino acid sequence set forth in SEQ ID NO: 689.

In several embodiments, the immunosuppressive effector comprises a peptide having at least 95% sequence identity to SEQ ID NO: 1014, 1015, 1016, or 1017. In several embodiments, the immunosuppressive effector comprises a peptide having at least 95% sequence identity to SEQ ID NO: 1014. In several embodiments, the immunosuppressive effector comprises the amino acid sequence set forth in SEQ ID NO: 1014. In several embodiments, the immunosuppressive effector comprises a peptide having at least 95% sequence identity to SEQ ID NO: 1015. In several embodiments, the immunosuppressive effector comprises the amino acid sequence set forth in SEQ ID NO: 1015. In several embodiments, the immunosuppressive effector comprises a peptide having at least 95% sequence identity to SEQ ID NO: 1016. In several embodiments, the immunosuppressive effector comprises the amino acid sequence set forth in SEQ ID NO: 1016. In several embodiments, the immunosuppressive effector comprises a peptide having at least 95% sequence identity to SEQ ID NO: 1017. In several embodiments, the immunosuppressive effector comprises the amino acid sequence set forth in SEQ ID NO:1017.

In several embodiments, the immunosuppressive effector is encoded by a nucleic acid having at least 95% sequence identity to SEQ ID NO: 830, 998, 1000, or 690. In several embodiments, the immunosuppressive effector is encoded by a nucleic acid having at least 95% sequence identity to SEQ ID NO: 830. In several embodiments, the immunosuppressive effector is encoded by a nucleic acid having at least 95% sequence identity to SEQ ID NO: 998. In several embodiments, the immunosuppressive effector is encoded by a nucleic acid having at least 95% sequence identity to SEQ ID NO: 1000. In several embodiments, the immunosuppressive effector is encoded by a nucleic acid having at least 95% sequence identity to SEQ ID NO: 690.

In several embodiments, the genetically engineered immune cells comprise genetically engineered Natural Killer (NK) cells, genetically engineered T cells, or combinations thereof. In several embodiments, the genetically engineered immune cells comprise genetically engineered Natural Killer (NK) cells. In several embodiments, the genetically engineered immune cells comprise genetically engineered T cells. In several embodiments, the genetically engineered immune cells comprise genetically engineered Natural Killer (NK) cells and genetically engineered T cells. Other immune cells as disclosed herein may also be used, including, for example, iPSC-derived cells. In several embodiments, the genetically engineered immune cells are suitable for use in allogeneic cancer cell therapy with reduced risk of graft versus host disease. Advantageously, in several embodiments, the genetically engineered immune cells are suitable for use in allogeneic cancer cell therapy with reduced risk of cytotoxic activity between the genetically engineered immune cells. Optionally, in several embodiments, at least a portion of the genetically engineered immune cells are engineered to express membrane bound IL-15. In some embodiments, at least a portion of the genetically engineered NK cells are engineered to express membrane bound IL-15. In some embodiments, at least a portion of the genetically engineered T cells are engineered to express membrane bound IL-15.

Also provided for herein are methods for the treatment of cancer in a subject, comprising administering to the subject genetically engineered immune cells according to the present disclosure. Further provided is the use of genetically engineered immune cells according to the present disclosure for the treatment of cancer and/or for the preparation of a medicament for the treatment of cancer. In some embodiments, the genetically engineered cells are for use in the treatment of cancer. In some embodiments, the genetically engineered immune cells are for use in the preparation of a medicament for the treatment of cancer.

Further provided for herein is a method of manufacturing a population of genetically engineered immune cells for cancer immunotherapy, comprising contacting a population of immune cells with an RNA-guided endonuclease to genetically edit one or more target sites in the genome of the immune cell to yield reduced levels of expression of a protein which is encoded by a gene which comprises an edited target site as compared to a non-edited immune cell, wherein the edit results in a decrease in the frequency of cell surface expression of major MHC I molecules within the population of genetically engineered immune cells, contacting the population of immune cells with a polynucleotide encoding a cytotoxic receptor comprising an extracellular ligand binding domain, a transmembrane domain, and a cytotoxic signaling complex; and optionally contacting the population of immune cells with an additional polynucleotide encoding at least one immunosuppressive effector, wherein the immunosuppressive effector is encoded by a nucleic acid or comprises a peptide having at least 85% sequence identity to one or more of the nucleotide or amino acid sequences of SEQ ID NOs: 683-894 or 997-1000, wherein the at least one immunosuppressive effector exerts suppressive effects on the cytotoxic activity of suppressive cells, and wherein the genetically engineered immune cells exhibit one or more of enhanced expansion capability, enhanced cytotoxicity against target cells, and enhanced persistence, as compared to cells that do not comprise said at least one immunosuppressive effector.

In several embodiments, the genetic edit is to one or more of UGT-1 (UGTA-1), TAPBPL (TAPBPR), TAPBP (Tapasin), TAP-1, TAP-2; ERp57; Calreticulin (CRT); Endoplasmic reticulum aminopeptidases ERAP1 and/or ERAP2, and/or one or more immunoproteasome components selected from the standard proteasome catalytic subunits β1, β2, and/or β5, and the inducible proteasome catalytic subunits LMP2, MECL-1, and/or LMP7.

In several embodiments, the genetic edit comprises an edit to UGT-1 (UGTA-1). In several embodiments, the genetic edit comprises an edit to TAPBPL (TAPBPR). In several embodiments, the genetic edit comprises an edit to TAPBP (Tapasin). In several embodiments, the genetic edit comprises an edit to TAP-1. In several embodiments, the genetic edit comprises an edit to TAP-2. In several embodiments, the genetic edit comprises an edit to ERp57. In several embodiments, the genetic edit comprises an edit to Calreticulin (CRT). In several embodiments, the genetic edit comprises an edit to Endoplasmic reticulum aminopeptidases ERAP1. In several embodiments, the genetic edit comprises an edit to ERAP2. In several embodiments, the genetic edit comprises an edit to an immunoproteasome component selected from the group consisting of standard proteasome catalytic subunits β1, β2, and β5, and the inducible proteasome catalytic subunits LMP2, MECL-1, and LMP7.

In several embodiments, an additional genetic edit is made to one or more of a CISH gene, a CBLB gene, a B2M gene, a CD70 gene, an adenosine receptor gene, an NKG2A gene, a CIITA gene, a TGFBR gene, or any combination thereof. In several embodiments, an additional genetic edit comprises an edit to a gene encoding CISH. In several embodiments, an additional genetic edit comprises an edit to a gene encoding CBLB. In several embodiments, an additional genetic edit comprises an edit to a gene encoding B2M. In several embodiments, an additional genetic edit comprises an edit to a gene encoding CD70. In several embodiments, an additional genetic edit comprises an edit to a gene encoding an adenosine receptor gene. In several embodiments, an additional genetic edit comprises an edit to a gene encoding NKG2A. In several embodiments, an additional genetic edit comprises an edit to a gene encoding CIITA. In several embodiments, an additional genetic edit comprises an edit to a gene encoding TGFBR.

In some embodiments, the genetic edit is to TAPBP and/or TAP2 and reduced expression of TAPBP and/or TAP2 enables the immune cells to be used in allogeneic cancer immunotherapy with reduced host versus graft rejection as compared to immune cells expressing endogenous levels of TAPBP and/or TAP2. In some embodiments, the genetic edit to TAPBP reduces expression (e.g., protein expression) of TAPBP. In some embodiments, the genetic edit to TAP2 reduces expression (e.g., protein expression) of TAP2. In some embodiments, the genetic edit reduces host versus graft rejection as compared to the absence of the genetic edit.

In several embodiments, the immunosuppressive effector is encoded by a nucleic acid or comprises a peptide having at least 85% sequence identity to one or more of the nucleotide or amino acid sequences of SEQ ID NOs: 683-894, or 997-1000 wherein the immunosuppressive effector comprises at least a portion of an HLA-E molecule, and/or wherein the immunosuppressive effector comprises at least a portion of CD47. In several embodiments, the immunosuppressive effector is encoded by a nucleic acid or comprises a peptide having at least 85% sequence identity to sequence set forth in any one of SEQ ID NOs: 683-894 and 997-1000. In several embodiments, the immunosuppressive effector comprises HLA-E or a portion thereof. In several embodiments, the immunosuppressive effector comprises CD47 or a portion thereof.

In several embodiments, HLA can be re-expressed, for example by expressing HLA-E and/or HLA-G. In several embodiments, the re-expression of the HLA is accomplished using a disulfide trap single chain trimer (dtSCT) to express HLA-E and/or HLA-G and, optionally, an immunosuppressive peptide, as well as B2M. In several embodiments, the re-expression of the HLA is accomplished using a disulfide trap single chain trimer (dtSCT) to express HLA-E. In several embodiments, the re-expression of the HLA is accomplished using a disulfide trap single chain trimer (dtSCT) to express HLA-G. In some embodiments, the re-expression of the HLA is accomplished using a disulfide trap single chain trimer (dtSCT) to express an immunosuppressive peptide. In some embodiments, the re-expression of the HLA is accomplished using a disulfide trap single chain trimer (dtSCT) to express B2M.

Provided for herein, in several embodiments, is a polynucleotide encoding a chimeric immunosuppressive construct comprising an HLA-G peptide, mature B2M and mature HLA-E. In several embodiments, such a construct comprises one or more linkers. In several embodiments, the immunosuppressive construct comprises a B2M signal peptide (at least about 85%, at least about 90%, at least about 94%, at least about 95%, at least about 96%, at least about 97%, at least about 98%, or at least about 99%, identical to SEQ ID NO: 1003) In several embodiments, the immunosuppressive construct comprises a B2M signal peptide (at least about 85%, at least about 90%, at least about 94%, at least about 95%, at least about 96%, at least about 97%, at least about 98%, or at least about 99%, identical to SEQ ID NO: 1018), an HLA-G peptide (amino acids 3-11 of HLA-G; at least about 85%, at least about 90%, at least about 94%, at least about 95%, at least about 96%, at least about 97%, at least about 98%, or at least about 99%, identical to SEQ ID NO: 1005), a disulfide-bridge containing linker (e.g., a GS linker comprising at least two cysteine residues; at least about 85%, at least about 90%, at least about 94%, at least about 95%, at least about 96%, at least about 97%, at least about 98%, or at least about 99%, identical to SEQ ID NO: 1009 or 1007), a mature B2M domain (at least about 85%, at least about 90%, at least about 94%, at least about 95%, at least about 96%, at least about 97%, at least about 98%, or at least about 99%, identical to SEQ ID NO: 1011), an additional linker (e.g., a GS linker; at least about 85%, at least about 90%, at least about 94%, at least about 95%, at least about 96%, at least about 97%, at least about 98%, or at least about 99%, identical to SEQ ID NO: 1009 or 1007), and a mature HLA-E domain (at least about 85%, at least about 90%, at least about 94%, at least about 95%, at least about 96%, at least about 97%, at least about 98%, or at least about 99%, identical to SEQ ID NO: 1013).

In several embodiments, the HLA re-expressing construct is encoded by a polynucleotide that encodes an amino acid sequence comprising SEQ ID NO: 829, 997, or 999. In several embodiments, the amino acid sequence shares at least about 90%, at least about 94%, at least about 95%, at least about 96%, at least about 97%, at least about 98%, or at least about 99%, sequence identity, homology and/or functional equivalence with SEQ ID NO: 829, 997, or 999. In several embodiments, the HLA re-expressing construct comprises the amino acid sequence set forth in SEQ ID NO: 829, 997, or 999. In several embodiments, the HLA re-expressing construct comprises the amino acid sequence set forth in SEQ ID NO: 829. In several embodiments, the HLA re-expressing construct comprises the amino acid sequence set forth in SEQ ID NO: 997. In several embodiments, the HLA re-expressing construct comprises the amino acid sequence set forth in SEQ ID NO: 999.

In several embodiments, the HLA re-expressing construct is encoded by a polynucleotide that encodes an amino acid sequence comprising SEQ ID NO: 1014, 1015, or 1016. In several embodiments, the amino acid sequence shares at least about 90%, at least about 94%, at least about 95%, at least about 96%, at least about 97%, at least about 98%, or at least about 99%, sequence identity, homology and/or functional equivalence with SEQ ID NO: 1014, 1015, or 1016. In several embodiments, the HLA re-expressing construct comprises the amino acid sequence set forth in SEQ ID NO: 1014, 1015, or 1016. In several embodiments, the HLA re-expressing construct comprises the amino acid sequence set forth in SEQ ID NO: 1014. In several embodiments, the HLA re-expressing construct comprises the amino acid sequence set forth in SEQ ID NO: 1015. In several embodiments, the HLA re-expressing construct comprises the amino acid sequence set forth in SEQ ID NO: 1016.

In several embodiments, the B2M signal peptide is encoded by a nucleic acid that is at least about 85%, at least about 90%, at least about 94%, at least about 95%, at least about 96%, at least about 97%, at least about 98%, or at least about 99%, identical to SEQ ID NO: 1001 or 1002. In some embodiments, the B2M signal peptide comprises the amino acid sequence set forth in SEQ ID NO: 1003. In some embodiments, the B2M signal peptide comprises the amino acid sequence set forth in SEQ ID NO: 1018.

In several embodiments, the HLA-G peptide is encoded by a nucleic acid that is at least about 85%, at least about 90%, at least about 94%, at least about 95%, at least about 96%, at least about 97%, at least about 98%, or at least about 99%, identical to SEQ ID NO: 1004. In some embodiments, the HLA-G peptide comprises the amino acid sequence set forth in SEQ ID NO: 1005.

In several embodiments, the disulfide-bridge containing linker is encoded by a nucleic acid that is at least about 85%, at least about 90%, at least about 94%, at least about 95%, at least about 96%, at least about 97%, at least about 98%, or at least about 99%, identical to SEQ ID NO: 1008 or 1006. In several embodiments, the disulfide-bridge continaing linker comprises the amino acid sequence set forth in SEQ ID NO: 1007 or 1009. In several embodiments, the disulfide-bridge continaing linker comprises the amino acid sequence set forth in SEQ ID NO: 1007. In several embodiments, the disulfide-bridge continaing linker comprises the amino acid sequence set forth in SEQ ID NO: 1009.

In several embodiments, the mature B2M is encoded by a nucleic acid that is at least about 85%, at least about 90%, at least about 94%, at least about 95%, at least about 96%, at least about 97%, at least about 98%, or at least about 99%, identical to SEQ ID NO: 1010. In several embodiments, the mature B2M comprises the amino acid sequence set forth in SEQ ID NO: 1011.

In several embodiments, an additional copy of the disulfide-bridge containing linker is use after the B2M and links the B2M with a mature HLA-E domain.

In several embodiments, the mature HLA-E domain is encoded by a nucleic acid that is at least about 85%, at least about 90%, at least about 94%, at least about 95%, at least about 96%, at least about 97%, at least about 98%, or at least about 99%, identical to SEQ ID NO: 1012. In several embodiments, the mature HLA-E domain comprises the amino acid sequence set forth in SEQ ID NO:1013.

Also provided for herein is a method of manufacturing a population of genetically engineered immune cells for cancer immunotherapy, comprising contacting a population of immune cells with an RNA-guided endonuclease to genetically edit one or more target sites in the genome of the immune cell to yield reduced levels of expression of a protein which is encoded by a gene which comprises an edited target site as compared to a non-edited immune cell, wherein the edit results in a decrease in the frequency of cell surface expression of MHC I molecules within the population of genetically engineered immune cells, contacting the population of immune cells with an additional polynucleotide encoding at least one immunosuppressive effector, wherein the immunosuppressive effector is encoded by a nucleic acid or comprises a peptide having at least 85% sequence identity to one or more of the nucleotide or amino acid sequences of SEQ ID NOs: 683-894 or 997-1000, wherein the at least one immunosuppressive effector exerts suppressive effects on the cytotoxic activity of suppressive cells, and wherein the genetically engineered immune cells exhibit one or more of enhanced expansion capability, enhanced cytotoxicity against target cells, and enhanced persistence, as compared to cells that do not comprise said at least one immunosuppressive effector.

Provided for herein is also a method of enhancing the persistence of immune cells for use in allogeneic therapy, comprising contacting a population of immune cells with an RNA-guided endonuclease to genetically edit one or more target sites in the genome of the immune cell to yield reduced levels of expression of a protein which is encoded by a gene which comprises an edited target site as compared to a non-edited immune cell, wherein the edit results in a decrease in the frequency of cell surface expression of MHC I molecules within the population of genetically engineered immune cells, and wherein the genetically edited immune cells are less likely to trigger cytotoxic effects from one or more of host NK or T cells, thereby exhibiting enhanced persistence, as compared to cells that do not comprise said gene edit.

An additional method of enhancing the persistence of immune cells for use in allogeneic therapy is provided and comprises contacting a population of immune cells with an RNA-guided endonuclease to genetically edit one or more target sites in the genome of the immune cell to yield reduced levels of expression of a protein which is encoded by a gene which comprises an edited target site as compared to a non-edited immune cell, wherein the edit is to a gene involved in antigen processing and/or MHC I complex assembly, and wherein the genetically edited immune cells are less likely to trigger cytotoxic effects from one or more of host NK or T cells, thereby exhibiting enhanced persistence, as compared to cells that do not comprise said gene edit.

In several embodiments, the genetic edit is to one or more of UGT-1 (UGTA-1), TAPBPL (TAPBPR), TAPBP (Tapasin), TAP-1, TAP-2; ERp57; Calreticulin (CRT); Endoplasmic reticulum aminopeptidases ERAP1 and/or ERAP2, and/or one or more immunoproteasome components selected from the standard proteasome catalytic subunits β1, β2, and/or β5, and the inducible proteasome catalytic subunits LMP2, MECL-1, and/or LMP7. In several embodiments, the genetic edit comprises an edit to UGT-1 (UGTA-1). In several embodiments, the genetic edit comprises an edit to TAPBPL (TAPBPR). In several embodiments, the genetic edit comprises an edit to TAPBP (Tapasin). In several embodiments, the genetic edit comprises an edit to TAP-1. In several embodiments, the genetic edit comprises an edit to TAP-2. In several embodiments, the genetic edit comprises an edit to ERp57. In several embodiments, the genetic edit comprises an edit to Calreticulin (CRT). In several embodiments, the genetic edit comprises an edit to Endoplasmic reticulum aminopeptidases ERAP1. In several embodiments, the genetic edit comprises an edit to ERAP2. In several embodiments, the genetic edit comprises an edit to an immunoproteasome component selected from the group consisting of standard proteasome catalytic subunits β1, β2, and β5, and the inducible proteasome catalytic subunits LMP2, MECL-1, and LMP7. In several embodiments, an additional genetic edit is made to one or more of a CISH gene, a CBLB gene, a B2M gene, a CD70 gene, an adenosine receptor gene, an NKG2A gene, a CIITA gene, a TGFBR gene, or any combination thereof. In several embodiments, the genetic edit comprises an edit to CISH. In several embodiments, the genetic edit comprises an edit to CBLB. In several embodiments, the genetic edit comprises an edit to B2M. In several embodiments, the genetic edit comprises an edit to CD70. In several embodiments, the genetic edit comprises an edit to an adenosine receptor gene. In several embodiments, the genetic edit comprises an edit to NKG2A. In several embodiments, the genetic edit comprises an edit to CIITA. In several embodiments, the genetic edit comprises an edit to TGFBR.

In several embodiments, the methods further comprise contacting the population of immune cells with a polynucleotide encoding a cytotoxic receptor. In several embodiments, the methods further comprise contacting the population of immune cells with a polynucleotide encoding a cytotoxic receptor comprising an extracellular ligand binding domain, a transmembrane domain, and a cytotoxic signaling complex. Depending on the embodiment, the population of immune cells comprises NK cells, T cells and/or NK and T cells.

Some embodiments relate to a method comprising administering an immune cell as described herein to a subject in need. In some embodiments, the immune cells are allogeneic to the subject. In several embodiments, the immune cells are obtained from a subject without a cancer. In some embodiments, the subject has cancer. In some embodiments, the administration treats, inhibits, or prevents progression of the cancer.

### BRIEF DESCRIPTION OF THE DRAWINGS

FIG. 1 depicts non-limiting examples of tumor-directed chimeric antigen receptors.

FIG. 2 depicts additional non-limiting examples of tumor-directed chimeric antigen receptors.

FIG. 3 depicts additional non-limiting examples of tumor-directed chimeric antigen receptors.

FIG. 4 depicts additional non-limiting examples of tumor-directed chimeric antigen receptors.

FIG. 5 depicts additional non-limiting examples of tumor-directed chimeric antigen receptors.

FIG. 6 depicts non-limiting examples of tumor-directed chimeric antigen receptors directed against non-limiting examples of tumor markers.

FIG. 7 depicts additional non-limiting examples of tumor-directed chimeric antigen receptors directed against non-limiting examples of tumor markers.

FIGS. 8A-8I schematically depict various pathways that are altered through the gene editing techniques disclosed herein. FIG. 8A shows a schematic of the inhibitory effects of TGF-beta release by tumor cells in the tumor microenvironment. FIG. 8B shows a schematic of the CIS/CISH negative regulatory pathways on IL-15 function. FIG. 8C depicts a non-limiting schematic process flow for generation of engineered non-alloreactive T cells and engineered NK cells for use in a combination therapy according to several embodiments disclosed herein. FIG. 8D shows a schematic of the signaling pathways that can lead to graft vs. host disease. FIG. 8E shows a schematic of how several embodiments disclosed herein can reduce and/or eliminate graft vs. host disease. FIG. 8F shows a schematic of the signaling pathways that can lead to host vs. graft rejection. FIG. 8G shows a schematic of several embodiments disclosed herein that can reduce and/or eliminate host vs. graft rejection. FIG. 8H shows a schematic of how edited immune cells can act against other edited immune cells in mixed cell product. FIG. 8I shows a schematic of how several embodiments disclosed herein can reduce and/or eliminate host immune effects against edited immune cells.

FIGS. 9A-9D show schematic depictions of non-limiting modifications made to CARs according to embodiments disclosed herein. FIG. 9A shows a non-limiting embodiment in which a single immunosuppressive effector is integrated into the hinge domain of the CAR. FIG. 9B shows a non-limiting embodiment in which multiple domains are integrated into the CAR, with a first immunosuppressive effector being of a different type than a second immunosuppressive effector. In some embodiments, a plurality of immunosuppressive effectors is included (e.g., 2, 3, 4, 5 or more), with the immunosuppressive effectors optionally being the same, or different, from any other immunosuppressive effector in the CAR. FIG. 9C shows a non-limiting embodiment wherein the immunosuppressive effector is positioned the target binding region (e.g., on the linker between the heavy and light chains of an scFv). FIG. 9D shows a non-limiting embodiment wherein the immunosuppressive effector is positioned at the N-terminus of a chimeric antigen receptor.

FIGS. 10A-10J show schematic depictions of non-limiting modifications made to immune cells, which are optionally allogeneic immune cells. FIG. 10A shows an immune cell engineered to express an immunosuppressive effector in a membrane-bound format, based on being tethered to a transmembrane protein (or fragment thereof). FIG. 10B shows an immune cell engineered to express multiple immunosuppressive effectors in a membrane-bound format. FIG. 10C shows an immune cell engineered to express multiple immunosuppressive effectors in a membrane-bound format, with each of the two effectors differing from one another. FIG. 10D shows an immune cell engineered to express multiple immunosuppressive effectors tethered to a single transmembrane protein. FIG. 10E shows an immune cell engineered to express multiple immunosuppressive effectors tethered to a single transmembrane protein, wherein the domains differ from one another. FIG. 10F shows an immune cell engineered to express multiple immunosuppressive effectors in a membrane-bound format, with each of the two effectors tethered to one of the transmembrane proteins differing from one another. FIG. 10G shows an immune cell engineered to express a membrane-bound immunosuppressive effector, as well as a CAR comprising one or more immunosuppressive effectors (though in some embodiments the CAR does not include an immunosuppressive effector domain). FIG. 10H shows an immune cell engineered to express two immunosuppressive effectors in a membrane-bound format, as well as a CAR comprising one or more immunosuppressive effectors. FIG. 10I shows an immune cell engineered to express a membrane-bound immunosuppressive effector, a CAR comprising one or more immunosuppressive effectors, and one or more of HLA-E and/or HLA-G. In some embodiments like that schematically depicted, the engineered cells are NK cells that have been gene edited to disrupt B2M expression. FIG. 10J shows an immune cell engineered to express multiple immunosuppressive effectors, a CAR comprising at least one immunosuppressive effector, as well as HLA-E, HLA-G, CD47, PD-L1, and/or the Poliovirus Receptor (PVR). While all are depicted on a single cell, any combination of the immunosuppressive effectors, or any one alone, shown in the Figures or disclosed herein, may be used.

FIGS. 11A-11JJ depict schematics of non-limiting embodiments of immunosuppressive effector constructs as provided for herein.

FIG. 12 depicts a non-limiting schematic of an immune cell engineered to express a chimeric UL18-B2M construct.

FIGS. 13A-13B depict schematic diagrams related to constructs disclosed herein. FIG. 13A shows a schematic diagram of various scenarios in which immunosuppression edits or constructs are made and the resultant self versus non-self outcome. FIG. 13B shows a schematic of a disulfide trap single chain trimer (dtSCT) used to express various immune evasion peptides (modified from Hansen et al. Current Protocols in Immunology, 2009).

FIG. 14 shows a schematic of the pathways by which HLA-E acts on NK cells.

FIGS. 15A-15B show schematics of various approaches to reduce immune responses against engineered therapeutic cells. FIG. 15A depicts a schematic of a CAR comprising a hypoimmune domain (HYPO) and various peptides or HLA sequences that are used to reduce immune responses against engineered therapeutic cells. FIG. 15B depicts various peptides and combinations of peptides that are used to reduce immune responses against engineered therapeutic cells.

FIG. 16 shows a non-limiting schematic of an experimental timeline for evaluation of the genetic modifications to NK and/or T cells.

FIGS. 17A-17D show data related to the expression of MHC I expression on T cells genetically edited at one or more non-limiting examples of target genes. FIG. 17A shows flow cytometry data for expression of selected genes by T cells from a first donor at five days after gene disruption. FIG. 17B shows the data of FIG. 17A tabulated. FIG. 17C shows flow cytometry data for expression of selected genes by T cells from a second donor at seven days after gene disruption. FIG. 17D shows the data of FIG. 17C tabulated.

FIGS. 18A-18D show data related to the expression of MHC I expression on T cells genetically edited at one or more non-limiting examples of target genes. FIG. 18A shows flow cytometry data for expression of selected genes by T cells from a first donor at nine days after gene disruption. FIG. 18B shows the data of FIG. 18A tabulated. FIG. 18C shows flow cytometry data for expression of selected genes by T cells from a second donor at nine days after gene disruption. FIG. 18D shows the data of FIG. 18C tabulated.

FIGS. 19A-19B show data assessing the editing of the indicated target genes. FIG. 19A shows agarose gel electrophoresis of DNA amplified from electroporated (EP) control T cells as compared to T cells edited at the indicated target gene. FIG. 19B shows similar data for other examples of target genes.

FIGS. 20A-20C show data related to the reduced expression of selected target genes in a first donor's T cells, the resultant HLA expression levels, and the percentages of NK cells and T cells present after one day of co-culturing the edited T cells with NK cells expressing a chimeric antigen receptor. FIG. 20A shows flow cytometry data for expression of selected genes by T cells from a first donor after gene disruption. FIG. 20B shows the data of FIG. 20A tabulated. FIG. 20C shows histograms showing the percentage of T cells and NK cells present in a cultured population one day after co-culturing.

FIGS. 21A-21C show data related to the reduced expression of selected target genes in a second donor's T cells, the resultant HLA expression levels, and the percentages of NK cells and T cells present after one day of co-culturing the edited T cells with NK cells expressing a chimeric antigen receptor. FIG. 21A shows flow cytometry data for expression of selected genes by T cells from a second donor after gene disruption. FIG. 21B shows the data of FIG. 21A tabulated. FIG. 21C shows histograms showing the percentage of T cells and NK cells present in a cultured population one day after co-culturing.

FIGS. 22A-22C show data related to the reduced expression of selected target genes in a first donor's T cells, the resultant HLA expression levels, and the percentages of NK cells and T cells present after three days of co-culturing the edited T cells with NK cells expressing a chimeric antigen receptor. FIG. 22A shows flow cytometry data for expression of selected genes by T cells from a first donor after gene disruption. FIG. 22B shows the data of FIG. 22A tabulated. FIG. 22C shows histograms showing the percentage of T cells and NK cells present in a cultured population three days after co-culturing.

FIGS. 23A-23C show data related to the reduced expression of selected target genes in a second donor's T cells, the resultant HLA expression levels, and the percentages of NK cells and T cells present after three days of co-culturing the edited T cells with NK cells expressing a chimeric antigen receptor. FIG. 23A shows flow cytometry data for expression of selected genes by T cells from a second donor after gene disruption. FIG. 23B shows the data of FIG. 23A tabulated. FIG. 23C shows histograms showing the percentage of T cells and NK cells present in a cultured population three days after co-culturing.

FIGS. 24A-24C show survival curves for T cells that are edited at the indicated target gene (or electroporation only (EP)) and cocultured with NK cells at the indicated NK:T ratio. FIG. 24A shows survival of T cells after co-culturing at a 2:1 NK:T cell ratio. FIG. 24B shows survival of T cells after co-culturing at a 1:1 NK:T cell ratio. FIG. 24C shows survival of T cells after co-culturing at a 1:2 NK:T cell ratio.

FIG. 25 shows a schematic of antigen processing, loading of MHC molecules and transport to the cell surface for antigen presentation.

FIGS. 26A-26C depict schematics of non-limiting embodiments of immunosuppressive effector constructs as provided for herein.

### DETAILED DESCRIPTION

Some embodiments of the methods and compositions provided herein relate to engineered immune cells and combinations of the same for use in immunotherapy. In several embodiments, the engineered cells are engineered in multiple ways, for example, to express a cytotoxicity-inducing receptor complex. As used herein, the term “cytotoxic receptor complexes” shall be given its ordinary meaning and shall also refer to (unless otherwise indicated), Chimeric Antigen Receptors (CAR), chimeric receptors (also called activating chimeric receptors in the case of NKG2D chimeric receptors). In several embodiments, the cells are further engineered to achieve a modification of the reactivity of the cells against non-tumor tissue. Several embodiments relate to the modification of T cells, through various genetic engineering methodologies, such that the resultant T cells have reduced and/or eliminated alloreactivity. Such non-alloreactive T cells can also be engineered to express a chimeric antigen receptor (CAR) that enables the non-alloreactive T cells to impart cytotoxic effects against tumor cells. In several embodiments, natural killer (NK) cells are also engineered to express a cytotoxicity-inducing receptor complex (e.g., a chimeric antigen receptor or chimeric receptor).

While autologous CAR T cell therapies have been developed and shown to exhibit substantial in vivo persistence and efficacy, the majority of patients treated with autologous CAR T cell therapy will experience cytokine release syndrome (CRS) or a neurotoxicity. Further, autologous CAR T cell therapies face numerous challenges, including the need to leukapherese and then manufacture a conforming CAR T cell product from patients who are often extremely sick, heavily pre-treated, or both. Manufacturing sufficient numbers of CAR T cells from such patients can be difficult, or in some cases, impossible. In addition, a potential patient may not survive the length of time it takes to manufacture the final CAR T cell product from the T cells obtained from the patient.

Allogeneic CAR T cell therapies manufactured from healthy donors can obviate many of these challenges. For example, manufacturing success rates for allogeneic CAR T cells may be higher due to better quality of incoming donor T cells. Allogeneic CAR T cell therapies can also be provided when a patient is in need, without having to wait for the patient's own cells to be manufactured. Thus, allogeneic CAR T cell therapies are being investigated for use as off-the-shelf products.

Despite the challenges that allogeneic CAR T cells may overcome, they are associated with their own set of obstacles. Specifically, allogeneic T cells can result in graft versus host disease (GvHD) or host versus graft disease (HvGD) due to the immunologic mismatch between donor cells and recipient patient cells. Solutions are therefore needed to overcome such challenges. In some cases, T cells are edited to eliminate cell surface expression of HLA class I molecules, such as by knocking out beta-2 microglobulin (B2M), which can reduce host T cell-mediated graft rejection. However, this approach can render administered cells (e.g., T cells) susceptible to graft rejection mediated by host and/or administered NK cells. Thus, alternative approaches are needed, particularly for cell therapy compositions comprising both NK and T cells. It is observed herein that knockout of genes associated with the secretory pathway by which peptide-loaded HLA molecules are transported to the plasma membrane of the cell. For example, observations described herein demonstrate that knockout of TAP-2 or TAPBP reduced, but did not eliminate, HLA I expression in T cell populations. Specifically, the amount of HLA I expressed by each of the T cells was reduced, despite nearly 100% of the population still expressing some amount of HLA I. By contrast, B2M knockout resulted in fewer cells actually expressing HLA I, but those that did still express HLA I expressed it near unedited levels. It was surpisingly observed that T cells knocked out for TAP-2 or TAPBP were maintained at a higher percentage in co-culture with NK cells, as compared to T cells knocked out for B2M in co-culture with NK cells. Thus, knock out of TAP-2 or TAPBP in T cells increased their persistence as compared to knockout of B2M, when present in combination with NK cells.

In several embodiments, combinations of these engineered immune cell types (e.g., NK and T cells) are used in immunotherapy, which results in both a rapid (NK-cell based) and persistent (T-cell based) anti-tumor effect, all while advantageously having little to no graft versus host disease. Some embodiments include methods of use of the compositions or cells in immunotherapy.

The term “anticancer effect” refers to a biological effect which can be manifested by various means, including but not limited to, a decrease in tumor volume, a decrease in the number of cancer cells, a decrease in the number of metastases, an increase in life expectancy, decrease in cancer cell proliferation, decrease in cancer cell survival, and/or amelioration of various physiological symptoms associated with the cancerous condition.

### Cell Types

Some embodiments of the methods and compositions provided herein relate to a cell such as an immune cell. In some embodiments, a T cell is engineered to express a chimeric receptor that binds to an antigen (e.g., an antigen expressed by a cancer cell). For example, an immune cell, such as a T cell, may be engineered to include a chimeric receptor such as a CD19-directed chimeric receptor, or engineered to include a nucleic acid encoding said chimeric receptor as described herein. In some embodiments, a NK cell is engineered to express a chimeric receptor that binds to an antigen (e.g., an antigen expressed by a cancer cell). Additional embodiments relate to engineering a second set of cells (e.g., NK cells) to express another cytotoxic receptor complex, such as an NKG2D chimeric receptor complex as disclosed herein. Thus, in some embodiments, combinations or compositions comprising both engineered T cells and engineered NK cells are contemplated. In some embodiments, the engineered T cells and the engineered NK cells express the same chimeric receptor. In some embodiments, the engineered T cells and the engineered NK cells express different chimeric receptors. In some embodiments, the engineered T cells and the engineered NK cells express chimeric receptors that bind to the same antigen (e.g., different epitopes of the same antigen). In some embodiments, the engineered T cells and the engineered NK cells express chimeric receptors that binds different antigens.

Still additional embodiments relate to the further genetic manipulation of T cells (e.g., donor T cells) to reduce, disrupt, minimize and/or eliminate the ability of the donor T cell to be alloreactive against recipient cells (graft versus host disease). For example, in some embodiments, T cells are engineered to reduce alloreactivity against recipient cells.

Traditional anti-cancer therapies relied on a surgical approach, radiation therapy, chemotherapy, or combinations of these methods. As research led to a greater understanding of some of the mechanisms of certain cancers, this knowledge was leveraged to develop targeted cancer therapies. Targeted therapy is a cancer treatment that employs certain drugs that target specific genes or proteins found in cancer cells or cells supporting cancer growth, (like blood vessel cells) to reduce or arrest cancer cell growth. More recently, genetic engineering has enabled approaches to be developed that harness certain aspects of the immune system to fight cancers. In some cases, a patient's own immune cells are modified to specifically eradicate that patient's type of cancer. Various types of immune cells can be used, such as T cells, Natural Killer (NK cells), or combinations thereof, as described in more detail below.

To facilitate cancer immunotherapies, there are provided for herein polynucleotides, polypeptides, and vectors that encode chimeric antigen receptors (CAR) that comprise a target binding moiety (e.g., an extracellular binder of a ligand, or a tumor marker-directed chimeric receptor, expressed by a cancer cell) and a cytotoxic signaling complex. For example, some embodiments include a polynucleotide, polypeptide, or vector that encodes, for example a chimeric antigen receptor directed against a tumor marker, for example, CD19, CD123, CD70, Her2, mesothelin, Claudin 6, BCMA, EGFR, among others, to facilitate targeting of an immune cell to a cancer and exerting cytotoxic effects on the cancer cell. Also provided are engineered immune cells (e.g., T cells or NK cells) expressing such CARs. In some embodiments, a chimeric antigen receptor binds to ligands of NKG2D. In some embodiments, a chimeric antigen receptor binds to CD19. In some embodiments, a chimeric antigen receptor binds to CD70. There are also provided herein, in several embodiments, polynucleotides, polypeptides, and vectors that encode a construct comprising an extracellular domain comprising two or more subdomains, e.g., first CD19-targeting subdomain comprising a CD19 binding moiety as disclosed herein and a second subdomain comprising a C-type lectin-like receptor and a cytotoxic signaling complex. Also provided are engineered immune cells (e.g., T cells or NK cells) expressing such bi-specific constructs. Methods of treating cancer and other uses of such cells for cancer immunotherapy are also provided for herein.

To facilitate cancer immunotherapies, there are also provided for herein polynucleotides, polypeptides, and vectors that encode chimeric receptors that comprise a target binding moiety (e.g., an extracellular binder of a ligand expressed by a cancer cell) and a cytotoxic signaling complex. For example, some embodiments include a polynucleotide, polypeptide, or vector that encodes, for example an activating chimeric receptor comprising an NKG2D extracellular domain that is directed against a tumor marker, for example, MICA, MICB, ULBP1, ULBP2, ULBP3, ULBP4, ULBP5, and ULBP6, among others, to facilitate targeting of an immune cell to a cancer and exerting cytotoxic effects on the cancer cell. In some embodiments, the chimeric receptor comprises an extracellular domain of NKG2D. Also provided are engineered immune cells (e.g., T cells or NK cells) expressing such chimeric receptors. There are also provided herein, in several embodiments, polynucleotides, polypeptides, and vectors that encode a construct comprising an extracellular domain comprising two or more subdomains, e.g., first and second ligand binding receptor and a cytotoxic signaling complex. Also provided are engineered immune cells (e.g., T cells or NK cells) expressing such bi-specific constructs (in some embodiments the first and second ligand binding domain target the same ligand). Methods of treating cancer and other uses of such cells for cancer immunotherapy are also provided for herein.

### Engineered Cells for Immunotherapy

In several embodiments, cells of the immune system are engineered to have enhanced cytotoxic effects against target cells, such as tumor cells. For example, a cell of the immune system may be engineered to include a tumor-directed chimeric receptor and/or a tumor-directed CAR as described herein. In several embodiments, white blood cells or leukocytes, are used, since their native function is to defend the body against growth of abnormal cells and infectious disease. There are a variety of types of white bloods cells that serve specific roles in the human immune system, and are therefore a preferred starting point for the engineering of cells disclosed herein. White blood cells include granulocytes and agranulocytes (presence or absence of granules in the cytoplasm, respectively). Granulocytes include basophils, eosinophils, neutrophils, and mast cells. Agranulocytes include lymphocytes and monocytes. Cells such as those that follow or are otherwise described herein may be engineered to include a chimeric receptor, such as an NKG2D chimeric receptor, and/or a CAR, such as a CD19-directed CAR, or a nucleic acid encoding the chimeric receptor or the CAR. In several embodiments, the cells are optionally engineered to co-express a membrane-bound interleukin 15 (mbIL15) co-stimulatory domain. In some embodiments, the immune cells engineered to express a chimeric receptor are engineered to bicistronically express a mbIL15 domain. As discussed in more detail below, in several embodiments, the cells, particularly T cells, are further genetically modified to reduce and/or eliminate the alloreactivity of the cells.

### Monocytes for Immunotherapy

In some embodiments, the immune cells comprise monocytes. Monocytes are a subtype of leukocyte. Monocytes can differentiate into macrophages and myeloid lineage dendritic cells. Monocytes are associated with the adaptive immune system and serve the main functions of phagocytosis, antigen presentation, and cytokine production. Phagocytosis is the process of uptake of cellular material, or entire cells, followed by digestion and destruction of the engulfed cellular material.

In some embodiments, a monocyte is positive for cell surface expression of a marker selected from among the group consisting of CCR2, CCR5, CD11c, CD14, CD16, CD62L, CD68+, CX3CR1, HLA-DR, or any combination thereof. In some embodiments, a monocyte is positive for cell surface expression of CD14. In some embodiments, a monocyte is positive for cell surface expression of CCR2. In some embodiments, a monocyte is positive for cell surface expression of CCR5. In some embodiments, a monocyte is positive for cell surface expression of CD62L.

In several embodiments, monocytes are used in connection with one or more additional engineered cells as disclosed herein. Some embodiments of the methods and compositions described herein relate to a monocyte that includes a tumor-directed CAR, or a nucleic acid encoding the tumor-directed CAR. In some embodiments, the monocytes express a CAR that binds to a tumor antigen, for example, CD19, CD123, CD70, Her2, mesothelin, Claudin 6, BCMA, or EGFR.

In some embodiments, the monocytes are engineered to a membrane-bound interleukin 15 (mbIL15) domain. In some embodiments, the monocytes engineered to express a chimeric receptor are also engineered to also express (e.g., bicistronically express) a membrane-bound interleukin 15 (mbIL15) domain. Thus, in some embodiments, the monocytes are engineered to bicistronically express the chimeric receptor and mbIL15. Several embodiments of the methods and compositions disclosed herein relate to monocytes engineered to express a CAR that targets a tumor marker, for example, CD19, CD123, CD70, Her2, mesothelin, Claudin 6, BCMA, EGFR, among any of the others disclosed herein, and a membrane-bound interleukin 15 (mbIL15) co-stimulatory domain. Several embodiments of the methods and compositions disclosed herein relate to monocytes engineered to express an activating chimeric receptor that targets a ligand on a tumor cell, for example, MICA, MICB, ULBP1, ULBP2, ULBP3, ULBP4, ULBP5, and ULBP6 (among others) and optionally a membrane-bound interleukin 15 (mbIL15) co-stimulatory domain.

In some embodiments, the monocytes are allogeneic cells. In some embodiments, the monocytes are obtained from a donor who does not have cancer.

### Lymphocytes for Immunotherapy

In some embodiments, the immune cells comprise lymphocytes. Lymphocytes, the other primary sub-type of leukocyte include T cells (cell-mediated, cytotoxic adaptive immunity), natural killer cells (cell-mediated, cytotoxic innate immunity), and B cells (humoral, antibody-driven adaptive immunity). While B cells are engineered according to several embodiments, disclosed herein, several embodiments also relate to engineered T cells or engineered NK cells (mixtures of T cells and NK cells are used in some embodiments, either from the same donor, or different donors). Thus, in some embodiments, the immune cells comprise T cells. In some embodiments, the immune cells comprise NK cells. In some embodiments, the immune cells comprise T cells and NK cells. In some embodiments, the immune cells comprise B cells.

In several embodiments, lymphocytes are used in connection with one or more additional engineered cells as disclosed herein. Some embodiments of the methods and compositions described herein relate to a lymphocyte that includes a tumor-directed CAR, or a nucleic acid encoding the tumor-directed CAR. In some embodiments, the lymphocytes express a CAR that binds to a tumor antigen, for example, CD19, CD123, CD70, Her2, mesothelin, Claudin 6, BCMA, or EGFR.

In some embodiments, the lymphocytes are engineered to a membrane-bound interleukin 15 (mbIL15) domain. In some embodiments, the lymphocytes engineered to express a chimeric receptor are also engineered to also express (e.g., bicistronically express) a membrane-bound interleukin 15 (mbIL15) domain. Thus, in some embodiments, lymphocytes are engineered to bicistronically express the chimeric receptor and mbIL15. Several embodiments of the methods and compositions disclosed herein relate to lymphocytes engineered to express a CAR that targets a tumor marker, for example, CD19, CD123, CD70, Her2, mesothelin, Claudin 6, BCMA, EGFR, among any of the others disclosed herein, and a membrane-bound interleukin 15 (mbIL15) co-stimulatory domain. Several embodiments of the methods and compositions disclosed herein relate to lymphocytes engineered to express an activating chimeric receptor that targets a ligand on a tumor cell, for example, MICA, MICB, ULBP1, ULBP2, ULBP3, ULBP4, ULBP5, and ULBP6 (among others) and optionally a membrane-bound interleukin 15 (mbIL15) co-stimulatory domain.

In some embodiments, the monocytes are allogeneic cells. In some embodiments, the monocytes are obtained from a donor who does not have cancer.

### T Cells for Immunotherapy

In some embodiments, the immune cells comprise T cells. T cells are distinguishable from other lymphocytes sub-types (e.g., B cells or NK cells) based on the presence of a T-cell receptor on the cell surface.

T cells can be divided into various different subtypes, including effector T cells, helper T cells, cytotoxic T cells, memory T cells, regulatory T cells, natural killer T cell, mucosal associated invariant T cells and gamma delta T cells. In some embodiments, a specific subtype of T cell is engineered. In some embodiments, a T cell is positive for cell surface expression of a marker selected from among the group consisting of CD3, CD4, and/or CD8. In some embodiments, a T cell is positive for cell surface expression of CD3. In some embodiments, a T cell is positive or cell surface expression of CD4. In some embodiments, a T cell is positive or cell surface expression of CD8.

In some embodiments, CD3+ T cells are engineered. In some embodiments, CD4+ T cells are engineered. In some embodiments, CD8+ T cells are engineered. In some embodiments, regulatory T cells are engineered. In some embodiments, gamma delta T cells are engineered. In some embodiments, a mixed pool of T cell subtypes is engineered. For example, in some embodiments, CD4+ and CD8+ T cells are engineered. In some embodiments, there is no specific selection of a type of T cells to be engineered to express the cytotoxic receptor complexes disclosed herein. In several embodiments, specific techniques, such as use of cytokine stimulation are used to enhance expansion/collection of T cells with a specific marker profile. For example, in several embodiments, activation of certain human T cells, e.g., CD4+ T cells, CD8+ T cells is achieved through use of CD3 and/or CD28 as stimulatory molecules.

In several embodiments, there is provided a method of treating or preventing cancer or an infectious disease, comprising administering a therapeutically effective amount of T cells expressing the cytotoxic receptor complex and/or a homing moiety as described herein. In several embodiments, there is provided a method of treating or preventing cancer or an infectious disease, comprising administering T cells expressing a cytotoxic receptor complex as described herein. In several embodiments, the engineered T cells are autologous cells, while in some embodiments, the T cells are allogeneic cells. In some embodiments, the T cells are allogeneic cells. In some embodiments, the T cells are obtained from a donor who does not have cancer.

Several embodiments of the methods and compositions disclosed herein relate to T cells engineered to express a CAR that targets a tumor marker, for example, CD19, CD123, CD70, Her2, mesothelin, Claudin 6, BCMA, EGFR, among any of the others as disclosed herein, and a membrane-bound interleukin 15 (mbIL15) co-stimulatory domain. In some embodiments, T cells express a CAR that binds to CD19. In some embodiments, T cells express a CAR that binds to CD70. Several embodiments of the methods and compositions disclosed herein relate to T cells engineered to express an activating chimeric receptor that targets a ligand on a tumor cell, for example, MICA, MICB, ULBP1, ULBP2, ULBP3, ULBP4, ULBP5, and ULBP6 (among others) and optionally a membrane-bound interleukin 15 (mbIL15) co-stimulatory domain. In some embodiments, T cells express a chimeric receptor that binds to a NKG2D ligand. In some embodiments, T cells express a chimeric receptor comprising an extracellular domain of NKG2D.

In some embodiments, the T cells are engineered to a membrane-bound interleukin 15 (mbIL15) domain. In some embodiments, the T cells engineered to express a chimeric receptor are also engineered to also express (e.g., bicistronically express) a membrane-bound interleukin 15 (mbIL15) domain. Thus, in some embodiments, the T cells are engineered to bicistronically express the chimeric receptor and mbIL15.

In some embodiments, the immune cells comprise T cells and NK cells (either from the same donor or from different donors).

### NK Cells for Immunotherapy

In some embodiments, the immune cells comprise natural killer (NK) cells. In several embodiments, NK cells are preferred because the natural cytotoxic potential of NK cells is relatively high. In several embodiments, it is unexpectedly beneficial that the engineered cells disclosed herein can further upregulate the cytotoxic activity of NK cells, leading to an even more effective activity against target cells (e.g., tumor or other diseased cells).

In some embodiments, a NK cell is positive for cell surface expression of a marker selected from among the group consisting of CCR7, CD16, CD56, CD57, CD11, CX3CR1, a Killer Ig-like receptor (KIR), NKp30, NKp44, NKp46, or any combination thereof. In some embodiments, a NK cell is positive for cell surface expression of CD16. In some embodiments, a NK cell is positive for cell surface expression of CD56. In some embodiments, a NK cell is positive for cell surface expression of a Killer Ig-like receptor.

In several embodiments, there is provided a method of treating or preventing cancer or an infectious disease, comprising administering a therapeutically effective amount of natural killer (NK) cells expressing the cytotoxic receptor complex and/or a homing moiety as described herein. In several embodiments, there is provided a method of treating or preventing cancer or an infectious disease, comprising administering NK cells expressing a cytotoxic receptor complex as described herein. In several embodiments, there is provided a method of treating or preventing cancer, comprising administering NK cells expressing a cytotoxic receptor complex as described herein. In several embodiments, there is provided a method of treating or preventing an infectious disease, comprising administering NK cells expressing a cytotoxic receptor complex as described herein. In several embodiments, the engineered NK cells are autologous cells, while in some embodiments, the NK cells are allogeneic cells. In some embodiments, the NK cells are allogeneic cells. In some embodiments, the NK cells are obtained from a donor who does not have cancer.

Some embodiments of the methods and compositions described herein relate to NK cells engineered to express a CAR that targets a tumor marker, for example, CD19, CD123, CD70, Her2, mesothelin, Claudin 6, BCMA, EGFR, among any of the others disclosed herein, and optionally a membrane-bound interleukin 15 (mbIL15) co-stimulatory domain. In some embodiments, NK cells express a CAR that binds to CD19. In some embodiments, T cells express a CAR that binds to CD70. Several embodiments of the methods and compositions disclosed herein relate to NK cells engineered to express an activating chimeric receptor that targets a ligand on a tumor cell, for example, MICA, MICB, ULBP1, ULBP2, ULBP3, ULBP4, ULBP5, and ULBP6 (among others) and optionally a membrane-bound interleukin 15 (mbIL15) co-stimulatory domain. In some embodiments, NK cells express a chimeric receptor that binds to a NKG2D ligand. In some embodiments, NK cells express a chimeric receptor comprising an extracellular domain of NKG2D.

In some embodiments, the NK cells are engineered to a membrane-bound interleukin 15 (mbIL15) domain. In some embodiments, the NK cells engineered to express a chimeric receptor are also engineered to also express (e.g., bicistronically express) a membrane-bound interleukin 15 (mbIL15) domain. Thus, in some embodiments, the NK cells are engineered to bicistronically express the chimeric receptor and mbIL15.

In some embodiments, the NK cells are used in combination with T cells. Thus, in some embodiments, the immune cells comprise T cells and NK cells (either from the same donor or from different donors).

In some embodiments, the NK cells are derived from cell line NK-92. NK-92 cells are derived from NK cells, but lack major inhibitory receptors displayed by normal NK cells, while retaining the majority of activating receptors. Some embodiments of NK-92 cells described herein related to NK-92 cell engineered to silence certain additional inhibitory receptors, for example, SMAD3, allowing for upregulation of interferon-γ (IFNγ), granzyme B, and/or perforin production. Additional information relating to the NK-92 cell line is disclosed in WO 1998/49268 and U.S. Patent Application Publication No. 2002-0068044 and incorporated in their entireties herein by reference.

NK-92 cells are used, in several embodiments, in combination with one or more of the other cell types disclosed herein. For example, in one embodiment, NK-92 cells are used in combination with NK cells as disclosed herein. In an additional embodiment, NK-92 cells are used in combination with T cells as disclosed herein.

### Hematopoietic Stem Cells for Cancer Immunotherapy

In some embodiments, hematopoietic stem cells (HSCs) are used in the methods of immunotherapy disclosed herein. In several embodiments, the cells are engineered to express a homing moiety and/or a cytotoxic receptor complex. HSCs are used, in several embodiments, to leverage their ability to engraft for long-term blood cell production, which could result in a sustained source of targeted anti-cancer effector cells, for example to combat cancer remissions. In several embodiments, this ongoing production helps to offset anergy or exhaustion of other cell types, for example due to the tumor microenvironment.

In some embodiments, a HSC is positive for cell surface expression of a marker selected from among the group consisting of CD34, CD59, and CD90. In some embodiments, a HSC is positive for cell surface expression of CD34. In some embodiments, a HSC is positive for cell surface expression of CD59. In some embodiments, a HSC is positive for cell surface expression of CD90.

In several embodiments allogeneic HSCs are used, while in some embodiments, autologous HSCs are used. In several embodiments, HSCs are used in combination with one or more additional engineered cell type disclosed herein. Some embodiments of the methods and compositions described herein relate to a stem cell, such as a hematopoietic stem cell engineered to express a CAR that targets a tumor marker, for example, CD19, CD123, CD70, Her2, mesothelin, Claudin 6, BCMA, EGFR, among any of the others disclosed herein, and optionally a membrane-bound interleukin 15 (mbIL15) co-stimulatory domain. Several embodiments of the methods and compositions disclosed herein relate to hematopoietic stem cells engineered to express an activating chimeric receptor that targets a ligand on a tumor cell, for example, MICA, MICB, ULBP1, ULBP2, ULBP3, ULBP4, ULBP5, and ULBP6 (among others) and optionally a membrane-bound interleukin 15 (mbIL15) co-stimulatory domain.

### Induced Pluripotent Stem Cells

In some embodiments, immune cells are derived (differentiated) from pluripotent stem cells (PSCs). In some embodiments, immune cells (e.g., NK and/or T cells) derived from induced pluripotent stem cells (iPSCs) are used in the method of immunotherapy disclosed herein. For example, in some embodiments, NK cells, T cells, or both are derived from iPSCs. iPSCs are used, in several embodiments, to leverage their ability to differentiate and derive into non-pluripotent cells, including, but not limited to, CD34 cells, hemogenic endothelium cells, HSCs (hematopoietic stem and progenitor cells), hematopoietic multipotent progenitor cells, T cell progenitors, NK cell progenitors, T cells, NKT cells, NK cells, and B cells comprising one or several genetic modifications at selected sites through differentiating iPSCs or less differentiated cells comprising the same genetic modifications at the same selected sites. In several embodiments, the iPSCs are used to generate iPSC-derived NK or T cells. In several embodiments, the iPSCs are used to generate iPSC-derived NK cells. In several embodiments, the iPSCs are used to generate iPSC-derived T cells.

In several embodiments, the cells are engineered to express a homing moiety and/or a cytotoxic receptor complex. In several embodiments, iPSCs are used in combination with one or more additional engineered cell type disclosed herein.

Several embodiments of the methods and compositions disclosed herein relate to induced pluripotent stem cells engineered to express an activating chimeric receptor that targets a ligand on a tumor cell, for example, CD19, CD123, CD70, Her2, mesothelin, Claudin 6, BCMA, or EGFR. In some embodiments, the iPSCs engineered to express a chimeric receptor are engineered to also express (e.g., bicistronically express) a membrane-bound interleukin 15 (mbIL15) co-stimulatory domain. Some embodiments of the methods and compositions described herein relate to a stem cell, such as an induced pluripotent stem cell engineered to express a CAR that targets a tumor marker, for example, CD19, CD123, CD70, Her2, mesothelin, Claudin 6, BCMA, EGFR, among any of the others disclosed herein, and optionally a membrane-bound interleukin 15 (mbIL15) co-stimulatory domain.

Several embodiments of the methods and compositions disclosed herein relate to induced pluripotent stem cells engineered to express an activating chimeric receptor that targets a ligand on a tumor cell, for example, MICA, MICB, ULBP1, ULBP2, ULBP3, ULBP4, ULBP5, and ULBP6 (among others). In some embodiments, the iPSCs engineered to express a chimeric receptor are engineered to also express (e.g., bicistronically express) a membrane-bound interleukin 15 (mbIL15) co-stimulatory domain. Several embodiments of the methods and compositions disclosed herein relate to induced pluripotent stem cells engineered to express an activating chimeric receptor that targets a ligand on a tumor cell, for example, MICA, MICB, ULBP1, ULBP2, ULBP3, ULBP4, ULBP5, and ULBP6 (among others) and optionally a membrane-bound interleukin 15 (mbIL15) co-stimulatory domain.

In several embodiments, the engineered iPSCs are differentiated into NK, T, or other immune cells, such as for use in a composition or method provided herein. In several embodiments, the engineered iPSCs are differentiated into NK cells. In several embodiments, the engineered iPSCs are differentiated into T cells. In several embodiments, the engineered iPSCs are differentiated into NK and T cells.

### Genetic Editing of Immune Cells

As discussed above, a variety of cell types can be utilized in cellular immunotherapy. Further, as elaborated on in more detail below, and shown in the Examples, genetic modifications can be made to these cells in order to enhance one or more aspects of their efficacy (e.g., cytotoxicity) and/or persistence (e.g., active life span). As discussed herein, in several embodiments NK cells are used for immunotherapy. In several embodiments provided for herein, gene editing of the NK cell can advantageously impart to the edited NK cell the ability to resist and/or overcome various inhibitory signals that are generated in the tumor microenvironment. It is known that tumors generate a variety of signaling molecules that are intended to reduce the anti-tumor effects of immune cells. As discussed in more detail below, in several embodiments, gene editing of the NK cell limits this tumor microenvironment suppressive effect on the NK cells, T cells, combinations of NK and T cells, or any edited/engineered immune cell provided for herein.

As discussed below, in several embodiments, gene editing is employed to reduce or knockout expression of target proteins, for example by disrupting the underlying gene encoding the protein.

In several embodiments, gene editing can reduce transcription of a target gene by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces transcription of a target gene by at least about 30%. In several embodiments, gene editing reduces transcription of a target gene by at least about 40%. In several embodiments, gene editing reduces transcription of a target gene by at least about 50%. In several embodiments, gene editing reduces transcription of a target gene by at least about 60%.

In several embodiments, gene editing reduces transcription of a target gene by at least about 70%. In several embodiments, gene editing reduces transcription of a target gene by at least about 80%. In several embodiments, gene editing reduces transcription of a target gene by at least about 90%. In several embodiments, the gene is completely knocked out, such that transcription of the target gene is undetectable.

In several embodiments, gene editing can reduce expression of a target protein by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of a target protein by at least about 30%. In several embodiments, gene editing reduces expression of a target protein by at least about 40%. In several embodiments, gene editing reduces expression of a target protein by at least about 50%. In several embodiments, gene editing reduces expression of a target protein by at least about 60%. In several embodiments, gene editing reduces expression of a target protein by at least about 70%. In several embodiments, gene editing reduces expression of a target protein by at least about 80%. In several embodiments, gene editing reduces expression of a target protein by at least about 90%. In several embodiments, the gene is completely knocked out, such that expression of the target protein is undetectable.

In several embodiments, gene editing is used to “knock in” or otherwise increase transcription of a target gene. In several embodiments, transcription of a target gene is increased by about 30%, about 40%, about 50%, about 60%, about 70%, about 75%, about 80%, about 85%, about 90%, about 95%, about 97%, about 98%, about 99%, or more (including any amount between those listed). In several embodiments, transcription of a target gene is increased by at least about 30%. In several embodiments, transcription of a target gene is increased by at least about 40%. In several embodiments, transcription of a target gene is increased by at least about 50%. In several embodiments, transcription of a target gene is increased by at least about 60%. In several embodiments, transcription of a target gene is increased by at least about 70%. In several embodiments, transcription of a target gene is increased by at least about 80%. In several embodiments, transcription of a target gene is increased by at least about 90%. In several embodiments, transcription of a target gene is increased by at least about 100%.

In several embodiments, gene editing is used to “knock in” or otherwise enhance expression of a target protein. In several embodiments, expression of a target protein can be enhanced by about 30%, about 40%, about 50%, about 60%, about 70%, about 75%, about 80%, about 85%, about 90%, about 95%, about 97%, about 98%, about 99%, or more (including any amount between those listed). In several embodiments, expression of a target protein is increased by at least about 30%. In several embodiments, expression of a target protein is increased by at least about 40%. In several embodiments, expression of a target protein is increased by at least about 50%. In several embodiments, expression of a target protein is increased by at least about 60%. In several embodiments, expression of a target protein is increased by at least about 70%. In several embodiments, expression of a target protein is increased by at least about 80%. In several embodiments, expression of a target protein is increased by at least about 90%. In several embodiments, expression of a target protein is increased by at least about 100%.

Unless indicated otherwise to the contrary, the sequences provided for guide RNAs that are recited using deoxyribonucleotides refer to the target DNA and shall be considered as also referencing those guides used in practice (e.g., employing ribonucleotides, where the ribonucleotide uracil is used in lieu of deoxyribonucleotide thymine or vice-versa where thymine is used in lieu of uracil, wherein both are complementary base pairs to adenine when reciting either an RNA or DNA sequence). For example, a gRNA with the sequence ATGCTCAATGCGTC (SEQ ID NO: 995) shall also refer to the following sequence AUGCUCAAUGCGUC (SEQ ID NO: 996) or a gRNA with sequence AUGCUCAAUGCGUC (SEQ ID NO: 996) shall also refer to the following sequence ATGCTCAATGCGTC (SEQ ID NO: 995).

As discussed in more detail below, a variety of approaches can be employed in a given embodiment to improve or alter one or more characteristics of immune cells for immunotherapy. Genetic editing can be used to reduce, eliminate (e.g., knockout), or increase expression of a target gene. For example, the transcription of the target gene and/or the translation of a protein encoded by the target gene (e.g., a target protein) can be reduced, eliminated (e.g., knocked out), or increased. The target gene can be implicated in the immune functionality of the cell, or be a part of a signaling pathway for which an increase or decrease in function is desired. Further detailed below are various gene targets. Various immunosuppressive approaches can be employed in some embodiments, in order to reduce the activity (e.g., cytotoxicity) of an immune cell against another cell(s) which is not a tumor cell (e.g., another cell within the population to be used for immunotherapy). Viral immunosuppressive peptides are disclosed herein. Additional disruptions (or deletions/partial replacements) of certain immune proteins can be used to increase persistence of a population of cells. In several embodiments, the increased persistence is a result of edits to that population of cells, while in some embodiments the increased persistence is because another population of cells was modified and therefore is less cytotoxic to the first population of cells. Changes or alterations in expression of native genes or proteins involved in immune function, such as HLA genes or proteins are used in some embodiments. Any combination of these approaches may also be used, depending on the embodiment.

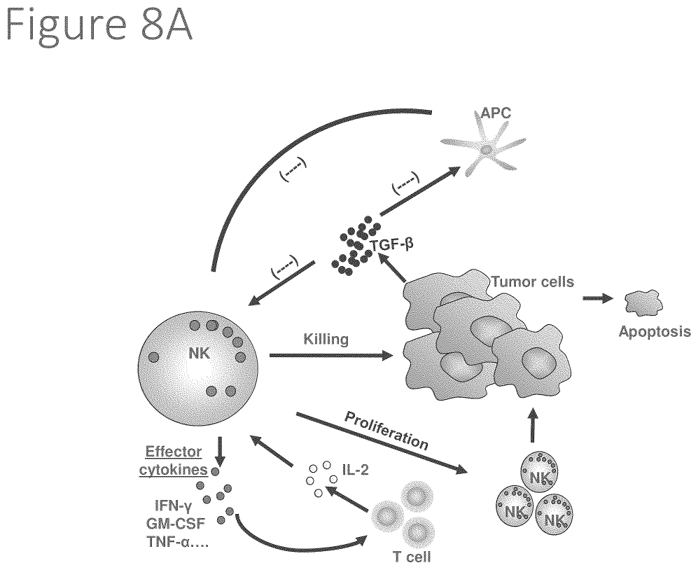

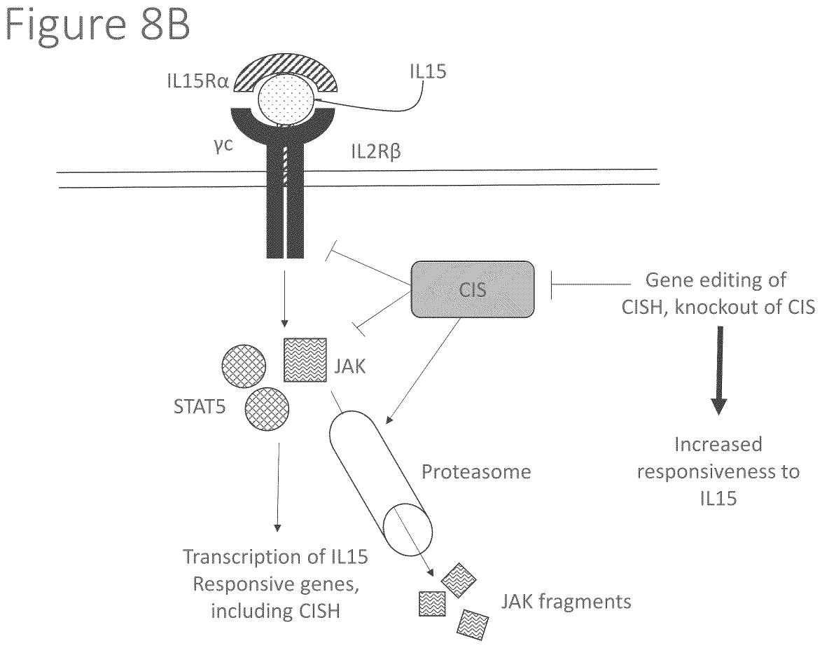

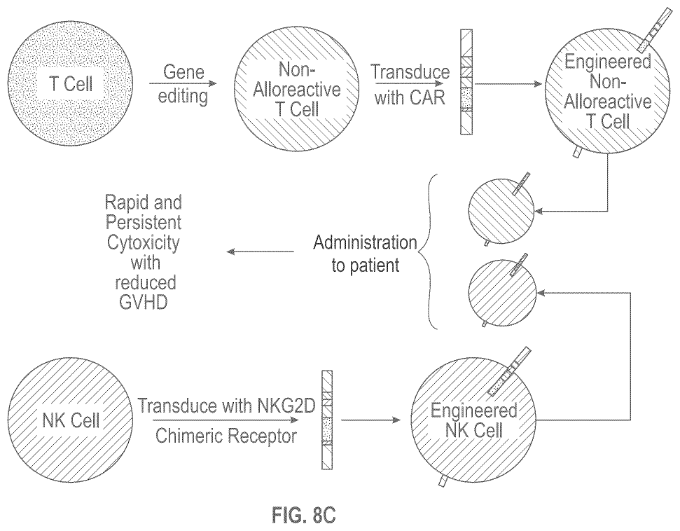

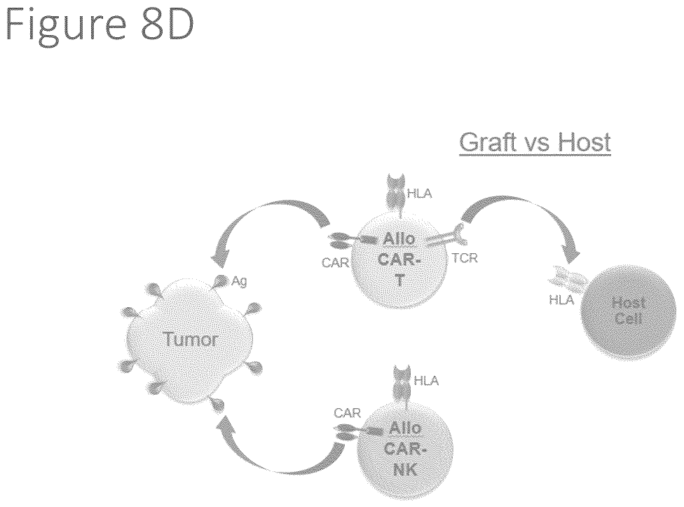

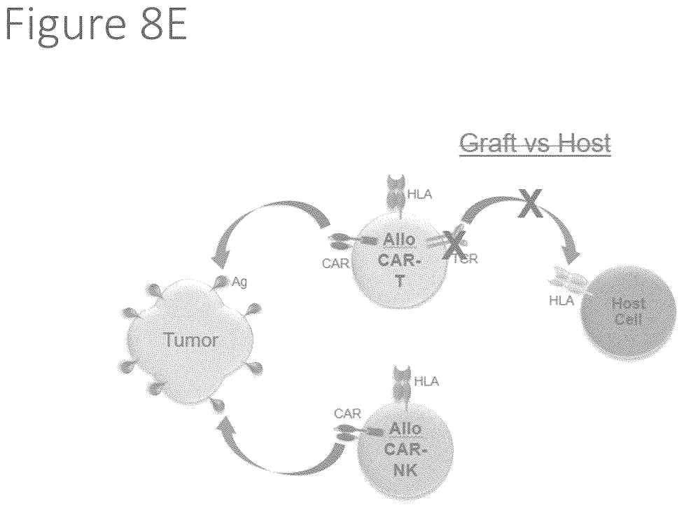

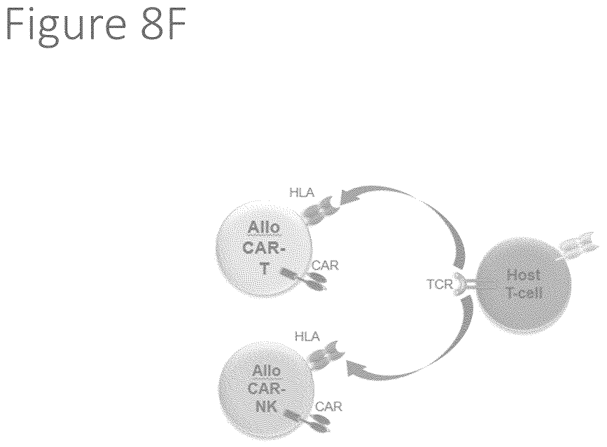

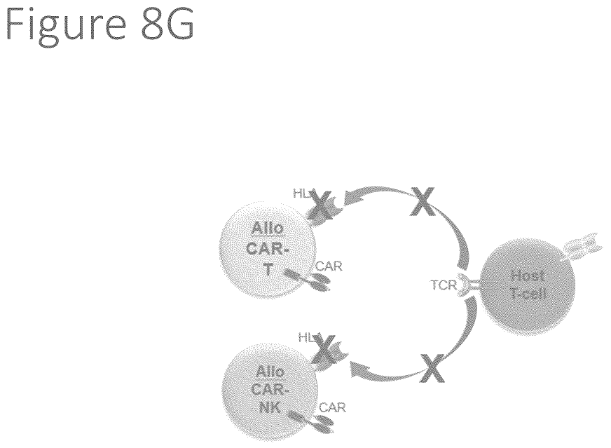

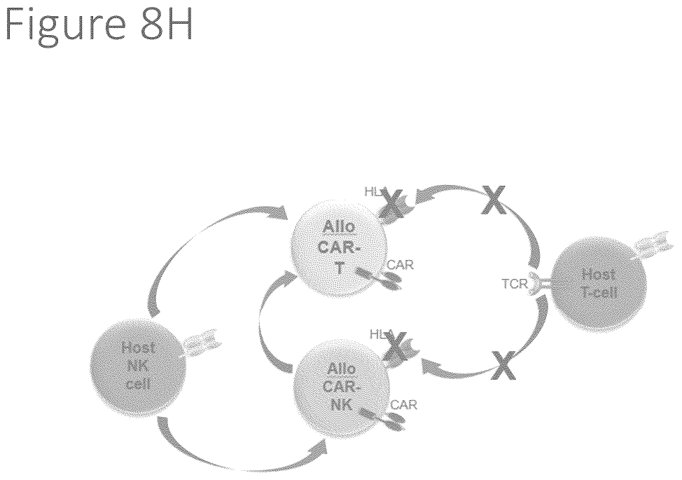

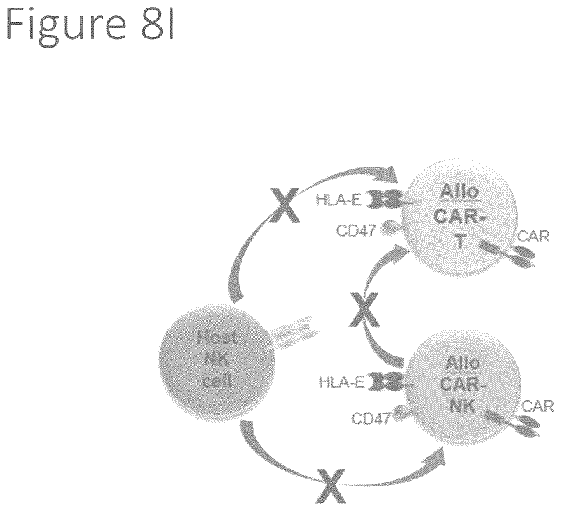

By way of non-limiting example, TGF-beta is one such cytokine released by tumor cells that results in immune suppression within the tumor microenvironment. That immune suppression reduces the ability of immune cells, even engineered CAR-immune cells is some cases, to destroy the tumor cells, thus allowing for tumor progression. In several embodiments, as discussed in detail below, immune checkpoints are disrupted through gene editing. In several embodiments, blockers of immune suppressing cytokines in the tumor microenvironment are used, including blockers of their release or competitive inhibitors that reduce the ability of the signaling molecule to bind and inhibit an immune cell. Such signaling molecules include, but are not limited to TGF-beta, IL10, arginase, inducible NOS, reactive-NOS, Arg1, Indoleamine 2,3-dioxygenase (IDO), and PGE₂. However, in additional embodiments, there are provided immune cells, such as NK cells, wherein the ability of the NK cell (or other cell) to respond to a given immunosuppressive signaling molecule is disrupted and/or eliminated. For example, in several embodiments, in several embodiments, NK cells or T cells are genetically edited to have reduced sensitivity to TGF-beta. TGF-beta is an inhibitor of NK cell function on at least the levels of proliferation and cytotoxicity. See, for example, FIG. 8A which schematically shows some of the inhibitory pathways by which TGF-beta reduces NK cell activity and/or proliferation. Thus, according to some embodiments, the expression of the TGF-beta receptor is knocked down or knocked out through gene editing, such that the edited NK cell is resistant to the immunosuppressive effects of TGF-beta in the tumor microenvironment. In several embodiments, the TGFB2 receptor (TGFBR2) is knocked down or knocked out through gene editing of the TGFBR2 gene, for example, by use of CRISPR-Cas editing. Small interfering RNA, antisense RNA, TALENs or zinc fingers are used in other embodiments. Other isoforms of the TGF-beta receptor (e.g., TGF-beta 1 and/or TGF-beta 3) are edited in some embodiments. In some embodiments TGF-beta receptors in T cells are knocked down through gene editing.

In accordance with additional embodiments, other modulators of one or more aspects of NK cell (or T cell) function are modulated through gene editing. A variety of cytokines impart either negative (as with TGF-beta above) or positive signals to immune cells. By way of non-limiting example, IL15 is a positive regulator of NK cells, which as disclosed herein, can enhance one or more of NK cell homing, NK cell migration, NK cell expansion/proliferation, NK cell cytotoxicity, and/or NK cell persistence. To keep NK cells in check under normal physiological circumstances, a cytokine-inducible SH2-containing protein (CIS, encoded by the CISH gene) acts as a critical negative regulator of IL15 signaling in NK cells. As discussed herein, IL15 biology impacts multiple aspects of NK cell functionality, including, but not limited to, proliferation/expansion, activation, cytotoxicity, persistence, homing, and migration, among others. Thus, according to several embodiments, editing CISH enhances the functionality of NK cells across multiple functionalities, leading to a more effective and long-lasting NK cell therapeutic. In several embodiments, inhibitors of CIS are used in conjunction with engineered NK cell administration. In several embodiments, the CIS expression is knocked down or knocked out through gene editing of the CISH gene, for example, by use of CRISPR-Cas editing. Small interfering RNA, antisense RNA, TALENs or zinc fingers are used in other embodiments. In some embodiments CIS expression in T cells is knocked down through gene editing.

In several embodiments, CISH gene editing endows an NK cell with enhanced ability to home to a target site. In several embodiments, CISH gene editing endows an NK cell with enhanced ability to migrate, e.g., within a tissue in response to, for example, chemoattractants, or away from repellants. In several embodiments, CISH gene editing endows an NK cell with enhanced ability to be activated, and thus exert, for example, anti-tumor effects. In several embodiments, CISH gene editing endows an NK cell with enhanced proliferative ability, which in several embodiments, allows for generation of robust NK cell numbers from a donor blood sample. In addition, in such embodiments, NK cells edited for CISH and engineered to express a CAR are more readily, robustly, and consistently expanded in culture. In several embodiments, CISH gene editing endows an NK cell with enhanced cytotoxicity. In several embodiments, the editing of CISH synergistically enhances the cytotoxic effects of engineered NK cells and/or engineered T cells that express a CAR.

In several embodiments, CISH gene editing activates or inhibits a wide variety of pathways. The CIS protein is a negative regulator of IL15 signaling by way of, for example, inhibiting JAK-STAT signaling pathways. These pathways would typically lead to transcription of IL15-responsive genes (including CISH). In several embodiments, knockdown of CISH disinhibits JAK-STAT (e.g., JAK1-STAT5) signaling and there is enhanced transcription of IL15-responsive genes. In several embodiments, knockout of CISH yields enhanced signaling through mammalian target of rapamycin (mTOR), with corresponding increases in expression of genes related to cell metabolism and respiration. In several embodiments, knockout of CISH yields IL15 induced increased expression of IL-2Rα (CD25), but not IL-15Rα or IL-2/15RP, enhanced NK cell membrane binding of IL15 and/or IL2, increased phosphorylation of STAT-3 and/or STAT-5, and elevated expression of the antiapoptotic proteins, such as Bcl-2. In several embodiments, CISH knockout results in IL15-induced upregulation of selected genes related to mitochondrial functions (e.g., electron transport chain and cellular respiration) and cell cycle. Thus, in several embodiments, knockout of CISH by gene editing enhances the NK cell cytotoxicity and/or persistence, at least in part via metabolic reprogramming. In several embodiments, negative regulators of cellular metabolism, such as TXNIP, are downregulated in response to CISH knockout. In several embodiments, promotors for cell survival and proliferation including BIRC5 (Survivin), TOP2A, CKS2, and RACGAP1 are upregulated after CISH knockout, whereas antiproliferative or proapoptotic proteins such as TGFB1, ATM, and PTCH1 are downregulated. In several embodiments, CISH knockout alters the state (e.g., activates or inactivates) signaling via or through one or more of CXCL-10, IL2, TNF, IFNg, IL13, IL4, Jnk, PRF1, STAT5, PRKCQ, IL2 receptor Beta, SOCS2, MYD88, STAT3, STAT1, TBX21, LCK, JAK3, IL& receptor, ABL1, IL9, STAT5A, STAT5B, Tcf7, PRDM1, and/or EOMES.

In several embodiments, gene editing of the immune cells can also provide unexpected enhancement in the expansion, persistence and/or cytotoxicity of the edited immune cell. As disclosed herein, engineered cells (e.g., those expressing a CAR) may also be edited, the combination of which provides for a robust cell for immunotherapy. In several embodiments, the edits allow for unexpectedly improved NK cell expansion, persistence and/or cytotoxicity. In several embodiments, knockout of CISH expression in NK cells removes a potent negative regulator of IL15-mediated signaling in NK cells, disinhibits the NK cells and allows for one or more of enhanced NK cell homing, NK cell migration, activation of NK cells, expansion, cytotoxicity and/or persistence. Additionally, in several embodiments, the editing can enhance NK and/or T cell function in the otherwise suppressive tumor microenvironment. In several embodiments, CISH gene editing results in enhanced NK cell expansion, persistence and/or cytotoxicity without requiring Notch ligand being provided exogenously.

As discussed above, T cells that are engineered to express a CAR or chimeric receptor are employed in several embodiments. Also as mentioned above, T cells express a T Cell Receptor (TCR) on their surface. As disclosed herein, in several embodiments, autologous immune cells are transferred back into the original donor of the cells. In such embodiments, immune cells, such as NK cells or T cells are obtained from patients, expanded, genetically modified (e.g., with a CAR or chimeric receptor) and/or optionally further expanded and re-introduced into the patient.

As disclosed herein, in several embodiments, allogeneic immune cells are transferred into a subject that is not the original donor of the cells. In such embodiments, immune cells, such as NK cells, T cells, or both, are obtained from a donor, expanded, genetically modified (e.g., with a CAR or chimeric receptor) and/or optionally further expanded and administered to the subject.

Allogeneic immunotherapy presents several hurdles to be overcome. In immune-competent hosts, the administered allogeneic cells are rapidly rejected, known as host versus graft rejection (HvG). This substantially limits the efficacy of the administered cells, particularly their persistence. In immune-incompetent hosts, allogeneic cells are able to engraft. However, if the administered cells comprise a T cell (several embodiments disclosed herein employ mixed populations of NK and T cells), the endogenous T cell receptor (TCR) specificities recognize the host tissue as foreign, resulting in graft versus host disease (GvHD). GvHD can lead to significant tissue damage in the host (cell recipient). Several embodiments disclosed herein address both of these hurdles, thereby allowing for effective and safe allogeneic immunotherapy. In several embodiments, gene edits can advantageously help to reduce and/or avoid graft vs. host disease (GvHD). A non-limiting embodiment of such an approach, using a mixed population of NK cell and T cells, is schematically illustrated in FIG. 8C, wherein the NK cells are engineered to express a CAR and the T cells are engineered to not only express a CAR, but also edited to render the T cells non-alloreactive. FIG. 8D schematically shows a mechanism by which GvHD occurs. An allogeneic T cell and an allogeneic NK cell, both engineered to express a CAR that targets the tumor, are introduced into a host. However, the T cell still bears the native T-cell receptor (TCR). This TCR recognizes the HLA type of the host cell as “non-self” and can exert cytotoxicity against host cells. FIG. 8E shows a non-limiting embodiment of how GvHD can be reduced or otherwise avoided through gene editing of the T cells. Briefly, as this approach is discussed in more detail below, gene editing can be performed in order to knockout the native TCR on T cells. Lacking a TCR, the allogeneic T cell cannot detect the “non-self” HLA of the host cells, and therefore is not triggered to exert cytotoxicity against host cells. Thus, in several embodiments T cells are subjected to gene editing to either reduce functionality of and/or reduce or eliminate expression of the native T cell. In several embodiments, CRISPR is used to knockout the TCR. These, and other, embodiments are discussed below.

T cell receptors (TCR) are cell surface receptors that participate in the activation of T cells in response to the presentation of an antigen. The TCR is made up of two different protein chains (it is a heterodimer). The majority of human T cells have TCRs that are made up of an alpha (α) chain and a beta (β) chain (encoded by separate genes). A small percentage of T cells have TCRs made up of gamma and delta (γ/δ) chains (the cells being known as gamma-delta T cells).

Rather than recognizing an intact antigen (as with immunoglobulins), T cells are activated by processed peptide fragments in association with an MHC molecule. This is known as MHC restriction. When the TCR recognizes disparities between the donor and recipient MHC, that recognition stimulates T cell proliferation and the potential development of GvHD. In some embodiments, the genes encoding either the TCRα (TRAC), TCRβ (TRBC), TCRγ (TRG), and/or the TCRδ (TCRD) are disrupted or otherwise modified to reduce the tendency of donor T cells to recognize disparities between donor and host MHC, thereby reducing recognition of alloantigen and GvHD. For example, disruption of TRAC can reduce or eliminate TCR expression.

T-cell mediated immunity involves a balance between co-stimulatory and inhibitory signals that serve to fine-tune the immune response. Inhibitory signals, also known as immune checkpoints, allow for avoidance of auto-immunity (e.g., self-tolerance) and also limit immune-mediated damage. Immune checkpoint protein expression is often altered (increased) by tumors, enhancing immune resistance in tumor cells and limiting immunotherapy efficacy. For example, CTLA4 downregulates the amplitude of T cell activation. In contrast, PD1 (in concert with its ligand PD-L1) limits T cell effector functions in peripheral tissue during an inflammatory response and also limits autoimmunity. Immune checkpoint blockade, in several embodiments, helps to overcome barriers to activation of functional cellular immunity. In several embodiments, antagonistic antibodies specific for inhibitory ligands on T cells including Cytotoxic-T-lymphocyte-associated antigen 4 (CTLA-4; also known as CD152) and programmed cell death protein 1 (PD1 or PDCD1 also known as CD279) are used to enhance immunotherapy. In several embodiments, antagonistic antibodies specific for programmed death ligand 1 (PD-L1, also known as B7-H1 or CD274) are used to enhance immunotherapy.

In several embodiments, there is provided genetically modified T cells that are non-alloreactive and highly active. In several embodiments, the T cells are further modified such that certain immune checkpoint genes are inactivated, and the immune checkpoint proteins are thus not expressed by the T cell. In several embodiments, this is done in the absence of manipulation or disruption of the CD3z signaling domain (e.g., the TCRs are still able initiate T cell signaling).

In several embodiments, genetic inactivation of TCRalpha and/or TCRbeta coupled with inactivation of immune checkpoint genes in T lymphocytes derived from an allogeneic donor significantly reduces the risk of GvHD. In several embodiments, this is done by eliminating at least a portion of one or more of the substituent protein chains (alpha, beta, gamma, and/or delta) responsible for recognition of MHC disparities between donor and recipient cells. In some embodiments, TCR expression is disrupted by knocking out TRAC. In several embodiments, this is done while still allowing for T cell proliferation and activity.

In some embodiments wherein allogeneic cells are administered, the receiving subject may receive some other adjunct treatment to support or otherwise enhance the function of the administered immune cells. In several embodiments, the subject may be pre-conditioned (e.g., with radiation or chemotherapy). In some embodiments, the adjunct treatment comprises administration of lymphocyte growth factors (such as IL-2).

Moreover, in several embodiments, editing can improve persistence of administered cells (whether NK cells, T cells, or otherwise) for example, by masking cells to the host immune response. In some cases, a recipient's immune cells will attack donor cells, especially from an allogeneic donor, known as Host vs. Graft disease (HvG). FIG. 8F shows a schematic representation of HvG, where the host T cells, with a native/functional TCR, identify HLA on donor T and/or donor NK cells as non-self. In such cases, the host T-cell TCR binding to allogeneic cell HLA leads to elimination of allogeneic cells, thus reducing the persistence of the donor engineered NK and/or T cells. Regarding HvG, to prevent rejection of administered allogeneic T cells, in some embodiments the subject receiving the cells requires suppression of their immune system. In several embodiments, glucocorticoids are used, and include, but are not limited to beclomethasone, betamethasone, budesonide, cortisone, dexamethasone, hydrocortisone, methylprednisolone, prednisolone, prednisone, triamcinolone, among others. Activation of the glucocorticoid receptor in recipient's own T cells alters expression of genes involved in the immune response and results in reduced levels of cytokine production, which translates to T cell anergy and interference with T cell activation (in the recipient). Other embodiments relate to administration of antibodies that can deplete certain types of the recipient's immune cells. One such target is CD52, which is expressed at high levels on T and B lymphocytes and lower levels on monocytes while being absent on granulocytes and bone marrow precursors. Treatment or pre-treatment of the recipient with Alemtuzumab, a humanized monoclonal antibody directed against CD52, has been shown to induce a rapid depletion of circulating lymphocytes and monocytes, thus lessening the probability of HvG, given the reduction in recipient immune cells. Immunosuppressive drugs may limit the efficacy of administered allogeneic engineered T cells. Therefore, as disclosed herein, several embodiments relate to genetically engineered allogeneic donor cells that are resistant to immunosuppressive treatment. In several embodiments, as discussed in more detail below, immune cells, such as NK cells and/or T cells are edited (in addition to being engineered to express a CAR) to extend their persistence by avoiding cytotoxic responses from host immune cells. In several embodiments, gene editing to remove one or more HLA molecules from the allogeneic NK and/or T cells reduces elimination by host T-cells. In several embodiments, the allogeneic NK and/or T cells are edited to knock out one or more of beta-2 microglobulin (an HLA Class I molecule) and CIITA (an HLA Class II molecule). FIG. 8G schematically depicts this approach.

In some embodiments of mixed (e.g., NK and T) allogeneic cell therapy, the populations of engineered cells actually target one another, for example when the therapeutic cells are edited to remove HLA molecules in order to avoid HvG. Such editing of, for example CAR T cells, can result in the vulnerability of the edited allogeneic CAR T cells to cytotoxic attack by the CAR NK cells as well as elimination by host NK cells. This is caused by the missing “self” inhibitory signals generally presented by KIR molecules. FIG. 8H schematically depicts this process. In several embodiments, gene editing can be used to knock in expression of one or more “masking” molecules which mask the allogeneic cells from the host immune system and from fratricide by other administered engineered cells. FIG. 8I schematically depicts this approach. In several embodiments, proteins can be expressed on the surface of the allogeneic cells to inhibit targeting by NKs (both engineered NKs and host NKs), which advantageously prolongs persistence of both allogeneic CAR-Ts and CAR-NKs. In several embodiments, gene editing is used to knock in CD47, expression of which effectively functions as a “don't eat me” signal. In several embodiments, gene editing is used to knock in expression of HLA-E. In several embodiments, gene editing is used to knock in a portion of HLA-E. HLA-E binds to both the inhibiting and activating receptors NKG2A and NKG2C, respectively, that exist on the surface of NK cells. However, NKG2A is expressed to a greater degree in most human NK cells, thus, in several embodiments, expression of HLA-E on engineered cells results in an inhibitory effect of NK cells (both host and donor) against such cells edited to (or naturally expressing) HLA-E. In addition, in several embodiments, one or more viral HLA homologs are knocked in such that they are expressed by the engineered NK and/or T cells, thus conferring on the cells the ability of viruses to evade the host immune system. In several embodiments, these approaches advantageously prolong persistence of both allogeneic CAR-Ts and CAR-NKs.

### Methods of Genetic Editing

In several embodiments, genetic editing (whether knock out or knock in) of any of the target genes (e.g., CISH, TGFBR2, TRAC, B2M, CIITA, CD47, HLA-E, or any other target gene disclosed herein), is accomplished through targeted introduction of DNA breakage, and a subsequent DNA repair mechanism. In several embodiments, double strand breaks of DNA are repaired by non-homologous end joining (NHEJ), wherein enzymes are used to directly join the DNA ends to one another to repair the break. NHEJ is an error-prone process. In general, in the absence of a repair template, the NHEJ process re-ligates the ends of the cleaved DNA strands, which frequently results in nucleotide deletions and insertions at the cleavage site. In several embodiments, however, double strand breaks are repaired by homology directed repair (HDR), which is advantageously more accurate, thereby allowing sequence specific breaks and repair. HDR uses a homologous sequence as a template for regeneration of missing DNA sequences at the break point, such as a vector with the desired genetic elements (e.g., an insertion element to disrupt the coding sequence of a TCR subunit) within a sequence that is homologous to the flanking sequences of a double strand break. This will result in the desired change (e.g., insertion) being inserted at the site of the DSB. The HDR pathway can occur by way of the canonical HDR pathway or the alternative HDR pathway. Unless otherwise indicated, the term “HDR” or “homology-directed repair” as used herein encompasses both canonical HDR and alternative HDR.

Canonical HDR or “canonical homology-directed repair” or cHDR,” are used interchangeably, and refers to the process of repairing DNA damage using a homologous nucleic acid (e.g., an endogenous homologous sequence, such as a sister chromatid; or an exogenous nucleic acid, such as a donor template). Canonical HDR typically acts when there has been a significant resection at the DSB, forming at least one single-stranded portion of DNA. In a normal cell, canonical HDR typically involves a series of steps such as recognition of the break, stabilization of the break, resection, stabilization of single-stranded DNA, formation of a DNA crossover intermediate, resolution of the crossover intermediate, and ligation. The canonical HDR process requires RAD51 and BRCA2, and the homologous nucleic acid, e.g., repair template, is typically double-stranded. In canonical HDR, a double-stranded polynucleotide, e.g., a double-stranded repair template, is introduced, which comprises a sequence that is homologous to the targeting sequence, and which will either be directly integrated into the targeting sequence or will be used as a template to insert the sequence, or a portion the sequence, of the repair template into the target gene. After resection at the break, repair can progress by different pathways, e.g., by the double Holliday junction model (also referred to as the double strand break repair, or DSBR, pathway), or by the synthesis-dependent strand annealing (SDSA) pathway.

In the double Holliday junction model, strand invasion occurs by the two single stranded overhangs of the targeting sequence to the homologous sequences in the double-stranded polynucleotde, e.g., double stranded donor template, which results in the formation of an intermediate with two Holliday junctions. The junctions migrate as new DNA is synthesized from the ends of the invading strand to fill the gap resulting from the resection. The end of the newly synthesized DNA is ligated to the resected end, and the junctions are resolved, resulting in the insertion at the targeting sequence, or a portion of the targeting sequence that includes the gene variant. Crossover with the polynucleotide, e.g., repair template, may occur upon resolution of the junctions.

In the SDSA pathway, only one single stranded overhang invades the polynucleotide, e.g., donor template, and new DNA is synthesized from the end of the invading strand to fill the gap resulting from resection. The newly synthesized DNA then anneals to the remaining single stranded overhang, new DNA is synthesized to fill in the gap, and the strands are ligated to produce the modified DNA duplex.

Alternative HDR, or “alternative homology-directed repair,” or “alternative HDR,” are used interchangeably, and refers, in some embodiments, to the process of repairing DNA damage using a homologous nucleic acid (e.g., an endogenous homologous sequence, such as a sister chromatid; or an exogenous nucleic acid, such as a repair template). Alternative HDR is distinct from canonical HDR in that the process utilizes different pathways from canonical HDR, and can be inhibited by the canonical HDR mediators, RAD51 and BRCA2. Moreover, alternative HDR is also distinguished by the involvement of a single-stranded or nicked homologous nucleic acid template, e.g., repair template, whereas canonical HDR generally involves a double-stranded homologous template. In the alternative HDR pathway, a single strand template polynucleotide, e.g., repairtemplate, is introduced. A nick, single strand break, or DSB at the cleavage site, for altering a desired target site, e.g., a gene variant in a target gene, is mediated by a nuclease molecule, and resection at the break occurs to reveal single stranded overhangs. Incorporation of the sequence of the template polynucleotide, e.g., repair template, to alter the target site of the DNA typically occurs by the SDSA pathway, as described herein. In some embodiments, HDR is carried out by introducing, into a cell, one or more agent(s) capable of inducing a DSB, and a repair template, e.g., a single-stranded oligonucleotide. The introducing can be carried out by any suitable delivery. The conditions under which HDR is allowed to occur can be any conditions suitable for carrying out HDR in a cell.

In several embodiments, gene editing is accomplished by one or more of a variety of engineered nucleases. In several embodiments, restriction enzymes are used, particularly when double strand breaks are desired at multiple regions. In several embodiments, a bioengineered nuclease is used. Depending on the embodiment, one or more of a Zinc Finger Nuclease (ZFN), transcription-activator like effector nuclease (TALEN), meganuclease and/or clustered regularly interspaced short palindromic repeats (CRISPR/Cas9) system are used to specifically edit the genes encoding one or more of the TCR subunits.

Meganucleases are characterized by their capacity to recognize and cut large DNA sequences (from 14 to 40 base pairs). In several embodiments, a meganuclease from the LAGLIDADG family is used, and is subjected to mutagenesis and screening to generate a meganuclease variant that recognizes a unique sequence(s), such as a specific site in a TCR subunit (e.g., TRAC), or CISH, or any other target gene disclosed herein. Target sites in a TCR subunit can readily be identified. Further information of target sites within a region of a TCR subunit can be found in US Patent Publication No. 2018/0325955, and US Patent Publication No. 2015/0017136, each of which is incorporated by reference herein in its entirety. In several embodiments, two or more meganucleases, or functions fragments thereof, are fused to create a hybrid enzyme that recognizes a desired target sequence within the target gene (e.g., CISH).

In contrast to meganucleases, ZFNs and TALEN function based on a non-specific DNA cutting catalytic domain which is linked to specific DNA sequence recognizing peptides such as zinc fingers or transcription activator-like effectors (TALEs). Advantageously, the ZFNs and TALENs thus allow sequence-independent cleavage of DNA, with a high degree of sequence-specificity in target recognition. Zinc finger motifs naturally function in transcription factors to recognize specific DNA sequences for transcription. The C-terminal part of each finger is responsible for the specific recognition of the DNA sequence. While the sequences recognized by ZFNs are relatively short, (e.g., ˜3 base pairs), in several embodiments, combinations of 2, 3, 4, 5, 6, 7, 8, 9, 10 or more zinc fingers whose recognition sites have been characterized are used, thereby allowing targeting of specific sequences, such as a portion of the TCR (or an immune checkpoints). The combined ZFNs are then fused with the catalytic domain(s) of an endonuclease, such as FokI (optionally a FokI heterodimer), in order to induce a targeted DNA break. Additional information on uses of ZFNs to edit a TCR subunit and/or immune checkpoints can be found in U.S. Pat. No. 9,597,357, which is incorporated by reference herein.

Transcription activator-like effector nucleases (TALENs) are specific DNA-binding proteins that feature an array of 33 or 34-amino acid repeats. Like ZFNs, TALENs are a fusion of a DNA cutting domain of a nuclease to TALE domains, which allow for sequence-independent introduction of double stranded DNA breaks with highly precise target site recognition. TALENs can create double strand breaks at the target site that can be repaired by error-prone non-homologous end-joining (NHEJ), resulting in gene disruptions through the introduction of small insertions or deletions. Advantageously, TALENs are used in several embodiments, at least in part due to their higher specificity in DNA binding, reduced off-target effects, and ease in construction of the DNA-binding domain.

CRISPRs (Clustered Regularly Interspaced Short Palindromic Repeats) are genetic elements that bacteria use as protection against viruses. The repeats are short sequences that originate from viral genomes and have been incorporated into the bacterial genome. Cas (CRISPR associated proteins) process these sequences and cut matching viral DNA sequences. By introducing plasmids containing Cas genes and specifically constructed CRISPRs into eukaryotic cells, the eukaryotic genome can be cut at any desired position. Additional information on CRISPR can be found in US Patent Publication No. 2014/0068797, which is incorporated by reference herein.

In several embodiments, CRISPR is used to manipulate the gene(s) encoding a target gene to be knocked out or knocked in, for example CISH, TGFBR2, TRAC, B2M, CIITA, CD47, HLA-E, etc. In several embodiments, CRISPR is used to edit one or more of the TCRs of a T cell and/or the genes encoding one or more immune checkpoints. In several embodiments, CRISPR is used to edit a gene encoding an immune checkpoint. In several embodiments, the immune checkpoint is selected from one or more of CTLA4 and PD1. In several embodiments, the immune checkpoint comprises CTLA4. In some embodiments, the immune checkpoint comprises PD-1. In some embodiments, the immune checkpoint comprises PD-L1. In several embodiments, CRISPR is used to edit a gene encoding a TCR subunit. In several embodiments, CRISPR is used to edit TRAC. In several embodiments, CRISPR is used to edit TRBC. In several embodiments, CRISPR is used to truncate one or more of TCRα, TCRβ, TCRγ, and TCRδ. In several embodiments, a TCR is truncated without impacting the function of the CD3z signaling domain of the TCR.

Depending on the embodiment and which target gene is to be edited, a Class 1 or Class 2 Cas is used. In several embodiments, a Class 1 Cas is used and the Cas type is selected from the following types: I, IA, IB, IC, ID, IE, IF, IU, III, IIIA, IIIB, IIIC, HID, IV IVA, IVB, and combinations thereof. In several embodiments, the Cas is selected from the group consisting of Cas3, Cas8a, Cas5, Cas8b, Cas8c, Cas10d, Cse1, Cse2, Csy1, Csy2, Csy3, GSU0054, Cas10, Csm2, Cmr5, Cas10, Csx11, Csx10, Csf1, and combinations thereof. In several embodiments, a Class 2 Cas is used and the Cas type is selected from the following types: II, IIA, IIB, IIC, V, VI, and combinations thereof. In several embodiments, the Cas is selected from the group consisting of Cas9, Csn2, Cas4, Cas12a (previously known as Cpf1), C2c1, C2c3, Cas13a (previously known as C2c2), Cas13b, Cas13c, CasX, CasY and combinations thereof. In some embodiments, the Cas is Cas9. In some embodiments, class 2 CasX is used, wherein CasX is capable of forming a complex with a guide nucleic acid and wherein the complex can bind to a target DNA, and wherein the target DNA comprises a non-target strand and a target strand. In some embodiments, class 2 CasY is used, wherein CasY is capable of binding and modifying a target nucleic acid and/or a polypeptide associated with target nucleic acid.

In several embodiments, as discussed above, editing of CISH advantageously imparts to the edited cells, particularly edited NK cells, enhanced expansion, cytotoxicity and/or persistence. Additionally, in several embodiments, the modification of the TCR comprises a modification to TCRα, but without impacting the signaling through the CD3 complex, allowing for T cell proliferation. In one embodiment, the TCRα is inactivated by expression of pre-Tα in the cells, thus restoring a functional CD3 complex in the absence of a functional alpha/beta TCR. As disclosed herein, the non-alloreactive modified T cells are also engineered to express a CAR to redirect the non-alloreactive T cells specificity towards tumor marker, but independent of MHC. Provided herein are are immune cells (e.g., NK or T cells) comprising any combination of genetic edits as described herein. Combinations of editing are used in several embodiments, such as knockout of the TCR (e.g., TRAC) and CISH in combination, or knock out of CISH and knock in of CD47, by way of non-limiting examples. In some embodiments, the genetic edits comprise edits to genes encoding TRAC and CISH. In some embodiments, the genetic edits comprise edits to genes encoding CISH and CD47.

### Reduction in MHC I or MHC II Expression

In several embodiments, immune cells are genetically edited to express non-native (e.g., reduced) levels of major histocompatibility complex class 1 molecules on the surface of the cells. As discussed elsewhere herein, other approaches result in elimination of the surface expression of MHC I molecules (e.g., a B2M knockout or a disruption/knockout of another gene encoding one or more portions of the MHC I complex).

Alternatively, in several embodiments, methods are used to reduce the frequency of expression of the MHC I complex within a population of cells, wherein each member of the population expresses some amount of MHC I molecules, but the average frequency of MHC I expression for individual cells within the population is reduced as compared to natural immune cells. In several embodiments, this approach allows a dual beneficial effect to be achieved, in that (i) the reduced frequency of MHC I expression serves to reduce the chances of host T cells attacking the engineered cells and (ii) the retention of some degree of MHC I expression (e.g., non-zero surface expression across the population or a portion of the population) reduces the chances of host NK cells eliminating the engineered cells as well as reducing the chances of fratricide within the engineered cells. In this manner, embodiments provided for herein enhance the persistence of engineered cells (NK cells, T cells, or a mixed NK/T population) for use in allogeneic therapy.

In several embodiments discussed elsewhere here, genetic edits to knockout expression of the B2M gene are provided for, which eliminates MHC I molecule expression on the cell surface. As an alternative, in several embodiments, B2M expression is reduced, but not eliminated by RNA interference. In several embodiments, microRNAs are used to target and reduce B2M expression. In several embodiments, small interfering RNAs are used to target and reduce B2M expression. In several embodiments, combinations of microRNAs and siRNAs are used. As a result of reducing the RNA encoding B2M, the reduction of B2M expression can advantageously be transient in nature.

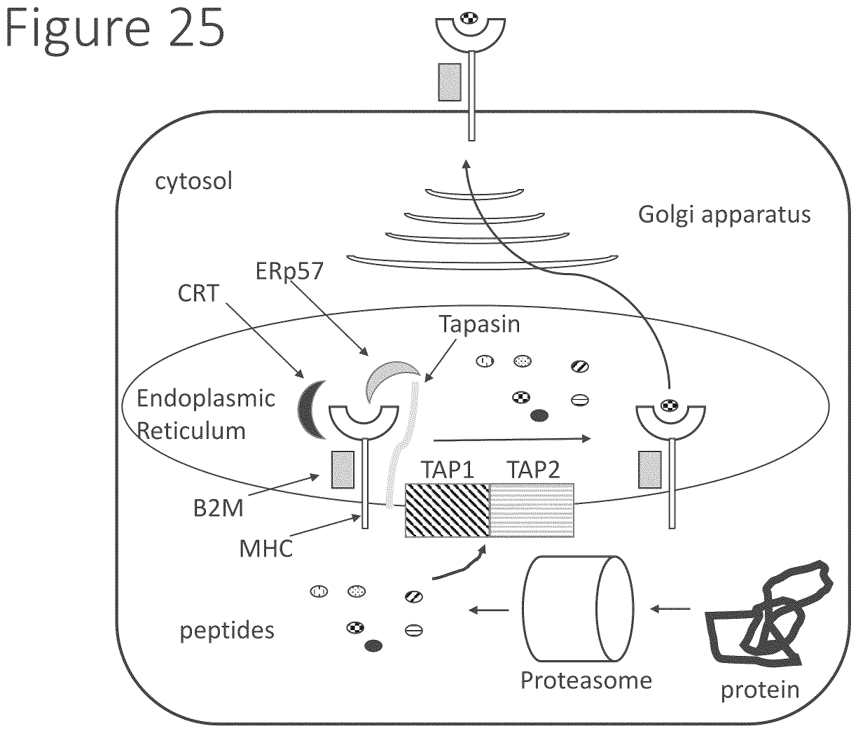

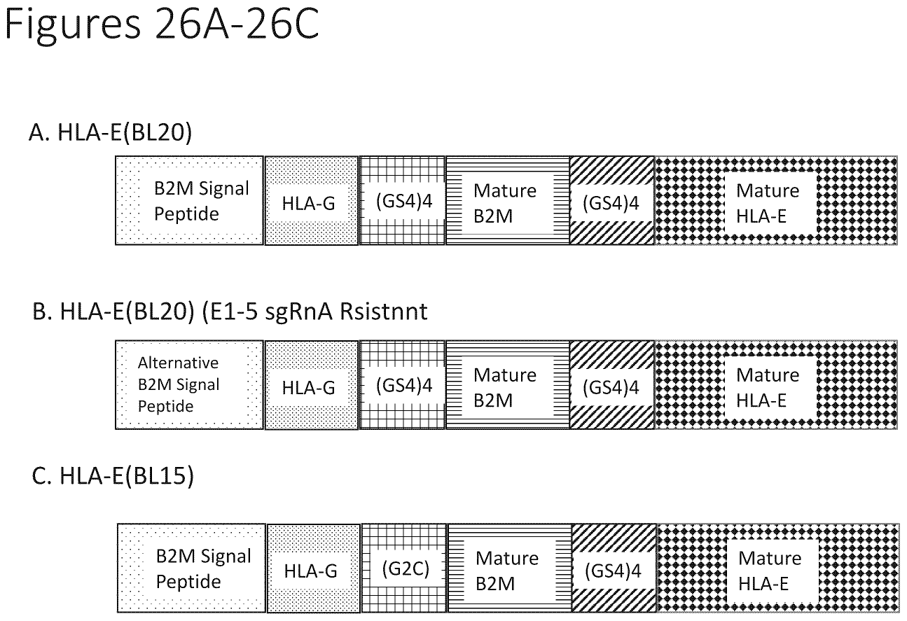

In additional embodiments, gene editing can be used to reduce the expression of one or more genes that encode proteins that are involved in antigen processing/MHC I assembly. FIG. 25 schematically depicts non-limiting embodiments of target proteins involved in this pathway, as does Example 1, below. A protein that is present in an immune cell can be processed by the proteasome to generate a population of small peptides. Two proteins, TAP1 and TAP2, function to translocate the peptides into the endoplasmic reticulum, where MHC I molecules are assembled. Calreticulin (CRT) is a calcium-binding chaperone that, in the ER, facilitates folding of MHC I molecules along with the related MHC I recruitment/assembly factor, Tapasin. The thiol oxidoreductase ERp57 has been found to interact with Tapasin, and in its absence, the recruitment of MHC class I molecules into this complex by Tapasin significantly reduced. In addition to Tapasin, an additional intracellular peptide editor TABPR (also called TABPL; not shown in FIG. 25) functions to select/exchange peptides for attachment to MHC I. Acting as a quality control protein, UDP-glucose:glycoprotein glucosyltransferase 1 (UGT1, also known as UGTA-1) recognizes misfolded proteins/peptides due to lack of glycosylation and re-glycosylates them, allowing proper folding. It has been determined that peptide formation still occurs in the absence of UGT-1 (there are redundancies in the folding process), surface level of MHC I is reduced, maturation and assembly are delayed, and peptide selection is impaired. After being loaded with an appropriately processed peptide, the MHC I/B2M/peptide molecule is trafficked through the Golgi apparatus and expressed on the surface of the cell. Thus, any of the genes involved in this processing/assembly/trafficking pathway are viable targets to reduce MHC I expression frequency. In some embodiments, one or more of UGT-1 (UGTA-1); TAPBPL (TAPBPR); TAPBP (Tapasin); TAP-1; TAP-2; ERp57; Calreticulin (CRT); Endoplasmic reticulum aminopeptidases (ERAP1, ERAP2) and/or immunoproteasome components are targeted for editing, for example using CRISPR. Further, the knockout of antigen presentation pathway genes may also result in altered presentation of antigen peptides, resulting in reduced recognition by T-cells, while still advantageously limiting the inhibitory function of NKs.

In some embodiments, in place of editing antigen presentation genes or in addition to such edits, various viral peptides are used to further reduce the frequency of MHC I expression. For example, herpesviruses aim to evade recognition and elimination by host cytotoxic T cells by expressing genes which interfere selectively with presentation of viral antigens by MHC I molecules. In several embodiments, one or more antigens from herpes simplex virus ICP47, human cytomegalovirus (HCMV) US3, or HCMV US2, US3, US6, US10 and/or US11 are expressed by engineered immune cells provided for herein in order to decrease presentation of viral proteins and help such cells avoid a host immune response.

Further examples of proteins that interfere with the MHC class I pathway are encoded by adenoviruses and retroviruses. Two non-limiting examples are the adenovirus E3/19K and the human immunodeficiency virus-1 (HIV-1) Nef gene products. In several embodiments, one or more antigens from adenovirus E3/19K and/or HIV-1 Nef are expressed by engineered immune cells provided for herein in order to decrease presentation of viral proteins and help such cells avoid a host immune response. Combinations of adenovirus, HCMV, HSV, and/or HIV genes are expressed in some embodiments to further enhance the ability of such engineered cells to evade host immune responses.

In several embodiments, the editing to reduce the frequency of MHC I expression by immune cells is used in combination with expression of viral peptides or other immune-suppressive proteins or peptides discussed herein (e.g., HLA-E, CD47, viral proteins/peptides, etc.).

In some embodiments, the immune cells are genetically edited to express reduced levels of MHC class II molecules on the surface of the cells. Major histocompatibility complex (MHC) class II transactivator (CIITA) is a master regulator of MHC class II gene expression, and also controls IFNgamma-induced MHC-II expression (Alfonso et al., Int. J. Mol. Sci, (2021) 22 (3): 1074). Inhibition of MHC class II, including for the purpose of evading host immune response, can therefore be achieved by disrupting expression of CIITA. In several embodiments, immune cells are genetically edited to reduce (e.g., knockout) expression of CIITA. In several embodiments, T cells are genetically edited to reduce (e.g., knockout) expression of CIITA, thereby reducing allogenic HLA-II-mediated immunogenicity.

Provided herein are are immune cells (e.g., NK or T cells) comprising any combination of genetic edits as described herein. Combinations of editing are used in several embodiments, such as knockout of B2M and CIITA.

### Engineering Enhanced Persistence Through Immunosuppressive Effector(s)

Additional cellular editing strategies are provided for herein that serve to further enhance the persistence of allogeneic cellular therapy products, such as allogeneic CAR-T cells and/or allogeneic CAR-NK cells. As discussed herein, there are various strategies that can be employed to reduce the tendency of an allogeneic cell therapy product to induce host cell-mediated graft rejection.

For example, in several embodiments the expression of B2M (encoded by B2M) is reduced and/or eliminated in order to reduce the host-mediated graft rejection. In several embodiments, B2M expression is disrupted and/or knocked out using a Crispr-Cas mediated approach (e.g., Cas9) as disclosed elsewhere herein, but with the use of one more of the following B2M-specific guide RNAs: SEQ ID 290-CGCGAGCACAGCTAAGGCCA; SEQ ID 291-GAGTAGCGCGAGCACAGCTA; SEQ ID 292-GCTACTCTCTCTTTCTGGCC; SEQ ID 293-GGCCGAGATGTCTCGCTCCG; SEQ ID 294-GGCCACGGAGCGAGACATCT; SEQ ID 295-CACAGCCCAAGATAGTTAAG; SEQ ID 296-AGTCACATGGTTCACACGGC; SEQ ID 297-AAGTCAACTTCAATGTCGGA; SEQ ID 298-ACTTGTCTTTCAGCAAGGAC; and SEQ ID 299-TGGGCTGTGACAAAGTCACA.

Loss of expression of B2M induces a complete loss of HLA expression, which can reduce, and in some embodiments, eliminate, the host T-cell mediated graft rejection. However, this can also render the administered cells susceptible to host NK-cell mediated graft rejection (as well as to rejection by administered engineered NK cells, when a mixed NK/T cell population is used). This, as discussed above, results in loss of the KIR inhibitory signals (e.g., “missing self” signals). See FIGS. 8E-8I.

In some embodiments various proteins can be expressed on the surface of a cell to be administered to an allogeneic recipient in order to inhibit host (or administered) NK cells from targeting the administered NK and or T cells. In several embodiments, approaches involve the expression of, for example HLA-E/HLA-G expression, CD47, or one or more viral peptide/proteins, and combinations of these (among other disclosed herein).

In several embodiments, the expression of ADORA2A (Adenosine 2a Receptor; encoded by ADORA2) is reduced and/or eliminated in order to increase overall activation in resultant T cells and/or NK cells, or other cell type provided for herein. In several embodiments, ADORA2A is disrupted and/or knocked out using one or more of the gene editing methods disclosed herein. In several embodiments, ADORA2A is disrupted and/or knocked out using a Crispr-Cas mediated approach (e.g., Cas9) as disclosed elsewhere herein, with the Cas nuclease guided by the use of one more of the following ADORA2A-specific guide RNAs: SEQ ID NO: 503-506.

In several embodiments, gene editing reduces transcription of ADORA2A by about 30%, about 40%, about 50%, about 60%, about 70%, about 75%, about 80%, about 85%, about 90%, about 95%, about 97%, about 98%, about 99%, or more (including any amount between those listedIn several embodiments, gene editing reduces transcription of ADORA2A by at least about 30%, In several embodiments, gene editing reduces transcription of ADORA2 by at least about 40%, In several embodiments, gene editing reduces transcription of ADORA2 by at least about 50%, In several embodiments, gene editing reduces transcription of ADORA2 by at least about 60%, In several embodiments, gene editing reduces transcription of ADORA2 by at least about 70%, In several embodiments, gene editing reduces transcription of ADORA2 by at least about 80%, In several embodiments, gene editing reduces transcription of ADORA2 by at least about 90%,

In several embodiments, gene editing can reduce expression of a target protein by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of ADORA2A by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of ADORA2A by at least about 30%, In several embodiments, gene editing reduces expression of ADORA2A by at least about 40%, In several embodiments, gene editing reduces expression of ADORA2A by at least about 50%, In several embodiments, gene editing reduces expression of ADORA2A by at least about 60%, In several embodiments, gene editing reduces expression of ADORA2A by at least about 70%, In several embodiments, gene editing reduces expression of ADORA2A by at least about 80%, In several embodiments, gene editing reduces expression of ADORA2A by at least about 90%,

Loss of expression of ADORA2A induces decreased sensitivity to adenosine, a well-established immunosuppressant for T cells and NK cells (Young et al., Cancer Res. (2018) 78(4):1003-16; Cekic and Linden, Cancer Res. (2014) 74(24):7239-49). Thus, according to several embodiments, gene editing ADORA2A increases the cytotoxicity, persistence, immune avoidance or otherwise enhances the efficacy of engineered NK, T, or other cell as disclosed herein.

The tumor microenvironment (TME), as suggested with the nomenclature, is the environment around a tumor, which includes the surrounding blood vessels and capillaries, immune cells circulating through or retained in the area, fibroblasts, various signaling molecules related by the tumor cells, the immune cells or other cells in the area, as well as the surrounding extracellular matrix. Various mechanisms are employed by tumors to evade detection and/or destruction by host immune cells, including modification of the TME. Tumors may alter the TME by releasing extracellular signals, promoting tumor angiogenesis or even inducing immune tolerance, in part by limiting immune cell entry in the TME and/or limiting reproduction/expansion of immune cells in the TME. The tumor can also modify the extracellular matrix (ECM), which can allow pathways to develop for tumor extravasation to new sites. Transforming Growth-Factor beta (TGFb) has beneficial effects when reducing inflammation and preventing autoimmunity. However, it can also function to inhibit anti-tumor immune responses, and thus, upregulated expression of TGFb has been implicated in tumor progression and metastasis (Pickup et al., Nat. Rev. Cancer (2013) 13 (11): 788-99). TGFb signaling can inhibit the cytotoxic function of NK cells by interacting with the TGFb receptor expressed by NK cells, for example the TGFb receptor isoform II (TGFBR2), encoded by TGFBR2. In accordance with several embodiments disclosed herein, the reduction or elimination of expression of TGFBR2 through gene editing (e.g., by CRISPr/Cas9 guided by a TGFBR2 guide RNA) interrupts the inhibitory effect of TGFb on NK cells.

In NK cells, TGFBR2 is a potent checkpoint in NK cell-mediated tumor immunity, while for T cells, knockout of TGFBR2 rescues CAR T cell exhaustion induced by TGF-β1 (Tang et al., JCI Insight (2020) 5 (4): e133977). Thus, according to several embodiments, gene editing TGFBR2 increases the cytotoxicity, persistence, immune avoidance or otherwise enhances the efficacy of engineered NK, T, or other cell as disclosed herein.

As discussed herein, the CRISPR/Cas9 system may be used to specifically target and reduce the expression of the TGFBR2 by NK cells. Various non-limiting examples of guide RNAs are summarized below.

TABLE 1
TGFb Receptor Type 2 Isoform Guide RNAs

| SEQ | | | |
|---|---|---|---|
| ID NO: | Name | Sequence | Target |
| 147 | TGFBR2-1 | CCCCTACCATGACTTTATTC | Exon 4 |
| 148 | TGFBR2-2 | ATTGCACTCATCAGAGCTAC | Exon 4 |
| 149 | TGFBR2-3 | AGTCATGGTAGGGGAGCTTG | Exon 4 |
| 150 | TGFBR2-4 | TGCTGGCGATACGCGTCCAC | Exon 1 |
| 151 | TGFBR2-5 | GTGAGCAATCCCCCGGGCGA | Exon 4 |
| 152 | TGFBR2-6 | AACGTGCGGTGGGATCGTGC | Exon 1 |

In several embodiments, the expression of TGFBR2 (encoded by TGFBR2) is reduced and/or eliminated in order to increase overall activation in resultant T cells and/or NK cells, or other cell type provided for herein. In several embodiments, TGFBR2 is disrupted and/or knocked out using one or more of the gene editing methods disclosed herein. In several embodiments, TGFBR2 is disrupted and/or knocked out using a Crispr-Cas mediated approach (e.g., Cas9) as disclosed elsewhere herein, with the Cas nuclease guided by the use of one more of the following TGFBR2-specific guide RNAs: SEQ ID NO: 544-547: SEQ ID 544-TGGGCAGTCCTATTACAGCT; SEQ ID 545-ATGATAGTCACTGACAACAA; SEQ ID 546-AGTTGCTCATGCAGGATTTC; SEQ ID NO 547-GAAGCCACAGGAAGTCTGTG.

In several embodiments, gene editing reduces transcription of TGFBR2 by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces transcription of TGFBR2 by at least about 30%, In several embodiments, gene editing reduces transcription of TGFBR2 by at least about 40%, In several embodiments, gene editing reduces transcription of TGFBR2 by at least about 50%, In several embodiments, gene editing reduces transcription of TGFBR2 by at least about 60%, In several embodiments, gene editing reduces transcription of TGFBR2 by at least about 70%, In several embodiments, gene editing reduces transcription of TGFBR2 by at least about 80%, In several embodiments, gene editing reduces transcription of TGFBR2 by at least about 90%.

In several embodiments, gene editing can reduce expression of a target protein by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of TGFBR2 by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of TGFBR2 by at least about 30%, In several embodiments, gene editing reduces expression of TGFBR2 by at least about 40%, In several embodiments, gene editing reduces expression of TGFBR2 by at least about 50%, In several embodiments, gene editing reduces expression of TGFBR2 by at least about 60%, In several embodiments, gene editing reduces expression of TGFBR2 by at least about 70%, In several embodiments, gene editing reduces expression of TGFBR2 by at least about 80%, In several embodiments, gene editing reduces expression of TGFBR2 by at least about 90%.

In accordance with additional embodiments, a disruption of, or elimination of, expression of a receptor, pathway or protein on an immune cell can result in the enhanced activity (e.g., cytotoxicity, persistence, etc.) of the immune cell against a target cancer cell. In several embodiments, this results from a disinhibition of the immune cell. Natural killer cells, express a variety of receptors, such particularly those within the Natural Killer Group 2 family of receptors. One such receptor, according to several embodiments disclosed herein, the NKG2D receptor, is used to generate cytotoxic signaling constructs that are expressed by NK cells and lead to enhanced anti-cancer activity of such NK cells. In addition, NK cells express the NKG2A receptor, which is an inhibitory receptor. One mechanism by which tumors develop resistance to immune cells is through the expression of peptide-loaded HLA Class I molecules (HLA-E), which suppresses the activity of NK cells through the ligation of the HLA-E with the NKG2A receptor. Thus, while one approach could be to block the interaction of the HLA-E with the expressed NKG2A receptors on NK cells, according to several embodiments disclosed herein, the expression of NKG2A is disrupted, which short circuits that inhibitory pathway and allows enhanced NK cell cytotoxicity.

Non-limiting examples of guide RNAs targeting NKG2A are shown below in Table 2.

TABLE 2
NKG2A Guide RNAs

| SEQ | | | |
|---|---|---|---|
| ID NO: | Name | Sequence | Target |
| 158 | NKG2A-1 | GGAGCTGATGGTAAATCTGC | Exon 4 |
| 159 | NKG2A-2 | TTGAAGGTTTAATTCCGCAT | Exon 3 |
| 160 | NKG2A-3 | AACAACTATCGTTACCACAG | Exon 4 |

In several embodiments, the expression of NKG2A (encoded by NKG2A also known as KLRC1) is reduced and/or eliminated in order to increase overall activation in resultant T cells and/or NK cells, or other cell type provided for herein. In several embodiments, NKG2A is disrupted and/or knocked out using one or more of the gene editing methods disclosed herein. In several embodiments, NKG2A is disrupted and/or knocked out using a Crispr-Cas mediated approach (e.g., Cas9) as disclosed elsewhere herein, with the Cas nuclease guided by the use of one more of the following NKG2A-specific guide RNAs: SEQ ID NO: 548-551: SEQ ID 548-GAAGCTCATTGTTGGGATCC; SEQ ID 549-AACAACTATCGTTACCACAG; SEQ ID NO 550-TGAACAGGAAATAACCTATG; SEQ ID NO 551-GGTTTTCGTTGCTGCCTCTT.

In several embodiments, gene editing reduces transcription of NKG2A by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces transcription of NKG2A by at least about 30%. In several embodiments, gene editing reduces transcription of NKG2A by at least about 40%. In several embodiments, gene editing reduces transcription of NKG2A by at least about 50%.

In several embodiments, gene editing reduces transcription of NKG2A by at least about 60%. In several embodiments, gene editing reduces transcription of NKG2A by at least about 70%. In several embodiments, gene editing reduces transcription of NKG2A by at least about 80%. In several embodiments, gene editing reduces transcription of NKG2A by at least about 90%.

In several embodiments, gene editing can reduce expression of a target protein by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of NKG2A by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of NKG2A by at least about 30%. In several embodiments, gene editing reduces expression of NKG2A by at least about 40%. In several embodiments, gene editing reduces expression of NKG2A by at least about 50%. In several embodiments, gene editing reduces expression of NKG2A by at least about 60%. In several embodiments, gene editing reduces expression of NKG2A by at least about 70%. In several embodiments, gene editing reduces expression of NKG2A by at least about 80%. In several embodiments, gene editing reduces expression of NKG2A by at least about 90%.

NKG2A binds to HLA-E and is recognized as an MHC-recognizing receptor. Since NKG2A is an inhibitor receptor, loss of expression of NKG2A induces increased activation of constituent cells. In NK and T cells, loss of NKG2A leads to increased activation and cytotoxicity against HLA-E expressing tumor cells (Kamiya et al., J. Clin. Invest. (2019) 129 (5): 2094-2106). Thus, according to several embodiments, gene editing NKG2A increases the cytotoxicity, persistence, immune avoidance or otherwise enhances the efficacy of engineered NK, T, or other cell as disclosed herein.

Interleukins, in particular interleukin-15, are important in NK cell function and survival. Suppressor of cytokine signaling (SOCS) proteins are negative regulators of cytokine release by NK cells. The protein tyrosine phosphatase CD45 is an important regulator of NK cell activity through Src-family kinase activity. CD45 expression is involved in ITAM-specific NK-cell functions and processes such as degranulation, cytokine production, and expansion (Hesslein et al., Blood (2011) 117(11):3087-95). Thus, knockout of CD45 expression should result in less effective NK cells. As discussed above, CRISPR/Cas9 was used to disrupt expression of CD45 (encoded by PTPRC) and SOCS2 (encoded by SOCS2), though in additional embodiments, other gene editing approaches can be used. Non-limiting examples of PTPRC and SOCS2-targeting guide RNAs are shown below in Table 3.

TABLE 3
PTPRC and SOCS2 Guide RNAs

| SEQ ID NO: | Name | Sequence | Target |
|---|---|---|---|
| 170 | PTPRC-1 | AGTGCTGGTGTTGGGCGCAC | Exon 25 |
| 171 | SOCS2-1 | GTGAACAGTGCCGTTCCGGGGGG | Exon 3 |
| 172 | SOCS2-2 | GGCACCGGTACATTTGTTAATGG | Exon 3 |
| 173 | SOCS2-3 | TTCGCCAGACGCGCCGCCTGCGG | Exon 2 |

In several embodiments, gene editing reduces transcription of PTPRC by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces transcription of PTPRC by at least about 30%. In several embodiments, gene editing reduces transcription of PTPRC by at least about 40%. In several embodiments, gene editing reduces transcription of PTPRC by at least about 50%. In several embodiments, gene editing reduces transcription of PTPRC by at least about 60%. In several embodiments, gene editing reduces transcription of PTPRC by at least about 70%. In several embodiments, gene editing reduces transcription of PTPRC by at least about 80%. In several embodiments, gene editing reduces transcription of PTPRC by at least about 90%.

In several embodiments, gene editing can reduce expression of a target protein by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of CD45 by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of CD45 by at least about 30%. In several embodiments, gene editing reduces expression of CD45 by at least about 40%. In several embodiments, gene editing reduces expression of CD45 by at least about 50%. In several embodiments, gene editing reduces expression of CD45 by at least about 60%. In several embodiments, gene editing reduces expression of CD45 by at least about 70%. In several embodiments, gene editing reduces expression of CD45 by at least about 80%. In several embodiments, gene editing reduces expression of CD45 by at least about 90%.

In several embodiments, the expression of Cytokine Signaling 2 (SOCS2) is reduced and/or eliminated in order to increase overall activation in resultant T cells and/or NK cells, or other cell type provided for herein. In several embodiments, SOCS2 is disrupted and/or knocked out using one or more of the gene editing methods disclosed herein. In several embodiments, SOCS2 is disrupted and/or knocked out using a Crispr-Cas mediated approach (e.g., Cas9) as disclosed elsewhere herein, with the Cas nuclease guided by the use of one more of the following SOCS2-specific guide RNAs: SEQ ID NO: 556-561: SEQ ID NO 556-CTTCGAATCGAATACCAAGA; SEQ ID NO 557-GCTTAAACAATTTGACAGTG; SEQ ID NO 558-CCAAGACGGAAAATTCAGAT; SEQ ID NO 559-CCAATCTGAATTTTCCGTCT; SEQ ID NO 560-CGGTCCAGCTGACGTCTTAA; SEQ ID NO 561-CATCTTGGTACTCAATCCGC.

In several embodiments, gene editing reduces transcription of SOCS2 by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces transcription of SOCS2 by at least about 30%. In several embodiments, gene editing reduces transcription of SOCS2 by at least about 40%. In several embodiments, gene editing reduces transcription of SOCS2 by at least about 50%. In several embodiments, gene editing reduces transcription of SOCS2 by at least about 60%. In several embodiments, gene editing reduces transcription of SOCS2 by at least about 70%. In several embodiments, gene editing reduces transcription of SOCS2 by at least about 80%. In several embodiments, gene editing reduces transcription of SOCS2 by at least about 90%.

In several embodiments, gene editing can reduce expression of a target protein by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of SOCS2 by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of SOCS2 by at least about 30%. In several embodiments, gene editing reduces expression of SOCS2 by at least about 40%. In several embodiments, gene editing reduces expression of SOCS2 by at least about 50%. In several embodiments, gene editing reduces expression of SOCS2 by at least about 60%. In several embodiments, gene editing reduces expression of SOCS2 by at least about 70%. In several embodiments, gene editing reduces expression of SOCS2 by at least about 80%. In several embodiments, gene editing reduces expression of SOCS2 by at least about 90%.

SOCS proteins are negative regulators of cytokine responses, and SOCS2 specifically negatively regulates the development of NK cells through inhibiting JAK2 activity. Loss of expression of SOCS2 in NK cells induces increased NK cell development and overall cytotoxicity (Kim et al., Scientific Reports (2017) 7:46153). Thus, according to several embodiments, gene editing SOCS2 increases the cytotoxicity, persistence, immune avoidance or otherwise enhances the efficacy of engineered NK, T, or other cell as disclosed herein.

In several embodiments, the expression of Casitas B-lineage lymphoma-b (Cbl-b; encoded by CBLB) is reduced and/or eliminated in order to increase overall activation in resultant T cells and/or NK cells, or other cell type provided for herein. In several embodiments, Cbl-b is disrupted and/or knocked out using one or more of the gene editing methods disclosed herein. In several embodiments, CBLB is disrupted and/or knocked out using a Crispr-Cas mediated approach (e.g., Cas9) as disclosed elsewhere herein, with the Cas nuclease guided by the use of one more of the following CBLB-specific guide RNAs: In several embodiments, the expression of Casitas B-lineage lymphoma-b (Cbl-b) is reduced and/or eliminated in order to increase overall activation in resultant T cells and NK cells. In several embodiments, CBLB is disrupted and/or knocked out using a Crispr-Cas mediated approach (e.g., Cas9) as disclosed elsewhere herein, but with the use of one more of the following Cbl-b-specific guide RNAs: SEQ ID NO: 552-555: SEQ ID 552-TCCCCGAAAAGGTCGAATTT; SEQ ID 553-ATCTGCGGCAGCTTGCTTAG; SEQ ID 554-GGGTATTATTGATGCTATTC; SEQ ID 555-GATTTCCTCCTCGACCACCA.

Further non-limiting examples of CBLB-targeting guide RNAs are shown below in Table 4.

TABLE 4
CBLB Guide RNAs

| SEQ | | | |
|---|---|---|---|
| ID NO: | Name | Sequence | Target |
| 164 | CBLB-1 | TAATCTGGTGGACCTCATGAAGG | Exon 5 |
| 165 | CBLB-2 | TCGGTTGGCAAACGTCCGAAAGG | Exon 10 |
| 166 | CBLB-3 | AGCAAGCTGCCGCAGATCGCAGG | Exon 2 |

In several embodiments, gene editing reduces transcription of CBLB by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces transcription of CBLB by at least about 30%. In several embodiments, gene editing reduces transcription of CBLB by at least about 40%. In several embodiments, gene editing reduces transcription of CBLB by at least about 50%. In several embodiments, gene editing reduces transcription of CBLB by at least about 60%. In several embodiments, gene editing reduces transcription of CBLB by at least about 70%. In several embodiments, gene editing reduces transcription of CBLB by at least about 80%. In several embodiments, gene editing reduces transcription of CBLB by at least about 90%.

In several embodiments, gene editing can reduce expression of a target protein (e.g., Cbl-b) by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of Cbl-b by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of Cbl-b by at least about 30%. In several embodiments, gene editing reduces expression of Cbl-b by at least about 40%. In several embodiments, gene editing reduces expression of Cbl-b by at least about 50%. In several embodiments, gene editing reduces expression of Cbl-b by at least about 60%. In several embodiments, gene editing reduces expression of Cbl-b by at least about 70%. In several embodiments, gene editing reduces expression of Cbl-b by at least about 80%. In several embodiments, gene editing reduces expression of Cbl-b by at least about 90%.

Cbl-b is an E3 ubiquitin ligase that negatively regulates T cell activation Loss of expression of Cbl-b in NK cells and T cells demonstrate increased antitumor immunity. Moreover, Cbl-b deficient T cells and NK cells are resistant to PD-L1/PD-1 mediated suppression (Fujiwara et al., Front. Immunol. (2017) 8: 42). Thus, according to several embodiments, gene editing Cbl-b increases the cytotoxicity, persistence, immune avoidance or otherwise enhances the efficacy of engineered NK, T, or other cell as disclosed herein.

Another E3 ubiquitin ligase, TRIpartite Motif-containing protein 29 (TRIM29; encoded by TRIM29), is a negative regulator of NK cell functions (Dou et al., J. Immunol. (2019) 203(4):873-80). TRIM29 is generally not expressed by resting NK cells, but is readily upregulated following activation (in particular by IL-12/IL-18 stimulation). As discussed herein, CRISPR/Cas9 can also be used to disrupt expression of TRIM29, though in additional embodiments, other gene editing approaches can be used. Non-limiting examples of TRIM29-targeting guide RNAs are shown below in Table 5.

TABLE 5
TRIM29 Guide RNAs

| SEQ | | | |
|---|---|---|---|
| ID NO: | Name | Sequence | Target |
| 167 | TRIM29-1 | GAACGGTAGGTCCCCTCTCGTGG | Exon 4 |
| 168 | TRIM29-2 | AGCTGCCTTGGACGACGGGCAGG | Exon 7 |
| 169 | TRIM29-3 | TGAGCCGTAACTTCATTGAGAGG | Exon 4 |

In several embodiments, gene editing reduces transcription of TRIM29 by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces transcription of TRIM29 by at least about 30%. In several embodiments, gene editing reduces transcription of TRIM29 by at least about 40%. In several embodiments, gene editing reduces transcription of TRIM29 by at least about 50%. In several embodiments, gene editing reduces transcription of TRIM29 by at least about 60%. In several embodiments, gene editing reduces transcription of TRIM29 by at least about 70%. In several embodiments, gene editing reduces transcription of TRIM29 by at least about 80%. In several embodiments, gene editing reduces transcription of TRIM29 by at least about 90%.

In several embodiments, gene editing can reduce expression of a target protein (e.g., TRIM29) by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of TRIM29 by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of TRIM29 by at least about 30%. In several embodiments, gene editing reduces expression of TRIM29 by at least about 40%. In several embodiments, gene editing reduces expression of TRIM29 by at least about 50%. In several embodiments, gene editing reduces expression of TRIM29 by at least about 60%. In several embodiments, gene editing reduces expression of TRIM29 by at least about 70%. In several embodiments, gene editing reduces expression of TRIM29 by at least about 80%. In several embodiments, gene editing reduces expression of TRIM29 by at least about 90%.

In several embodiments, the expression of Beta-2 Microglobulin (B2-microglobulin; encoded by B2M) is reduced and/or eliminated in order to increase overall activation in resultant T cells and/or NK cells, or other cell type provided for herein. In several embodiments, B2M is disrupted and/or knocked out using one or more of the gene editing methods disclosed herein. In several embodiments, B2M is disrupted and/or knocked out using a Crispr-Cas mediated approach (e.g., Cas9) as disclosed elsewhere herein, with the Cas nuclease guided by the use of one more of the following B2M-specific guide RNAs: SEQ ID NO: 290-299: SEQ ID 290-CGCGAGCACAGCTAAGGCCA; SEQ ID 291-GAGTAGCGCGAGCACAGCTA; SEQ ID 292-GCTACTCTCTCTTTCTGGCC; SEQ ID 293-GGCCGAGATGTCTCGCTCCG; SEQ ID 294-GGCCACGGAGCGAGACATCT; SEQ ID 295-CACAGCCCAAGATAGTTAAG; SEQ ID 296-AGTCACATGGTTCACACGGC; SEQ ID 297-AAGTCAACTTCAATGTCGGA; SEQ ID 298-ACTTGTCTTTCAGCAAGGAC; SEQ ID 299-TGGGCTGTGACAAAGTCACA.

In several embodiments, gene editing reduces transcription of B2M by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces transcription of B2M by at least about 30%. In several embodiments, gene editing reduces transcription of B2M by at least about 40%. In several embodiments, gene editing reduces transcription of B2M by at least about 50%. In several embodiments, gene editing reduces transcription of B2M by at least about 60%. In several embodiments, gene editing reduces transcription of B2M by at least about 70%. In several embodiments, gene editing reduces transcription of B2M by at least about 80%. In several embodiments, gene editing reduces transcription of B2M by at least about 90%.

In several embodiments, gene editing can reduce expression of a target protein by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of B2M by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of B2M by at least about 30%. In several embodiments, gene editing reduces expression of B2M by at least about 40%. In several embodiments, gene editing reduces expression of B2M by at least about 50%. In several embodiments, gene editing reduces expression of B2M by at least about 60%. In several embodiments, gene editing reduces expression of B2M by at least about 70%. In several embodiments, gene editing reduces expression of B2M by at least about 80%. In several embodiments, gene editing reduces expression of B2M by at least about 90%.

Loss of expression of B2-microglobulin induces greatly reduced levels of MHC class I molecules, and in both NK cells and T cells, reduction of B2-microglobulin can modulate overall cell recognition of autologous and allogenic cells. Thus, according to several embodiments, gene editing B2M increases the cytotoxicity, persistence, immune avoidance or otherwise enhances the efficacy of engineered NK, T, or other cell as disclosed herein.

In several embodiments, the expression of T cell immunoreceptor with Ig and ITIM domains (TIGIT; encoded by TIGIT) is reduced and/or eliminated in order to increase overall activation in resultant T cells and/or NK cells, or other cell type provided for herein. In several embodiments, TIGIT is disrupted and/or knocked out using one or more of the gene editing methods disclosed herein. In several embodiments, TIGIT is disrupted and/or knocked out using a Crispr-Cas mediated approach (e.g., Cas9) as disclosed elsewhere herein, with the Cas nuclease guided by the use of one more of the following TIGIT-specific guide RNAs: SEQ ID NO: 507-510: SEQ ID NO 507-GTACTCCCCTGTATCGTTCA; SEQ ID NO 508-TGGGGCCACTCGATCCTTGA; SEQ ID NO 509-ACCTATCATACGTATCCTGG; SEQ ID NO 510-AGTGTACGTCCCATCAGGGT.

In several embodiments, gene editing reduces transcription of TIGIT by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces transcription of TIGIT by at least about 30%. In several embodiments, gene editing reduces transcription of TIGIT by at least about 40%. In several embodiments, gene editing reduces transcription of TIGIT by at least about 50%. In several embodiments, gene editing reduces transcription of TIGIT by at least about 60%. In several embodiments, gene editing reduces transcription of TIGIT by at least about 70%. In several embodiments, gene editing reduces transcription of TIGIT by at least about 80%. In several embodiments, gene editing reduces transcription of TIGIT by at least about 90%.

In several embodiments, gene editing can reduce expression of a target protein by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of TIGIT by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of TIGIT by at least about 30%. In several embodiments, gene editing reduces expression of TIGIT by at least about 40%. In several embodiments, gene editing reduces expression of TIGIT by at least about 50%. In several embodiments, gene editing reduces expression of TIGIT by at least about 60%. In several embodiments, gene editing reduces expression of TIGIT by at least about 70%. In several embodiments, gene editing reduces expression of TIGIT by at least about 80%. In several embodiments, gene editing reduces expression of TIGIT by at least about 90%.

TIGIT is a checkpoint receptor associated with T cell and NK cell exhaustion. Loss of expression of TIGIT in NK cells prevents NK cell exhaustion and promotes NK cell-dependent tumor immunity (Zhang et al., Nat. Immunol. (2018) 19(7):723-32). Loss of expression of TIGIT in T cells can similarly lead to downstream activation of resultant T cells. Thus, according to several embodiments, gene editing TIGIT increases the cytotoxicity, persistence, immune avoidance or otherwise enhances the efficacy of engineered NK, T, or other cell as disclosed herein.

In several embodiments, the expression of Programmed cell death protein-1 (PD-1; encoded by PDCD1) is reduced and/or eliminated in order to increase overall activation in resultant T cells and/or NK cells, or other cell type provided for herein. In several embodiments, PDCD1 is disrupted and/or knocked out using one or more of the gene editing methods disclosed herein. In several embodiments, PDCD1 is disrupted and/or knocked out using a Crispr-Cas mediated approach (e.g., Cas9) as disclosed elsewhere herein, with the Cas nuclease guided by the use of one more of the following PDCD1-specific guide RNAs: SEQ ID NO: 511-514: SEQ ID NO 511-ATGTGGAAGTCACGCCCGTT; SEQ ID NO 512-GCAGTTGTGTGACACGGAAG; SEQ ID NO 513-CAGCTTGTCCAACTGGTCGG; SEQ ID NO 514-AGTTGAGCTGGCAATCAGGG.

In several embodiments, gene editing reduces transcription of PDCD1 by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces transcription of PDCD1 by at least about 30%. In several embodiments, gene editing reduces transcription of PDCD1 by at least about 40%. In several embodiments, gene editing reduces transcription of PDCD1 by at least about 50%. In several embodiments, gene editing reduces transcription of PDCD1 by at least about 60%. In several embodiments, gene editing reduces transcription of PDCD1 by at least about 70%. In several embodiments, gene editing reduces transcription of PDCD1 by at least about 80%. In several embodiments, gene editing reduces transcription of PDCD1 by at least about 90%.

In several embodiments, gene editing can reduce expression of a target protein by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of PD-1 by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of PD-1 by at least about 30%. In several embodiments, gene editing reduces expression of PD-1 by at least about 40%. In several embodiments, gene editing reduces expression of PD-1 by at least about 50%. In several embodiments, gene editing reduces expression of PD-1 by at least about 60%. In several embodiments, gene editing reduces expression of PD-1 by at least about 70%. In several embodiments, gene editing reduces expression of PD-1 by at least about 80%. In several embodiments, gene editing reduces expression of PD-1 by at least about 90%.

PD-1 plays an inhibitory role in immune regulation and down-regulates overall function by suppressing immune cell activity. Loss of expression of PD-1 in NK cells increases overall cytotoxicity due to increased secretion of interferon-gamma, granzyme B, and perforin (Niu et al., Int. J. Med. Sci. (2020) 17 (13): 1964-73). Similarly, T cells with loss of expression of PD-1 demonstrate increased cytotoxicity and overall caspase activation (Zhao et al., Ocotarget (2018) 9(4):5208-15). Thus, according to several embodiments, gene editing PDCD1 increases the cytotoxicity, persistence, immune avoidance or otherwise enhances the efficacy of engineered NK, T, or other cell as disclosed herein.

In several embodiments, the expression of T-cell immunoglobulin and mucin-domain containing-3 (TIM-3; encoded by HAVCR2) is reduced and/or eliminated in order to increase overall activation in resultant T cells and/or NK cells, or other cell type provided for herein. In several embodiments, HAVCR2 is disrupted and/or knocked out using one or more of the gene editing methods disclosed herein. In several embodiments, HAVCR2 is disrupted and/or knocked out using a Crispr-Cas mediated approach (e.g., Cas9) as disclosed elsewhere herein, with the Cas nuclease guided by the use of one more of the following HAVCR2-specific guide RNAs: SEQ ID NO:515-518: SEQ ID NO 515-AGAAGTGGAATACAGAGCGG; SEQ ID NO 516-AATGTGACTCTAGCAGACAG; SEQ ID NO 517-CTAAATGGGGATTTCCGCAA; SEQ ID NO 518-GAGTCACATTCTCTATGGTC.

In several embodiments, gene editing reduces transcription of HAVCR2 by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces transcription of HAVCR2 by at least about 30%. In several embodiments, gene editing reduces transcription of HAVCR2 by at least about 40%. In several embodiments, gene editing reduces transcription of HAVCR2 by at least about 50%. In several embodiments, gene editing reduces transcription of HAVCR2 by at least about 60%. In several embodiments, gene editing reduces transcription of HAVCR2 by at least about 70%. In several embodiments, gene editing reduces transcription of HAVCR2 by at least about 80%. In several embodiments, gene editing reduces transcription of HAVCR2 by at least about 90%.

In several embodiments, gene editing can reduce expression of a target protein by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of TIM-3 by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of TIM-3 by at least about 30%. In several embodiments, gene editing reduces expression of TIM-3 by at least about 40%. In several embodiments, gene editing reduces expression of TIM-3 by at least about 50%. In several embodiments, gene editing reduces expression of TIM-3 by at least about 60%. In several embodiments, gene editing reduces expression of TIM-3 by at least about 70%. In several embodiments, gene editing reduces expression of TIM-3 by at least about 80%. In several embodiments, gene editing reduces expression of TIM-3 by at least about 90%.

TIM-3 is an inhibitory receptor involved in immune checkpoint function. Loss of expression of TIM-3 increases overall cytotoxicity in engineered NK and T cells as well as decreased exhaustion of NK cells and T cells, leading to increased effector function of constituent cells lacking TIM-3 expression (Pires de Silva et al., Cancer Imunol. Res. (2014) 2(5):410-22). Thus, according to several embodiments, gene editing HAVCR2 increases the cytotoxicity, persistence, immune avoidance or otherwise enhances the efficacy of engineered NK, T, or other cell as disclosed herein.

In several embodiments, the expression of CD38 (encoded by CD38) is reduced and/or eliminated in order to increase overall activation in resultant T cells and/or NK cells, or other cell type provided for herein. In several embodiments, CD38 is disrupted and/or knocked out using one or more of the gene editing methods disclosed herein. In several embodiments, CD38 is disrupted and/or knocked out using a Crispr-Cas mediated approach (e.g., Cas9) as disclosed elsewhere herein, with the Cas nuclease guided by the use of one more of the following CD38-specific guide RNAs: SEQ ID NO:519-522: SEQ ID NO 519-TGTACTTGACGCATCGCGCC; SEQ ID NO 520-TACTGACGCCAAGACAGAGT; SEQ ID NO 521-TATCAGCCACTAATGAAGTT; SEQ ID NO 522-TGTAGACTGCCAAAGTGTAT.

In several embodiments, gene editing reduces transcription of CD38 by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces transcription of CD38 by at least about 30%. In several embodiments, gene editing reduces transcription of CD38 by at least about 40%. In several embodiments, gene editing reduces transcription of CD38 by at least about 50%. In several embodiments, gene editing reduces transcription of CD38 by at least about 60%. In several embodiments, gene editing reduces transcription of CD38 by at least about 70%. In several embodiments, gene editing reduces transcription of CD38 by at least about 80%. In several embodiments, gene editing reduces transcription of CD38 by at least about 90%.

In several embodiments, gene editing can reduce expression of a target protein by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of CD38 by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of CD38 by at least about 30%. In several embodiments, gene editing reduces expression of CD38 by at least about 40%. In several embodiments, gene editing reduces expression of CD38 by at least about 50%. In several embodiments, gene editing reduces expression of CD38 by at least about 60%. In several embodiments, gene editing reduces expression of CD38 by at least about 70%. In several embodiments, gene editing reduces expression of CD38 by at least about 80%. In several embodiments, gene editing reduces expression of CD38 by at least about 90%.

CD38 plays a role in the maturation cycle of immune cells, and blood cancers can often present upregulated CD38. Loss of CD38 expression on constituent NK cells allows for greater cytotoxicity due to decreased fratricide (Nagai et al., Blood (2019) 134 (suppl. 1): 870). Wild-type NK cells self-express CD38, leading to downstream self-targeting effects in wild-type NK cells. For T cells, loss of CD38 expression for constituent T cells leads to increased cytotoxicity. Thus, according to several embodiments, gene editing CD38 increases the cytotoxicity, persistence, immune avoidance or otherwise enhances the efficacy of engineered NK, T, or other cell as disclosed herein.

In several embodiments, the expression of T cell receptor alpha (TCRα or TRAC; encoded by TRAC) is reduced and/or eliminated in order to increase overall activation in resultant T cells and/or NK cells, or other cell type provided for herein. In several embodiments, TRAC is disrupted and/or knocked out using one or more of the gene editing methods disclosed herein. In several embodiments, TRAC is disrupted and/or knocked out using a Crispr-Cas mediated approach (e.g., Cas9) as disclosed elsewhere herein, with the Cas nuclease guided by the use of one more of the following TRAC-specific guide RNAs: SEQ ID NO:566-569: SEQ ID NO 566-TTGCCGGCTGAGAACCAGAT; SEQ ID NO 567-GAGAAGTAGCAGCCATGTAC; SEQ ID NO 568-GCCCATAGGTGAAGGCGTCT; SEQ ID NO 569-CCAATCATGCTGCTGGTGGA.

In several embodiments, gene editing reduces transcription of TRAC by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces transcription of TRAC by at least about 30%. In several embodiments, gene editing reduces transcription of TRAC by at least about 40%. In several embodiments, gene editing reduces transcription of TRAC by at least about 50%. In several embodiments, gene editing reduces transcription of TRAC by at least about 60%. In several embodiments, gene editing reduces transcription of TRAC by at least about 70%. In several embodiments, gene editing reduces transcription of TRAC by at least about 80%. In several embodiments, gene editing reduces transcription of TRAC by at least about 90%.

In several embodiments, gene editing can reduce expression of a target protein by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of TRAC by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of TRAC by at least about 30%. In several embodiments, gene editing reduces expression of TRAC by at least about 40%. In several embodiments, gene editing reduces expression of TRAC by at least about 50%. In several embodiments, gene editing reduces expression of TRAC by at least about 60%. In several embodiments, gene editing reduces expression of TRAC by at least about 70%. In several embodiments, gene editing reduces expression of TRAC by at least about 80%. In several embodiments, gene editing reduces expression of TRAC by at least about 90%.

T cell receptors (TCR) are protein complexes found on T cells responsible for recognizing MHC molecules. A TCR is comprised of TCR alpha and beta subunits or TCR delta and gamma subunits. Loss of certain TCRs and preferential expression of other TCRs can lead to increased cytotoxicity in engineered cells due to increased selective targeting and recognition by constituent cells. Thus, according to several embodiments, gene editing TCR (e.g., by knockout of TRAC), increases the cytotoxicity, persistence, immune avoidance or otherwise enhances the efficacy of engineered NK, T, or other cell as disclosed herein.

In several embodiments, the expression of CISH (encoded by CISH, also known as CIS and CIS-1) is reduced and/or eliminated in order to increase overall activation in resultant T cells and/or NK cells, or other cell type provided for herein. In several embodiments, CISH is disrupted and/or knocked out using one or more of the gene editing methods disclosed herein. As discussed herein, CRISPr/CAs9 can be used to disrupt expression of CISH, though in additional embodiments, other gene editing approaches can be used. Non-limiting examples of CISH-targeting guide RNAs are shown below in Table 6.

TABLE 6
CISH Guide RNAs

| SEQ | | | |
|---|---|---|---|
| ID NO: | Name | Sequence | Target |
| 153 | CISH-1 | CTCACCAGATTCCCGAAGGT | Exon 2 |
| 154 | CISH-2 | CCGCCTTGTCATCAACCGTC | Exon 3 |
| 155 | CISH-3 | TCTGCGTTCAGGGGTAAGCG | Exon 1 |
| 156 | CISH-4 | GCGCTTACCCCTGAACGCAG | Exon 1 |
| 157 | CISH-5 | CGCAGAGGACCATGTCCCCG | Exon 1 |

In several embodiments, CISH is disrupted and/or knocked out using a Crispr-Cas mediated approach (e.g., Cas9) as disclosed elsewhere herein, with the Cas nuclease guided by the use of one more of the following CISH-specific guide RNAs: SEQ ID NO: 562-565, or other guide disclosed herein: SEQ ID NO 566-TTGCCGGCTGAGAACCAGAT; SEQ ID NO 567-GAGAAGTAGCAGCCATGTAC; SEQ ID NO 568-GCCCATAGGTGAAGGCGTCT; SEQ ID NO 569-CCAATCATGCTGCTGGTGGA.

In several embodiments, gene editing reduces transcription of CISH by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces transcription of CISH by at least about 30%. In several embodiments, gene editing reduces transcription of CISH by at least about 40%. In several embodiments, gene editing reduces transcription of CISH by at least about 50%. In several embodiments, gene editing reduces transcription of CISH by at least about 60%. In several embodiments, gene editing reduces transcription of CISH by at least about 70%. In several embodiments, gene editing reduces transcription of CISH by at least about 80%. In several embodiments, gene editing reduces transcription of CISH by at least about 90%.

In several embodiments, gene editing can reduce expression of a target protein by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of CISH by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of CISH by at least about 30%. In several embodiments, gene editing reduces expression of CISH by at least about 40%. In several embodiments, gene editing reduces expression of CISH by at least about 50%. In several embodiments, gene editing reduces expression of CISH by at least about 60%. In several embodiments, gene editing reduces expression of CISH by at least about 70%. In several embodiments, gene editing reduces expression of CISH by at least about 80%. In several embodiments, gene editing reduces expression of CISH by at least about 90%.

In CD8+ T cells, CISH actively silences TCR signaling to maintain tumor tolerance, and CISH has been shown to be a downstream negative regulator of IL-15 receptor signaling (Palmer et al., J. Exp. Med. (2015) 212 (12): 2095-2113). In NK and T cells, CISH plays a role in checkpoint maturation and proliferation (Delconte et al., Nature Immunol (2016) 17: 816-24). Thus, according to several embodiments, gene editing CISH increases the cytotoxicity, persistence, immune avoidance or otherwise enhances the efficacy of engineered NK, T, or other cell as disclosed herein.

In several embodiments, the expression of CEACAM1 (encoded by CEACAM1) is reduced and/or eliminated in order to increase overall activation in resultant T cells and/or NK cells, or other cell type provided for herein. In several embodiments, CEACAM1 is disrupted and/or knocked out using one or more of the gene editing methods disclosed herein. In several embodiments, CEACAM1 is disrupted and/or knocked out using a Crispr-Cas mediated approach (e.g., Cas9) as disclosed elsewhere herein, with the Cas nuclease guided by the use of one more of the following CEACAM1-specific guide RNAs: SEQ ID NO: 497-499: SEQ ID NO 497-GACTGAGTTATTGGCGTGGC; SEQ ID NO 498-GAATGTTCCATTGATAAGCC; SEQ ID NO 499-GAGAGGCTGAGGTTTGCCCC.

In several embodiments, gene editing reduces transcription of CEACAM1 by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces transcription of CEACAM1 by at least about 30%. In several embodiments, gene editing reduces transcription of CEACAM1 by at least about 40%. In several embodiments, gene editing reduces transcription of CEACAM1 by at least about 50%. In several embodiments, gene editing reduces transcription of CEACAM1 by at least about 60%. In several embodiments, gene editing reduces transcription of CEACAM1 by at least about 70%. In several embodiments, gene editing reduces transcription of CEACAM1 by at least about 80%. In several embodiments, gene editing reduces transcription of CEACAM1 by at least about 90%.

In several embodiments, gene editing can reduce expression of a target protein by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of CEACAM1 by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of CEACAM1 by at least about 30%. In several embodiments, gene editing reduces expression of CEACAM1 by at least about 40%. In several embodiments, gene editing reduces expression of CEACAM1 by at least about 50%. In several embodiments, gene editing reduces expression of CEACAM1 by at least about 60%. In several embodiments, gene editing reduces expression of CEACAM1 by at least about 70%. In several embodiments, gene editing reduces expression of CEACAM1 by at least about 80%. In several embodiments, gene editing reduces expression of CEACAM1 by at least about 90%.

CEACAM1 is an immune checkpoint for both NK and T cells and can inhibit lysis of CEACAM1-bearing tumor cell lines. Loss of expression of CEACAM1 can increase overall cytotoxicity for NK and T cells (Markel et al., J. Clin. Oncol. (2016) 34 (suppl. 15): 3044). Thus, according to several embodiments, gene editing CEACAM1 increases the cytotoxicity, persistence, immune avoidance or otherwise enhances the efficacy of engineered NK, T, or other cell as disclosed herein.

In several embodiments, the expression of DDIT4 (encoded by DDIT4) is reduced and/or eliminated in order to increase overall activation in resultant T cells and/or NK cells, or other cell type provided for herein. In several embodiments, DDIT4 is disrupted and/or knocked out using one or more of the gene editing methods disclosed herein. In several embodiments, DDIT4 is disrupted and/or knocked out using a Crispr-Cas mediated approach (e.g., Cas9) as disclosed elsewhere herein, with the Cas nuclease guided by the use of one more of the following DDIT4-specific guide RNAs: SEQ ID NO: 500-502: SEQ ID NO 500-CCTCACCATGCCTAGCCTTT; SEQ ID NO 501-CGATCTGGGGTGGGAGTTCG; SEQ ID NO 502-GTTTGACCGCTCCACGAGCC.

In several embodiments, gene editing reduces transcription of DDIT4 by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces transcription of DDIT4 by at least about 30%. In several embodiments, gene editing reduces transcription of DDIT4 by at least about 40%. In several embodiments, gene editing reduces transcription of DDIT4 by at least about 50%. In several embodiments, gene editing reduces transcription of DDIT4 by at least about 60%. In several embodiments, gene editing reduces transcription of DDIT4 by at least about 70%. In several embodiments, gene editing reduces transcription of DDIT4 by at least about 80%. In several embodiments, gene editing reduces transcription of DDIT4 by at least about 90%.

In several embodiments, gene editing can reduce expression of a target protein by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of DDIT4 by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of DDIT4 by at least about 30%. In several embodiments, gene editing reduces expression of DDIT4 by at least about 40%. In several embodiments, gene editing reduces expression of DDIT4 by at least about 50%. In several embodiments, gene editing reduces expression of DDIT4 by at least about 60%. In several embodiments, gene editing reduces expression of DDIT4 by at least about 70%. In several embodiments, gene editing reduces expression of DDIT4 by at least about 80%. In several embodiments, gene editing reduces expression of DDIT4 by at least about 90%.

In NK and T cells, DDIT4 is a negative regulator of mTORC1, which itself enhances IL-15 mediated survival and proliferation of NK cells. Moreover, DDIT4 is upregulated by oxidative stress conditions as is common in tumor microenvironments. Loss of DDIT4 function in engineered cells may increase overall glucose metabolism leading to enhanced proliferation, as well as increasing overall NK or T cell cytotoxicity. Thus, according to several embodiments, gene editing DDIT4 increases the cytotoxicity, persistence, immune avoidance or otherwise enhances the efficacy of engineered NK, T, or other cell as disclosed herein.

In several embodiments, the expression of MAPKAP Kinase 3 (MAPKAPK3; encoded by MAPKAPK3) is reduced and/or eliminated in order to increase overall activation in resultant T cells and/or NK cells, or other cell type provided for herein. In several embodiments, MAPKAPK3 is disrupted and/or knocked out using one or more of the gene editing methods disclosed herein. In several embodiments, MAPKAPK3 is disrupted and/or knocked out using a Crispr-Cas mediated approach (e.g., Cas9) as disclosed elsewhere herein, with the Cas nuclease guided by the use of one more of the following MAPKAPK3-specific guide RNAs: SEQ ID NO: 494-496: SEQ ID NO 494-CTCTGCTGTTTCACCATCCA; SEQ ID NO 495-CCCGGCTTGGGCGGTGCTCC; SEQ ID NO 496-CGACTACCAGTTGTCCAAGC.

In several embodiments, gene editing reduces transcription of MAPKAPK3 by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces transcription of MAPKAPK3 by at least about 30%. In several embodiments, gene editing reduces transcription of MAPKAPK3 by at least about 40%. In several embodiments, gene editing reduces transcription of MAPKAPK3 by at least about 50%. In several embodiments, gene editing reduces transcription of MAPKAPK3 by at least about 60%. In several embodiments, gene editing reduces transcription of MAPKAPK3 by at least about 70%. In several embodiments, gene editing reduces transcription of MAPKAPK3 by at least about 80%. In several embodiments, gene editing reduces transcription of MAPKAPK3 by at least about 90%.

In several embodiments, gene editing can reduce expression of a target protein by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of MAPKAPK3 by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of MAPKAPK3 by at least about 30%. In several embodiments, gene editing reduces expression of MAPKAPK3 by at least about 40%. In several embodiments, gene editing reduces expression of MAPKAPK3 by at least about 50%. In several embodiments, gene editing reduces expression of MAPKAPK3 by at least about 60%. In several embodiments, gene editing reduces expression of MAPKAPK3 by at least about 70%. In several embodiments, gene editing reduces expression of MAPKAPK3 by at least about 80%. In several embodiments, gene editing reduces expression of DDIT4 by at least about 90%.

MAPKAP Kinase 3 in expressed in both NK and T cells. Loss of MAPKAPK3 in engineered cells is expected to increase cytotoxicity, cytokine secretion, and overall NK signaling. Thus, according to several embodiments, gene editing MAPKAPK3 increases the cytotoxicity, persistence, immune avoidance or otherwise enhances the efficacy of engineered NK, T, or other cell as disclosed herein.

In several embodiments, the expression of SMAD3 (encoded by SMAD3) is reduced and/or eliminated in order to increase overall activation in resultant T cells and/or NK cells, or other cell type provided for herein. In several embodiments, SMAD3 is disrupted and/or knocked out using one or more of the gene editing methods disclosed herein. In several embodiments, SMAD3 is disrupted and/or knocked out using a Crispr-Cas mediated approach (e.g., Cas9) as disclosed elsewhere herein, with the Cas nuclease guided by the use of one more of the following SMAD3-specific guide RNAs: SEQ ID NO: 491-493: SEQ ID NO 491-CCGATCGTGAAGCGCCTGCT; SEQ ID NO 492-CGAGAAGGCGGTCAAGAGCC; SEQ ID NO 493-CTTGGTGTTGACGTTCTGCG.

In several embodiments, gene editing reduces transcription of SMAD3 by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces transcription of SMAD3 by at least about 30%. In several embodiments, gene editing reduces transcription of SMAD3 by at least about 40%. In several embodiments, gene editing reduces transcription of SMAD3 by at least about 50%.

In several embodiments, gene editing reduces transcription of SMAD3 by at least about 60%. In several embodiments, gene editing reduces transcription of SMAD3 by at least about 70%. In several embodiments, gene editing reduces transcription of SMAD3 by at least about 80%. In several embodiments, gene editing reduces transcription of SMAD3 by at least about 90%.

In several embodiments, gene editing can reduce expression of a target protein by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of SMAD3 by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of SMAD3 by at least about 30%. In several embodiments, gene editing reduces expression of SMAD3 by at least about 40%. In several embodiments, gene editing reduces expression of SMAD3 by at least about 50%. In several embodiments, gene editing reduces expression of SMAD3 by at least about 60%. In several embodiments, gene editing reduces expression of SMAD3 by at least about 70%. In several embodiments, gene editing reduces expression of SMAD3 by at least about 80%. In several embodiments, gene editing reduces expression of SMAD3 by at least about 90%.

SMAD3 is a downstream mediator of TGF-Beta and Activin A signaling. Inhibition of activin A provides an effective downstream TGFBR knockout. Smad3 silenced NK cells demonstrate increased proliferation and differentiation, as well as increased cytotoxicity in engineered T and NK cells (Tang et al., Nat. Commun. (2017) 8: 14677). Thus, according to several embodiments, gene editing SMAD3 increases the cytotoxicity, persistence, immune avoidance or otherwise enhances the efficacy of engineered NK, T, or other cell as disclosed herein.

### Viral Immunosuppressive Peptides

Many viral infections occur, at least in part, due to the ability of a virus to evade an host immune system, either by camouflage or suppression of host immune reactivity. In vitro studies have shown that particular aspects of certain viruses have such immunosuppressive effects, for example, retroviral transmembrane envelope proteins, such as the p15E protein. Additionally, studies have identified conserved regions of sequences across multiple viral types, see for example Table 7, which includes, among others, a 17 amino acid conserved sequence, CKS-17 (SEQ ID NO: 199), and the nucleic acid encoding the same (SEQ ID NO: 216).

TABLE 7
Viral Peptides

| SEQ ID | | |
|---|---|---|
| NO: | Name | Sequence |
| 199 | CKS-17 | LQNRRGLDLLFLKEGGL |
| 200 | MoLV p15E | LQNRRGLDLLFLKEGGLCAALKEECCF |
| 201 | FLC p15E | LQNRRGLDLLFLKEGGLCAALKEECCF |
| 202 | AKV p15E | LQNRRGLDLLFLKEGGLCAALKEECCF |
| 203 | GLV p15E | LQNRRGLDLLFLKEGGLCAALKEECCF |
| 204 | MMCF p15E | LQNRRGLDLLFLKEGGLCAALKEECCF |
| 205 | AMCF p15E | LQNRRGLDLLFLKEGGLCAALKEECCF |
| 206 | FeLV p15E | LQNRRGLDLLFLQEGGLCAALKEECCF |
| 207 | MPMV gp20 | LQNRRGLDLLTAEQGGICLALQEKCCF |
| 208 | REV-A GP20 | LQNRRGLDLLTAEQGGICLALQEKCCF |
| 209 | HTLV-I gp21 | AQNRRGLDLLFWEQGGLCKALQEQCRF |
| 210 | HTLV-II gp21 | AQNRRGLDLLFWEQGGLCKAIQEQCCF |
| 211 | HIV-1 NEF | MTYKAAVDLSHFLKEKGGL |
| 212 | HIV-1 gp41 | LQARVLAVERYLKDQQL |
| 213 | B26 | LQNKRGLDLLFLX₁X₂GGL |
| | | (X₁ = K/E, X₂ = E/K) |
| 214 | BaEV | LQNRRGLDLLTAEQGGX₁ |
| | | (X₁ = L/I) |
| 215 | ERV-9, 4-1 | X₁QNRX₂X₃LX₄X₅X₆X₇AX₈X₉X₁₀GX₁₁ |
| | | (X₁ = L/Y, X₂ = */L, X₃ = G/A, X₄ = D/N, X₅ = L/Y, X₆ = L/S, |
| | | X₇ = L/T, X₈ = E/Q/A, X₉ = E/K, X₁₀ = G/E, X₁₁ = L/V |
| 216 | CKS-17 | ctgcaaaacagaaggggattagacctgctttttcttaaagagggagggctc |
| 1019 | HIV-1 NEF | TYKAAVDLSHFLKEKGGL |

In several embodiments, one or more of such viral immunosuppressive peptides (also referred to as viral peptides) are used to confer resistance to inactivation of engineered immune cells by host NK cells. Various approaches can be undertaken depending on the embodiment and the cell type to be used.

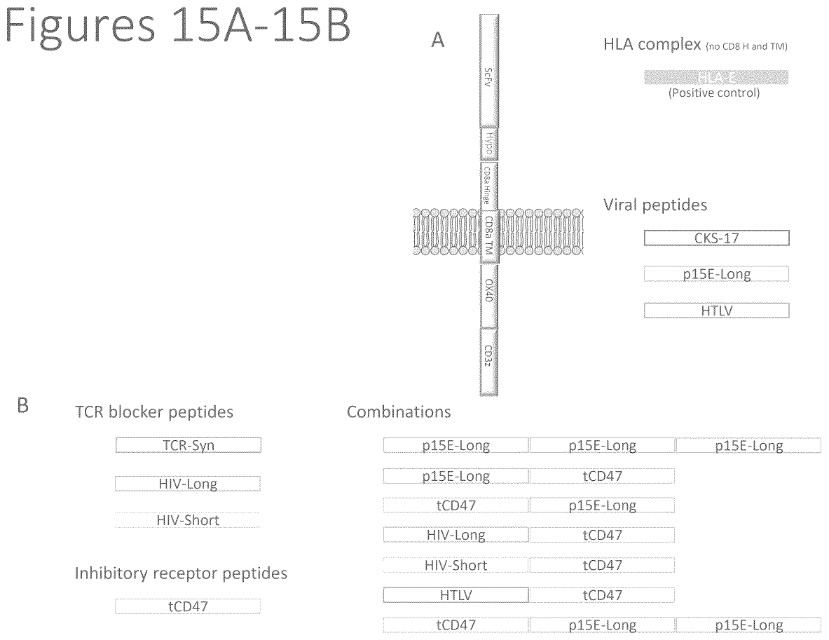

As discussed herein, various immunosuppressive constructs are provided for in order to confer on, for example an NK or a T cell in a mixed NK and T cell therapeutic population, reduced immunogenicity vis-à-vis the other therapeutic cells, as well as vis-à-vis host T cells. In several embodiments, the immunosuppressive constructs and resulting engineered (and/or edited) cells are less susceptible to fratricide from another member of the therapeutic cell population, and also reduce risk of graft versus host and host versus graft side effects. FIGS. 15A and 15B show non-limiting schematics of how an immunosuppressive construct according to embodiments provided for herein might be constructed. Table 8 shows the corresponding SEQ ID NOS for certain non-limiting immunosuppressive constructs provided for herein.

TABLE 8
Selected Immunosuppressive Constructs Sequence Identifiers

| | | SEQ | |
|---|---|---|---|
| SEQ ID | | ID | |
| NO | Construct Identifier | NO | Construct Identifier |
| 683 | UL18 trimer_SS VMAPRTLFL_IRES_GFP | 791 | GP41fptm_sfGFP_aa |
| 684 | UL18 trimer_SS VMAPRTLFL_IRES_GFP | 792 | GP41fptm_sfGFP |
| 685 | UL18 trimer_SS VMAPRTLFL-AA [no Flag or | 793 | GP41fptm_aa_No-GFP |
| | GFP] | | |
| 686 | UL18 trimer_SS VMAPRTLFL-NT [no Flag or | 794 | GP41fptm_NT_No_GFP |
| | GFP] | | |
| 687 | HLA-E trimer_SS VMAPRTLFL_IRES_GFP | 795 | CKS17-8aTM_sfGFP_aa |
| 688 | HLA-E trimer_SS VMAPRTLFL | 796 | CKS17-8aTM_sfGFP |
| 689 | HLA-E trimer_SS VMAPRTLFL-AA-[no flag or | 797 | CKS17-8aTM_aa_No_GFP |
| | gfp] | | |
| 690 | HLA-E trimer_SS VMAPRTLFL [no flag or gfp] | 798 | CKS17-8aTM_NT_No_GFP |
| 691 | CKS17-8aHTM_aa | 799 | CKS_HTLV-8aTM_sfGFP_aa |
| 692 | CKS17-8aHTM | 800 | CKS_HTLV-8aTM_NT_No_GFP |
| 693 | CKS17-8aHTM_aa [no flag or gfp] | 801 | CKS_HTLV-8aTM_aa_No_GFP |
| 694 | CKS17-8aHTM [no flag or gfp] | 802 | CKS_HTLV-8aTM_sfGFP |
| 695 | p15E-L-8aHTM_IRES_GFP | 803 | CKS_LMP-8aTM_sfGFP_aa |
| 696 | p15E-L-8aHTM_IRES_GFP | 804 | CKS_LMP-8aTM_sfGFP |
| 697 | p15E-L-8aHTM [no flag/gfp] | 805 | CKS_LMP-8aTM_aa_no_GFP |
| 698 | p15E-L-8aHTM_[no flag/gfp] | 806 | CKS_LMP-8aTM_NT_No_GFP |
| 699 | HTLV-8aHTM_IRES_GFP | 807 | CKS_GP41fptm_sfGFP_aa |
| 700 | HTLV-8aHTM_IRES_GFP | 808 | CKS_GP41fptm_sfGFP |
| 701 | HTLV-8aHTM_[no flag/gfp] | 809 | CKS_GP41fptm_aa_No_GFP |
| 702 | HTLV-8aHTM_[no flag/gfp] | 810 | CKS_GP41fptm_NT_No_GFP |
| 703 | HIV_L-8aHTM_IRES_GFP | 811 | LMP_S-CD8HTM_sfGFP_aa |
| 704 | HIV_L-8aHTM_IRES_GFP | 812 | LMP_S-CD8HTM_sfGFP |
| 705 | HIV_L-8aHTM_[no flag/gfp] | 813 | LMP_S-CD8HTM_aa_No_GFP |
| 706 | HIV_L-8aHTM_[no flag/gfp] | 814 | LMP_S-CD8HTM_NT_No_GFP |
| 707 | HIV_S-8aHTM_IRES_GFP | 815 | LMP_CD47tm162_sfGFP_aa |
| 708 | HIV_S-8aHTM_IRES_GFP | 816 | LMP_CD47tm162_sfGFP |
| 709 | HIV_S-8aHTM_no flag/gfp | 817 | LMP_CD47tm162_aa_No_GFP |
| 710 | HIV_S-8aHTM_no flag/gfp | 818 | LMP_CD47tm162_NT_No_GFP |
| 711 | TCR Synthetic 3x-8aHTM_IRES_GFP | 819 | p15E_CD47tm162_sfGFP_aa |
| 712 | TCR Synthetic 3x-8aHTM_IRES_GFP | 820 | p15E_CD47tm162_sfGFP |
| 713 | TCR Synthetic 3x-8aHTM_no flag/gfp | 821 | p15E_CD47tm162_aa_No_GFP |
| 714 | TCR Synthetic 3x-8aHTM_no flag/gfp | 823 | CD47_GP41fptm_sfGFP_aa |
| 715 | tCD47-8aHTM_IRES_GFP | 824 | CD47_GP41fptm_sfGFP |
| 716 | tCD47-8aHTM_IRES_GFP | 825 | CD47_GP41fptm_aa_No_GFP |
| 717 | tCD47-8aHTM_no flag/gfp | 826 | CD47_GP41fptm_NT_No_GFP |
| 718 | tCD47-8aHTM_no flag/gfp | 827 | HLA-E_STE20_sfGFP_aa |
| 719 | p15Ex3-8aHTM_IRES_GFP | 828 | HLA-E_STE20_sfGFP |
| 720 | p15Ex3-8aHTM_IRES_GFP | 829 | HLA-E_STE20_aa_NO_GFP |
| | | | (a.k.a. HLA-E (PBL20) |
| 721 | p15Ex3-8aHTM_no flag/gfp | 830 | HLA-E_STE20_NT_NO_GFP |
| | | | (a.k.a. HLA-E (PBL20) |
| 722 | p15Ex3-8aHTM_no flag/gfp | 831 | anti-SIRPa |
| | | | agonist_vHL_sfGFP_aa |
| 723 | p15E_tCD47-8aHTM_IRES_GFP | 832 | anti-SIRPa agonist_vHL_sfGFP |
| 724 | p15E_tCD47-8aHTM_IRES_GFP | 833 | anti-SIRPa |
| | | | agonist_vHL_aa_NO_GFP |
| 725 | p15E_tCD47-8aHTM_no flag/gfp | 834 | anti-SIRPa |
| | | | agonist_vHL_NT_NO_GFP |
| 726 | p15E_tCD47-8aHTM_no flag/gfp | 835 | HTLV1_fGP62_sfGFP_aa |
| 727 | tCD47_p15E-8aHTM_IRES_GFP | 836 | HTLV1_fGP62_sfGFP |
| 728 | tCD47_p15E-8aHTM_IRES_GFP | 837 | HTLV1_fGP62_aa_NO_GFP |
| 729 | tCD47_p15E-8aHTM_no flag gfp | 838 | HTLV1_fGP62_NO_GFP |
| 730 | tCD47_p15E-8aHTM_no flag/gfp | 839 | HTLV1_GP21_sfGFP_aa |
| 731 | HIV_L_tCD47-8aHTM_IRES_GFP | 840 | HTLV1_GP21_sfGFP |
| 732 | HIV_L_tCD47-8aHTM_IRES_GFP | 841 | HTLV1_GP21_aa_NO_GFP |
| 733 | HIV_L_tCD47-8aHTM_no flag/gfp | 842 | HTLV1_GP21_NO_GFP |
| 734 | HIV_L_tCD47-8aHTM_no flag/gfp | 843 | LASV_fGP2_sfGFP_aa |
| 735 | HIV_S_tCD47-8aHTM_IRES_GFP | 844 | LASV_fGP2_sfGFP |
| 736 | HIV_S_tCD47-8aHTM_IRES_GFP | 845 | LASV_fGP2_aa_NO_GFP- |
| 737 | HIV_S_tCD47-8aHTM_no flag/gfp | 846 | LASV_fGP2_NT_NO_GFP |
| 738 | HIV_S_tCD47-8aHTM_no flag/gfp | 847 | SEBOV_fGP_sfGFP_aa |
| 739 | HTLV_tCD47-8aHTM_IRES_GFP | 848 | SEBOV_fGP_sfGFP |
| 740 | HTLV_tCD47-8aHTM_IRES_GFP | 849 | SEBOV_fGP_aa_NO_GFP |
| 741 | HTLV_tCD47-8aHTM_no flag/gfp | 850 | SEBOV_fGP_NT_NO_GFP |
| 742 | HTLV_tCD47-8aHTM_no flag/gfp | 851 | SEBOV_GP2_sfGFP_aa |
| 743 | tCD47_p15Ex2-8aHTM_IRES_GFP | 852 | SEBOV_GP2_sfGFP |
| 744 | tCD47_p15Ex2-8aHTM_IRES_GFP | 853 | SEBOV_GP2_aa_NO_GFP |
| 745 | tCD47_p15Ex2-8aHTM_noflag/gfp | 854 | SEBOV_GP2_NT_NO_GFP |
| 746 | tCD47_p15Ex2-8aHTM_no flag/gfp | 855 | SCoV_S2_sfGFP_aa |
| 747 | CKS17-8aHTM_eGFP_aa | 856 | SCoV_S2_sfGFP |
| 748 | CKS17-8aHTM_eGFP | 857 | SCoV_S2_aa_NO_GFP |
| 749 | CKS17-8aHTM_aa_noGFP | 858 | SCOV_S2_NT_NO_GFP |
| 750 | CKS17-8aHTM_NT_no GFP | 859 | LGALS3BP_sfGFP_aa |
| 751 | CD47-eGFP_aa | 860 | LGALS3BP_sfGFP |
| 752 | CD47-eGFP | 861 | LGALS3BP_aa_NO_GFP |
| 753 | CD47-_aa_no_GGFP | 862 | LGALS3BP_NT_No_GFP |
| 754 | CD47-NT_no_GFP | 863 | CD24_sfGFP_aa |
| 755 | fGP41_HIVtm_sfGFP_aa | 864 | CD24_sfGFP |
| 756 | fGP41_HIVtm_sfGFP | 865 | CD24_aa_NO_GFP |
| 757 | fGP41_HIVtm_aa_no_GFP | 866 | CD24_NT_NO_GFP |
| 758 | fGP41_HIVtm_NT_no_GFP | 867 | HCV_E2_sfGFP_aa |
| 759 | p15Etm_sfGFP_aa | 868 | HCV_E2_sfGFP |
| 760 | p15Etm_sfGFP | 869 | HCV_E2_aa_NO_GFP |
| 761 | p15Etm_aa_no_GFP | 870 | HCV_E2_NT_NO_GFP |
| 762 | p15Etm_NT_noGFP | 871 | anti-SIRPa |
| | | | agonist_vLH_sfGFP_aa |
| 763 | CD43_TM_sfGFP_aa | 872 | anti-SIRPa agonist_vLH_sfGFP |
| 764 | CD43_TM_sfGFP | 873 | anti-SIRPa |
| | | | agonist_vLH_aa_NO_GFP |
| 765 | CD43_TM_aa_No_GFP | 874 | anti-SIRPa |
| | | | agonist_vLH_NT_NO_GFP |
| 766 | CD43_TM_NT_no_GFP | 875 | CEACAM1_sfGFP_aa |
| 767 | LMP1_TM-sfGFP_aa | 876 | CEACAM1_sfGFP |
| 768 | LMP1_TM-sfGFP | 877 | CEACAMI_aa_NO_GFP |
| 769 | LMP1_TM_aa_no_GFP | 878 | CEACAM1_NT_NO_GFP |
| 770 | LMP1_TM-NT_No-GFP | 879 | CD155tm_3M_sfGFP_aa |
| 771 | fgD_TM_sfGFP_aa | 880 | CD155tm_3M_sfGFP |
| 772 | fgD_TM_sfGFP | 881 | CD155tm_3M_aa_NO_GFP |
| 773 | fgD_TM_AA-NO_GFP | 882 | CD155tm_3M_NT_NO_GFP |
| 774 | fgD_TM_NT_NO_GFP | 883 | CD31tm_sfGFP_aa |
| 775 | fLLT1_TM-sfGFP_aa | 884 | CD31tm_sfGFP |
| 776 | fLLT1_TM-sfGFP | 885 | CD31tm_aa_NO_GFP |
| 777 | fLLT1_TM-_aa_No_GFP | 886 | CD31tm_NT_NO_GFP |
| 778 | fLLT1_TM-NT_No_GFP | 887 | CD111tm_sfGFP_aa |
| 779 | CD47tm162_sfGFP_aa | 888 | CD111tm_sfGFP |
| 780 | CD47tm162_sfGFP | 889 | CD111tm_aa_NO_GFP |
| 781 | CD47tm162_aa_No_GFP | 890 | CD111tm_NT_NO_GFP |
| 782 | CD47tm162_NT_NoGFP | 891 | CD200tm_sfGFP_aa |
| 783 | LMP_L-CD8HTM_sfGFP_aa | 892 | CD200tm_sfGFP |
| 784 | LMP_L-CD8HTM_sfGFP | 893 | CD200tm_aa_NO_GFP |
| 785 | LMP_L-CD8HTM_aa_No_GFP | 894 | CD200tm_NO_GFP |
| 786 | LMP_L-CD8HTM_NT_No_GFP | 895 | CD8a signal peptide AA |
| 787 | LALLFWLx5-CD8HTM_sfGFP_aa | 896 | CD8a signal peptide NT |
| 788 | LALLFWLx5-CD8HTM_sfGFP | 997 | HLA-E_(PBL20)_(E1-5 sgRNA |
| | | | resistant)_AA |
| 789 | LALLFWLx5-CD8HTM_aa_no_GFP | 998 | HLA-E_(PBL20)_(E1-5 sgRNA |
| | | | resistant)_DNA |
| 790 | LALLFWLx5-CD8HTM_NT_No_GFP | 999 | HLA-E_(PBL15)_AA |
| 822 | p15E_CD47tm162_NT_No_GFP | 1000 | HLA-E_(PBL15)_DNA |
| 1020 | UL18 trimer_SS VMAPRTLFL-AA [no Flag or | 1017 | HLA-E trimer_SS VMAPRTLFL- |
| | GFP] | | AA-[no flag or gfp] |
| 1021 | HLA-E_STE20_sfGFP_aa | 1014 | HLA-E_STE20_aa_NO_GFP |
| | | | (a.k.a. HLA-E (PBL20) |
| 1022 | CD24_sfGFP_aa | 1023 | CD24_aa_NO_GFP |
| 1015 | HLA-E_(PBL20)_(E1-5 sgRNA resistant)_AA | 1016 | HLA-E_(PBL15)_AA |

It is contemplated that any of the amino acid sequences provided herein may be provided with or without a signal sequence (e.g., a CD8a signal sequence, such as MALPVTALLLPLALLLHAARP). It is also contemplated that any of the amino acid sequences provided herein may be provided with or without an initial methionine (M) residue.

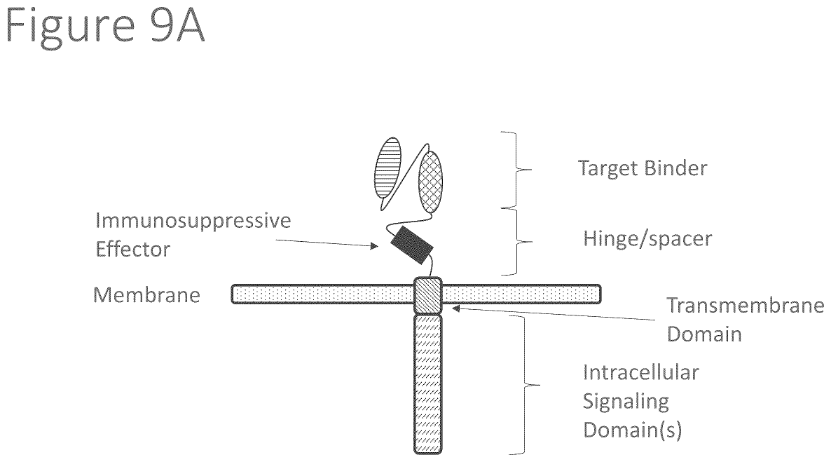

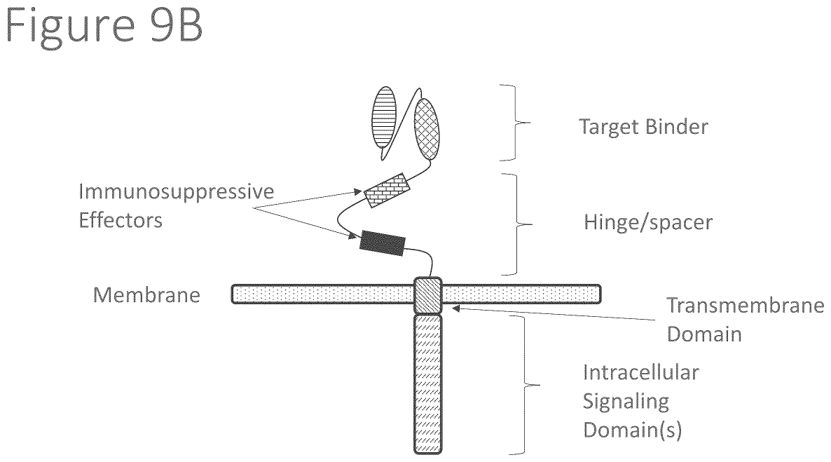

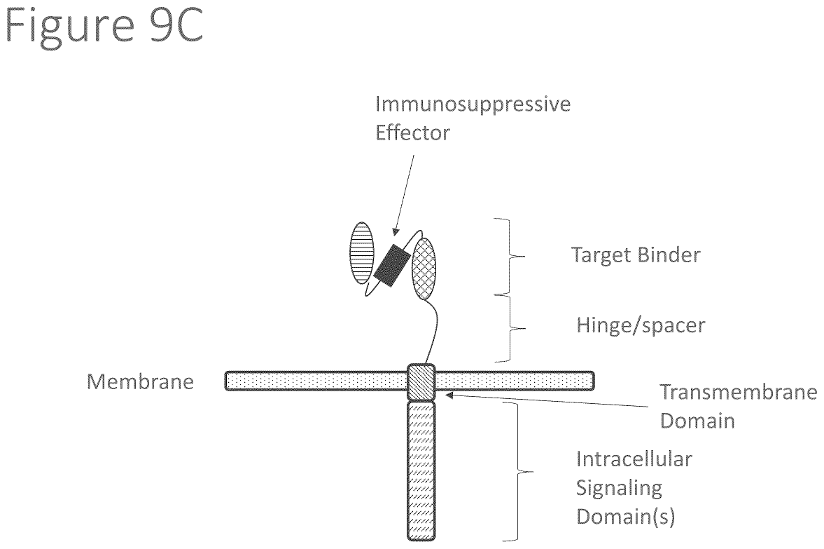

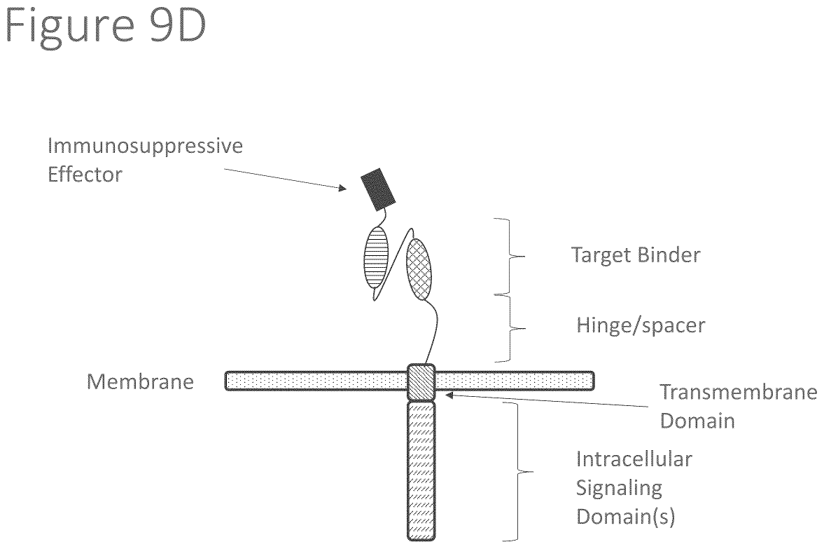

In some embodiments, one or more viral immunosuppressive peptides are integrated into a chimeric antigen receptor. According to several embodiments, the one or more viral immunosuppressive peptides are integrated into a chimeric antigen receptor that is then expressed by a population of immune cells to be used in treating a patient. In several embodiments, the immune cells are allogeneic to the patient. In some embodiments, the immune cells comprise NK cells. In some embodiments, the immune cells comprise T cells. In some embodiments, the immune cells comprise NK cells and T cells. In several embodiments, a combination of NK cells and T are engineered to express one or more CARs that comprise one or more viral immunosuppressive peptide. FIG. 9A depicts a non-limiting embodiment of a viral immunosuppressive peptide that is incorporated into a CAR (identified generically as “Immunosuppressive effector Domain”, which shall be understood to refer to any of the viral immunosuppressive peptides or other immunosuppressive peptides/proteins disclosed herein, unless otherwise specified). Shown in FIG. 9A, the viral immunosuppressive peptide is integrated into the hinge/spacer domain of a CAR comprising a target binder (such as an scFv), a hinge (also referred to herein as a spacer), a transmembrane domain and one or more intracellular signaling domains. Depending on the embodiment, more than one (e.g., two, three, four, five, or more) viral immunosuppressive peptides can be introduced into the hinge/spacer region of the CAR. In several such embodiments, as schematically depicted in FIG. 9B, when two (or more) viral immunosuppressive peptides are used, they can be of a different sequence or type. For example, in several embodiments, a CKS-17 peptide is integrated into the hinge region of the CAR in conjunction with, for example, a REV-A peptide. In several embodiments, the inclusion of two or more viral immunosuppressive peptides creates a synergistic immunosuppressive effect.

Depending on the embodiment, the length of the hinge/spacer region can be altered. In several embodiments, a CD8alpha hinge/spacer region is use, but, in some embodiments, a longer or a shorter spacer is used. The spacer can be, depending on the embodiment, an IgG1, IgG2, IgG3, IgG4, or CD28 spacer domain or be derived from IgG1, IgG2, IgG3, IgG4, CD28, or can be a fully synthetic sequence. In several embodiments, IgG-based spacers are edited to reduce or eliminate the ability of the spacer to bring Fc-receptor bearing cells, which can advantageously reduce off-target activation of immune cells (such as those bearing the immunosuppressive effectors as disclosed herein). Non-limiting editing approaches include, but are not limited to, deletion of the heavy chain constant 2 (CH2) domain to abrogate binding to the Fc receptor, or mutating certain amino acids that are essential to Fc receptor binding. In several embodiments, a longer spacer advantageously allows for enhances targeting of certain membrane-proximal epitopes expressed by cancer cells and exposure of the immunosuppressive effector such that it can interact with host and/or administered immune cells to reduce unwanted suppression of the therapeutic cells. In several embodiments, a single hinge region can be made longer by including multiple hinge-encoding sequences, e.g., two, three, four, or more hinges. In several embodiments, wherein multiple hinge regions are used, they can be of the same type (e.g., three CD8a hinges) or can vary (e.g., one CD8a hinge, one CD28 hinge, and a IgG1 hinge). In several embodiments, a shorter hinge is used, wherein the shorter hinge limits the ability of host phosphatases (like CD45) to attenuate signaling of a CAR expressed by the engineered immune cell. In several embodiments, the hinge region comprises one or more of SEQ ID NOs: 479-487. In some embodiments, the hinge region comprises an amino acid sequence with at least about 80%, at least about 85%, at least about 90%, at least about 94%, at least about 95%, at least about 96%, at least about 97%, at least about 98%, or at least about 99%, sequence identity, homology and/or functional equivalence with any of SEQ ID NOs: 479-487: SEQ ID NO 479-EPKSCDKTHTCPPCP; SEQ ID NO 480-ERKCCVECPPCP; SEQ ID NO 481-ELKTPLGDTHTCPRCPEPKSCDTPPPCPRCPEPKSCDTPPPCPRCPEKSCDTPPPCPRCP; SEQ ID NO 482-ESKYGPPCPSCP; SEQ ID NO 483-ESKYGPPCPPCP; SEQ ID NO 484-YGPPCPPCP; SEQ ID NO 485-KYGPPCPPCP; SEQ ID NO 486-EVVKYGPPCPPCP; SEQ ID NO 487-IEVMYPPPYLDNEKSNGTIIHVKGKHLCPSPLFPGPSKP.

FIG. 9C depicts an additional approach according to several embodiments disclosed herein, namely the engineering of the viral immunosuppressive peptide into the linker between the heavy chain and the light chain of an scFv. in additional embodiments, where a target binder that is not an scFv (e.g., a full antibody), the viral immunosuppressive peptide can be integrated into any region of that binder that allows for exposure of the vial immunosuppressive peptide as well as maintenance of target binding capability. As with integration into the hinge region, engineering the viral immunosuppressive peptide into the linker region can be done with a single peptide, or with multiple peptides. An additional approach is shown in FIG. 9D, with the viral immunosuppressive peptide engineered into the N-terminal region of the chimeric antigen receptor. As with the other approaches, in several embodiments multiple viral immunosuppressive peptides can be used. Additionally, combinations of these positions within the CAR can be used. For example, a viral immunosuppressive peptide (or more than one) can be positioned in the hinge region in combination with, for example a viral immunosuppressive peptide (or more than one) positioned in the linker region of an scFv and/or at the N-terminus of the CAR. In several embodiments, the positions allow the viral immunosuppressive peptides to be exposed such that they can interact with, and thus suppress, host immune cell activity that would otherwise reduce the efficacy of the engineered immune cell expressing the CAR.

In several embodiments, the engineered CAR comprises one or more copies of one or more of the following amino acid or DNA sequences: SEQ ID NO: 199-216, 1019, 220-221, 225-226, 230-231, 235-236, 240-241, 245-246, 250-251, 273-274, 278, 280, 288, or 289. As discussed above, those sequences (or individual sequence) can be positioned in the hinge region, the N-terminal region or within the target binder region (e.g., within the linker of an scFv). In several embodiments, the CAR comprises an amino acid sequence or DNA sequence with at least about 80%, at least about 85%, at least about 90%, at least about 94%, at least about 95%, at least about 96%, at least about 97%, at least about 98%, or at least about 99%, sequence identity, homology and/or functional equivalence with any of SEQ ID NOs: SEQ ID NO: 199-216, 1019, 220-221, 225-226, 230-231, 235-236, 240-241, 245-246, 250-251, 273-274, 278, 280, 288, or 289.

In several embodiments, the engineered cells provided for herein comprise a chimeric receptor that targets NKG2D ligands, wherein the CAR comprises an amino acid of SEQ ID NO: 174, or comprises an amino acid sequence with at least about 80%, at least about 85%, at least about 90%, at least about 94%, at least about 95%, at least about 96%, at least about 97%, at least about 98%, or at least about 99%, sequence identity, homology and/or functional equivalence to SEQ ID NO: 174 with one or more copies of one or more of the following viral immunosuppressive amino acid sequences: SEQ ID NO: 199-216, 220, 225, 230, 235, 240, 245, 250, 273, 280, 288, or 289 integrated into the sequence of SEQ ID NO: 174. In addition to, or in place of the integrated viral immunosuppressive amino acids, the CAR optionally comprises an amino acid of SEQ ID NO: 174, or comprises an amino acid sequence with at least about 80%, at least about 85%, at least about 90%, at least about 94%, at least about 95%, at least about 96%, at least about 97%, at least about 98%, or at least about 99%, sequence identity, homology and/or functional equivalence to SEQ ID NO: 174, with one or more of the viral immunosuppressive sequences expressed on the cell in a membrane-bound format, as discussed below.

In several embodiments, the engineered cells provided for herein comprise a chimeric receptor that targets NKG2D ligands, wherein the CAR comprises an amino acid of SEQ ID NO: 1024, or comprises an amino acid sequence with at least about 80%, at least about 85%, at least about 90%, at least about 94%, at least about 95%, at least about 96%, at least about 97%, at least about 98%, or at least about 99%, sequence identity, homology and/or functional equivalence to SEQ ID NO: 1024 with one or more copies of one or more of the following viral immunosuppressive amino acid sequences: SEQ ID NO: 199-216, 220, 225, 230, 235, 240, 245, 250, 273, 280, 288, or 289 integrated into the sequence of SEQ ID NO: 1024. In addition to, or in place of the integrated viral immunosuppressive amino acids, the CAR optionally comprises an amino acid of SEQ ID NO: 1024, or comprises an amino acid sequence with at least about 80%, at least about 85%, at least about 90%, at least about 94%, at least about 95%, at least about 96%, at least about 97%, at least about 98%, or at least about 99%, sequence identity, homology and/or functional equivalence to SEQ ID NO: 1024, with one or more of the viral immunosuppressive sequences expressed on the cell in a membrane-bound format, as discussed below.

In several embodiments, the engineered cells provided for herein comprise a chimeric receptor that targets NKG2D ligands, wherein the CAR comprises an amino acid of SEQ ID NO: 899, or comprises an amino acid sequence with at least about 80%, at least about 85%, at least about 90%, at least about 94%, at least about 95%, at least about 96%, at least about 97%, at least about 98%, or at least about 99%, sequence identity, homology and/or functional equivalence to SEQ ID NO: 899 with one or more copies of one or more of the following viral immunosuppressive amino acid sequences: SEQ ID NO: 199-216, 220, 225, 230, 235, 240, 245, 250, 273, 280, 288, or 289 integrated into the sequence of SEQ ID NO: 899. In addition to, or in place of the integrated viral immunosuppressive amino acids, the CAR optionally comprises an amino acid of SEQ ID NO: 899, or comprises an amino acid sequence with at least about 80%, at least about 85%, at least about 90%, at least about 94%, at least about 95%, at least about 96%, at least about 97%, at least about 98%, or at least about 99%, sequence identity, homology and/or functional equivalence to SEQ ID NO: 899, with one or more of the viral immunosuppressive sequences expressed on the cell in a membrane-bound format, as discussed below.

In several embodiments, the engineered cells provided for herein comprise a chimeric receptor that targets NKG2D ligands, wherein the CAR comprises an amino acid of SEQ ID NO: 1025, or comprises an amino acid sequence with at least about 80%, at least about 85%, at least about 90%, at least about 94%, at least about 95%, at least about 96%, at least about 97%, at least about 98%, or at least about 99%, sequence identity, homology and/or functional equivalence to SEQ ID NO: 1025 with one or more copies of one or more of the following viral immunosuppressive amino acid sequences: SEQ ID NO: 199-216, 220, 225, 230, 235, 240, 245, 250, 273, 280, 288, or 289 integrated into the sequence of SEQ ID NO: 1025. In addition to, or in place of the integrated viral immunosuppressive amino acids, the CAR optionally comprises an amino acid of SEQ ID NO: 1025, or comprises an amino acid sequence with at least about 80%, at least about 85%, at least about 90%, at least about 94%, at least about 95%, at least about 96%, at least about 97%, at least about 98%, or at least about 99%, sequence identity, homology and/or functional equivalence to SEQ ID NO: 1025, with one or more of the viral immunosuppressive sequences expressed on the cell in a membrane-bound format, as discussed below.

In several embodiments, the engineered cells provided for herein comprise a CAR that targets CD19, wherein the CAR comprises an amino acid of SEQ ID NO: 178, or comprises an amino acid sequence with at least about 80%, at least about 85%, at least about 90%, at least about 94%, at least about 95%, at least about 96%, at least about 97%, at least about 98%, or at least about 99%, sequence identity, homology and/or functional equivalence to SEQ ID NO: 178 with one or more copies of one or more of the following viral immunosuppressive amino acid sequences: SEQ ID NO: 199-216, 220, 225, 230, 235, 240, 245, 250, 273, 280, 288, or 289 integrated into the sequence of SEQ ID NO: 178. In addition to, or in place of the integrated viral immunosuppressive amino acid, the CAR optionally comprises an amino acid of SEQ ID NO: 178, or comprises an amino acid sequence with at least about 80%, at least about 85%, at least about 90%, at least about 94%, at least about 95%, at least about 96%, at least about 97%, at least about 98%, or at least about 99%, sequence identity, homology and/or functional equivalence to SEQ ID NO: 178, with one or more of the viral immunosuppressive sequences expressed on the cell in a membrane-bound format, as discussed below.

In several embodiments, the engineered cells provided for herein comprise a CAR that targets CD19, wherein the CAR comprises an amino acid of SEQ ID NO: 1026, or comprises an amino acid sequence with at least about 80%, at least about 85%, at least about 90%, at least about 94%, at least about 95%, at least about 96%, at least about 97%, at least about 98%, or at least about 99%, sequence identity, homology and/or functional equivalence to SEQ ID NO: 1026 with one or more copies of one or more of the following viral immunosuppressive amino acid sequences: SEQ ID NO: 199-216, 220, 225, 230, 235, 240, 245, 250, 273, 280, 288, or 289 integrated into the sequence of SEQ ID NO: 1026. In addition to, or in place of the integrated viral immunosuppressive amino acid, the CAR optionally comprises an amino acid of SEQ ID NO: 1026, or comprises an amino acid sequence with at least about 80%, at least about 85%, at least about 90%, at least about 94%, at least about 95%, at least about 96%, at least about 97%, at least about 98%, or at least about 99%, sequence identity, homology and/or functional equivalence to SEQ ID NO: 1026, with one or more of the viral immunosuppressive sequences expressed on the cell in a membrane-bound format, as discussed below.

In several embodiments, the engineered cells provided for herein comprise a CAR that targets CD19, wherein the CAR comprises an amino acid of SEQ ID NO: 901, or comprises an amino acid sequence with at least about 80%, at least about 85%, at least about 90%, at least about 94%, at least about 95%, at least about 96%, at least about 97%, at least about 98%, or at least about 99%, sequence identity, homology and/or functional equivalence to SEQ ID NO: 901 with one or more copies of one or more of the following viral immunosuppressive amino acid sequences: SEQ ID NO: 199-216, 220, 225, 230, 235, 240, 245, 250, 273, 280, 288, or 289 integrated into the sequence of SEQ ID NO: 901. In addition to, or in place of the integrated viral immunosuppressive amino acid, the CAR optionally comprises an amino acid of SEQ ID NO: 901, or comprises an amino acid sequence with at least about 80%, at least about 85%, at least about 90%, at least about 94%, at least about 95%, at least about 96%, at least about 97%, at least about 98%, or at least about 99%, sequence identity, homology and/or functional equivalence to SEQ ID NO: 901, with one or more of the viral immunosuppressive sequences expressed on the cell in a membrane-bound format, as discussed below.

In several embodiments, the engineered cells provided for herein comprise a CAR that targets CD19, wherein the CAR comprises an amino acid of SEQ ID NO: 1027, or comprises an amino acid sequence with at least about 80%, at least about 85%, at least about 90%, at least about 94%, at least about 95%, at least about 96%, at least about 97%, at least about 98%, or at least about 99%, sequence identity, homology and/or functional equivalence to SEQ ID NO: 1027 with one or more copies of one or more of the following viral immunosuppressive amino acid sequences: SEQ ID NO: 199-216, 220, 225, 230, 235, 240, 245, 250, 273, 280, 288, or 289 integrated into the sequence of SEQ ID NO: $$$. In addition to, or in place of the integrated viral immunosuppressive amino acid, the CAR optionally comprises an amino acid of SEQ ID NO: 1027, or comprises an amino acid sequence with at least about 80%, at least about 85%, at least about 90%, at least about 94%, at least about 95%, at least about 96%, at least about 97%, at least about 98%, or at least about 99%, sequence identity, homology and/or functional equivalence to SEQ ID NO: 1027, with one or more of the viral immunosuppressive sequences expressed on the cell in a membrane-bound format, as discussed below.

In several embodiments, the engineered cells provided for herein comprise a CAR that targets CD19, wherein the CAR is encoded by a nucleic acid sequence comprising SEQ ID NO: 466, or comprises an nucleic acid sequence with at least about 80%, at least about 85%, at least about 90%, at least about 94%, at least about 95%, at least about 96%, at least about 97%, at least about 98%, or at least about 99%, sequence identity, homology and/or functional equivalence to SEQ ID NO: 466 with one or more copies of one or more of the following viral immunosuppressive amino acid sequences: SEQ ID NO: 199-216, 220, 225, 230, 235, 240, 245, 250, 273, 280, 288, or 289 integrated into the sequence encoded by SEQ ID NO: 466. In addition to, or in place of the integrated viral immunosuppressive amino acids, the CAR optionally comprises a CAR encoded by SEQ ID NO: 466, or comprises an amino acid sequence with at least about 80%, at least about 85%, at least about 90%, at least about 94%, at least about 95%, at least about 96%, at least about 97%, at least about 98%, or at least about 99%, sequence identity, homology and/or functional equivalence to the sequence encoded by SEQ ID NO: 466, with one or more of the viral immunosuppressive sequences expressed on the cell in a membrane-bound format, as discussed below.

In several embodiments, the engineered cells provided for herein comprise a CAR that targets CD70. In several embodiments, the CAR comprises the amino acid sequence set forth in any of SEQ ID NOs: 383-465 and 912-994. In several embodiments, the engineered cells provided for herein comprise a CAR that targets CD70, wherein the CAR comprises an amino acid of any of SEQ ID NOs: 383-465 or 912-994, or comprises an amino acid sequence with at least about 80%, at least about 85%, at least about 90%, at least about 94%, at least about 95%, at least about 96%, at least about 97%, at least about 98%, or at least about 99%, sequence identity, homology and/or functional equivalence to any of SEQ ID NO: 383-465 or 912-994 with one or more copies of one or more of the following viral immunosuppressive amino acid sequences: SEQ ID NO: 199-216, 220, 225, 230, 235, 240, 245, 250, 273, 280, 288, or 289 integrated into the sequence of any of SEQ ID NO: 383-465 or 912-994. In addition to, or in place of the integrated viral immunosuppressive amino acid, the CAR optionally comprises an amino acid of any of SEQ ID NO: 383-465 or 912-994, or comprises an amino acid sequence with at least about 80%, at least about 85%, at least about 90%, at least about 94%, at least about 95%, at least about 96%, at least about 97%, at least about 98%, or at least about 99%, sequence identity, homology and/or functional equivalence to SEQ ID NO: 383-465 or 912-994, with one or more of the viral immunosuppressive sequences expressed on the cell in a membrane-bound format, as discussed below. It is contemplated that any of the amino acid sequences provided herein may be provided with or without a signal sequence (e.g., a CD8a signal sequence, such as MALPVTALLLPLALLLHAARP). It is also contemplated that any of the amino acid sequences provided herein may be provided with or without an initial methionine (M) residue.

In addition to incorporation of the viral immunosuppressive peptides being positioned in the CAR, the peptides can be coupled to a domain that allows them to be expressed as a membrane bound viral immunosuppressive peptide.

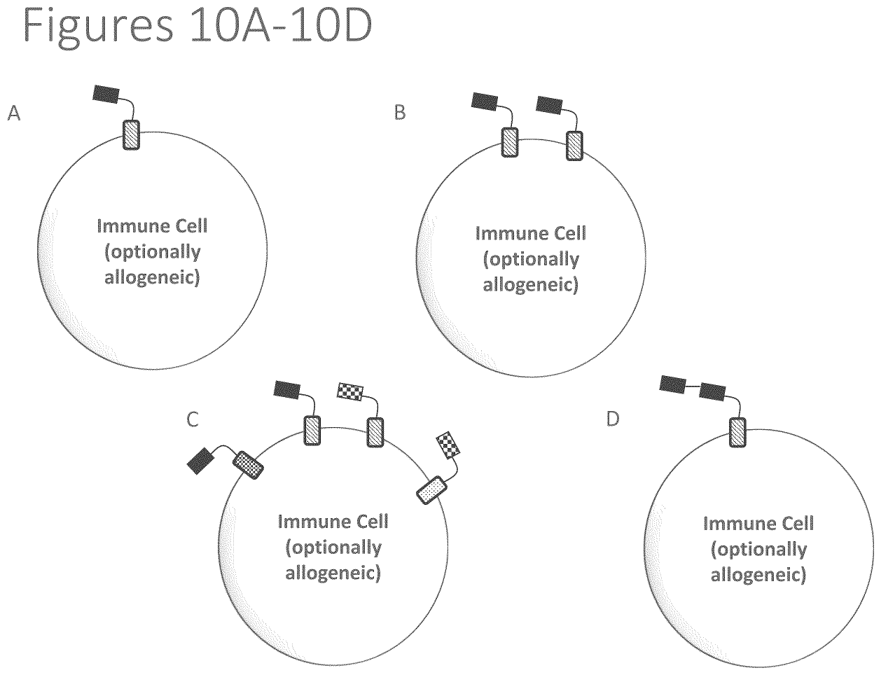

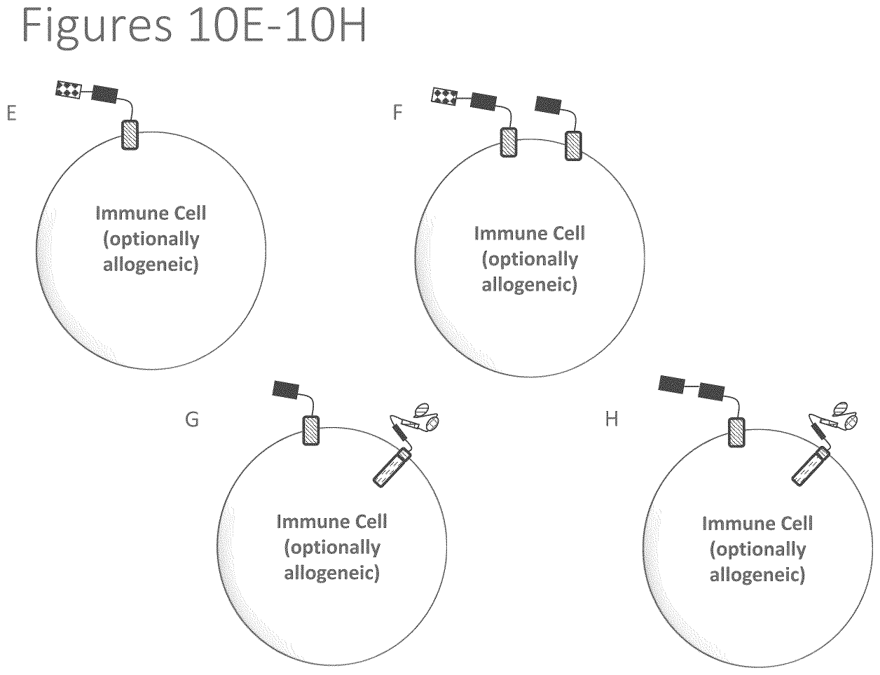

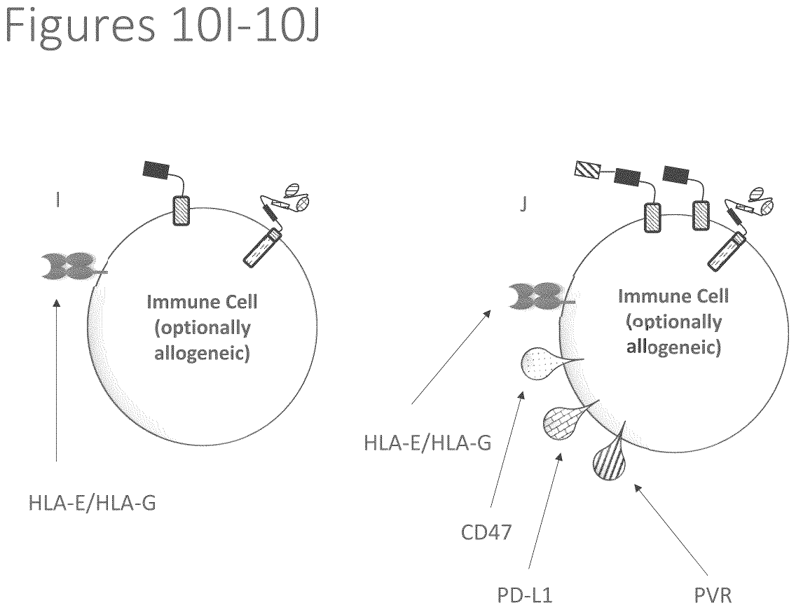

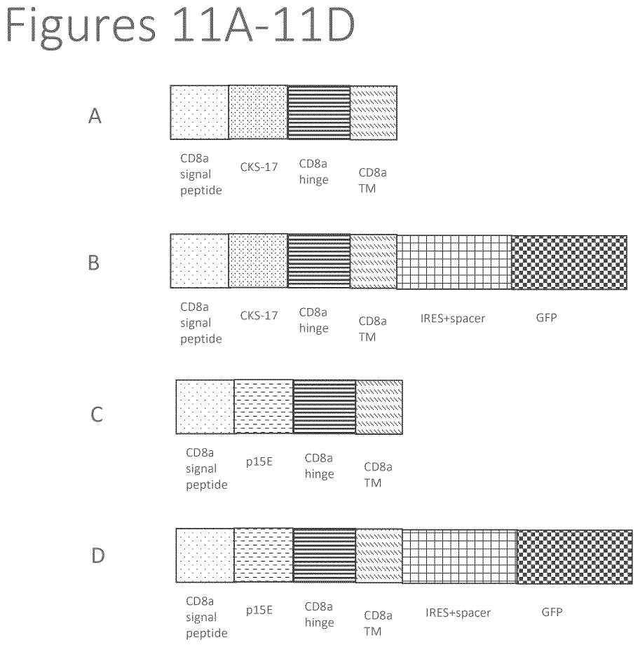

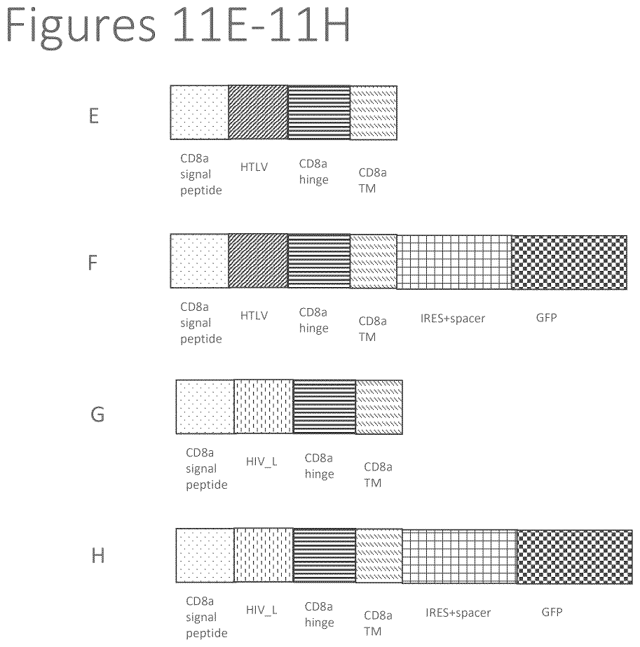

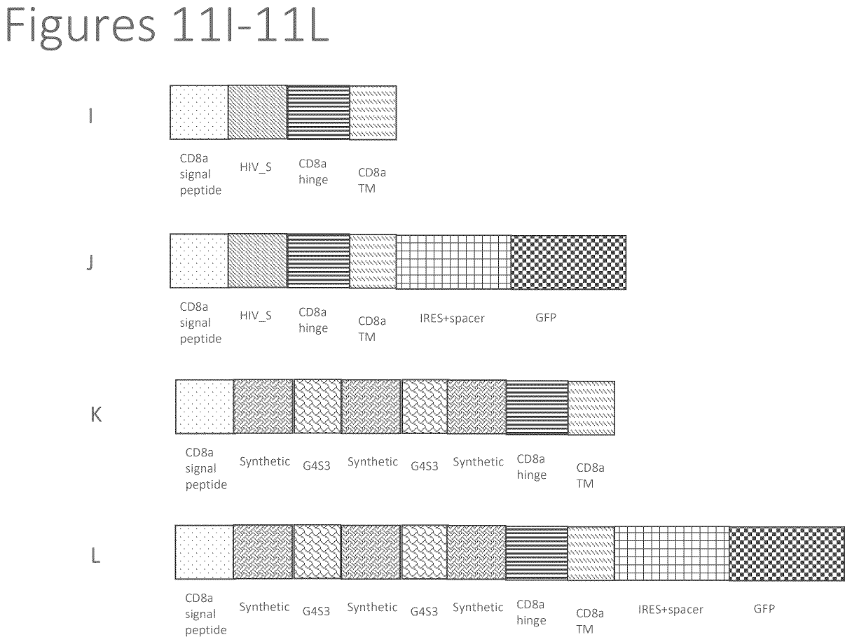

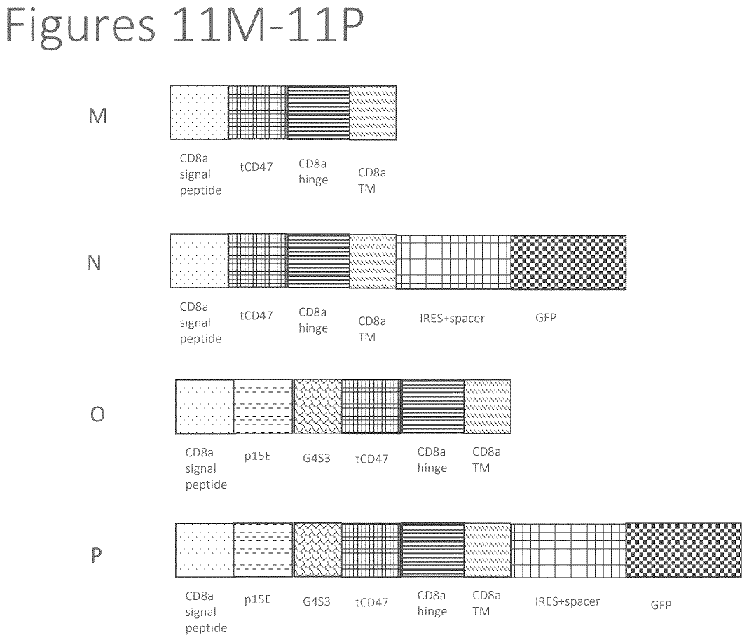

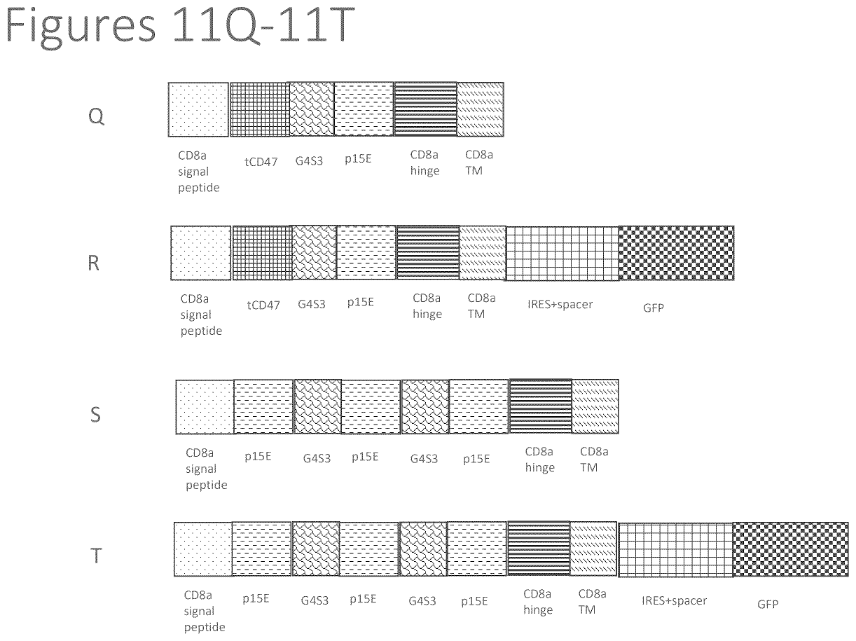

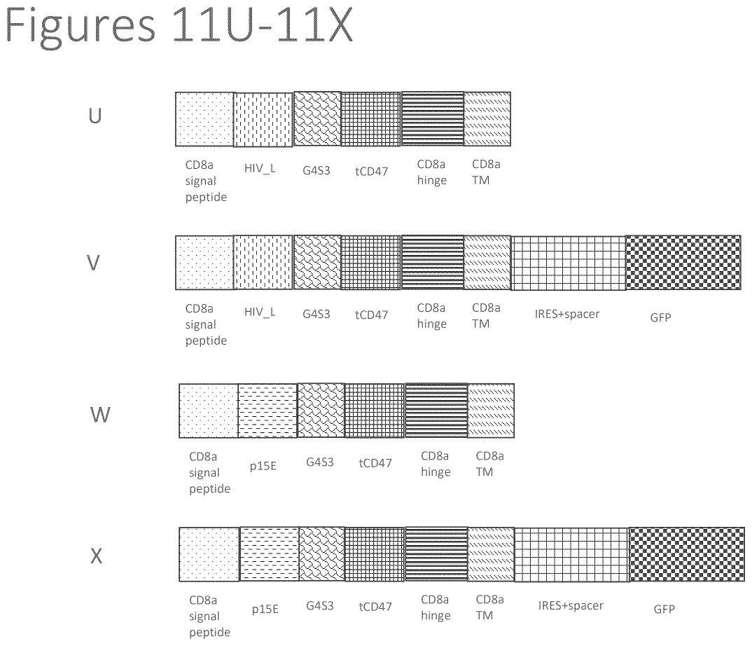

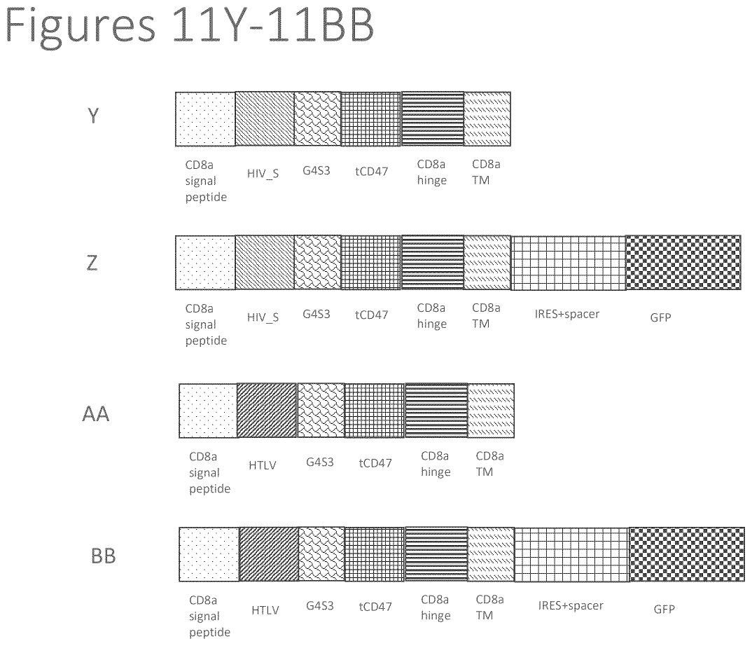

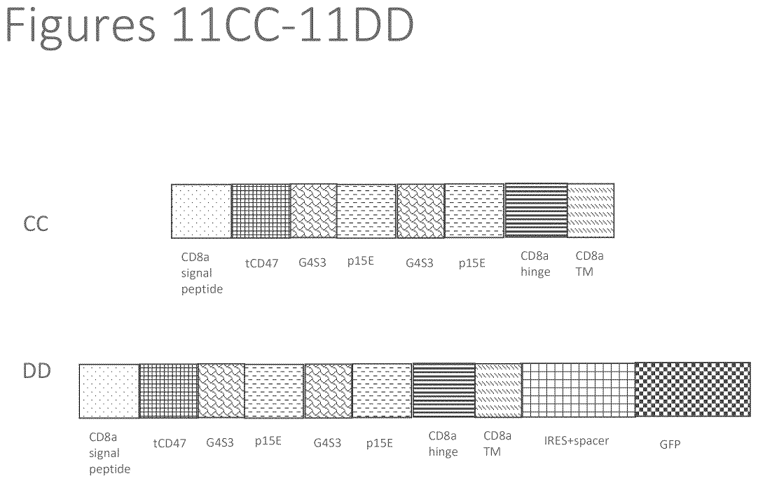

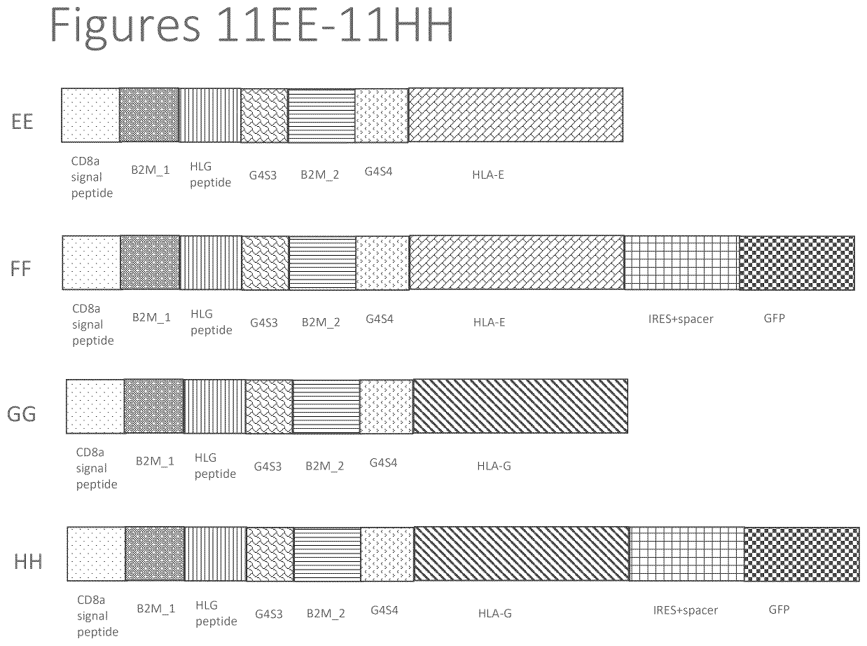

FIGS. 10A-10J show non-limiting schematic depictions of embodiments disclosed herein. FIG. 10A shows a single viral immunosuppressive peptide coupled to a transmembrane protein. FIG. 10B shows two individual viral immunosuppressive peptides, each coupled to a transmembrane protein. FIG. 10C shows a plurality of viral immunosuppressive peptides in a membrane-bound format. As shown, and described herein, both the viral immunosuppressive peptide and the transmembrane domain can vary, or can be the same. In other words, provided for herein are combinations such as viral immunosuppressive peptide (VIP) 1: transmembrane protein (TM) 1; VIP2:TM1; VIP2:TM2; VIP1:TM2; VIP3:TM1; VIP1:TM3, and the like, including VIPX:TMY.

FIG. 10D shows an additional non-limiting embodiment wherein multiple viral immunosuppressive peptides are coupled to a single transmembrane protein, with the viral immunosuppressive peptides being the same. FIG. 106E shows an additional non-limiting embodiment with wherein multiple viral immunosuppressive peptides are coupled to a single transmembrane protein, with the viral immunosuppressive peptides being distinct from one another. FIG. 10F shows a further non-limiting embodiment wherein multiple membrane-bound constructs are expressed on a single immune cell, one coupled to a single viral immunosuppressive peptide, and the other coupled to multiple viral immunosuppressive peptide. While not illustrated, it shall be appreciated that the transmembrane domains may differ from one another when multiple constructs are expressed by an individual cell.

Various transmembrane proteins can be used, such as one or more of CD8α, CD4, CD3ε, CD3γ, CD3δ, CD3ζ, CD28, CD137, glycophorin A, glycophorin D, nicotinic acetylcholine receptor, a GABA receptor, FcεRIγ, and a T-cell receptor. In several embodiments, a portion of one or more of these domains (e.g., a transmembrane domain) is used to anchor or otherwise tether the viral immunosuppressive peptide(s) to the immune cell surface. In several embodiments, the transmembrane protein comprises a CD8α transmembrane domain. In several embodiments, the CD8α transmembrane domain comprises the amino acid sequence of SEQ ID NO: 4 (IYIWAPLAGTCGVLLLSLVIT), or a sequence with at least about 80%, at least about 85%, about 90%, about 95%, about 96%, about 97%, about 98% or about 99% sequence identity with SEQ ID NO: 4. In several embodiments, the CD8α transmembrane domain is encoded by the nucleic acid sequence of SEQ ID NO: 3, or a sequence with at least about 80%, at least about 85%, about 90%, about 95%, about 96%, about 97%, about 98% or about 99% sequence identity with SEQ ID NO: 3. In several embodiments, a hinge or other linker is used to couple the viral immunosuppressive peptide to the transmembrane protein. In several embodiments, a CD8α is used. In several embodiments, the CD8α hinge comprises the amino acid sequence of SEQ ID NO: 2 (TTTPAPRPPTPAPTIASQPLSLRPEACRPAAGGAVHTRGLDFACD), or a sequence with at least about 80%, at least about 85%, about 90%, about 95%, about 96%, about 97%, about 98% or about 99% sequence identity with SEQ ID NO: 2. In several embodiments, the CD8α hinge comprises the amino acid sequence of SEQ ID NO: 1, or a sequence with at least about 80%, at least about 85%, about 90%, about 95%, about 96%, about 97%, about 98% or about 99% sequence identity with SEQ ID NO: 1.

FIG. 10G shows an additional schematic of a non-limiting embodiment provided for herein. This embodiment comprises an immune cell engineered to express a CAR as well as a membrane-bound viral immunosuppressive peptide. FIG. 10G shows a non-limiting embodiment wherein the immune cell expresses a CAR along with a plurality of viral immunosuppressive peptides coupled to a transmembrane domain. Also provided for are immune cells (e.g., NK cells, T cells or combinations there) expressing a CAR and multiple membrane bound viral immunosuppressive peptides (e.g., as in FIG. 10F). In several embodiments, the CAR comprises one or more viral immunosuppressive peptides, while some embodiments involve expression of a CAR without a viral immunosuppressive peptide. As shown in these schematic figures, the immune cells engineered, in several embodiments, are allogeneic cells. In several embodiments, allogeneic NK cells are used. In several embodiments, allogeneic T cells are used. In several embodiments, combinations (e.g., a mixed population) of allogeneic NK cell and allogeneic T cells are used.

In several embodiments, there is provided a polynucleotide encoding a synthetic CKS-17 viral immunosuppressive peptide. In several embodiments, the CKS-17 viral immunosuppressive peptide comprises the amino acid sequence set forth in SEQ ID NO:199 (LQNRRGLDLLFLKEGGL). In several embodiments, the polynucleotide encodes an amino acid sequence comprising SEQ ID NO: 199. In several embodiments, the polynucleotide encodes a sequence that shares at least about 80%, at least about 85%, at least about 90%, at least about 94%, at least about 95%, at least about 96%, at least about 97%, at least about 98%, or at least about 99%, sequence identity, homology and/or functional equivalence with SEQ ID NO: 199. In several embodiments, the polynucleotide comprises SEQ ID NO: 216 or shares at least about 80%, at least about 85%, at least about 90%, at least about 94%, at least about 95%, at least about 96%, at least about 97%, at least about 98%, or at least about 99%, sequence identity with SEQ ID NO: 216. In several embodiments, provided for is a membrane-bound synthetic CKS-17 viral immunosuppressive peptide. In several embodiments, the synthetic CKS-17 viral immunosuppressive peptide is coupled to a CD8α transmembrane protein and/or CD8α hinge domain. In several embodiments, the membrane-bound synthetic CKS-17 viral immunosuppressive peptide construct comprises one or more linker sequences. In several embodiments, the membrane-bound synthetic CKS-17 viral immunosuppressive peptide comprises a CD8α signal peptide, synthetic CKS-17, a CD8α hinge, and a CD8α transmembrane domain. In several embodiments, the membrane-bound synthetic CKS-17 viral immunosuppressive peptide construct is encoded by a polynucleotide that encodes an amino acid sequence comprising SEQ ID NO: 1028 (LQNRRGLDLLFLKEGGLTTTPAPRPPTPAPTIASQPLSLRPEACRPAAGGAVHTRGLDFACDIYIWAPLAGT CGVLLLSLVITLYC) (e.g., SEQ ID NO: 218) or SEQ ID NO: 1029 (LQNRRGLDLLFLKEGGLTTTPAPRPPTPAPTIASQPLSLRPEACRPAAGGAVHTRGLDFACDIYIWAPLAGT CGVLLLSLVITLYC) (e.g., SEQ ID NO: 693). In several embodiments, the polynucleotide encodes a sequence that shares at least about 80%, at least about 85%, at least about 90%, at least about 94%, at least about 95%, at least about 96%, at least about 97%, at least about 98%, or at least about 99%, sequence identity, homology and/or functional equivalence with SEQ ID NO: 218 or SEQ ID NO: 693. In several embodiments, the polynucleotide comprises SEQ ID NO: 219 (or SEQ ID NO: 694) or shares at least about 80%, at least about 85%, at least about 90%, at least about 94%, at least about 95%, at least about 96%, at least about 97%, at least about 98%, or at least about 99%, sequence identity with SEQ ID NO: 219 (or SEQ ID NO: 694). Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, the polynucleotide encoding membrane-bound synthetic CKS-17 and GFP comprises SEQ ID NO: 217 or shares at least about 80%, at least about 85%, at least about 90%, at least about 94%, at least about 95%, at least about 96%, at least about 97%, at least about 98%, or at least about 99%, sequence identity with SEQ ID NO: 217. In several embodiments, the polynucleotide encoding membrane-bound synthetic CKS-17, a FLAG tag, and GFP comprises SEQ ID NO: 692 or shares at least about 80%, at least about 85%, at least about 90%, at least about 94%, at least about 95%, at least about 96%, at least about 97%, at least about 98%, or at least about 99%, sequence identity with SEQ ID NO: 692. In several embodiments, the polynucleotide encoding membrane-bound synthetic CKS-17, a FLAG tag, and GFP encodes the amino acid sequence of SEQ ID NO: 691 or an amino acid sequence that shares at least about 80%, at least about 85%, at least about 90%, at least about 94%, at least about 95%, at least about 96%, at least about 97%, at least about 98%, or at least about 99%, sequence identity with SEQ ID NO: 691.

In several embodiments, the membrane-bound synthetic CKS-17 viral immunosuppressive peptide construct comprises the amino acid sequence set forth in SEQ ID NO: 1028 or 1029. In several embodiments, the membrane-bound synthetic CKS-17 viral immunosuppressive peptide construct comprises an amino acid sequence with at least about 80%, at least about 85%, at least about 90%, at least about 94%, at least about 95%, at least about 96%, at least about 97%, at least about 98%, or at least about 99%, sequence identity to SEQ ID NO: 1028 or SEQ ID NO: 1029.

In several embodiments, there is provided a polynucleotide encoding a p15E viral immunosuppressive peptide. In several embodiments, the p15E viral immunosuppressive peptide comprises the amino acid sequence set forth in SEQ ID NO:220 (LQNRRGLDLLFLKEGGLCAALKEECCFY). In several embodiments, the polynucleotide encodes an amino acid sequence comprising SEQ ID NO: 220. In several embodiments, the polynucleotide encodes a sequence that shares at least about 80%, at least about 85%, at least about 90%, at least about 94%, at least about 95%, at least about 96%, at least about 97%, at least about 98%, or at least about 99%, sequence identity, homology and/or functional equivalence with SEQ ID NO: 220. In several embodiments, the polynucleotide comprises SEQ ID NO: 221 or shares at least about 80%, at least about 85%, at least about 90%, at least about 94%, at least about 95%, at least about 96%, at least about 97%, at least about 98%, or at least about 99%, sequence identity with SEQ ID NO: 221. In several embodiments, provided for is a membrane-bound p15E viral immunosuppressive peptide. In several embodiments, the p15E viral immunosuppressive peptide is coupled to a CD8α transmembrane protein and/or CD8α hinge domain. In several embodiments, the membrane-bound p15E viral immunosuppressive peptide construct comprises one or more linker sequences. In several embodiments, the membrane-bound p15E viral immunosuppressive peptide comprises a CD8α signal peptide, p15E, a CD8α hinge, and a CD8α transmembrane domain. In several embodiments, the membrane-bound p15E viral immunosuppressive peptide construct is encoded by a polynucleotide that encodes an amino acid sequence comprising SEQ ID NO: 1030 (LQNRRGLDLLFLKEGGLCAALKEECCFTTTPAPRPPTPAPTIASQPLSLRPEACRPAAGGAVHTRGLDFACD IYIWAPLAGTCGVLLLSLVITLYC) (e.g., SEQ ID NO: 223) or SEQ ID NO: 1031 (LQNRRGLDLLFLKEGGLCAALKEECCFYTTTPAPRPPTPAPTIASQPLSLRPEACRPAAGGAVHTRGLDFAC DIYIWAPLAGTCGVLLLSLVITLYC) (e.g., SEQ ID NO: 697). In several embodiments, the polynucleotide encodes a sequence that shares at least about 90%, at least about 94%, at least about 95%, at least about 96%, at least about 97%, at least about 98%, or at least about 99%, sequence identity, homology and/or functional equivalence with SEQ ID NO: 1030 (or SEQ ID NO: 223 or SEQ ID NO: 697). In several embodiments, the polynucleotide comprises SEQ ID NO: 1031 (or SEQ ID NO: 224 or SEQ ID NO: 698) or shares at least about 80%, at least about 85%, at least about 90%, at least about 94%, at least about 95%, at least about 96%, at least about 97%, at least about 98%, or at least about 99%, sequence identity with SEQ ID NO: 1031 (or SEQ ID NO: 224 or SEQ ID NO: 698). Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, the polynucleotide encoding mbp15E and GFP comprises SEQ ID NO: 222 or shares at least about 80%, at least about 85%, at least about 90%, at least about 94%, at least about 95%, at least about 96%, at least about 97%, at least about 98%, or at least about 99%, sequence identity with SEQ ID NO: 222. In several embodiments, the polynucleotide encoding mbp15E, a FLAG tag, and GFP comprises SEQ ID NO: 696 or shares at least about 80%, at least about 85%, at least about 90%, at least about 94%, at least about 95%, at least about 96%, at least about 97%, at least about 98%, or at least about 99%, sequence identity with SEQ ID NO: 696. In several embodiments, the membrane-bound p15E viral immunosuppressive peptide construct including GFP and a FLAG tag is encoded by a polynucleotide that encodes an amino acid sequence comprising SEQ ID NO: 695). In several embodiments, the polynucleotide encodes a sequence that shares at least about 90%, at least about 94%, at least about 95%, at least about 96%, at least about 97%, at least about 98%, or at least about 99%, sequence identity, homology and/or functional equivalence with SEQ ID NO: 695).

In several embodiments, the membrane-bound p15E viral immunosuppressive peptide construct comprises the amino acid sequence set forth in SEQ ID NO: 1030 or 1031. In several embodiments, the membrane-bound p15E viral immunosuppressive peptide construct comprises an amino acid sequence with at least about 80%, at least about 85%, at least about 90%, at least about 94%, at least about 95%, at least about 96%, at least about 97%, at least about 98%, or at least about 99%, sequence identity to SEQ ID NO: 1030 or SEQ ID NO: 1031.

In several embodiments, there is provided a polynucleotide encoding a HTLV viral immunosuppressive peptide. In several embodiments, the HTLV viral immunosuppressive peptide comprises the amino acid sequence set forth in SEQ ID NO:225 (AQNRRGLDLLFWEQGGLCKALQEQCRFP). In several embodiments, the polynucleotide encodes an amino acid sequence comprising SEQ ID NO: 225. In several embodiments, the polynucleotide encodes a sequence that shares at least about 80%, at least about 85%, at least about 90%, at least about 94%, at least about 95%, at least about 96%, at least about 97%, at least about 98%, or at least about 99%, sequence identity, homology and/or functional equivalence with SEQ ID NO: 225. In several embodiments, the polynucleotide comprises SEQ ID NO: 226 or shares at least about 80%, at least about 85%, at least about 90%, at least about 94%, at least about 95%, at least about 96%, at least about 97%, at least about 98%, or at least about 99%, sequence identity with SEQ ID NO: 226. In several embodiments, provided for is a membrane-bound HTLV viral immunosuppressive peptide. In several embodiments, the HTLV viral immunosuppressive peptide is coupled to a CD8α transmembrane protein and/or CD8α hinge domain. In several embodiments, the membrane-bound HTLV viral immunosuppressive peptide construct comprises one or more linker sequences. In several embodiments, the membrane-bound HTLV viral immunosuppressive peptide comprises a CD8α signal peptide, HTLV-1 (Gp21), a CD8α hinge, a and a CD8α transmembrane domain. In several embodiments, the membrane-bound HTLV viral immunosuppressive peptide construct is encoded by a polynucleotide that encodes an amino acid sequence comprising SEQ ID NO: 1032 (AQNRRGLDLLFWEQTTTPAPRPPTPAPTIASQPLSLRPEACRPAAGGAVHTRGLDFACDIYIWAPLAGTCG VLLLSLVITLYC), (e.g., SEQ ID NO: 228) or SEQ ID NO:1033 (AQNRRGLDLLFWEQGGLCKALQEQCRFPTTTPAPRPPTPAPTIASQPLSLRPEACRPAAGGAVHTRGLDFA CDIYIWAPLAGTCGVLLLSLVITLYC), (e.g., SEQ ID NO: 701). In several embodiments, the polynucleotide encodes an amino acid sequence that shares at least about 80%, at least about 85%, at least about 90%, at least about 94%, at least about 95%, at least about 96%, at least about 97%, at least about 98%, or at least about 99%, sequence identity, homology and/or functional equivalence with SEQ ID NO: 228 (or SEQ ID NO: 701). In several embodiments, the polynucleotide comprises SEQ ID NO: 229 (or SEQ ID NO: 702) or shares at least about 90%, at least about 94%, at least about 95%, at least about 96%, at least about 97%, at least about 98%, or at least about 99%, sequence identity with SEQ ID NO: 229 (or SEQ ID NO: 702). Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, the polynucleotide encoding membrane-bound HTLV and GFP comprises SEQ ID NO: 227 or shares at least about 80%, at least about 85%, at least about 90%, at least about 94%, at least about 95%, at least about 96%, at least about 97%, at least about 98%, or at least about 99%, sequence identity with SEQ ID NO: 227. In several embodiments, the polynucleotide encoding membrane-bound HTLV, a FLAG tag, and GFP comprises SEQ ID NO: 700 or shares at least about 80%, at least about 85%, at least about 90%, at least about 94%, at least about 95%, at least about 96%, at least about 97%, at least about 98%, or at least about 99%, sequence identity with SEQ ID NO: 700. In several embodiments, the membrane-bound HTLV, FLAG tag, GFP construct comprises the amino acid sequence of SEQ ID NO: 699 or shares at least about 80%, at least about 85%, at least about 90%, at least about 94%, at least about 95%, at least about 96%, at least about 97%, at least about 98%, or at least about 99%, sequence identity with SEQ ID NO: 699.

In several embodiments, the membrane-bound HTLV viral immunosuppressive peptide construct comprises the amino acid sequence set forth in SEQ ID NO: 1032 or 1033. In several embodiments, the membrane-bound HTLV viral immunosuppressive peptide construct comprises an amino acid sequence with at least about 80%, at least about 85%, at least about 90%, at least about 94%, at least about 95%, at least about 96%, at least about 97%, at least about 98%, or at least about 99%, sequence identity to SEQ ID NO: 1032 or SEQ ID NO: 1033.

In several embodiments, there is provided a polynucleotide encoding a modified HIV Gp41 viral immunosuppressive peptide. In some embodiments, the modified HIV Gp41 viral immunosuppressive peptide comprises the amino acid sequence set forth in SEQ ID NO:230 (GALFLGFLGAAGSTMGAASVTLTVQARQLLSGIVQQQSNLLRAIEAQQHMLQLTVWGIKQLQARVLAVE RYLKDQ). In several embodiments, the polynucleotide encodes an amino acid sequence comprising SEQ ID NO: 230. In several embodiments, the polynucleotide encodes a sequence that shares at least about 80%, at least about 85%, at least about 90%, at least about 94%, at least about 95%, at least about 96%, at least about 97%, at least about 98%, or at least about 99%, sequence identity, homology and/or functional equivalence with SEQ ID NO: 230. In several embodiments, the polynucleotide comprises SEQ ID NO: 231 or shares at least about 80%, at least about 85%, at least about 90%, at least about 94%, at least about 95%, at least about 96%, at least about 97%, at least about 98%, or at least about 99%, sequence identity with SEQ ID NO: 231. In several embodiments, provided for is a membrane-bound modified HIV Gp41 viral immunosuppressive peptide. In several embodiments, the modified HIV Gp41 viral immunosuppressive peptide is coupled to a CD8α transmembrane protein and/or CD8α hinge domain. In several embodiments, the membrane-bound modified HIV Gp41 viral immunosuppressive peptide construct comprises one or more linker sequences. In several embodiments, the membrane-bound modified HIV Gp41 viral immunosuppressive peptide comprises a CD8α signal peptide, modified HIV Gp41, a CD8α hinge, a and a CD8α transmembrane domain. In several embodiments, the membrane-bound modified HIV Gp41 viral immunosuppressive peptide construct is encoded by a polynucleotide that encodes an amino acid sequence comprising SEQ ID NO: 1034 (GALFLGFLGAAGSTMGAASVTLTVQARQLLSGIVQQQSNLLRAIEAQQHMLQLTVWGIKQLQARVLAVE RYLKDQTTTPAPRPPTPAPTIASQPLSLRPEACRPAAGGAVHTRGLDFACDIYIWAPLAGTCGVLLLSLVITL YC), (e.g., SEQ ID NO: 233) or SEQ ID NO: 1035 (GALFLGFLGAAGSTMGAASVTLTVQARLLLSGIVQQQNNLLRAIEAQQHLLQLTVWGIKQLQARVLAVE RYLRDQTTTPAPRPPTPAPTIASQPLSLRPEACRPAAGGAVHTRGLDFACDIYIWAPLAGTCGVLLLSLVITL YC), (e.g., SEQ ID NO: 705). In several embodiments, the polynucleotide encodes a sequence that shares at least about 90%, at least about 94%, at least about 95%, at least about 96%, at least about 97%, at least about 98%, or at least about 99%, sequence identity, homology and/or functional equivalence with SEQ ID NO: 233 (or SEQ ID NO: 705). In several embodiments, the polynucleotide comprises SEQ ID NO: 234 (or SEQ ID NO: 706) or shares at least about 80%, at least about 85%, at least about 90%, at least about 94%, at least about 95%, at least about 96%, at least about 97%, at least about 98%, or at least about 99%, sequence identity with SEQ ID NO: 234 (or SEQ ID NO: 706). Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, the polynucleotide encoding membrane-bound modified HIV Gp41 and GFP comprises SEQ ID NO: 232 or shares at least about 80%, at least about 85%, at least about 90%, at least about 94%, at least about 95%, at least about 96%, at least about 97%, at least about 98%, or at least about 99%, sequence identity with SEQ ID NO: 232. In several embodiments, the polynucleotide encoding membrane-bound modified HIV Gp41, a FLAG tag, and GFP comprises SEQ ID NO: 704 or shares at least about 80%, at least about 85%, at least about 90%, at least about 94%, at least about 95%, at least about 96%, at least about 97%, at least about 98%, or at least about 99%, sequence identity with SEQ ID NO: 704. In several embodiments, the membrane-bound modified HIV Gp41, FLAG tag, GFP construct comprises the amino acid sequence of SEQ ID NO: 703 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 703.

In several embodiments, there is provided a polynucleotide encoding a truncated HIV Gp41 viral immunosuppressive peptide. In some embodiments, the truncated HIV Gp41 viral immunosuppressive peptide comprises the amino acid sequence set forth in SEQ ID NO:235 (LQARILAVERYLKD). In several embodiments, the polynucleotide encodes an amino acid sequence comprising SEQ ID NO: 235. In several embodiments, the polynucleotide encodes a sequence that shares at least about 80%, at least about 85%, at least about 90%, at least about 94%, at least about 95%, at least about 96%, at least about 97%, at least about 98%, or at least about 99%, sequence identity, homology and/or functional equivalence with SEQ ID NO: 235. In several embodiments, the polynucleotide comprises SEQ ID NO: 236 or shares at least about 80%, at least about 85%, at least about 90%, at least about 94%, at least about 95%, at least about 96%, at least about 97%, at least about 98%, or at least about 99%, sequence identity with SEQ ID NO: 236. In several embodiments, provided for is a membrane-bound HIV Gp41 viral immunosuppressive peptide. In several embodiments, the HIV Gp41 viral immunosuppressive peptide is coupled to a CD8α transmembrane protein and/or CD8α hinge domain. In several embodiments, the membrane-bound HIV Gp41 viral immunosuppressive peptide construct comprises one or more linker sequences. In several embodiments, the membrane-bound HIV Gp41 viral immunosuppressive peptide comprises a CD8α signal peptide, HIV Gp41, a CD8α hinge, a and a CD8α transmembrane domain. In several embodiments, the membrane-bound HIV Gp41 viral immunosuppressive peptide construct is encoded by a polynucleotide that encodes an amino acid sequence comprising SEQ ID NO: 1036 (LQARILAVERYLKDTTTPAPRPPTPAPTIASQPLSLRPEACRPAAGGAVHTRGLDFACDIYIWAPLAGTCGV LLLSLVITLYC), (e.g., SEQ ID NO: 238) or SEQ ID NO: 1037 (LQARILAVERYLKDTTTPAPRPPTPAPTIASQPLSLRPEACRPAAGGAVHTRGLDFACDIYIWAPLAGTCGV LLLSLVITLYC), (e.g., SEQ ID NO: 709). In several embodiments, the polynucleotide encodes a sequence that shares at least about 80%, at least about 85%, at least about 90%, at least about 94%, at least about 95%, at least about 96%, at least about 97%, at least about 98%, or at least about 99%, sequence identity, homology and/or functional equivalence with SEQ ID NO: 238 (or SEQ ID NO: 709). In several embodiments, the polynucleotide comprises SEQ ID NO: 239 (or SEQ ID NO: 710) or shares at least about 80%, at least about 85%, at least about 90%, at least about 94%, at least about 95%, at least about 96%, at least about 97%, at least about 98%, or at least about 99%, sequence identity with SEQ ID NO: 239 (or SEQ ID NO: 710). Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, the polynucleotide encoding membrane-bound HIV Gp41 and GFP comprises SEQ ID NO: 237 or shares at least about 80%, at least about 85%, at least about 90%, at least about 94%, at least about 95%, at least about 96%, at least about 97%, at least about 98%, or at least about 99%, sequence identity with SEQ ID NO: 237. In several embodiments, the polynucleotide encoding membrane-bound HIV Gp41, a FLAG tag, and GFP comprises SEQ ID NO: 708 or shares at least about 80%, at least about 85%, at least about 90%, at least about 94%, at least about 95%, at least about 96%, at least about 97%, at least about 98%, or at least about 99%, sequence identity with SEQ ID NO: 708. In several embodiments, the membrane-bound truncated HIV Gp41, FLAG tag, GFP construct comprises the amino acid sequence of SEQ ID NO: 707 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 707.

In several embodiments, there is provided a polynucleotide encoding a synthetic viral immunosuppressive peptide. In some embodiments, the synthetic viral immunosuppressive peptide comprises the amino acid sequence set forth in SEQ ID NO:240 (AGFGLLLGF). In several embodiments, the polynucleotide encodes an amino acid sequence comprising SEQ ID NO: 240. In several embodiments, the polynucleotide encodes a sequence that shares at least about 80%, at least about 85%, at least about 90%, at least about 94%, at least about 95%, at least about 96%, at least about 97%, at least about 98%, or at least about 99%, sequence identity, homology and/or functional equivalence with SEQ ID NO: 240. In several embodiments, the polynucleotide comprises SEQ ID NO: 241 or shares at least about 80%, at least about 85%, at least about 90%, at least about 94%, at least about 95%, at least about 96%, at least about 97%, at least about 98%, or at least about 99%, sequence identity with SEQ ID NO: 241. In several embodiments, provided for is a membrane-bound synthetic viral immunosuppressive peptide. In several embodiments, the synthetic viral immunosuppressive peptide is coupled to a CD8α transmembrane protein and/or CD8α hinge domain. In several embodiments, the membrane-bound synthetic viral immunosuppressive peptide construct comprises one or more linker sequences. In several embodiments, the membrane-bound synthetic viral immunosuppressive peptide comprises a CD8α signal peptide, a synthetic viral immunosuppressive peptide trimer, a CD8α hinge, a and a CD8α transmembrane domain. In several embodiments, the membrane-bound synthetic viral immunosuppressive peptide construct is encoded by a polynucleotide that encodes an amino acid sequence comprising SEQ ID NO: 1038 (AGFGLLLGFGGGGSGGGGSGGGGSAGFGLLLGFGGGGSGGGGSGGGGSAGFGLLLGFTTTPAPRPPTPAP TIASQPLSLRPEACRPAAGGAVHTRGLDFACDIYIWAPLAGTCGVLLLSLVITLYC), (e.g., SEQ ID NO: 243) or SEQ ID NO: 1039 (AGFGLLLGFGGGGSGGGGSGGGGSAGFGLLLGFGGGGSGGGGSGGGGSAGFGLLLGFTTTPAPRPPTPAP TIASQPLSLRPEACRPAAGGAVHTRGLDFACDIYIWAPLAGTCGVLLLSLVITLYC), (e.g., SEQ ID NO: 713). In several embodiments, the polynucleotide encodes a sequence that shares at least about 80%, at least about 85%, at least about 90%, at least about 94%, at least about 95%, at least about 96%, at least about 97%, at least about 98%, or at least about 99%, sequence identity, homology and/or functional equivalence with SEQ ID NO: 243 (or SEQ ID NO: 713). In several embodiments, the polynucleotide comprises SEQ ID NO: 244 (or SEQ ID NO: 714) or shares at least about 80%, at least about 85%, at least about 90%, at least about 94%, at least about 95%, at least about 96%, at least about 97%, at least about 98%, or at least about 99%, sequence identity with SEQ ID NO: 244 (or SEQ ID NO: 714). Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, the polynucleotide encoding membrane-bound synthetic viral immunosuppressive peptide and GFP comprises SEQ ID NO: 242 or shares at least about 80%, at least about 85%, at least about 90%, at least about 94%, at least about 95%, at least about 96%, at least about 97%, at least about 98%, or at least about 99%, sequence identity with SEQ ID NO: 242. In several embodiments, the polynucleotide encoding membrane-bound synthetic viral immunosuppressive peptide, a FLAG tag and GFP comprises SEQ ID NO: 712 or shares at least about 80%, at least about 85%, at least about 90%, at least about 94%, at least about 95%, at least about 96%, at least about 97%, at least about 98%, or at least about 99%, sequence identity with SEQ ID NO: 712. In several embodiments, membrane-bound synthetic viral immunosuppressive peptide, FLAG tag, GFP construct comprises the amino acid of SEQ ID NO: 712 or shares at least about 80%, at least about 85%, at least about 90%, at least about 94%, at least about 95%, at least about 96%, at least about 97%, at least about 98%, or at least about 99%, sequence identity with SEQ ID NO: 712.

In several embodiments, viral fusion peptides, or variants thereof, are used. By way of example, HIV initiates an immune evasive response by fusing to a target cell via a fusion peptide, a portion of which interacts with the T cell receptor on host T cells and suppresses their activation. In several embodiments, a portion of a viral fusion peptide is used. For example, in several embodiments, an amino acid sequence comprising residues 5 to 13 of the HIV fusion peptide are used in an immunosuppressive effector as disclosed herein (e.g., incorporated into a CAR at one or more extracellular locations, or with one or more copies coupled to a transmembrane domain). In several embodiments, that amino acid sequence comprises SEQ ID NO: 467. In several embodiments, the amino acid sequence shares at least about 80%, at least about 85%, at least about 90%, at least about 94%, at least about 95%, at least about 96%, at least about 97%, at least about 98%, or at least about 99%, sequence identity, homology and/or functional equivalence with SEQ ID NO: 467. In several embodiments, a modified viral fusion protein is used in an immunosuppressive effector as disclosed herein (e.g., incorporated into a CAR at one or more extracellular locations, or with one or more copies coupled to a transmembrane domain). In several embodiments, that amino acid sequence comprises SEQ ID NO: 468. In several embodiments, the amino acid sequence shares at least about 80%, at least about 85%, at least about 90%, at least about 94%, at least about 95%, at least about 96%, at least about 97%, at least about 98%, or at least about 99%, sequence identity, homology and/or functional equivalence with SEQ ID NO: 468. In several embodiments, one or more consensus motifs derived from a viral fusion protein are used in an immunosuppressive effector as disclosed herein (e.g., incorporated into a CAR at one or more extracellular locations, or with one or more copies coupled to a transmembrane domain). In several embodiments, that amino acid consensus sequence comprises GXXXG (SEQ ID NO: 473) or AXXXG (SEQ ID NO: 474), where each X independently is any amino acid. In several embodiments, the amino acid sequence shares at least about 80%, at least about 85%, at least about 90%, at least about 94%, at least about 95%, at least about 96%, at least about 97%, at least about 98%, or at least about 99%, sequence identity, homology and/or functional equivalence with SEQ ID NO: 473 or 474, while maintaining the consensus motif. In several embodiments, these motifs are particularly advantageous in that they are suppressive towards T-cells and not NKs. Thus, in several embodiments, these peptides allow engineered NK cells to be developed without gene editing to reduce/knock out B2M expression and still effect functional reduction in host versus graft rejection (e.g., through T cell suppression alone).

In several embodiments, there is provided a polynucleotide encoding a p15E viral immunosuppressive trimeric peptide. In some embodiments, the p15E viral immunosuppressive peptide comprises the amino acid sequence set forth in SEQ ID NO:250 (LQNRRGLDLLFLKEGGLCAALKEECCFY). In several embodiments, the polynucleotide encodes an amino acid sequence comprising SEQ ID NO: 250. In several embodiments, the polynucleotide encodes a sequence that shares at least about 80%, at least about 85%, at least about 90%, at least about 94%, at least about 95%, at least about 96%, at least about 97%, at least about 98%, or at least about 99%, sequence identity, homology and/or functional equivalence with SEQ ID NO: 250. In several embodiments, the polynucleotide comprises SEQ ID NO: 251 or shares at least about 80%, at least about 85%, at least about 90%, at least about 94%, at least about 95%, at least about 96%, at least about 97%, at least about 98%, or at least about 99%, sequence identity with SEQ ID NO: 251. In several embodiments, provided for is a membrane-bound p15E viral immunosuppressive trimeric peptide. In several embodiments, the p15E viral immunosuppressive trimeric peptide is coupled to a CD8α transmembrane protein and/or CD8α hinge domain. In several embodiments, the membrane-bound p15E viral immunosuppressive trimeric peptide construct comprises one or more linker sequences (e.g., in between the peptide repeats). In several embodiments, the membrane-bound p15E viral immunosuppressive trimeric peptide comprises a CD8α signal peptide, a first p15E peptide, a linker (e.g., a GS linker), a second p15E peptide, a linker (e.g., a second GS linker), a third p15E peptide, a CD8α hinge, and a CD8α transmembrane domain. In several embodiments, the membrane-bound p15E viral immunosuppressive peptide construct is encoded by a polynucleotide that encodes an amino acid sequence comprising SEQ ID NO: 1040 (LQNRRGLDLLFLKEGGLCAALKEECCFYGGGGSGGGGSGGGGSLQNRRGLDLLFLKEGGLCAALKEECC FYGGGGSGGGGSGGGGSLQNRRGLDLLFLKEGGLCAALKEECCFYTTTPAPRPPTPAPTIASQPLSLRPEAC RPAAGGAVHTRGLDFACDIYIWAPLAGTCGVLLLSLVITLYC), (e.g., SEQ ID NO: 253) or SEQ ID NO: 1041 (LQNRRGLDLLFLKEGGLCAALKEECCFYGGGGSGGGGSGGGGSLQNRRGLDLLFLKEGGLCAALKEECC FYGGGGSGGGGSGGGGSLQNRRGLDLLFLKEGGLCAALKEECCFYTTTPAPRPPTPAPTIASQPLSLRPEAC RPAAGGAVHTRGLDFACDIYIWAPLAGTCGVLLLSLVITLYC), (e.g., SEQ ID NO: 721). In several embodiments, the polynucleotide encodes a sequence that shares at least about 80%, at least about 85%, at least about 90%, at least about 94%, at least about 95%, at least about 96%, at least about 97%, at least about 98%, or at least about 99%, sequence identity, homology and/or functional equivalence with SEQ ID NO: 253 (or SEQ ID NO: 721). In several embodiments, the polynucleotide comprises SEQ ID NO: 254 (or SEQ ID NO: 722) or shares at least about 80%, at least about 85%, at least about 90%, at least about 94%, at least about 95%, at least about 96%, at least about 97%, at least about 98%, or at least about 99%, sequence identity with SEQ ID NO: 254 (or SEQ ID NO: 722). Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, the polynucleotide encoding membrane-bound trimeric p15E and GFP comprises SEQ ID NO: 252 or shares at least about 80%, at least about 85%, at least about 90%, at least about 94%, at least about 95%, at least about 96%, at least about 97%, at least about 98%, or at least about 99%, sequence identity with SEQ ID NO: 252. In several embodiments, the polynucleotide encoding membrane-bound trimeric p15E, a FLAG tag, and GFP comprises SEQ ID NO: 720 or shares at least about 80%, at least about 85%, at least about 90%, at least about 94%, at least about 95%, at least about 96%, at least about 97%, at least about 98%, or at least about 99%, sequence identity with SEQ ID NO: 720. In several embodiments, the membrane-bound trimeric p15E, FLAG tag, GFP construct comprises the amino acid sequence of SEQ ID NO: 719 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 719.

Chimeric proteins that include one or more viral immunosuppressive peptides are also used, in several embodiments. One such chimeric protein comprises the human cytomegalovirus class I MHC homolog, UL18. As described in more detail below, in several embodiments, UL18 is incorporated into a CAR, or otherwise expressed (e.g., through chimeric UL18-B2M or in a dimer/trimer construct) in order to evade host-NK cell cytotoxicity through the UL18 binding to the NK cell inhibitory receptor LIR-1.

Additionally, certain sequence domains or even particular residues within viral immunosuppressive peptides can facilitate further engineering of chimeric antigen receptors and their expression. For example, certain residues could be used to engineer CAR dimers, e.g., through disulfide bonding. Such an approach could be utilized to supplement, or replace, the use of bi-specific CARs. For example, rather than engineering a single CAR with, by way of example an anti-CD19 binder and a binder of NKG2D ligands, two separate CARs are engineered, each with cysteine residues positioned in a manner that allowed for the formation of di-sulfide bridges between the two CARs and thus the self-assembly of a dimerized CAR in vivo.

Advantageously, the use of the viral immunosuppressive peptides can not only serve to help mute the host NK cell response against administered engineered cells, but it can be used for other purposes as well. For example, in some embodiments, an antibody directed to a viral peptide is administered to a subject who has been dosed with a cell product expressing a CAR that comprises one or more viral immunosuppressive peptides. The antibody functions to bind the viral peptide and induce depletion of the CAR-expressing cells (e.g., via antibody-based immune response), thus serving as a safety mechanism or a route to end a treatment. Similarly, during manufacture of an immune cell comprising a CAR with an included viral immunosuppressive peptide, antibody-based detection of the viral immunosuppressive peptide can be used to determine CAR expression levels.

### CD47 and Other Immunosuppressive Peptides, and Combinations with Viral Immunosuppressive Peptides

In addition to viral immunosuppressive peptides, other immunosuppressive peptides or polypeptides are provided for herein. Like the viral immunosuppressive peptides, these polypeptides may be included in one or more regions of a CAR, or can be expressed in a membrane-bound format. These additional immunosuppressive polypeptides can also be used in connection with one or more viral immunosuppressive peptides (see, e.g., FIGS. 9 and 10 and description of viral peptides above).

In several embodiments endogenous “self” signals are re-purposed to impart immune evasiveness to engineered immune cells. One such “self” protein is CD47, which impedes phagocytosis (e.g., by macrophages) through signaling through the phagocyte receptor CD172a. In several embodiments, one or more domains (or sub-domains) of CD47 are incorporated into a CAR and/or expressed in an immune cell in a membrane-bound configuration. In several embodiments, the expression of CD47 (in whole or in part) functions to impart to the engineered immune cell the ability to reduce or avoid phagocytosis by host immune cells, thereby enhancing the persistence (and thus functional life-span) of the engineered immune cells.

Other immunosuppressive peptides are also provided for herein. In several embodiments, PD-L1 (also known as CD274, PDL1, or PDCD1L1) or an immunosuppressive portion thereof is expressed by an engineered immune cell through incorporation into a CAR and/or in a membrane-bound fashion. In several embodiments, one or more TIGIT ligands, including but not limited to PVR (also known as CD155, NECL5, or NECL-5) and CD113 (also known as PROM1 or prominin 1) or an immunosuppressive portion of is expressed by an engineered immune cell through incorporation into a CAR and/or in a membrane-bound fashion. In several embodiments, CD200 or an immunosuppressive portion thereof is expressed by an engineered immune cell through incorporation into a CAR and/or in a membrane-bound fashion. In several embodiments, CD276 (also known as B7-H3) or an immunosuppressive portion thereof is expressed by an engineered immune cell through incorporation into a CAR and/or in a membrane-bound fashion. In several embodiments, B7-H4 (also known as VTCN1, B7S1, or B7X)) or an immunosuppressive portion thereof is expressed by an engineered immune cell through incorporation into a CAR and/or in a membrane-bound fashion. In several embodiments, HVEM (also known as TNFSF14, CD270 or ATAR) or an immunosuppressive portion thereof is expressed by an engineered immune cell through incorporation into a CAR and/or in a membrane-bound fashion. In several embodiments, CEACAM5 (also known as CEA) or an immunosuppressive portion thereof is expressed by an engineered immune cell through incorporation into a CAR and/or in a membrane-bound fashion. In several embodiments, Galectin-9 (also known as LGALS9) or an immunosuppressive portion thereof is expressed by an engineered immune cell through incorporation into a CAR and/or in a membrane-bound fashion. In several embodiments, the expression of these immunosuppressive proteins (in whole or in part) functions to impart to the engineered immune cell the ability to reduce or avoid immune clearance by host immune cells (or other engineered immune cells), thereby enhancing the persistence (and thus functional life-span) of the engineered immune cells.

As discussed above with respect to the viral immunosuppressive peptides, the engineered immune cells, in several embodiments, are allogeneic cells. In several embodiments, allogeneic NK cells are used. In several embodiments, allogeneic T cells are used. In several embodiments, combinations (e.g., a mixed population) of allogeneic NK cell and allogeneic T cells are used.

In several embodiments, the engineered CAR comprises one or more copies of one or more of the following amino acid sequences: SEQ ID NO: 245, 280, 285, 286, 288, 289, 1042 or 1043. In several embodiments, the CAR includes an immunosuppressive fragment of SEQ ID NO: 287 or 1044. As discussed above, those sequences (or individual sequence) can be positioned in the hinge region, the N-terminal region or within the target binder region (e.g., within the linker of an scFv). In several embodiments, the CAR comprises an amino acid sequence with at least about 80%, at least about 85%, at least about 90%, at least about 94%, at least about 95%, at least about 96%, at least about 97%, at least about 98%, or at least about 99%, sequence identity, homology and/or functional equivalence with any of SEQ ID NOs: SEQ ID NO: 245, 280, 285, 286, 288, 289, 1042, or 1043 or an immunosuppressive fragment of SEQ ID NO: 287.

In addition to incorporation of the viral immunosuppressive peptides being positioned in the CAR, these non-viral immunosuppressive polypeptides can be coupled to a domain that allows them to be expressed as a membrane bound polypeptides.

Various transmembrane proteins can be used, such as one or more of CD8α, CD4, CD3ε, CD3γ, CD3δ, CD3ζ, CD28, CD137, glycophorin A, glycophorin D, nicotinic acetylcholine receptor, a GABA receptor, FcεRIγ, and a T-cell receptor. In several embodiments, a portion of one or more of these domains (e.g., a transmembrane domain) is used to anchor or otherwise tether the immunosuppressive peptide(s) to the immune cell surface. In several embodiments, the transmembrane protein comprises a CD8α transmembrane domain. In several embodiments, the CD8α transmembrane domain comprises the amino acid sequence of SEQ ID NO: 4, or a sequence with at least about 80%, about 85%, about 90%, about 95%, about 96%, about 97%, about 98% or about 99% sequence identity with SEQ ID NO: 4. In several embodiments, the CD8α transmembrane domain is encoded by the nucleic acid sequence of SEQ ID NO: 3, or a sequence with at least about 80%, about 85%, about 90%, about 95%, about 96%, about 97%, about 98% or about 99% sequence identity with SEQ ID NO: 3. In several embodiments, a hinge or other linker is used to couple the immunosuppressive peptide to the transmembrane protein. In several embodiments, a CD8α is used. In several embodiments, the CD8α hinge comprises the amino acid sequence of SEQ ID NO: 2, or a sequence with at least about 80%, about 85%, about 90%, about 95%, about 96%, about 97%, about 98% or about 99% sequence identity with SEQ ID NO: 2. In several embodiments, the CD8α hinge comprises the amino acid sequence of SEQ ID NO: 1, or a sequence with at least about 80%, about 85%, about 90%, about 95%, about 96%, about 97%, about 98% or about 99% sequence identity with SEQ ID NO: 1.

In several embodiments, there is provided a polynucleotide encoding a truncated CD47 immunosuppressive peptide. In some embodiments, the truncated CD47 immunsuppressive peptide comprises the amino acid sequence set forth in SEQ ID NO:245 (GNYTCEVTELTREGETIIELK). In several embodiments, the polynucleotide encodes an amino acid sequence comprising SEQ ID NO: 245. In several embodiments, the polynucleotide encodes a sequence that shares at least about 80%, at least about 85%, at least about 90%, at least about 94%, at least about 95%, at least about 96%, at least about 97%, at least about 98%, or at least about 99%, sequence identity, homology and/or functional equivalence with SEQ ID NO: 245. In several embodiments, the polynucleotide comprises SEQ ID NO: 246 or shares at least about 80%, at least about 85%, at least about 90%, at least about 94%, at least about 95%, at least about 96%, at least about 97%, at least about 98%, or at least about 99%, sequence identity with SEQ ID NO: 246. In several embodiments, provided for is a membrane-bound truncated CD47 immunosuppressive peptide. In several embodiments, the truncated CD47 immunosuppressive peptide is coupled to a CD8α transmembrane protein and/or CD8α hinge domain. In several embodiments, the membrane-bound truncated CD47 immunosuppressive peptide construct comprises one or more linker sequences. In several embodiments, the membrane-bound truncated CD47 immunosuppressive peptide comprises a CD8α signal peptide, a truncated CD47 peptide (e.g., positions 110-130 of the extracellular domain), a CD8α hinge, and a CD8α transmembrane domain. In several embodiments, the membrane-bound synthetic truncated CD47 immunosuppressive peptide construct is encoded by a polynucleotide that encodes an amino acid sequence comprising SEQ ID NO:1045 (GNYTCEVTELTREGETIIELKTTTPAPRPPTPAPTIASQPLSLRPEACRPAAGGAVHTRGLDFACDIYIWAPL AGTCGVLLLSLVITLYC), (e.g., SEQ ID NO: 248) or SEQ ID NO:1046 (GNYTCEVTELTREGETIIELKTTTPAPRPPTPAPTIASQPLSLRPEACRPAAGGAVHTRGLDFACDIYIWAPL AGTCGVLLLSLVITLYC), (e.g., SEQ ID NO: 717). In several embodiments, the polynucleotide encodes a sequence that shares at least about 80%, at least about 85%, at least about 90%, at least about 94%, at least about 95%, at least about 96%, at least about 97%, at least about 98%, or at least about 99%, sequence identity, homology and/or functional equivalence with SEQ ID NO: 248 or SEQ ID NO: 717). In several embodiments, the polynucleotide comprises SEQ ID NO: 249 (or SEQ ID NO: 718) or shares at least about 80%, at least about 85%, at least about 90%, at least about 94%, at least about 95%, at least about 96%, at least about 97%, at least about 98%, or at least about 99%, sequence identity with SEQ ID NO: 249 (or SEQ ID NO: 718). Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, the polynucleotide encoding membrane-bound truncated CD47 and GFP comprises SEQ ID NO: 247 or shares at least about 80%, at least about 85%, at least about 90%, at least about 94%, at least about 95%, at least about 96%, at least about 97%, at least about 98%, or at least about 99%, sequence identity with SEQ ID NO: 247. In several embodiments, the polynucleotide encoding membrane-bound truncated CD47, a FLAG tag and GFP comprises SEQ ID NO: 716 or shares at least about 80%, at least about 85%, at least about 90%, at least about 94%, at least about 95%, at least about 96%, at least about 97%, at least about 98%, or at least about 99%, sequence identity with SEQ ID NO: 716. In several embodiments, the membrane-bound truncated CD47, FLAG tag, GFP construct comprises the amino acid sequence of SEQ ID NO: 715 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 715.

As discussed above, any of the viral immunosuppressive peptides may be used in combination with one or more non-viral immunosuppressive peptides.

In several embodiments, there is provided a polynucleotide encoding a p15E viral immunosuppressive peptide truncated CD47 construct (p15E_tCD47). In several embodiments, provided for is a membrane-bound p15E_tCD47 construct. In several embodiments, the truncated CD47 peptide comprises a peptide corresponding to positions 110-130 of the extracellular domain of CD47. In several embodiments, the p15E_tCD47 construct is coupled to a CD8α transmembrane protein and/or CD8α hinge domain. In several embodiments, the membrane-bound p15E_tCD47 construct comprises one or more linker sequences. In several embodiments, the membrane-bound p15E_tCD47 construct comprises a CD8α signal peptide, a p15E peptide, a linker (e.g., a GS linker), a truncated CD47 peptide, CD8α hinge, and a CD8α transmembrane domain. In several embodiments, the membrane-bound p15E_tCD47 construct is encoded by a polynucleotide that encodes an amino acid sequence comprising SEQ ID NO: 1047 (LQNRRGLDLLFLKEGGLCAALKEECCFYGGGGSGGGGSGGGGSGNYTCEVTELTREGETIIELKTTTPAPR PPTPAPTIASQPLSLRPEACRPAAGGAVHTRGLDFACDIYIWAPLAGTCGVLLLSLVITLYC), (e.g., SEQ ID NO: 256) or SEQ ID NO: 1048 (LQNRRGLDLLFLKEGGLCAALKEECCFYGGGGSGGGGSGGGGSGNYTCEVTELTREGETIIELKTTTPAPR PPTPAPTIASQPLSLRPEACRPAAGGAVHTRGLDFACDIYIWAPLAGTCGVLLLSLVITLYC), (e.g., SEQ ID NO: 725). In several embodiments, the polynucleotide encodes a sequence that shares at least about 80%, at least about 85%, at least about 90%, at least about 94%, at least about 95%, at least about 96%, at least about 97%, at least about 98%, or at least about 99%, sequence identity, homology and/or functional equivalence with SEQ ID NO: 256 (or SEQ ID NO: 725). In several embodiments, the polynucleotide comprises SEQ ID NO: 257 (or SEQ ID NO: 726) or shares at least about 80%, at least about 85%, at least about 90%, at least about 94%, at least about 95%, at least about 96%, at least about 97%, at least about 98%, or at least about 99%, sequence identity with SEQ ID NO: 257 (or SEQ ID NO: 726). Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, the polynucleotide encoding the membrane-bound p15E_tCD47 construct and GFP comprises SEQ ID NO: 255 or shares at least about 80%, at least about 85%, at least about 90%, at least about 94%, at least about 95%, at least about 96%, at least about 97%, at least about 98%, or at least about 99%, sequence identity with SEQ ID NO: 255. In several embodiments, the polynucleotide encoding the membrane-bound p15E_tCD47 construct, a FLAG tag, and GFP comprises SEQ ID NO: 724 or shares at least about 80%, at least about 85%, at least about 90%, at least about 94%, at least about 95%, at least about 96%, at least about 97%, at least about 98%, or at least about 99%, sequence identity with SEQ ID NO: 724. In several embodiments, the membrane-bound p15E_tCD47, FLAG tag, GFP construct comprises the amino acid sequence of SEQ ID NO: 723 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 723.

In several embodiments, there is provided a polynucleotide encoding a p15E viral immunosuppressive peptide truncated CD47 construct (tCD47_p15E), a swap of the peptides of the construct just described. In several embodiments, provided for is a membrane-bound tCD47_p15E construct. In several embodiments, the truncated CD47 peptide comprises a peptide corresponding to positions 110-130 of the extracellular domain of CD47. In several embodiments, the tCD47_p15E construct is coupled to a CD8α transmembrane protein and/or CD8α hinge domain. In several embodiments, the membrane-bound tCD47_p15E construct comprises one or more linker sequences. In several embodiments, the membrane-bound tCD47_p15E construct comprises a CD8α signal peptide, a truncated CD47 peptide, a linker (e.g., a GS linker), a p15E peptide, a CD8α hinge, and a CD8α transmembrane domain. In several embodiments, the membrane-bound tCD47_p15E construct is encoded by a polynucleotide that encodes an amino acid sequence comprising SEQ ID NO: 1049 (GNYTCEVTELTREGETIIELKGGGGSGGGGSGGGGSLQNRRGLDLLFLKEGGLCAALKEECCFYTTTPAPR PPTPAPTIASQPLSLRPEACRPAAGGAVHTRGLDFACDIYIWAPLAGTCGVLLLSLVITLYC), (e.g., SEQ ID NO: 259) or SEQ ID NO: 1050 (GNYTCEVTELTREGETIIELKGGGGSGGGGSGGGGSLQNRRGLDLLFLKEGGLCAALKEECCFYTTTPAPR PPTPAPTIASQPLSLRPEACRPAAGGAVHTRGLDFACDIYIWAPLAGTCGVLLLSLVITLYC), (e.g., SEQ ID NO: 729). In several embodiments, the polynucleotide encodes a sequence that shares at least about 80%, at least about 85%, at least about 90%, at least about 94%, at least about 95%, at least about 96%, at least about 97%, at least about 98%, or at least about 99%, sequence identity, homology and/or functional equivalence with SEQ ID NO: 259 (or SEQ ID NO: 729). In several embodiments, the polynucleotide comprises SEQ ID NO: 260 (or SEQ ID NO: 730) or shares at least about 80%, at least about 85%, at least about 90%, at least about 94%, at least about 95%, at least about 96%, at least about 97%, at least about 98%, or at least about 99%, sequence identity with SEQ ID NO: 260 (or SEQ ID NO: 730). Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, the polynucleotide encoding the membrane-bound tCD47_p15E construct and GFP comprises SEQ ID NO: 258 or shares at least about 80%, at least about 85%, at least about 90%, at least about 94%, at least about 95%, at least about 96%, at least about 97%, at least about 98%, or at least about 99%, sequence identity with SEQ ID NO: 258. In several embodiments, the polynucleotide encoding the membrane-bound tCD47_p15E construct, a FLAG tag, and GFP comprises SEQ ID NO: 728 or shares at least about 80%, at least about 85%, at least about 90%, at least about 94%, at least about 95%, at least about 96%, at least about 97%, at least about 98%, or at least about 99%, sequence identity with SEQ ID NO: 728. In several embodiments, the membrane-bound tCD47_p15E, FLAG tag, GFP construct comprises the amino acid sequence of SEQ ID NO: 727 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 727.

In several embodiments, there is provided a polynucleotide encoding a modified HIV Gp41 viral immunosuppressive peptide truncated CD47 construct (HIV_L_tCD47). In several embodiments, provided for is a membrane-bound HIV_L_tCD47 construct. In several embodiments, the truncated CD47 peptide comprises a peptide corresponding to positions 110-130 of the extracellular domain of CD47. In several embodiments, the HIV_L_tCD47 construct is coupled to a CD8α transmembrane protein and/or CD8α hinge domain. In several embodiments, the membrane-bound HIV_L_tCD47 construct comprises one or more linker sequences. In several embodiments, the membrane-bound HIV_L_tCD47 construct comprises a CD8α signal peptide, a modified HIV Gp41 viral immunosuppressive peptide, a linker (e.g., a GS linker), a truncated CD47 peptide, a CD8α hinge, and a CD8α transmembrane domain. In several embodiments, the membrane-bound HIV_L_tCD47 construct is encoded by a polynucleotide that encodes an amino acid sequence comprising SEQ ID NO: 1051 (GALFLGFLGAAGSTMGAASVTLTVQARQLLSGIVQQQSNLLRAIEAQQHMLQLTVWGIKQLQARVLAVE RYLKDQGGGGSGGGGSGGGGSGNYTCEVTELTREGETIIELKTTTPAPRPPTPAPTIASQPLSLRPEACRPAA GGAVHTRGLDFACDIYIWAPLAGTCGVLLLSLVITLYC), (e.g., SEQ ID NO: 262) or SEQ ID NO: 1052 (GALFLGFLGAAGSTMGAASVTLTVQARLLLSGIVQQQNNLLRAIEAQQHLLQLTVWGIKQLQARVLAVE RYLRDQGGGGSGGGGSGGGGSGNYTCEVTELTREGETIIELKTTTPAPRPPTPAPTIASQPLSLRPEACRPAA GGAVHTRGLDFACDIYIWAPLAGTCGVLLLSLVITLYC), (e.g., SEQ ID NO: 733). In several embodiments, the polynucleotide encodes a sequence that shares at least about 80%, at least about 85%, at least about 90%, at least about 94%, at least about 95%, at least about 96%, at least about 97%, at least about 98%, or at least about 99%, sequence identity, homology and/or functional equivalence with SEQ ID NO: 262 (or SEQ ID NO: 733). In several embodiments, the polynucleotide comprises SEQ ID NO: 263 (or SEQ ID NO: 734) or shares at least about 80%, at least about 85%, at least about 90%, at least about 94%, at least about 95%, at least about 96%, at least about 97%, at least about 98%, or at least about 99%, sequence identity with SEQ ID NO: 263 (or SEQ ID NO: 734). Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, the polynucleotide encoding the membrane-bound HIV_L_tCD47 construct and GFP comprises SEQ ID NO: 261 or shares at least about 80%, at least about 85%, at least about 90%, at least about 94%, at least about 95%, at least about 96%, at least about 97%, at least about 98%, or at least about 99%, sequence identity with SEQ ID NO: 261. In several embodiments, the polynucleotide encoding the membrane-bound HIV_L_tCD47 construct, a FLAG tag, and GFP comprises SEQ ID NO: 732 or shares at least about 80%, at least about 85%, at least about 90%, at least about 94%, at least about 95%, at least about 96%, at least about 97%, at least about 98%, or at least about 99%, sequence identity with SEQ ID NO: 732. In several embodiments, the membrane-bound HIV_L_tCD47, FLAG tag, GFP construct comprises the amino acid sequence of SEQ ID NO: 731 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 731.

In several embodiments, there is provided a polynucleotide encoding an HIV Gp41 viral immunosuppressive peptide truncated CD47 construct (HIV_S_tCD47). In several embodiments, provided for is a membrane-bound HIV_S_tCD47 construct. In several embodiments, the truncated CD47 peptide comprises a peptide corresponding to positions 110-130 of the extracellular domain of CD47. In several embodiments, the HIV_S_tCD47 construct is coupled to a CD8α transmembrane protein and/or CD8α hinge domain. In several embodiments, the membrane-bound HIV_S_tCD47 construct comprises one or more linker sequences. In several embodiments, the membrane-bound HIV_S_tCD47 construct comprises a CD8α signal peptide, an HIV Gp41 viral immunosuppressive peptide, a linker (e.g., a GS linker), a truncated CD47 peptide, a CD8α hinge, and a CD8α transmembrane domain. In several embodiments, the membrane-bound HIV_S_tCD47 construct is encoded by a polynucleotide that encodes an amino acid sequence comprising SEQ ID NO: 1053 (LQARILAVERYLKDGGGGSGGGGSGGGGSGNYTCEVTELTREGETIIELKTTTPAPRPPTPAPTIASQPLSLR PEACRPAAGGAVHTRGLDFACDIYIWAPLAGTCGVLLLSLVITLYC), (e.g., SEQ ID NO: 265) or SEQ ID NO: 1054 (LQARILAVERYLKDGGGGSGGGGSGGGGSGNYTCEVTELTREGETIIELKTTTPAPRPPTPAPTIASQPLSLR PEACRPAAGGAVHTRGLDFACDIYIWAPLAGTCGVLLLSLVITLYC), (e.g., SEQ ID NO: 737). In several embodiments, the polynucleotide encodes a sequence that shares at least about 80%, at least about 85%, at least about 90%, at least about 94%, at least about 95%, at least about 96%, at least about 97%, at least about 98%, or at least about 99%, sequence identity, homology and/or functional equivalence with SEQ ID NO: 265 (or SEQ ID NO: 737). In several embodiments, the polynucleotide comprises SEQ ID NO: 266 (or SEQ ID NO: 738) or shares at least about 80%, at least about 85%, at least about 90%, at least about 94%, at least about 95%, at least about 96%, at least about 97%, at least about 98%, or at least about 99%, sequence identity with SEQ ID NO: 266 (or SEQ ID NO: 738). Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, the polynucleotide encoding the membrane-bound HIV_S_tCD47 construct and GFP comprises SEQ ID NO: 264 or shares at least about 80%, at least about 85%, at least about 90%, at least about 94%, at least about 95%, at least about 96%, at least about 97%, at least about 98%, or at least about 99%, sequence identity with SEQ ID NO: 264. In several embodiments, the polynucleotide encoding the membrane-bound HIV_S_tCD47 construct, a FLAG tag, and GFP comprises SEQ ID NO: 736 or shares at least about 80%, at least about 85%, at least about 90%, at least about 94%, at least about 95%, at least about 96%, at least about 97%, at least about 98%, or at least about 99%, sequence identity with SEQ ID NO: 736. In several embodiments, the membrane-bound HIV_S_tCD47, FLAG tag, GFP construct comprises the amino acid sequence of SEQ ID NO: 735 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 735.

In several embodiments, there is provided a polynucleotide encoding an HTLV viral immunosuppressive peptide truncated CD47 construct (HTLV_tCD47). In several embodiments, provided for is a membrane-bound HTLV_tCD47 construct. In several embodiments, the truncated CD47 peptide comprises a peptide corresponding to positions 110-130 of the extracellular domain of CD47. In several embodiments, the HTLV_tCD47 construct is coupled to a CD8α transmembrane protein and/or CD8α hinge domain. In several embodiments, the membrane-bound HTLV_tCD47 construct comprises one or more linker sequences. In several embodiments, the membrane-bound HTLV_tCD47 construct comprises a CD8α signal peptide, an HTLV viral immunosuppressive peptide, a linker (e.g., a GS linker), a truncated CD47 peptide, a CD8α hinge, and a CD8α transmembrane domain. In several embodiments, the membrane-bound HTLV_tCD47 construct is encoded by a polynucleotide that encodes an amino acid sequence comprising SEQ ID NO: 1055 (AQNRRGLDLLFWEQGGLCKALQEQCRFPGGGGSGGGGSGGGGSGNYTCEVTELTREGETIIELKTTTPAP RPPTPAPTIASQPLSLRPEACRPAAGGAVHTRGLDFACDIYIWAPLAGTCGVLLLSLVITLYC), (e.g., SEQ ID NO: 268) or SEQ ID NO: 1056 (AQNRRGLDLLFWEQGGLCKALQEQCRFPGGGGSGGGGSGGGGSGNYTCEVTELTREGETIIELKTTTPAP RPPTPAPTIASQPLSLRPEACRPAAGGAVHTRGLDFACDIYIWAPLAGTCGVLLLSLVITLYC), (e.g., SEQ ID NO: 741). In several embodiments, the polynucleotide encodes a sequence that shares at least about 80%, at least about 85%, at least about 90%, at least about 94%, at least about 95%, at least about 96%, at least about 97%, at least about 98%, or at least about 99%, sequence identity, homology and/or functional equivalence with SEQ ID NO: 268 (or SEQ ID NO: 741). In several embodiments, the polynucleotide comprises SEQ ID NO: 269 (or SEQ ID NO: 742) or shares at least about 80%, at least about 85%, at least about 90%, at least about 94%, at least about 95%, at least about 96%, at least about 97%, at least about 98%, or at least about 99%, sequence identity with SEQ ID NO: 269 (or SEQ ID NO: 742). Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, the polynucleotide encoding the membrane-bound HTLV_tCD47 construct and GFP comprises SEQ ID NO: 267 or shares at least about 80%, at least about 85%, at least about 90%, at least about 94%, at least about 95%, at least about 96%, at least about 97%, at least about 98%, or at least about 99%, sequence identity with SEQ ID NO: 267. In several embodiments, the polynucleotide encoding the membrane-bound HTLV_tCD47 construct, a FLAG tag, and GFP comprises SEQ ID NO: 740 or shares at least about 80%, at least about 85%, at least about 90%, at least about 94%, at least about 95%, at least about 96%, at least about 97%, at least about 98%, or at least about 99%, sequence identity with SEQ ID NO: 740. In several embodiments, the membrane-bound HTLV_tCD47, FLAG tag, GFP construct comprises the amino acid sequence of SEQ ID NO: 739 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 739.

In several embodiments, there is provided a polynucleotide encoding a truncated CD47-p15E-dimer viral immunosuppressive peptide truncated CD47 construct (tCD47_p15Ex2). In several embodiments, provided for is a membrane-bound tCD47_p15Ex2 construct. In several embodiments, the truncated CD47 peptide comprises a peptide corresponding to positions 110-130 of the extracellular domain of CD47. In several embodiments, the tCD47_p15Ex2 construct is coupled to a CD8α transmembrane protein and/or CD8α hinge domain. In several embodiments, the membrane-bound tCD47_p15Ex2 construct comprises one or more linker sequences. In several embodiments, the membrane-bound tCD47_p15Ex2 construct comprises a CD8α signal peptide, a truncated CD47 peptide, a linker (e.g., a GS linker), a first p15E peptide, a linker (e.g., a GS linker), a second p15E peptide, a CD8α hinge, and a CD8α transmembrane domain. In several embodiments, the membrane-bound tCD47_p15Ex2 construct is encoded by a polynucleotide that encodes an amino acid sequence comprising SEQ ID NO: 1057 (GNYTCEVTELTREGETIIELKGGGGSGGGGSGGGGSLQNRRGLDLLFLKEGGLCAALKEECCFYGGGGSG GGGSGGGGSLQNRRGLDLLFLKEGGLCAALKEECCFYTTTPAPRPPTPAPTIASQPLSLRPEACRPAAGGAV HTRGLDFACDIYIWAPLAGTCGVLLLSLVITLYC), (e.g., SEQ ID NO: 271) or SEQ ID NO: 1058 (GNYTCEVTELTREGETIIELKGGGGSGGGGSGGGGSLQNRRGLDLLFLKEGGLCAALKEECCFYGGGGSG GGGSGGGGSLQNRRGLDLLFLKEGGLCAALKEECCFYTTTPAPRPPTPAPTIASQPLSLRPEACRPAAGGAV HTRGLDFACDIYIWAPLAGTCGVLLLSLVITLYC), (e.g., SEQ ID NO: 745). In several embodiments, the polynucleotide encodes a sequence that shares at least about 80%, at least about 85%, at least about 90%, at least about 94%, at least about 95%, at least about 96%, at least about 97%, at least about 98%, or at least about 99%, sequence identity, homology and/or functional equivalence with SEQ ID NO: 271 (or SEQ ID NO: 745). In several embodiments, the polynucleotide comprises SEQ ID NO: 272 (or SEQ ID NO: 746) or shares at least about 80%, at least about 85%, at least about 90%, at least about 94%, at least about 95%, at least about 96%, at least about 97%, at least about 98%, or at least about 99%, sequence identity with SEQ ID NO: 272 (or SEQ ID NO: 746). Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, the polynucleotide encoding the membrane-bound tCD47_p15Ex2 construct and GFP comprises SEQ ID NO: 270 or shares at least about 80%, at least about 85%, at least about 90%, at least about 94%, at least about 95%, at least about 96%, at least about 97%, at least about 98%, or at least about 99%, sequence identity with SEQ ID NO: 270. In several embodiments, the polynucleotide encoding the membrane-bound tCD47_p15Ex2 construct, a FLAG tag, and GFP comprises SEQ ID NO: 744 or shares at least about 80%, at least about 85%, at least about 90%, at least about 94%, at least about 95%, at least about 96%, at least about 97%, at least about 98%, or at least about 99%, sequence identity with SEQ ID NO: 744. In several embodiments, the membrane-bound tCD47_p15Ex2, FLAG tag, GFP construct comprises the amino acid sequence of SEQ ID NO: 743 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 743.

Additionally, according to several embodiments, combinations and/or replicates of various viral peptides are provided for. See FIGS. 15A and 15B, for example. FIG. 15A shows a non-limiting schematic of construct according to embodiments disclosed herein, in which a domain designed to reduce cytotoxic effects on a cell expressing such a construct (also referred to as a hypoimmune domain) is coupled to at least a transmembrane domain or (as shown in the non-limiting schematic), in some embodiments, integrated into a CAR construct. In several embodiments, viral peptides such as those disclosed herein are integrated into the CAR (e.g., CKS-17, p15E, including p15E-long, and/or HTLV). As shown in FIG. 15B, multimeric formats are provided for, in several embodiments, such as doublet or triplet of p15E (including p15E-long). Viral and non-viral peptides may also be used in combination, such as any of a synthetic TCR blocking peptide, HIV TCR blocking peptides (including truncated formats), an inhibitory receptor peptide (such as tCD47) with any of the other viral peptides disclosed herein. Non-limiting examples include p15E-tCD47, tCD47-p15E, HIV-long-TCD47, HIV-short-tCD47, HTLV-tCD47, and tCD47-p15E (doublet or triplet, optionally long format).

In several embodiments, there is provided a polynucleotide encoding a synthetic CKS-17 viral immunosuppressive peptide. In several embodiments, provided for is a membrane-bound synthetic CKS-17 viral immunosuppressive peptide. In several embodiments, the synthetic CKS-17 viral immunosuppressive peptide is coupled to a CD8α transmembrane protein and/or CD8α hinge domain. In several embodiments, the membrane-bound synthetic CKS-17 viral immunosuppressive peptide construct comprises one or more linker sequences. In several embodiments, the membrane-bound synthetic CKS-17 viral immunosuppressive peptide comprises a CD8α signal peptide, synthetic CKS-17, a CD8α hinge, and a CD8α transmembrane domain. In some embodiments, the membrane-bound CKS-17 viral immunosuppressive peptide comprises the amino acid sequence set forth in SEQ ID NO:1059 In several embodiments, the membrane-bound synthetic CKS-17 viral immunosuppressive peptide construct is encoded by a polynucleotide that encodes an amino acid sequence comprising SEQ ID NO: 1059 (LQNRRGLDLLFLKEGGLCTTTPAPRPPTPAPTIASQPLSLRPEACRPAAGGAVHTRGLDFACDIYIWAPLAG TCGVLLLSLVITLYC). In several embodiments, the polynucleotide encodes a sequence that shares at least about 80%, at least about 85%, at least about 90%, at least about 94%, at least about 95%, at least about 96%, at least about 97%, at least about 98%, or at least about 99%, sequence identity, homology and/or functional equivalence with SEQ ID NO:1059 (LQNRRGLDLLFLKEGGLCTTTPAPRPPTPAPTIASQPLSLRPEACRPAAGGAVHTRGLDFACDIYIWAPLAG TCGVLLLSLVITLYC.). In several embodiments, the polynucleotide comprises SEQ ID NO: 750 or shares at least about 80%, at least about 85%, at least about 90%, at least about 94%, at least about 95%, at least about 96%, at least about 97%, at least about 98%, or at least about 99%, sequence identity with SEQ ID NO: 750. In several embodiments, the polynucleotide encoding membrane-bound synthetic CKS-17 also encodes GFP (without an IRES or a linker) and comprises SEQ ID NO: 748 or shares at least about 80%, at least about 85%, at least about 90%, at least about 94%, at least about 95%, at least about 96%, at least about 97%, at least about 98%, or at least about 99%, sequence identity with SEQ ID NO: 748. In several embodiments, the membrane-bound synthetic CKS-17-GFP construct comprises SEQ ID NO: 747 or shares at least about 80%, at least about 85%, at least about 90%, at least about 94%, at least about 95%, at least about 96%, at least about 97%, at least about 98%, or at least about 99%, sequence identity with SEQ ID NO: 747.

In several embodiments, a truncated CD47 immunosuppressive peptide is employed. In some embodiments, the truncated CD47 immunosuppressive peptide comprises the amino acid sequence set forth in SEQ ID NO:1060. In several embodiments, there is provided a polynucleotide that encodes an amino acid sequence comprising SEQ ID NO: 753. In several embodiments, the polynucleotide encodes a sequence that shares at least about 80%, at least about 85%, at least about 90%, at least about 94%, at least about 95%, at least about 96%, at least about 97%, at least about 98%, or at least about 99%, sequence identity, homology and/or functional equivalence with SEQ ID NO: 753. In several embodiments, the polynucleotide comprises SEQ ID NO: 753 or shares at least about 80%, at least about 85%, at least about 90%, at least about 94%, at least about 95%, at least about 96%, at least about 97%, at least about 98%, or at least about 99%, sequence identity with SEQ ID NO: 754. Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, the polynucleotide encoding truncated CD47 and GFP comprises SEQ ID NO: 752 or shares at least about 80%, at least about 85%, at least about 90%, at least about 94%, at least about 95%, at least about 96%, at least about 97%, at least about 98%, or at least about 99%, sequence identity with SEQ ID NO: 752. In several embodiments, the truncated CD47-GFP construct comprises the amino acid sequence of SEQ ID NO: 751 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 751.

In several embodiments, there is provided a polynucleotide encoding an HIV Gp41 viral immunosuppressive peptide construct that includes a native HIV transmembrane domain (fGP41_HIVtm). In several embodiments, the HIV Gp41 viral immunosuppressive peptide is coupled to a CD8α signal peptide (e.g., for expression purposes, the sequence of which is MALPVTALLLPLALLLHAARP (SEQ ID NO 895) at the amino acid level and ATGGCACTCCCCGTAACTGCTCTGCTGCTGCCGTTGGCATTGCTCCTGCACGCCGCACGCCCG (SEQ ID NO 896) at the nucleotide level). In some embodiments, the HIV GP41 viral immunosuppressive peptide comprises the amino acid sequence set forth in SEQ ID NO:1061. In several embodiments, the HIV Gp41 viral immunosuppressive construct comprises a CD8α signal peptide and HIV Gp41 and is encoded by a polynucleotide that encodes an amino acid sequence comprising SEQ ID NO:757. In several embodiments, the polynucleotide encodes a sequence that shares at least about 80%, at least about 85%, at least about 90%, at least about 94%, at least about 95%, at least about 96%, at least about 97%, at least about 98%, or at least about 99%, sequence identity, homology and/or functional equivalence with SEQ ID NO: 757. In several embodiments, the polynucleotide comprises SEQ ID NO: 758 or shares at least about 80%, at least about 85%, at least about 90%, at least about 94%, at least about 95%, at least about 96%, at least about 97%, at least about 98%, or at least about 99%, sequence identity with SEQ ID NO: 758. Optionally, the polynucleotide encodes a detectable tag (e.g., a FLAG tag) and/or can comprise an internal ribosome entry site (IRES) that allows for expression of an additional protein, such as a detectable tag (e.g., GFP). n several embodiments, a linker (e.g., a GS linker) is positioned between the Gp41 domain and the tag. In several embodiments, the polynucleotide encoding HIV Gp41 and GFP comprises SEQ ID NO: 756 or shares at least about 80%, at least about 85%, at least about 90%, at least about 94%, at least about 95%, at least about 96%, at least about 97%, at least about 98%, or at least about 99%, sequence identity with SEQ ID NO: 756. In several embodiments, the fGP41_HIVtm-GFP construct comprises that amino acid sequence of SEQ ID NO: 755 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 755.

In several embodiments, there is provided a polynucleotide encoding a p15E transmembrane domain immunosuppressive peptide (p15Etm_sf). In several embodiments, the p15E transmembrane domain is coupled to a CD8α signal peptide (e.g., for expression purposes, the sequence of which is provided for separately herein). In several embodiments, the p15E transmembrane domain comprises the amino acid sequence set forth in SEQ ID NO: 1062. In several embodiments, the p15E transmembrane domain immunosuppressive construct comprises a CD8α signal peptide and the p15E transmembrane domain and is encoded by a polynucleotide that encodes an amino acid sequence comprising SEQ ID NO: 761. In several embodiments, the polynucleotide encodes a sequence that shares at least about 90%, at least about 94%, at least about 95%, at least about 96%, at least about 97%, at least about 98%, or at least about 99%, sequence identity, homology and/or functional equivalence with SEQ ID NO:761. In several embodiments, the polynucleotide comprises SEQ ID NO: 762 or shares at least about 80%, at least about 85%, at least about 90%, at least about 94%, at least about 95%, at least about 96%, at least about 97%, at least about 98%, or at least about 99%, sequence identity with SEQ ID NO: 762. Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, a linker (e.g., a GS linker) is positioned between the p15E transmembrane domain and the tag. In several embodiments, the polynucleotide encoding the p15E transmembrane domain and GFP comprises SEQ ID NO: 760 or shares at least about 80%, at least about 85%, at least about 90%, at least about 94%, at least about 95%, at least about 96%, at least about 97%, at least about 98%, or at least about 99%, sequence identity with SEQ ID NO: 760. In several embodiments, the p15E transmembrane domain-GFP construct comprises the amino acid sequence of SEQ ID NO: 759 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 759.

In several embodiments, there is provided a polynucleotide encoding a CD43-derived immunosuppressive peptide (fCD43_TM). In several embodiments, the CD43 peptide is coupled to a CD8α signal peptide (e.g., for expression purposes, the sequence of which is provided for separately herein). In several embodiments, the CD43 immunosuppressive construct comprises the amino acid sequence set forth in SEQ ID NO: 1063. In several embodiments, the CD43 immunosuppressive construct comprises a CD8α signal peptide and the CD43 peptide and is encoded by a polynucleotide that encodes an amino acid sequence comprising SEQ ID NO: 765. In several embodiments, the amino acid sequence shares at least about 90%, at least about 94%, at least about 95%, at least about 96%, at least about 97%, at least about 98%, or at least about 99%, sequence identity, homology and/or functional equivalence with SEQ ID NO: 765. In several embodiments, the polynucleotide comprises SEQ ID NO: 766 or shares at least about 80%, at least about 85%, at least about 90%, at least about 94%, at least about 95%, at least about 96%, at least about 97%, at least about 98%, or at least about 99%, sequence identity with SEQ ID NO: 766. Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, a linker (e.g., a GS linker) is positioned between the CD43 and the tag. In several embodiments, the polynucleotide encoding fCD43 and GFP comprises SEQ ID NO: 764 or shares at least about 80%, at least about 85%, at least about 90%, at least about 94%, at least about 95%, at least about 96%, at least about 97%, at least about 98%, or at least about 99%, sequence identity with SEQ ID NO: 764. In several embodiments, the CD43-GFP construct comprises the amino acid sequence of SEQ ID NO: 763 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 763.

In several embodiments, there is provided a polynucleotide encoding a latent membrane protein 1 (LMP1) peptide of the Epstein-Barr Virus (LMP1_TM). In several embodiments, the LMP1 peptide is coupled to a CD8α signal peptide (e.g., for expression purposes, the sequence of which is provided for separately herein). In several embodiments, the LMP1 immunosuppressive construct comprises the amino acid sequence set forth in SEQ ID NO: 1064. In several embodiments, the LMP1 immunosuppressive construct comprises a CD8α signal peptide and the LMP1 peptide and is encoded by a polynucleotide that encodes an amino acid sequence comprising SEQ ID NO: 769. In several embodiments, the amino acid sequence shares at least about 90%, at least about 94%, at least about 95%, at least about 96%, at least about 97%, at least about 98%, or at least about 99%, sequence identity, homology and/or functional equivalence with SEQ ID NO: 769. In several embodiments, the polynucleotide comprises SEQ ID NO: 770 or shares at least about 80%, at least about 85%, at least about 90%, at least about 94%, at least about 95%, at least about 96%, at least about 97%, at least about 98%, or at least about 99%, sequence identity with SEQ ID NO: 770. Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, a linker (e.g., a GS linker) is positioned between the LMP1 and the tag. In several embodiments, the polynucleotide encoding fLMP1 and GFP comprises SEQ ID NO: 768 or shares at least about 80%, at least about 85%, at least about 90%, at least about 94%, at least about 95%, at least about 96%, at least about 97%, at least about 98%, or at least about 99%, sequence identity with SEQ ID NO: 768. In several embodiments, the LMP1-GFP construct comprises the amino acid sequence of SEQ ID NO: 767 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 767.

In several embodiments, there is provided a polynucleotide encoding a glycoprotein D (gD) of the Herpes Simplex Virus (fgD_TM). In several embodiments, the gD peptide is coupled to a CD8α signal peptide (e.g., for expression purposes, the sequence of which is provided for separately herein). In several embodiments, the gD immunoresuppressive construct comprises the amino acid sequence set forth in SEQ ID NO:1065. In several embodiments, the gD immunosuppressive construct comprises a CD8α signal peptide and the gD peptide and is encoded by a polynucleotide that encodes an amino acid sequence comprising SEQ ID NO: 773. In several embodiments, the amino acid sequence shares at least about 90%, at least about 94%, at least about 95%, at least about 96%, at least about 97%, at least about 98%, or at least about 99%, sequence identity, homology and/or functional equivalence with SEQ ID NO: 773. In several embodiments, the polynucleotide comprises SEQ ID NO: 774 or shares at least about 80%, at least about 85%, at least about 90%, at least about 94%, at least about 95%, at least about 96%, at least about 97%, at least about 98%, or at least about 99%, sequence identity with SEQ ID NO: 774. Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, a linker (e.g., a GS linker) is positioned between the gD and the tag. In several embodiments, the polynucleotide encoding gD and GFP comprises SEQ ID NO: 772 or shares at least about 80%, at least about 85%, at least about 90%, at least about 94%, at least about 95%, at least about 96%, at least about 97%, at least about 98%, or at least about 99%, sequence identity with SEQ ID NO: 772. In several embodiments, the gD-GFP construct comprises the amino acid sequence of SEQ ID NO: 771 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 771.

In several embodiments, there is provided a polynucleotide encoding a lectin-like transcript 1 (LLT1), which, when interacting with CD161, inhibits Natural Killer cell activation and contributes to tumor cell immunosuppressive properties. In several embodiments, the LLT1 peptide is coupled to a CD8α signal peptide (e.g., for expression purposes, the sequence of which is provided for separately herein). In several embodiments, the LLT1 immunosuppressive construct comprises the amino acid sequence set forth in SEQ ID NO:1066. In several embodiments, the LLT1 immunosuppressive construct comprises a CD8α signal peptide and the LLT1 peptide and is encoded by a polynucleotide that encodes an amino acid sequence comprising SEQ ID NO: 777. In several embodiments, the amino acid sequence shares at least about 90%, at least about 94%, at least about 95%, at least about 96%, at least about 97%, at least about 98%, or at least about 99%, sequence identity, homology and/or functional equivalence with SEQ ID NO: 777. In several embodiments, the polynucleotide comprises SEQ ID NO: 778 or shares at least about 80%, at least about 85%, at least about 90%, at least about 94%, at least about 95%, at least about 96%, at least about 97%, at least about 98%, or at least about 99%, sequence identity with SEQ ID NO: 778. Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, a linker (e.g., a GS linker) is positioned between the LLT1 and the tag. In several embodiments, the polynucleotide encoding LLT1 and GFP comprises SEQ ID NO: 776 or shares at least about 80%, at least about 85%, at least about 90%, at least about 94%, at least about 95%, at least about 96%, at least about 97%, at least about 98%, or at least about 99%, sequence identity with SEQ ID NO: 776. In several embodiments, the LLT1-GFP construct comprises the amino acid sequence of SEQ ID NO: 775 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 775.

In several embodiments, there is provided a polynucleotide encoding at least a portion of the extracellular and transmembrane domains of CD47 (CD47tm162). In several embodiments, the CD47 domains are coupled to a CD8α signal peptide (e.g., for expression purposes, the sequence of which is provided for separately herein). In several embodiments, the CD47 immunosuppressive construct comprises the amino acid sequence set forth in SEQ ID NO: 1067. In several embodiments, the CD47 immunosuppressive construct comprises a CD8α signal peptide and the CD47 extracellular and transmembrane domains (positions 19 to 162 of UniProtKB-Q08722) and is encoded by a polynucleotide that encodes an amino acid sequence comprising SEQ ID NO: 781. In several embodiments, the amino acid sequence shares at least about 90%, at least about 94%, at least about 95%, at least about 96%, at least about 97%, at least about 98%, or at least about 99%, sequence identity, homology and/or functional equivalence with SEQ ID NO: 781. In several embodiments, the polynucleotide comprises SEQ ID NO: 782 or shares at least about 80%, at least about 85%, at least about 90%, at least about 94%, at least about 95%, at least about 96%, at least about 97%, at least about 98%, or at least about 99%, sequence identity with SEQ ID NO: 782. Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, a linker (e.g., a GS linker) is positioned between the CD47 domains and the tag. In several embodiments, the polynucleotide encoding CD47 and GFP comprises SEQ ID NO: 780 or shares at least about 80%, at least about 85%, at least about 90%, at least about 94%, at least about 95%, at least about 96%, at least about 97%, at least about 98%, or at least about 99%, sequence identity with SEQ ID NO: 780. In several embodiments, the CD47tm162-GFP construct comprises the amino acid sequence of SEQ ID NO: 779 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 779.

In several embodiments, there is provided a polynucleotide encoding at least a portion of the latent membrane protein (LMP) oncogene of the Epstein-Barr virus (LMP_L_CD8HTM). In several embodiments, the LMP domain is coupled to a CD8α signal peptide (e.g., for expression purposes, the sequence of which is provided for separately herein). In several embodiments, the LMP immunosuppressive construct comprises the amino acid sequence set forth in SEQ ID NO:1068. In several embodiments, the LMP immunosuppressive construct comprises a CD8α signal peptide, the LMP_L domain, a CD8α hinge, and a CD8α transmembrane domain and is encoded by a polynucleotide that encodes an amino acid sequence comprising SEQ ID NO: 785. In several embodiments, the amino acid sequence shares at least about 90%, at least about 94%, at least about 95%, at least about 96%, at least about 97%, at least about 98%, or at least about 99%, sequence identity, homology and/or functional equivalence with SEQ ID NO: 785. In several embodiments, the polynucleotide comprises SEQ ID NO: 786 or shares at least about 80%, at least about 85%, at least about 90%, at least about 94%, at least about 95%, at least about 96%, at least about 97%, at least about 98%, or at least about 99%, sequence identity with SEQ ID NO: 786. Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, a linker (e.g., a GS linker) is positioned between the CD47 domains and the tag. In several embodiments, the polynucleotide encoding the LMP_L domain and GFP comprises SEQ ID NO: 784 or shares at least about 80%, at least about 85%, at least about 90%, at least about 94%, at least about 95%, at least about 96%, at least about 97%, at least about 98%, or at least about 99%, sequence identity with SEQ ID NO: 784. In several embodiments, the LMP_L_CD8HTM-GFP construct comprises the amino acid sequence of SEQ ID NO: 783 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 783.

In several embodiments, there is provided a polynucleotide encoding a synthetic immunosuppressive construct comprising a multimeric repeat (e.g., dimer, trimer, quatramer, pentamer, etc.) of a peptide with immunosuppressive properties (LALLFWLx5-CD8HTM). In several embodiments, the synthetic domain is coupled to a CD8α signal peptide (e.g., for expression purposes, the sequence of which is provided for separately herein). In several embodiments, the synthetic immunosuppressive construct comprises the amino acid sequence set forth in SEQ ID NO:1069. In several embodiments, the synthetic immunosuppressive construct comprises a CD8α signal peptide, the five repeats of the synthetic peptide, a CD8α hinge, and a CD8α transmembrane domain and is encoded by a polynucleotide that encodes an amino acid sequence comprising SEQ ID NO: 789. In several embodiments, the amino acid sequence shares at least about 90%, at least about 94%, at least about 95%, at least about 96%, at least about 97%, at least about 98%, or at least about 99%, sequence identity, homology and/or functional equivalence with SEQ ID NO: 789. In several embodiments, the polynucleotide comprises SEQ ID NO: 790 or shares at least about 80%, at least about 85%, at least about 90%, at least about 94%, at least about 95%, at least about 96%, at least about 97%, at least about 98%, or at least about 99%, sequence identity with SEQ ID NO: 790. Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, a linker (e.g., a GS linker) is positioned between the transmembrane domain and the tag. In several embodiments, the polynucleotide encoding the synthetic immunosuppressive peptide and GFP comprises SEQ ID NO: 788 or shares at least about 80%, at least about 85%, at least about 90%, at least about 94%, at least about 95%, at least about 96%, at least about 97%, at least about 98%, or at least about 99%, sequence identity with SEQ ID NO: 788. In several embodiments, the synthetic immunosuppressive peptide-GFP construct comprises the amino acid sequence of SEQ ID NO: 787 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 787.

In several embodiments, there is provided a polynucleotide encoding at least a portion of the N-terminal fusion peptide (FP) of the human immunodeficiency virus (HIV)-1 envelope glycoprotein (Env) gp41 (GP41fp). In several embodiments, the GP41fp is coupled to a CD8α signal peptide (e.g., for expression purposes, the sequence of which is provided for separately herein). In several embodiments, the GP41fp immunosuppressive construct comprises the amino acid sequence set forth in SEQ ID NO:1070. In several embodiments, the GP41fp immunosuppressive construct comprises a CD8α signal peptide, the GP41fp peptide, and at least a portion of the GP41 transmembrane domain and is encoded by a polynucleotide that encodes an amino acid sequence comprising SEQ ID NO: 793. In several embodiments, the amino acid sequence shares at least about 90%, at least about 94%, at least about 95%, at least about 96%, at least about 97%, at least about 98%, or at least about 99%, sequence identity, homology and/or functional equivalence with SEQ ID NO: 793. In several embodiments, the polynucleotide comprises SEQ ID NO: 794 or shares at least about 80%, at least about 85%, at least about 90%, at least about 94%, at least about 95%, at least about 96%, at least about 97%, at least about 98%, or at least about 99%, sequence identity with SEQ ID NO: 794. Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, a linker (e.g., a GS linker) is positioned between the transmembrane domain and the tag. In several embodiments, the polynucleotide encoding the GP41fp and GFP comprises SEQ ID NO: 792 or shares at least about 80%, at least about 85%, at least about 90%, at least about 94%, at least about 95%, at least about 96%, at least about 97%, at least about 98%, or at least about 99%, sequence identity with SEQ ID NO: 792. In several embodiments, the GP41fp-GFP construct comprises the amino acid sequence of SEQ ID NO: 791 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 791.

In several embodiments, there is provided a polynucleotide encoding a CKS-17 viral immunosuppressive peptide. In several embodiments, provided for is a membrane-bound CKS-17 viral immunosuppressive peptide. In several embodiments, the CKS-17 viral immunosuppressive peptide is coupled to a CD8α transmembrane protein and/or CD8α hinge domain. In several embodiments, the membrane-bound CKS-17 viral immunosuppressive peptide construct comprises one or more linker sequences. In several embodiments, the membrane-bound CKS-17 viral immunosuppressive peptide comprises a CD8α signal peptide, synthetic CKS-17, a CD8α hinge, and a CD8α transmembrane domain. In several embodiments, the membrane-bound CKS-17 viral immunosuppressive construct comprises the amino acid sequence set forth in SEQ ID NO:1071. In several embodiments, the membrane-bound CKS-17 viral immunosuppressive peptide construct is encoded by a polynucleotide that encodes an amino acid sequence comprising SEQ ID NO: 797. In several embodiments, the polynucleotide encodes a sequence that shares at least about 80%, at least about 85%, at least about 90%, at least about 94%, at least about 95%, at least about 96%, at least about 97%, at least about 98%, or at least about 99%, sequence identity, homology and/or functional equivalence with SEQ ID NO: 797. In several embodiments, the polynucleotide comprises SEQ ID NO: 798 or shares at least about 80%, at least about 85%, at least about 90%, at least about 94%, at least about 95%, at least about 96%, at least about 97%, at least about 98%, or at least about 99%, sequence identity with SEQ ID NO: 798. Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, the polynucleotide encoding membrane-bound synthetic CKS-17 and GFP comprises SEQ ID NO: 796 or shares at least about 80%, at least about 85%, at least about 90%, at least about 94%, at least about 95%, at least about 96%, at least about 97%, at least about 98%, or at least about 99%, sequence identity with SEQ ID NO: 796. In several embodiments, the membrane-bound synthetic CKS-17-GFP construct comprises SEQ ID NO: 795 or shares at least about 80%, at least about 85%, at least about 90%, at least about 94%, at least about 95%, at least about 96%, at least about 97%, at least about 98%, or at least about 99%, sequence identity with SEQ ID NO: 795.

In several embodiments, there is provided a polynucleotide encoding a chimeric immunosuppressive construct comprising a CKS-17 domain and an HTLV-1 (Gp21) domain. In several embodiments, provided for is a membrane-bound immunosuppressive construct comprising a CKS-17 domain and a an HTLV-1 (Gp21) domain (CKS_HTLV-8aTM). In several embodiments, the CKS_HTLV immunosuppressive peptide is coupled to a CD8α transmembrane protein and/or CD8α hinge domain. In several embodiments, the membrane-bound CKS_HTLV viral immunosuppressive peptide construct comprises one or more linker sequences. In several embodiments, the membrane-bound CKS_HTLV viral immunosuppressive peptide comprises a CD8α signal peptide, a CKS-17 peptide, an HTLV(Gp21) peptide, a CD8α hinge, and a CD8α transmembrane domain. In several embodiments, the membrane-bound CKS_HLTV immunosuppressive construct comprises the amino acid sequence set forth in SEQ ID NO:1072. In several embodiments, the membrane-bound CKS_HTLV immunosuppressive peptide construct is encoded by a polynucleotide that encodes an amino acid sequence comprising SEQ ID NO: 801. In several embodiments, the polynucleotide encodes a sequence that shares at least about 80%, at least about 85%, at least about 90%, at least about 94%, at least about 95%, at least about 96%, at least about 97%, at least about 98%, or at least about 99%, sequence identity, homology and/or functional equivalence with SEQ ID NO: 801. In several embodiments, the polynucleotide comprises SEQ ID NO: 802 or shares at least about 80%, at least about 85%, at least about 90%, at least about 94%, at least about 95%, at least about 96%, at least about 97%, at least about 98%, or at least about 99%, sequence identity with SEQ ID NO: 802. Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, the polynucleotide encoding membrane-bound CKS_HTLV and GFP comprises SEQ ID NO: 800 or shares at least about 80%, at least about 85%, at least about 90%, at least about 94%, at least about 95%, at least about 96%, at least about 97%, at least about 98%, or at least about 99%, sequence identity with SEQ ID NO: 800. In several embodiments, the membrane-bound CKS_HTLV-GFP construct comprises an amino acid sequence of SEQ ID NO: 799 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 799.

In several embodiments, there is provided a polynucleotide encoding a chimeric immunosuppressive construct comprising a CKS-17 domain and an LMP domain. In several embodiments, provided for is a membrane-bound immunosuppressive construct comprising a CKS-17 domain and a an LMP domain (CKS_LMP-8aTM). In several embodiments, the CKS_LMP immunosuppressive peptide is coupled to a CD8α transmembrane protein and/or CD8α hinge domain. In several embodiments, the membrane-bound CKS_LMP viral immunosuppressive peptide construct comprises one or more linker sequences. In several embodiments, the membrane-bound CKS_LMP viral immunosuppressive construct comprises a CD8α signal peptide, a CKS-17 peptide, an LMP peptide, a CD8α hinge, and a CD8α transmembrane domain. In several embodiments, the membrane-bound CKS_LAMP immunosuppressive construct comprises the amino acid sequence set forth in SEQ ID NO:1073. In several embodiments, the membrane-bound CKS_LMP immunosuppressive construct is encoded by a polynucleotide that encodes an amino acid sequence comprising SEQ ID NO: 805. In several embodiments, the polynucleotide encodes a sequence that shares at least about 80%, at least about 85%, at least about 90%, at least about 94%, at least about 95%, at least about 96%, at least about 97%, at least about 98%, or at least about 99%, sequence identity, homology and/or functional equivalence with SEQ ID NO: 805. In several embodiments, the polynucleotide comprises SEQ ID NO: 806 or shares at least about 80%, at least about 85%, at least about 90%, at least about 94%, at least about 95%, at least about 96%, at least about 97%, at least about 98%, or at least about 99%, sequence identity with SEQ ID NO: 806. Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, the polynucleotide encoding membrane-bound CKS_LMP and GFP comprises SEQ ID NO: 804 or shares at least about 80%, at least about 85%, at least about 90%, at least about 94%, at least about 95%, at least about 96%, at least about 97%, at least about 98%, or at least about 99%, sequence identity with SEQ ID NO: 804. In several embodiments, the membrane-bound CKS_LMP-GFP construct comprises an amino acid sequence of SEQ ID NO: 803 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 803.

In several embodiments, there is provided a polynucleotide encoding a chimeric immunosuppressive construct comprising a CKS-17 domain and a GP41fp domain. In several embodiments, provided for is a membrane-bound immunosuppressive construct comprising a CKS-17 domain and an GP41fp domain (CKS_GP41fptm). In several embodiments, the membrane-bound CKS_GP41fptm viral immunosuppressive peptide construct comprises one or more linker sequences. In several embodiments, the membrane-bound CKS_GP41fptm viral immunosuppressive construct comprises a CD8α signal peptide, a first portion of the GP41fp (amino acids 1-16), a CKS-17 peptide, a second portion of the GP41fp (amino acids 17-70), and a gp41 transmembrane domain. In several embodiments, the membrane-bound CKX_GP41fptm immunosuppressive construct comprises the amino acid sequence set forth in SEQ ID NO:1074 In several embodiments, the membrane-bound CKS_GP41fptm immunosuppressive construct is encoded by a polynucleotide that encodes an amino acid sequence comprising SEQ ID NO: 809. In several embodiments, the polynucleotide encodes a sequence that shares at least about 80%, at least about 85%, at least about 90%, at least about 94%, at least about 95%, at least about 96%, at least about 97%, at least about 98%, or at least about 99%, sequence identity, homology and/or functional equivalence with SEQ ID NO: 809. In several embodiments, the polynucleotide comprises SEQ ID NO: 810 or shares at least about 80%, at least about 85%, at least about 90%, at least about 94%, at least about 95%, at least about 96%, at least about 97%, at least about 98%, or at least about 99%, sequence identity with SEQ ID NO: 810. Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, the polynucleotide encoding membrane-bound CKS_GP41fptm and GFP comprises SEQ ID NO: 808 or shares at least about 80%, at least about 85%, at least about 90%, at least about 94%, at least about 95%, at least about 96%, at least about 97%, at least about 98%, or at least about 99%, sequence identity with SEQ ID NO: 808. In several embodiments, the membrane-bound CKS_GP41fptm-GFP construct comprises an amino acid sequence of SEQ ID NO: 807 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 807.

In several embodiments, there is provided a polynucleotide encoding a truncated portion of the latent membrane protein 1 (LMP1) peptide of the Epstein-Barr Virus (LMP_S). In several embodiments, the LMP_S peptide is coupled to a CD8α signal peptide (e.g., for expression purposes, the sequence of which is provided for separately herein). In several embodiments, provided for is a membrane-bound LMP_S immunosuppressive construct comprising an LMP_S peptide coupled to a CD8α transmembrane protein and/or CD8α hinge domain. In several embodiments, the membrane-bound LMP_S immunosuppressive construct comprises one or more linker sequences. In several embodiments, the membrane-bound LMP_S immunosuppressive construct comprises the amino acid sequence set forth in SEQ ID NO:1075. In several embodiments, the membrane-bound LMP_S immunosuppressive construct comprises a CD8α signal peptide, an LMP_S peptide, an HTLV(Gp21) peptide, a CD8α hinge, and a CD8α transmembrane domain and is encoded by a polynucleotide that encodes an amino acid sequence comprising SEQ ID NO: 813. In several embodiments, the amino acid sequence shares at least about 90%, at least about 94%, at least about 95%, at least about 96%, at least about 97%, at least about 98%, or at least about 99%, sequence identity, homology and/or functional equivalence with SEQ ID NO: 813. In several embodiments, the polynucleotide comprises SEQ ID NO: 814 or shares at least about 80%, at least about 85%, at least about 90%, at least about 94%, at least about 95%, at least about 96%, at least about 97%, at least about 98%, or at least about 99%, sequence identity with SEQ ID NO: 814. Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, a linker (e.g., a GS linker) is positioned between the LMP_S and the tag. In several embodiments, the polynucleotide encoding LMP_S and GFP comprises SEQ ID NO: 812 or shares at least about 80%, at least about 85%, at least about 90%, at least about 94%, at least about 95%, at least about 96%, at least about 97%, at least about 98%, or at least about 99%, sequence identity with SEQ ID NO: 812. In several embodiments, the LMP_S-GFP construct comprises the amino acid sequence of SEQ ID NO: 811 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 811.

In several embodiments, there is provided a polynucleotide encoding a chimeric immunosuppressive construct comprising at least a portion of the latent membrane protein 1 (LMP1) peptide of the Epstein-Barr Virus (LMP_L) coupled with at least a portion of the extracellular and transmembrane domains of CD47 (CD47tm162), together referred to as LMP_CD47tm162. In several embodiments, the LMP_CD47tm162 is coupled to a CD8α signal peptide (e.g., for expression purposes, the sequence of which is provided for separately herein). In several embodiments, the LMP_CD47tm162 construct is membrane-bound based on the portion of the CD47 transmembrane domain and comprises one or more linker sequences. In several embodiments, the membrane-bound LMP_CD47tm162 immunosuppressive construct comprises the amino acid sequence set forth in SEQ ID NO:1076. In several embodiments, the membrane-bound LMP_CD47tm162 immunosuppressive construct comprises a CD8α signal peptide, an LMP_L peptide, and a CD47tm162 peptide, and is encoded by a polynucleotide that encodes an amino acid sequence comprising SEQ ID NO: 817. In several embodiments, the amino acid sequence shares at least about 90%, at least about 94%, at least about 95%, at least about 96%, at least about 97%, at least about 98%, or at least about 99%, sequence identity, homology and/or functional equivalence with SEQ ID NO: 817. In several embodiments, the polynucleotide comprises SEQ ID NO: 818 or shares at least about 80%, at least about 85%, at least about 90%, at least about 94%, at least about 95%, at least about 96%, at least about 97%, at least about 98%, or at least about 99%, sequence identity with SEQ ID NO: 818. Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, a linker (e.g., a GS linker) is positioned between the LMP_CD47tm162 and the tag. In several embodiments, the polynucleotide encoding LMP_CD47tm162 and GFP comprises SEQ ID NO: 816 or shares at least about 80%, at least about 85%, at least about 90%, at least about 94%, at least about 95%, at least about 96%, at least about 97%, at least about 98%, or at least about 99%, sequence identity with SEQ ID NO: 816. In several embodiments, the LMP_CD47tm162-GFP construct comprises the amino acid sequence of SEQ ID NO: 815 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 815.

In several embodiments, there is provided a polynucleotide encoding a chimeric immunosuppressive construct comprising at least a portion of a p15E viral peptide coupled with at least a portion of the extracellular and transmembrane domains of CD47 (CD47tm162), together referred to as p15E_CD47tm162. In several embodiments, the p15E_CD47tm162 is coupled to a CD8α signal peptide (e.g., for expression purposes, the sequence of which is provided for separately herein). In several embodiments, the p15E_CD47tm162 construct is membrane-bound based on the portion of the CD47 transmembrane domain and comprises one or more linker sequences. In several embodiments, the membrane-bound p15E_CD47tm162 immunosuppressive construct comprises the amino acid sequence set forth in SEQ ID NO:1077. In several embodiments, the membrane-bound p15E_CD47tm162 immunosuppressive construct comprises a CD8α signal peptide, a p15E peptide, and a CD47tm162 peptide, and is encoded by a polynucleotide that encodes an amino acid sequence comprising SEQ ID NO: 821. In several embodiments, the amino acid sequence shares at least about 90%, at least about 94%, at least about 95%, at least about 96%, at least about 97%, at least about 98%, or at least about 99%, sequence identity, homology and/or functional equivalence with SEQ ID NO: 821. In several embodiments, the polynucleotide comprises SEQ ID NO: 822 or shares at least about 80%, at least about 85%, at least about 90%, at least about 94%, at least about 95%, at least about 96%, at least about 97%, at least about 98%, or at least about 99%, sequence identity with SEQ ID NO: 822. Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, a linker (e.g., a GS linker) is positioned between the p15E_CD47tm162 and the tag. In several embodiments, the polynucleotide encoding p15E_CD47tm162 and GFP comprises SEQ ID NO: 820 or shares at least about 80%, at least about 85%, at least about 90%, at least about 94%, at least about 95%, at least about 96%, at least about 97%, at least about 98%, or at least about 99%, sequence identity with SEQ ID NO: 820. In several embodiments, the p15E_CD47tm162-GFP construct comprises the amino acid sequence of SEQ ID NO: 819 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 819.

In several embodiments, there is provided a polynucleotide encoding a chimeric immunosuppressive construct comprising at least one domain derived from HIV Gp41 and at least one domain from CD47 (CD47_GP41fptm). In several embodiments, the CD47_GP41fptm is coupled to a CD8α signal peptide (e.g., for expression purposes, the sequence of which is provided for separately herein). In several embodiments, the CD47_GP41fptm construct is membrane-bound based on a gp41 transmembrane domain. In several embodiments, the membrane-bound CD47_GP41fptm immunosuppressive construct comprises the amino acid sequence set forth in SEQ ID NO:1078. In several embodiments, the membrane-bound CD47_GP41fptm immunosuppressive construct comprises a CD8α signal peptide, a first GP41 domain (amino acids 1-16 of the extracellular domain), a CD47 domain (amino acids 2-141), a second GP41 domain (amino acids 17-70 of the extracellular domain), and a GP41 transmembrane domain and is encoded by a polynucleotide that encodes an amino acid sequence comprising SEQ ID NO: 825. In several embodiments, the amino acid sequence shares at least about 90%, at least about 94%, at least about 95%, at least about 96%, at least about 97%, at least about 98%, or at least about 99%, sequence identity, homology and/or functional equivalence with SEQ ID NO: 825. In several embodiments, the polynucleotide comprises SEQ ID NO: 826 or shares at least about 80%, at least about 85%, at least about 90%, at least about 94%, at least about 95%, at least about 96%, at least about 97%, at least about 98%, or at least about 99%, sequence identity with SEQ ID NO: 826. Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, a linker (e.g., a GS linker) is positioned between the CD47_GP41fptm and the tag. In several embodiments, the polynucleotide encoding CD47_GP41fptm and GFP comprises SEQ ID NO: 824 or shares at least about 80%, at least about 85%, at least about 90%, at least about 94%, at least about 95%, at least about 96%, at least about 97%, at least about 98%, or at least about 99%, sequence identity with SEQ ID NO: 824. In several embodiments, the CD47_GP41fptm-GFP construct comprises the amino acid sequence of SEQ ID NO: 823 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 823.

In several embodiments, there is provided a polynucleotide encoding an immunosuppressive construct comprising at least a portion of an antibody that targets a signal-regulatory protein α (SIRPα) (anti-SIRPa agonist_vHL), which is an innate immune checkpoint expressed on dendritic cells, macrophages, monocytes and neutrophils. In several embodiments, the constructs are engineered to be membrane-bound. In several embodiments, the anti-SIRPa agonist_vHL is coupled to a CD8α signal peptide (e.g., for expression purposes, the sequence of which is provided for separately herein). In several embodiments, the anti-SIRPa agonist_vHL construct is membrane-bound based on a CD8α hinge region and/or a CD8α transmembrane domain. In several embodiments, the membrane-bound anti-SIRPa agonist_vHL immunosuppressive construct comprises the amino acid sequence set forth in SEQ ID NO:1079 In several embodiments, the membrane-bound anti-SIRPa agonist_vHL immunosuppressive construct comprises a CD8α signal peptide, an anti-SIRPa heavy chain, a linker and anti-SIRPα light chain, a CD8α hinge region and, a CD8α transmembrane domain and is encoded by a polynucleotide that encodes an amino acid sequence comprising SEQ ID NO: 833. In several embodiments, the amino acid sequence shares at least about 90%, at least about 94%, at least about 95%, at least about 96%, at least about 97%, at least about 98%, or at least about 99%, sequence identity, homology and/or functional equivalence with SEQ ID NO: 833. In several embodiments, the polynucleotide comprises SEQ ID NO: 834 or shares at least about 80%, at least about 85%, at least about 90%, at least about 94%, at least about 95%, at least about 96%, at least about 97%, at least about 98%, or at least about 99%, sequence identity with SEQ ID NO: 834. Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, a linker (e.g., a GS linker) is positioned between the anti-SIRPa agonist_vHL and the tag. In several embodiments, the polynucleotide encoding anti-SIRPa agonist_vHL and GFP comprises SEQ ID NO: 832 or shares at least about 80%, at least about 85%, at least about 90%, at least about 94%, at least about 95%, at least about 96%, at least about 97%, at least about 98%, or at least about 99%, sequence identity with SEQ ID NO: 832. In several embodiments, the anti-SIRPa agonist_vHL-GFP construct comprises the amino acid sequence of SEQ ID NO: 831 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 831.

In several embodiments, there is provided a polynucleotide encoding an immunosuppressive construct comprising at least one domain derived from HTLV GP21 (HTLV1_GP21). In several embodiments, the HTLV1_GP21 is coupled to a CD8α signal peptide (e.g., for expression purposes, the sequence of which is provided for separately herein). In several embodiments, the HTLV_GP21 immunosuppressive construct comprises the amino acid sequence set forth in SEQ ID NO:1080. In several embodiments, the HTLV1_GP21 comprises a CD8α signal peptide and a GP21 transmembrane domain and is encoded by a polynucleotide that encodes an amino acid sequence comprising SEQ ID NO: 841. In several embodiments, the amino acid sequence shares at least about 90%, at least about 94%, at least about 95%, at least about 96%, at least about 97%, at least about 98%, or at least about 99%, sequence identity, homology and/or functional equivalence with SEQ ID NO: 841. In several embodiments, the polynucleotide comprises SEQ ID NO: 842 or shares at least about 80%, at least about 85%, at least about 90%, at least about 94%, at least about 95%, at least about 96%, at least about 97%, at least about 98%, or at least about 99%, sequence identity with SEQ ID NO: 842. Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, a linker (e.g., a GS linker) is positioned between the HTLV1_GP21 and the tag. In several embodiments, the polynucleotide encoding HTLV1_GP21 and GFP comprises SEQ ID NO: 840 or shares at least about 80%, at least about 85%, at least about 90%, at least about 94%, at least about 95%, at least about 96%, at least about 97%, at least about 98%, or at least about 99%, sequence identity with SEQ ID NO: 840. In several embodiments, the HTLV1_fGP62-GFP construct comprises the amino acid sequence of SEQ ID NO: 839 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 839.

In several embodiments, there is provided a polynucleotide encoding an immunosuppressive construct comprising at least one domain derived from a Lassa virus membrane glycoprotein 2 (LASV_fGP2). In several embodiments, the LASV_fGP2 immunosuppressive construct comprises the amino acid sequence set forth in SEQ ID NO:1081. In several embodiments, the LASV_fGP2 is coupled to a CD8α signal peptide (e.g., for expression purposes, the sequence of which is provided for separately herein). In several embodiments, the LASV_fGP2 comprises a CD8α signal peptide and at least a portion of a Lassa Virus GP2 domain, including a transmembrane region and is encoded by a polynucleotide that encodes an amino acid sequence comprising SEQ ID NO: 845. In several embodiments, the amino acid sequence shares at least about 90%, at least about 94%, at least about 95%, at least about 96%, at least about 97%, at least about 98%, or at least about 99%, sequence identity, homology and/or functional equivalence with SEQ ID NO: 845. In several embodiments, the polynucleotide comprises SEQ ID NO: 846 or shares at least about 80%, at least about 85%, at least about 90%, at least about 94%, at least about 95%, at least about 96%, at least about 97%, at least about 98%, or at least about 99%, sequence identity with SEQ ID NO: 846. Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, a linker (e.g., a GS linker) is positioned between the HTLV1_GP21 and the tag. In several embodiments, the polynucleotide encoding LASV_fGP2 and GFP comprises SEQ ID NO: 844 or shares at least about 80%, at least about 85%, at least about 90%, at least about 94%, at least about 95%, at least about 96%, at least about 97%, at least about 98%, or at least about 99%, sequence identity with SEQ ID NO: 844. In several embodiments, the LASV_fGP2-GFP construct comprises the amino acid sequence of SEQ ID NO: 843 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 843.

In several embodiments, there is provided a polynucleotide encoding an immunosuppressive construct comprising at least one domain derived from a Sudan ebolavirus envelope glycoprotein (SEBOV_fGP). In several embodiments, the SEBOV_fGP is coupled to a CD8α signal peptide (e.g., for expression purposes, the sequence of which is provided for separately herein). In several embodiments, the SEBOV_fGP comprises a CD8α signal peptide and at least two domains from the ebolavirus envelope. In several embodiments, the SEBOV_fGP comprises a full GP1 glycoprotein sequence followed by a truncated form of GP1 (known as GP2, resulting from proteolysis of the GP1 domain). In several embodiments, the SEBOV_fGP immunosuppressive construct comprises the amino acid sequence set forth in SEQ ID NO:1082. In several embodiments, the GP2 domain includes a transmembrane domain. In several embodiments, the SEBOV_fGP is encoded by a polynucleotide that encodes an amino acid sequence comprising SEQ ID NO: 849. In several embodiments, the amino acid sequence shares at least about 90%, at least about 94%, at least about 95%, at least about 96%, at least about 97%, at least about 98%, or at least about 99%, sequence identity, homology and/or functional equivalence with SEQ ID NO: 849. In several embodiments, the polynucleotide comprises SEQ ID NO: 850 or shares at least about 80%, at least about 85%, at least about 90%, at least about 94%, at least about 95%, at least about 96%, at least about 97%, at least about 98%, or at least about 99%, sequence identity with SEQ ID NO: 850. Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, a linker (e.g., a GS linker) is positioned between the SEBOV_fGP and the tag. In several embodiments, the polynucleotide encoding SEBOV_fGP and GFP comprises SEQ ID NO: 848 or shares at least about 80%, at least about 85%, at least about 90%, at least about 94%, at least about 95%, at least about 96%, at least about 97%, at least about 98%, or at least about 99%, sequence identity with SEQ ID NO: 848. In several embodiments, the SEBOV_fGP-GFP construct comprises the amino acid sequence of SEQ ID NO: 847 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 847.

In several embodiments, there is provided a polynucleotide encoding an immunosuppressive construct comprising at least one domain derived from a Sudan ebolavirus envelope glycoprotein (SEBOV_GP2). In several embodiments, the SEBOV_GP2 is coupled to a CD8α signal peptide (e.g., for expression purposes, the sequence of which is provided for separately herein). In several embodiments, the SEBOV_GP2 comprises a CD8α signal peptide and a GP2 domain, resulting from proteolysis of the GP1 domain. In several embodiments, the GP2 domain includes a transmembrane domain. In several embodiments, the SEBOV_GP2 immunosuppressive construct comprises the amino acid sequence set forth in SEQ ID NO:1083. In several embodiments, the SEBOV_GP2 is encoded by a polynucleotide that encodes an amino acid sequence comprising SEQ ID NO: 853. In several embodiments, the amino acid sequence shares at least about 90%, at least about 94%, at least about 95%, at least about 96%, at least about 97%, at least about 98%, or at least about 99%, sequence identity, homology and/or functional equivalence with SEQ ID NO: 853. In several embodiments, the polynucleotide comprises SEQ ID NO: 854 or shares at least about 80%, at least about 85%, at least about 90%, at least about 94%, at least about 95%, at least about 96%, at least about 97%, at least about 98%, or at least about 99%, sequence identity with SEQ ID NO: 854. Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, a linker (e.g., a GS linker) is positioned between the SEBOV_GP2 and the tag. In several embodiments, the polynucleotide encoding SEBOV_GP2 and GFP comprises SEQ ID NO: 852 or shares at least about 80%, at least about 85%, at least about 90%, at least about 94%, at least about 95%, at least about 96%, at least about 97%, at least about 98%, or at least about 99%, sequence identity with SEQ ID NO: 852. In several embodiments, the SEBOV_GP2-GFP construct comprises the amino acid sequence of SEQ ID NO: 851 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 851.

In several embodiments, there is provided a polynucleotide encoding an immunosuppressive construct comprising at least one domain derived from a Sars-CoV-2 spike protein (SCoV_S2). In several embodiments, the SCoV_S2 is coupled to a CD8α signal peptide (e.g., for expression purposes, the sequence of which is provided for separately herein). In several embodiments, the SCoV_S2 comprises a CD8α signal peptide and a an S2 spike protein, including a transmembrane domain. In several embodiments, the SDCoV_S2 immunosuppressive construct comprises the amino acid sequence set forth in SEQ ID NO:1084. In several embodiments, the SCoV_S2 is encoded by a polynucleotide that encodes an amino acid sequence comprising SEQ ID NO: 857. In several embodiments, the amino acid sequence shares at least about 90%, at least about 94%, at least about 95%, at least about 96%, at least about 97%, at least about 98%, or at least about 99%, sequence identity, homology and/or functional equivalence with SEQ ID NO: 857. In several embodiments, the polynucleotide comprises SEQ ID NO: 858 or shares at least about 80%, at least about 85%, at least about 90%, at least about 94%, at least about 95%, at least about 96%, at least about 97%, at least about 98%, or at least about 99%, sequence identity with SEQ ID NO: 858. Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, a linker (e.g., a GS linker) is positioned between the SCoV_S2 and the tag. In several embodiments, the polynucleotide encoding SCoV_S2 and GFP comprises SEQ ID NO: 856 or shares at least about 80%, at least about 85%, at least about 90%, at least about 94%, at least about 95%, at least about 96%, at least about 97%, at least about 98%, or at least about 99%, sequence identity with SEQ ID NO: 856. In several embodiments, the SCoV_S2-GFP construct comprises the amino acid sequence of SEQ ID NO: 855 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 855.

In several embodiments, there is provided a polynucleotide encoding an immunosuppressive construct comprising at least a portion of a Galectin-3-binding protein (LGALS3BP). In several embodiments, the constructs are engineered to be membrane-bound. In several embodiments, the LGALS3BP is coupled to a CD8α signal peptide (e.g., for expression purposes, the sequence of which is provided for separately herein). In several embodiments, LGALS3BP construct is membrane-bound based on a CD8α hinge region and/or a CD8α transmembrane domain. In several embodiments, the LGALS3BP immunosuppressive construct comprises the amino acid sequence set forth in SEQ ID NO:1085 In several embodiments, the membrane-bound LGALS3BP immunosuppressive construct comprises a CD8α signal peptide, a Galectin 3 binding protein, and a CD8α transmembrane domain and is encoded by a polynucleotide that encodes an amino acid sequence comprising SEQ ID NO: 861. In several embodiments, the amino acid sequence shares at least about 90%, at least about 94%, at least about 95%, at least about 96%, at least about 97%, at least about 98%, or at least about 99%, sequence identity, homology and/or functional equivalence with SEQ ID NO: 861. In several embodiments, the polynucleotide comprises SEQ ID NO: 862 or shares at least about 80%, at least about 85%, at least about 90%, at least about 94%, at least about 95%, at least about 96%, at least about 97%, at least about 98%, or at least about 99%, sequence identity with SEQ ID NO: 862. Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, a linker (e.g., a GS linker) is positioned between the LGALS3BP and the tag. In several embodiments, the polynucleotide encoding LGALS3BP and GFP comprises SEQ ID NO: 860 or shares at least about 80%, at least about 85%, at least about 90%, at least about 94%, at least about 95%, at least about 96%, at least about 97%, at least about 98%, or at least about 99%, sequence identity with SEQ ID NO: 860. In several embodiments, the LGALS3BP-GFP construct comprises the amino acid sequence of SEQ ID NO: 859 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 859.

In several embodiments, there is provided a polynucleotide encoding an immunosuppressive construct comprising at least a portion of CD24. In several embodiments, the constructs are engineered to be membrane-bound. In several embodiments, the CD24 immunosuppressive construct comprises the amino acid sequence set forth in SEQ ID NO:1086. In several embodiments, the CD24 construct comprises a CD24 signal peptide and a CD24 protein and is encoded by a polynucleotide that encodes an amino acid sequence comprising SEQ ID NO: 865. In several embodiments, the amino acid sequence shares at least about 90%, at least about 94%, at least about 95%, at least about 96%, at least about 97%, at least about 98%, or at least about 99%, sequence identity, homology and/or functional equivalence with SEQ ID NO: 865. In several embodiments, the polynucleotide comprises SEQ ID NO: 866 or shares at least about 80%, at least about 85%, at least about 90%, at least about 94%, at least about 95%, at least about 96%, at least about 97%, at least about 98%, or at least about 99%, sequence identity with SEQ ID NO: 866. Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, a T2A domain is positioned between the CD24 and the tag. In several embodiments, the polynucleotide encoding CD24 and GFP comprises SEQ ID NO: 864 or shares at least about 80%, at least about 85%, at least about 90%, at least about 94%, at least about 95%, at least about 96%, at least about 97%, at least about 98%, or at least about 99%, sequence identity with SEQ ID NO: 864. In several embodiments, the CD24-GFP construct comprises the amino acid sequence of SEQ ID NO: 863 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 863.

In several embodiments, there is provided a polynucleotide encoding an immunosuppressive construct comprising at least a portion of a Hepatitis C envelope glycoprotein (HCV_E2). In several embodiments, the HCV_E2 immunosuppressive construct comprises the amino acid sequence set forth in SEQ ID NO:1087. In several embodiments, the HCV_E2 is coupled to a CD8α signal peptide (e.g., for expression purposes, the sequence of which is provided for separately herein). In several embodiments, the HCV_E2 immunosuppressive construct comprises a CD8α signal peptide, and a E2 protein of HCV and is encoded by a polynucleotide that encodes an amino acid sequence comprising SEQ ID NO: 869. In several embodiments, the amino acid sequence shares at least about 90%, at least about 94%, at least about 95%, at least about 96%, at least about 97%, at least about 98%, or at least about 99%, sequence identity, homology and/or functional equivalence with SEQ ID NO: 869. In several embodiments, the polynucleotide comprises SEQ ID NO: 870 or shares at least about 80%, at least about 85%, at least about 90%, at least about 94%, at least about 95%, at least about 96%, at least about 97%, at least about 98%, or at least about 99%, sequence identity with SEQ ID NO: 870. Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, a linker (e.g., a GS linker) is positioned between the HCV_E2 and the tag. In several embodiments, the polynucleotide encoding HCV_E2 and GFP comprises SEQ ID NO: 868 or shares at least about 80%, at least about 85%, at least about 90%, at least about 94%, at least about 95%, at least about 96%, at least about 97%, at least about 98%, or at least about 99%, sequence identity with SEQ ID NO: 868. In several embodiments, the HCV_E2-GFP construct comprises the amino acid sequence of SEQ ID NO: 867 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 867.

In several embodiments, there is provided a polynucleotide encoding an immunosuppressive construct comprising at least a portion of an antibody that targets a signal-regulatory protein α (SIRPα) (anti-SIRPa agonist_vLH). In several embodiments, the constructs are engineered to be membrane-bound. In several embodiments, the anti-SIRPa agonist_vLH is coupled to a CD8α signal peptide (e.g., for expression purposes, the sequence of which is provided for separately herein). In several embodiments, the anti-SIRPa agonist_vLH construct is membrane-bound based on a CD8α hinge region and/or a CD8α transmembrane domain. In several embodiments, the membrane-bound anti-SIRPa agonist_vLH immunosuppressive construct comprises the amino acid sequence set forth in SEQ ID NO:1088. In several embodiments, the membrane-bound anti-SIRPa agonist_vLH immunosuppressive construct comprises a CD8α signal peptide, an anti-SIRPα light chain, a linker, anti-SIRPα heavy chain, a CD8α hinge region and, a CD8α transmembrane domain and is encoded by a polynucleotide that encodes an amino acid sequence comprising SEQ ID NO: 873. In several embodiments, the amino acid sequence shares at least about 90%, at least about 94%, at least about 95%, at least about 96%, at least about 97%, at least about 98%, or at least about 99%, sequence identity, homology and/or functional equivalence with SEQ ID NO: 873. In several embodiments, the polynucleotide comprises SEQ ID NO: 874 or shares at least about 80%, at least about 85%, at least about 90%, at least about 94%, at least about 95%, at least about 96%, at least about 97%, at least about 98%, or at least about 99%, sequence identity with SEQ ID NO: 874. Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, a linker (e.g., a GS linker) is positioned between the anti-SIRPa agonist_vLH and the tag. In several embodiments, the polynucleotide encoding anti-SIRPa agonist_vLH and GFP comprises SEQ ID NO: 872 or shares at least about 80%, at least about 85%, at least about 90%, at least about 94%, at least about 95%, at least about 96%, at least about 97%, at least about 98%, or at least about 99%, sequence identity with SEQ ID NO: 872. In several embodiments, the anti-SIRPa agonist_vLH-GFP construct comprises the amino acid sequence of SEQ ID NO: 871 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 871.

In several embodiments, there is provided a polynucleotide encoding an immunosuppressive construct comprising at least CEA Cell Adhesion Molecule 1-derived domain (CEACAM1). In several embodiments, the constructs are engineered to be membrane-bound. In several embodiments, the CEACAM1 is coupled to a CD8α signal peptide (e.g., for expression purposes, the sequence of which is provided for separately herein). In several embodiments, the CEACAM1 immunosuppressive construct comprises a CD8α signal peptide and a CEACAM1-derived protein. In several embodiments, the CEACAM1 immunosuppressive construct comprises the amino acid sequence set forth in SEQ ID NO:1089. In several embodiments, the CEACAM1 immunosuppressive construct is encoded by a polynucleotide that encodes an amino acid sequence comprising SEQ ID NO: 877. In several embodiments, the amino acid sequence shares at least about 90%, at least about 94%, at least about 95%, at least about 96%, at least about 97%, at least about 98%, or at least about 99%, sequence identity, homology and/or functional equivalence with SEQ ID NO: 877. In several embodiments, the polynucleotide comprises SEQ ID NO: 878 or shares at least about 80%, at least about 85%, at least about 90%, at least about 94%, at least about 95%, at least about 96%, at least about 97%, at least about 98%, or at least about 99%, sequence identity with SEQ ID NO: 878. Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, a linker (e.g., a GS linker) is positioned between the CEACAM1 and the tag. In several embodiments, the polynucleotide encoding the CEACAM1 and GFP comprises SEQ ID NO: 876 or shares at least about 80%, at least about 85%, at least about 90%, at least about 94%, at least about 95%, at least about 96%, at least about 97%, at least about 98%, or at least about 99%, sequence identity with SEQ ID NO: 876. In several embodiments, the CEACAM1-GFP construct comprises the amino acid sequence of SEQ ID NO: 875 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 875.

In several embodiments, there is provided a polynucleotide encoding an immunosuppressive construct comprising CD155 transmembrane domain (CD155tm_3M). In several embodiments, the CD155tm_3M is coupled to a CD8α signal peptide (e.g., for expression purposes, the sequence of which is provided for separately herein). In several embodiments, the CD155tm_3M immunosuppressive construct comprises a CD8α signal peptide and a CD155-derived protein. In several embodiments, the CD155tm_3M immunosuppressive construct comprises the amino acid sequence set forth in SEQ ID NO:1090. In several embodiments, the CD155tm_3M immunosuppressive construct is encoded by a polynucleotide that encodes an amino acid sequence comprising SEQ ID NO: 881. In several embodiments, the amino acid sequence shares at least about 90%, at least about 94%, at least about 95%, at least about 96%, at least about 97%, at least about 98%, or at least about 99%, sequence identity, homology and/or functional equivalence with SEQ ID NO: 881. In several embodiments, the polynucleotide comprises SEQ ID NO: 882 or shares at least about 80%, at least about 85%, at least about 90%, at least about 94%, at least about 95%, at least about 96%, at least about 97%, at least about 98%, or at least about 99%, sequence identity with SEQ ID NO: 882. Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, a linker (e.g., a GS linker) is positioned between the CD155tm_3M and the tag. In several embodiments, the polynucleotide encoding the CD155tm_3M and GFP comprises SEQ ID NO: 880 or shares at least about 80%, at least about 85%, at least about 90%, at least about 94%, at least about 95%, at least about 96%, at least about 97%, at least about 98%, or at least about 99%, sequence identity with SEQ ID NO: 880. In several embodiments, the CD155tm_3M-GFP construct comprises the amino acid sequence of SEQ ID NO: 879 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 879.

In several embodiments, there is provided a polynucleotide encoding an immunosuppressive construct comprising CD31 transmembrane domain (CD31tm). In several embodiments, the CD31tm is coupled to a CD8α signal peptide (e.g., for expression purposes, the sequence of which is provided for separately herein). In several embodiments, the CD31tm immunosuppressive construct comprises a CD8α signal peptide and a CD31-derived protein. In several embodiments, the CD31tm immunosuppressive construct comprises the amino acid sequence set forth in SEQ ID NO:1091. In several embodiments, the CD31tm immunosuppressive construct is encoded by a polynucleotide that encodes an amino acid sequence comprising SEQ ID NO: 885. In several embodiments, the amino acid sequence shares at least about 90%, at least about 94%, at least about 95%, at least about 96%, at least about 97%, at least about 98%, or at least about 99%, sequence identity, homology and/or functional equivalence with SEQ ID NO: 885. In several embodiments, the polynucleotide comprises SEQ ID NO: 886 or shares at least about 80%, at least about 85%, at least about 90%, at least about 94%, at least about 95%, at least about 96%, at least about 97%, at least about 98%, or at least about 99%, sequence identity with SEQ ID NO: 886. Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, a linker (e.g., a GS linker) is positioned between the CD31tm and the tag. In several embodiments, the polynucleotide encoding the CD31tm and GFP comprises SEQ ID NO: 884 or shares at least about 80%, at least about 85%, at least about 90%, at least about 94%, at least about 95%, at least about 96%, at least about 97%, at least about 98%, or at least about 99%, sequence identity with SEQ ID NO: 884. In several embodiments, the CD31tm-GFP construct comprises the amino acid sequence of SEQ ID NO: 883 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 883.

In several embodiments, there is provided a polynucleotide encoding an immunosuppressive construct comprising CD111 transmembrane domain (CD111tm). In several embodiments, the CD111tm is coupled to a CD8α signal peptide (e.g., for expression purposes, the sequence of which is provided for separately herein). In several embodiments, the CD111tm immunosuppressive construct comprises a CD8α signal peptide and a CD111tm-derived protein. In several embodiments, the CD111tm immunosuppressive construct comprises the amino acid sequence set forth in SEQ ID NO:1092. In several embodiments, the CD111tm immunosuppressive construct is encoded by a polynucleotide that encodes an amino acid sequence comprising SEQ ID NO: 889. In several embodiments, the amino acid sequence shares at least about 90%, at least about 94%, at least about 95%, at least about 96%, at least about 97%, at least about 98%, or at least about 99%, sequence identity, homology and/or functional equivalence with SEQ ID NO: 889. In several embodiments, the polynucleotide comprises SEQ ID NO: 890 or shares at least about 80%, at least about 85%, at least about 90%, at least about 94%, at least about 95%, at least about 96%, at least about 97%, at least about 98%, or at least about 99%, sequence identity with SEQ ID NO: 890. Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, a linker (e.g., a GS linker) is positioned between the CD111tm and the tag. In several embodiments, the polynucleotide encoding the CD111tm and GFP comprises SEQ ID NO: 888 or shares at least about 80%, at least about 85%, at least about 90%, at least about 94%, at least about 95%, at least about 96%, at least about 97%, at least about 98%, or at least about 99%, sequence identity with SEQ ID NO: 888. In several embodiments, the CD111tm-GFP construct comprises the amino acid sequence of SEQ ID NO: 887 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 887.

In several embodiments, there is provided a polynucleotide encoding an immunosuppressive construct comprising CD200 transmembrane domain (CD200tm). In several embodiments, the CD200tm is coupled to a CD8α signal peptide (e.g., for expression purposes, the sequence of which is provided for separately herein). In several embodiments, the CD200tm immunosuppressive construct comprises a CD8α signal peptide and a CD200tm-derived protein. In several embodiments, the CD200tm immunosuppressive construct comprises the amino acid sequence set forth in SEQ ID NO:1093. In several embodiments, the CD200tm immunosuppressive construct is encoded by a polynucleotide that encodes an amino acid sequence comprising SEQ ID NO: 893. In several embodiments, the amino acid sequence shares at least about 90%, at least about 94%, at least about 95%, at least about 96%, at least about 97%, at least about 98%, or at least about 99%, sequence identity, homology and/or functional equivalence with SEQ ID NO: 893. In several embodiments, the polynucleotide comprises SEQ ID NO: 894 or shares at least about 80%, at least about 85%, at least about 90%, at least about 94%, at least about 95%, at least about 96%, at least about 97%, at least about 98%, or at least about 99%, sequence identity with SEQ ID NO: 894. Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, a linker (e.g., a GS linker) is positioned between the CD200tm and the tag. In several embodiments, the polynucleotide encoding the CD200tm and GFP comprises SEQ ID NO: 892 or shares at least about 80%, at least about 85%, at least about 90%, at least about 94%, at least about 95%, at least about 96%, at least about 97%, at least about 98%, or at least about 99%, sequence identity with SEQ ID NO: 892. In several embodiments, the CD200tm-GFP construct comprises the amino acid sequence of SEQ ID NO: 891 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 891.

In several embodiments, the engineered cells provided for herein comprise a chimeric receptor that targets NKG2D ligands, wherein the CAR comprises an amino acid of SEQ ID NO: 1024 (LFNQEVQIPLTESYCGPCPKNWICYKNNCYQFFDESKNWYESQASCMSQNASLLKVYSKEDQDLLKLVKS YHWMGLVHIPTNGSWQWEDGSILSPNLLTIIEMQKGDCALYASSFKGYIENCSTPNTYICMQRTVTTTPAPR PPTPAPTIASQPLSLRPEACRPAAGGAVHTRGLDFACDIYIWAPLAGTCGVLLLSLVITLYCRRDQRLPPDAH KPPGGGSFRTPIQEEQADAHSTLAKIRVKFSRSADAPAYQQGQNQLYNELNLGRREEYDVLDKRRGRDPE MGGKPRRKNPQEGLYNELQKDKMAEAYSEIGMKGERRRGKGHDGLYQGLSTATKDTYDALHMQALPPR (e.g., SEQ ID NO: 174) or SEQ ID NO:1025 (LFNQEVQIPLTESYCGPCPKNWICYKNNCYQFFDESKNWYESQASCMSQNASLLKVYSKEDQDLLKLVKS YHWMGLVHIPTNGSWQWEDGSILSPNLLTIIEMQKGDCALYASSFKGYIENCSTPNTYICMQRTVTTTPAPR PPTPAPTIASQPLSLRPEACRPAAGGAVHTRGLDFACDIYIWAPLAGTCGVLLLSLVITLYCRRDQRLPPDAH KPPGGGSFRTPIQEEQADAHSTLAKIRVKFSRSADAPAYQQGQNQLYNELNLGRREEYDVLDKRRGRDPE MGGKPRRKNPQEGLYNELQKDKMAEAYSEIGMKGERRRGKGHDGLYQGLSTATKDTYDALHMQALPPR), (e.g., SEQ ID NO:899), or comprises an amino acid sequence with at least about 80%, at least about 85%, at least about 90%, at least about 94%, at least about 95%, at least about 96%, at least about 97%, at least about 98%, or at least about 99%, sequence identity, homology and/or functional equivalence to SEQ ID NO: 1024, 1025, 174 or 899 and one or more copies of one or more of the following membrane-bound immunosuppressive amino acid sequences: SEQ ID NO: 218, 223, 228, 233, 238, 243, 248, 253, 256, 259, 262, 265, 268, and 271.

In several embodiments, the engineered cells provided for herein comprise a CAR that targets CD19, wherein the CAR comprises an amino acid of SEQ ID NO: 1026 (DIQMTQSPSSLSASVGDRVTITCRASQDISKYLNWYQQKPGGTVKLLIYHTSRLHSGVPSRFSGSGSGTDFT LTISSLQPEDIATYFCQQGNTLPYTFGGGTKLEITGGGGSGGGGSGGGGSQVQLQESGPGLVKPSQTLSLTC TVSGVSLPDYGVSWIRQPPGKGLEWIGVIWGSETTYYNSALKSRLTISKDNSKNQVSLKLSSVTAADTAVY YCAKHYYYGGSYAMDYWGQGTSVTVSSTTTPAPRPPTPAPTIASQPLSLRPEACRPAAGGAVHTRGLDFA CDIYIWAPLAGTCGVLLLSLVITLYCRRDQRLPPDAHKPPGGGSFRTPIQEEQADAHSTLAKIRVKFSRSADA PAYQQGQNQLYNELNLGRREEYDVLDKRRGRDPEMGGKPRRKNPQEGLYNELQKDKMAEAYSEIGMKG ERRRGKGHDGLYQGLSTATKDTYDALHMQALPPRGSGEGRGSLLTCGDVEENPGPMALPVTALLLPLALL LHAARPNWVNVISDLKKIEDLIQSMHIDATLYTESDVHPSCKVTAMKCFLLELQVISLESGDASIHDTVENLI ILANNSLSSNGNVTESGCKECEELEEKNIKEFLQSFVHIVQMFINTSTTTPAPRPPTPAPTIASQPLSLRPEACR PAAGGAVHTRGLDFACDIYIWAPLAGTCGVLLLSLVITLYC), (e.g., SEQ ID NO: 178) or SEQ ID NO:1027 (DIQMTQSPSSLSASVGDRVTITCRASQDISKYLNWYQQKPGGTVKLLIYHTSRLHSGVPSRFSGSGSGTDFT LTISSLQPEDIATYFCQQGNTLPYTFGGGTKLEITGGGGSGGGGSGGGGSQVQLQESGPGLVKPSQTLSLTC TVSGVSLPDYGVSWIRQPPGKGLEWIGVIWGSETTYYNSALKSRLTISKDNSKNQVSLKLSSVTAADTAVY YCAKHYYYGGSYAMDYWGQGTSVTVSSTTTPAPRPPTPAPTIASQPLSLRPEACRPAAGGAVHTRGLDFA CDIYIWAPLAGTCGVLLLSLVITLYCRRDQRLPPDAHKPPGGGSFRTPIQEEQADAHSTLAKIRVKFSRSADA PAYQQGQNQLYNELNLGRREEYDVLDKRRGRDPEMGGKPRRKNPQEGLYNELQKDKMAEAYSEIGMKG ERRRGKGHDGLYQGLSTATKDTYDALHMQALPPR), (e.g., SEQ ID NO:901), or comprises an amino acid sequence with at least about 80%, at least about 85%, at least about 90%, at least about 94%, at least about 95%, at least about 96%, at least about 97%, at least about 98%, or at least about 99%, sequence identity, homology and/or functional equivalence to SEQ ID NO: 178 or 901 and one or more copies of one or more of the following membrane-bound immunosuppressive amino acid sequences: SEQ ID NO: 218, 223, 228, 233, 238, 243, 248, 253, 256, 259, 262, 265, 268, and 271.

In several embodiments, the engineered cells provided for herein comprise a CAR that targets CD19, wherein the CAR is encoded by SEQ ID NO: 466, or comprises an nucleicacid sequence with at least about 80%, at least about 85%, at least about 90%, at least about 94%, at least about 95%, at least about 96%, at least about 97%, at least about 98%, or at least about 99%, sequence identity, homology and/or functional equivalence to the sequence encoded by SEQ ID NO: 466 and one or more copies of one or more of the following membrane-bound immunosuppressive amino acid sequences: SEQ ID NO: 218, 223, 228, 233, 238, 243, 248, 253, 256, 259, 262, 265, 268, and 271.

In several embodiments, the engineered cells provided for herein comprise a CAR that targets CD70, wherein the CAR comprises an amino acid of any of SEQ ID NOs: 383-465 or 912-994, or comprises an amino acid sequence with at least about 80%, at least about 85%, at least about 90%, at least about 94%, at least about 95%, at least about 96%, at least about 97%, at least about 98%, or at least about 99%, sequence identity, homology and/or functional equivalence to any of SEQ ID NO: 383-465 or 912-994 and one or more copies of one or more of the following membrane-bound immunosuppressive amino acid sequences: SEQ ID NO: 218, 223, 228, 233, 238, 243, 248, 253, 256, 259, 262, 265, 268, and 271.

It is contemplated that any of the amino acid sequences provided herein may be provided with or without a signal sequence (e.g., a CD8α signal sequence, such as MALPVTALLLPLALLLHAARP). It is also contemplated that any of the amino acid sequences provided herein may be provided with or without an initial methionine (M) residue.

### HLA-Modification

In several embodiments, immune cells are engineered to alter their HLA expression profile, order to interact with one or more inhibitory receptors on host immune cells. In several embodiments, the alteration of the HLA expression profile of engineered cells functions to impart to the engineered immune cell the ability to reduce or avoid cytotoxicity or other immune clearance by host immune cells (or other engineered immune cells), thereby enhancing the persistence (and thus functional life-span) of the engineered immune cells.

Human leukocyte antigen-E (HLA-E) is a major ligand for the natural killer inhibitory receptor CD94/NKG2A that is expressed on NK cells. When HLA-E is bound by the CD94/NKG2A complex, an inhibitory signaling cascade is initiated, resulting in reduced NK cell activity. Thus, expression of HLA-E can dampen the cytotoxic effects of host NK cells against an engineered immune cell (e.g., expressing a CAR and/or immunosuppressive peptides). Likewise, HLA-G also plays an important role in inhibiting natural killer (NK) cell function, not only in the maintenance of fetal-maternal immune tolerance but also in the context of organ or tissue transplantation. It also plays a role in the immune escape of tumors from host immune cells. HLA-G can inhibit the function of many immune cells such as NK cells, CD4+ and CD8+ T cells, and dendritic cells by binding to cell surface-expressed receptors, including immunoglobulin-like transcript 2 (ILT2), ILT4 and killer cell immunoglobulin-like receptor 2DL4 (KIR2DL4). In several embodiments, immune cells as disclosed herein are engineered to express HLA-E and/or HLA-G in order to suppress host NK cell (or other engineered NK cells administered) against the engineered immune cells.

Non-limiting embodiments of such an approach are schematically depicted in FIGS. 10I-10J. FIG. 10I shows an engineered immune cell according to embodiments disclosed herein that expresses a CAR comprising an immunosuppressive domain as well as a membrane-bound immunosuppressive construct. In some embodiments, expression of HLA-E and/or HLA-G is in connection with a CAR and not a membrane-bound immunosuppressive construct, or optionally with a CAR that does not include an immunosuppressive domain. FIG. 10J schematically depicts another non-limiting embodiment wherein HLA-E and/or HLA-G are co-expressed with one or more membrane-bound immunosuppressive constructs, optionally a CAR (with or without an integrated immunosuppressive domain) and optionally one or more additional immunosuppressive proteins (non-limiting examples of with include, but are not limited to CD47, PD-L1, and the poliovirus receptor (PVR, also known as CD155). As discussed above, each of CD47, PD-L1 and CD155 can operate to reduce activity of immune cells (e.g., host immune cells and/or other administered engineered immune cells). Any combination of these molecules, or any others disclosed herein can be used.

In several embodiments, such as when B2M expression is reduced or knocked out, endogenous HLA expression is lost, and in several embodiments, specific re-expression of HLA-E and or HLA-G can aid in reduce NK cell activity against immune cells engineered to express HLA-E and or HLA-G. In several embodiments, this re-expression is coupled with gene editing to reduce NKG2A expression on NK cells to be administered, which limits the suppressive effect of HLA-E on the therapeutic cells themselves. In several embodiments, HLA-E expression is specifically introduced only on T cells. In several embodiments, those T cells operate to suppress NK cell activity via interaction with the NKG2A receptor on NK cells. In some embodiments, the activity of the engineered allogeneic NK cells is suppressed temporarily. In several embodiments, the temporary suppression of engineered allogeneic NK cell activity reduces the risk of NK cell exhaustion, which prolongs the persistence of the engineered allogeneic NK cells.

In several embodiments, there is provided a polynucleotide encoding HLA-E. In several embodiments, the polynucleotide encodes an HLA-E amino acid sequence comprising SEQ ID NO: 273: HSLKYFHTSVSRPGRGEPRFISVGYVDDTQFVRFDNDAASPRMVPRAPWMEQEGSEYWDRETRSARDTAQ IFRVNLRTLRGYYNQSEAGSHTLQWMHGCELGPDRRFLRGYEQFAYDGKDYLTLNEDLRSWTAVDTAAQ ISEQKSNDASEAEHQRAYLEDTCVEWLHKYLEKGKETLLHLEPPKTHVTHHPISDHEATLRCWALGFYPAE ITLTWQQDGEGHTQDTELVETRPAGDGTFQKWAAVVVPSGEEQRYTCHVQHEGLPEPVTLRWKPASQPTI PIVGIIAGLVLLGSVVSGAVVAAVIWRKKSSGGKGGSYYKAEWSDSAQGSESHSL.

In several embodiments, the polynucleotide encodes a sequence that shares at least about 80%, at least about 85%, at least about 90%, at least about 94%, at least about 95%, at least about 96%, at least about 97%, at least about 98%, or at least about 99%, sequence identity, homology and/or functional equivalence with SEQ ID NO: 273. In several embodiments, the polynucleotide comprises SEQ ID NO: 274 or shares at least about 90%, at least about 94%, at least about 95%, at least about 96%, at least about 97%, at least about 98%, or at least about 99%, sequence identity with SEQ ID NO: 274. In several embodiments, the encoded HLA-E is an HLA-E single chain trimer (SCT) composed of a canonical HLA-E binding peptide, mature human beta2-microglobulin, and mature HLA-E heavy chain (HLA-E trimer_SS). In several embodiments, the construct comprises a CD8α signal peptide, a first B2M sequence, an HLG peptide leader sequence, a linker (e.g., a GS linker), a second B2M sequence, a linker (e.g., a second GS linker), and an HLA-E sequence. In several embodiments, the HLA-E trimer_SS construct is encoded by a polynucleotide that encodes an amino acid sequence comprising SEQ ID NO: 1094 (SRSVALAVLALLSLSGLEAVMAPRTLFLGGGGSGGGGSGGGGSIQRTPKIQVYSRHPAENGKSNFLNCYVS GFHPSDIEVDLLKNGERIEKVEHSDLSFSKDWSFYLLYYTEFTPTEKDEYACRVNHVTLSQPKIVKWDRDM GGGGSGGGGSGGGGSGGGGSGSHSLKYFHTSVSRPGRGEPRFISVGYVDDTQFVRFDNDAASPRMVPRAP WMEQEGSEYWDRETRSARDTAQIFRVNLRTLRGYYNQSEAGSHTLQWMHGCELGPDRRFLRGYEQFAYD GKDYLTLNEDLRSWTAVDTAAQISEQKSNDASEAEHQRAYLEDTCVEWLHKYLEKGKETLLHLEPPKTHV THHPISDHEATLRCWALGFYPAEITLTWQQDGEGHTQDTELVETRPAGDGTFQKWAAVVVPSGEEQRYTC HVQHEGLPEPVTLRWKPASQPTIPIVGIIAGLVLLGSVVSGAVVAAVIWRKKSSGGKGGSYYKAEWSDSAQ GSESHSL), (e.g., SEQ ID NO: 276) or SEQ ID NO:1017 (SRSVALAVLALLSLSGLEAVMAPRTLFLGGGGSGGGGSGGGGSIQRTPKIQVYSRHPAENGKSNFLNCYVS GFHPSDIEVDLLKNGERIEKVEHSDLSFSKDWSFYLLYYTEFTPTEKDEYACRVNHVTLSQPKIVKWDRDM GGGGSGGGGSGGGGSGGGGSGSHSLKYFHTSVSRPGRGEPRFISVGYVDDTQFVRFDNDAASPRMVPRAP WMEQEGSEYWDRETRSARDTAQIFRVNLRTLRGYYNQSEAGSHTLQWMHGCELGPDRRFLRGYEQFAYD GKDYLTLNEDLRSWTAVDTAAQISEQKSNDASEAEHQRAYLEDTCVEWLHKYLEKGKETLLHLEPPKTHV THHPISDHEATLRCWALGFYPAEITLTWQQDGEGHTQDTELVETRPAGDGTFQKWAAVVVPSGEEQRYTC HVQHEGLPEPVTLRWKPASQPTIPIVGIIAGLVLLGSVVSGAVVAAVIWRKKSSGGKGGSYYKAEWSDSAQ GSESHSL), (e.g., SEQ ID NO: 689). In several embodiments, the polynucleotide encodes a sequence that shares at least about 80%, at least about 85%, at least about 90%, at least about 94%, at least about 95%, at least about 96%, at least about 97%, at least about 98%, or at least about 99%, sequence identity, homology and/or functional equivalence with SEQ ID NO: 276 or SEQ ID NO: 689. In several embodiments, the polynucleotide comprises SEQ ID NO: 277 (or SEQ ID NO: 690) or shares at least about 80%, at least about 85%, at least about 90%, at least about 94%, at least about 95%, at least about 96%, at least about 97%, at least about 98%, or at least about 99%, sequence identity with SEQ ID NO: 277 or SEQ ID NO: 690. Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, the polynucleotide encoding the HLA-E trimer_SS construct and GFP comprises SEQ ID NO: 275 or shares at least about 80%, at least about 85%, at least about 90%, at least about 94%, at least about 95%, at least about 96%, at least about 97%, at least about 98%, or at least about 99%, sequence identity with SEQ ID NO: 275. In several embodiments, the polynucleotide encoding the HLA-E trimer_SS construct, a FLAG tag, and GFP comprises SEQ ID NO: 688 or shares at least about 80%, at least about 85%, at least about 90%, at least about 94%, at least about 95%, at least about 96%, at least about 97%, at least about 98%, or at least about 99%, sequence identity with SEQ ID NO: 688. In several embodiments, the HLA-E trimer_SS construct with a FLAG tag and GFP is encoded by a polynucleotide that encodes an amino acid sequence comprising SEQ ID NO: 687, or having at least about 80%, at least about 85%, at least about 90%, at least about 94%, at least about 95%, at least about 96%, at least about 97%, at least about 98%, or at least about 99%, sequence identity with SEQ ID NO: 687.

In several embodiments, similar constructs are provided for HLA-G wherein the HLA-G trimer_SS construct comprises a CD8α signal peptide, a first B2M sequence, an HLG peptide leader sequence, a linker (e.g., a GS linker), a second B2M sequence, a linker (e.g., a second GS linker), and an HLA-G sequence. In several embodiments, the HLA-G trimer_SS construct is encoded by a polynucleotide that encodes an amino acid sequence comprising SEQ ID NO: 1095 (SRSVALAVLALLSLSGLEAVMAPRTLFLGGGGSGGGGSGGGGSIQRTPKIQVYSRHPAENGKSNFLNCYVS GFHPSDIEVDLLKNGERIEKVEHSDLSFSKDWSFYLLYYTEFTPTEKDEYACRVNHVTLSQPKIVKWDRDM GGGGSGGGGSGGGGSGGGGSGSLLSGALTLTETWAGSHSMRYFSAAVSRPGRGEPRFIAMGYVDDTQFV RFDSDSACPRMEPRAPWVEQEGPEYWEEETRNTKAHAQTDRMNLQTLRGYYNQSEASSHTLQWMIGCDL GSDGRLLRGYEQYAYDGKDYLALNEDLRSWTAADTAAQISKRKCEAANVAEQRRAYLEGTCVEWLHRY LENGKEMLQRADPPKTHVTHHPVFDYEATLRCWALGFYPAEIILTWQRDGEDQTQDVELVETRPAGDGTF QKWAAVVVPSGEEQRYTCHVQHEGLPEPLMLRWKQSSLPTIPIMGIVAGLVVLAAVVTGAAVAAVLWRK KSSD), (e.g., SEQ ID NO: 279). In several embodiments, the polynucleotide encodes a sequence that shares at least about 80%, at least about 85%, at least about 90%, at least about 94%, at least about 95%, at least about 96%, at least about 97%, at least about 98%, or at least about 99%, sequence identity, homology and/or functional equivalence with SEQ ID NO: 279. In several embodiments, the HLA-G polypeptide comprises SEQ ID NO:278. In several embodiments, the HLA-G polypeptide comprises a functional portion of SEQ ID NO: 278.

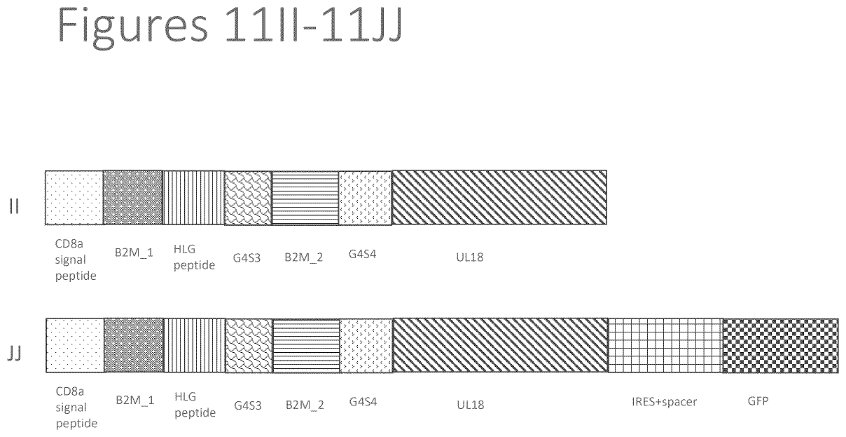

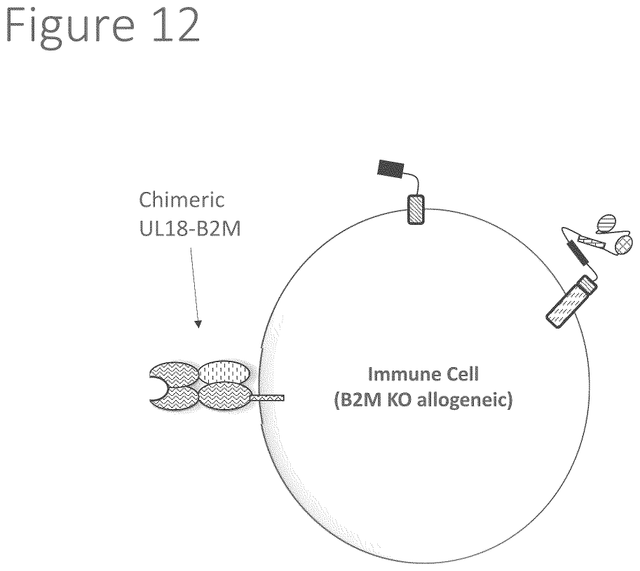

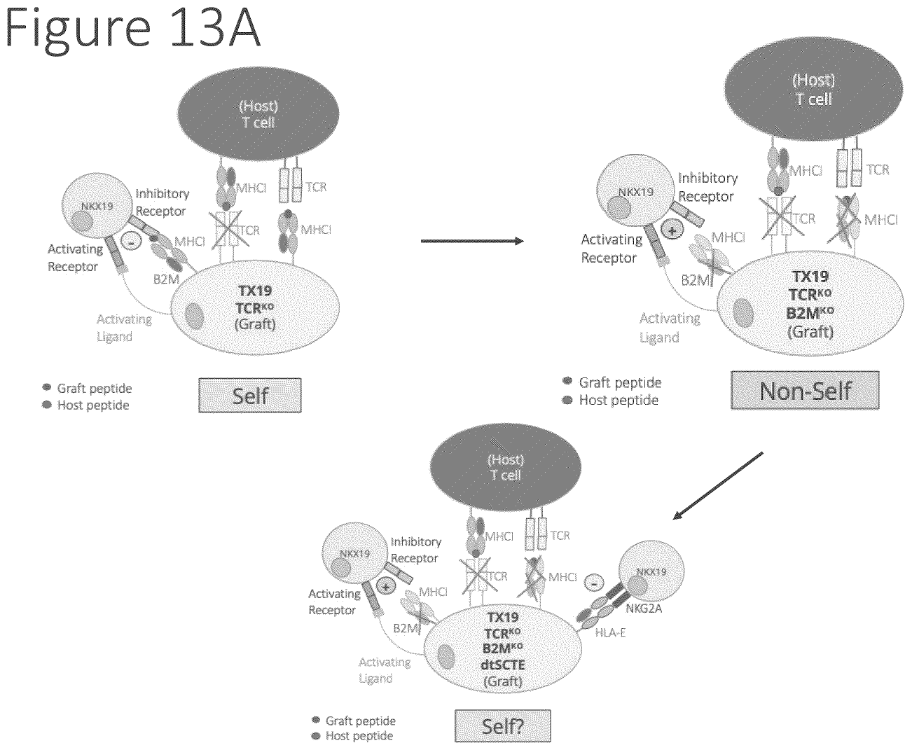

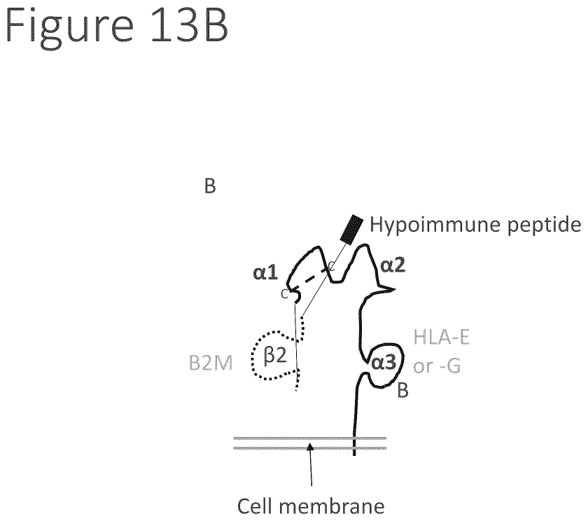

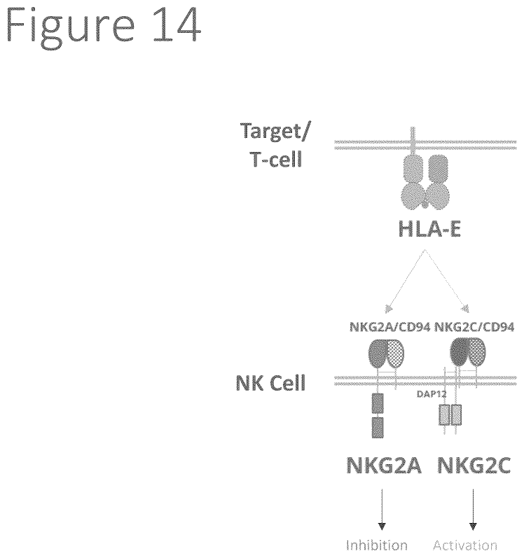

In the context of viral infection, MHC class I-restricted CD8+ cytotoxic T lymphocytes are recruited to control viral infections. These cytotoxic T lymphocytes recognize and lyse virus-infected cells through engagement of the lymphocyte T cell receptor with MHC class I molecules that present viral antigens on the surface of infected cells. MHC class I heavy chain associates with beta-2 microglobulin (B2M) to form a heterodimer, which constitutes part of the MHC class I peptide-loading complex. Human cytomegalovirus has evolved several gene products of the unique short region protein, US2, US3, US6, and US11, which interfere with antigen presentation and cell surface expression of MHC class I molecules. While interference in antigen presentation and MHC class I down-regulation on the cell surface allows infected cells to evade virus-specific cytotoxic T lymphocytes, the down-regulation of MHC class I molecules renders the virally infected cells more susceptible to host NK cells. To counteract this, human cytomegalovirus encodes multiple genes that function to evade NK-mediated cell lysis of infected cells, one of which is UL18. UL18 binds LIR-1, an NK cell inhibitory receptor. UL18 shares a high level of amino acid sequence identity with MHC class I and therefore UL18 can act as an MHCI surrogate and associate with B2M. Thus, in several embodiments, a chimeric UL18-B2M construct (see a non-limiting schematic at FIG. 12) is expressed on engineered cells as disclosed herein, enabling UL18 interaction with the LIR-1 receptor on NK cells (either host or administered) and reduce the cytotoxic activity of those NK cells against the engineered UL18-expressing immune cell.

In several embodiments, there is provided a polynucleotide encoding UL18. In several embodiments, the UL18-encoding polynucleotide encodes an amino acid sequence comprising SEQ ID NO: 1042 (HVLRYGYTGIFDDTSHMTLTVVGIFDGQHFFTYHVNSSDKASSRANGTISWMANVSAAYPTYLDGERAK GDLIFNQTEQNLLELEIALGYRSQSVLTWTHECNTTENGSFVAGYEGFGWDGETLMELKDNLTLWTGPNY EISWLKQNKTYIDGKIKNISEGDTTIQRNYLKGNCTQWSVIYSGFQTPVTHPVVKGGVRNQNDNRAEAFCT SYGFFPGEINITFIHYGNKAPDDSEPQCNPLLPTFDGTFHQGCYVAIFCNQNYTCRVTHGNWTVEIPISVTSP DDSSSGEVPDHPTANKRYNTMTISSVLLALLLCALLFAFLHYFTTLKQYLRNLAFAWRYRKVRSS) (e.g., SEQ ID NO: 280). In several embodiments, the polynucleotide encodes a sequence that shares at least about 80%, at least about 85%, at least about 90%, at least about 94%, at least about 95%, at least about 96%, at least about 97%, at least about 98%, or at least about 99%, sequence identity, homology and/or functional equivalence with SEQ ID NO: 280. In several embodiments, the polynucleotide comprises SEQ ID NO: 281 or shares at least about 80%, at least about 85%, at least about 90%, at least about 94%, at least about 95%, at least about 96%, at least about 97%, at least about 98%, or at least about 99%, sequence identity with SEQ ID NO: 281. In several embodiments, the encoded UL18 is a chimeric UL18-B2M single chain trimer (SCT) composed of a canonical HLA-E binding peptide, mature human beta2-microglobulin, and UL18. In several embodiments, the chimeric UL18-B2M construct comprises a CD8α signal peptide, a first B2M sequence, an HLG peptide leader sequence, a linker (e.g., a GS linker), a second B2M sequence, a linker (e.g., a second GS linker), an a UL18 sequence. In several embodiments, the chimeric UL18-B2M construct is encoded by a polynucleotide that encodes an amino acid sequence comprising SEQ ID NO: 1096 (SRSVALAVLALLSLSGLEAVMAPRTLFLGGGGSGGGGSGGGGSIQRTPKIQVYSRHPAENGKSNFLNCYVS GFHPSDIEVDLLKNGERIEKVEHSDLSFSKDWSFYLLYYTEFTPTEKDEYACRVNHVTLSQPKIVKWDRDM GGGGSGGGGSGGGGSGGGGSGSMHVLRYGYTGIFDDTSHMTLTVVGIFDGQHFFTYHVNSSDKASSRAN GTISWMANVSAAYPTYLDGERAKGDLIFNQTEQNLLELEIALGYRSQSVLTWTHECNTTENGSFVAGYEGF GWDGETLMELKDNLTLWTGPNYEISWLKQNKTYIDGKIKNISEGDTTIQRNYLKGNCTQWSVIYSGFQTPV THPVVKGGVRNQNDNRAEAFCTSYGFFPGEINITFIHYGNKAPDDSEPQCNPLLPTFDGTFHQGCYVAIFCN QNYTCRVTHGNWTVEIPISVTSPDDSSSGEVPDHPTANKRYNTMTISSVLLALLLCALLFAFLHYFTTLKQY LRNLAFAWRYRKVRSS), (e.g., SEQ ID NO: 283). In several embodiments, the polynucleotide encodes a sequence that shares at least about 80%, at least about 85%, at least about 90%, at least about 94%, at least about 95%, at least about 96%, at least about 97%, at least about 98%, or at least about 99%, sequence identity, homology and/or functional equivalence with SEQ ID NO: 283 or SEQ ID NO: 686. In several embodiments, the polynucleotide comprises SEQ ID NO: 284 (or SEQ ID NO: 685) or shares at least about 80%, at least about 85%, at least about 90%, at least about 94%, at least about 95%, at least about 96%, at least about 97%, at least about 98%, or at least about 99%, sequence identity with SEQ ID NO: 284 (or SEQ ID NO: 685). Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, the polynucleotide encoding the chimeric UL18-B2M construct and GFP comprises SEQ ID NO: 292 or shares at least about 80%, at least about 85%, at least about 90%, at least about 94%, at least about 95%, at least about 96%, at least about 97%, at least about 98%, or at least about 99%, sequence identity with SEQ ID NO: 292. In several embodiments, the polynucleotide encoding the chimeric UL18-B2M construct, a FLAG tag and GFP comprises SEQ ID NO: 684 or shares at least about 80%, at least about 85%, at least about 90%, at least about 94%, at least about 95%, at least about 96%, at least about 97%, at least about 98%, or at least about 99%, sequence identity with SEQ ID NO: 684. In additional embodiments, the polynucleotide encoding the chimeric UL18-B2M construct with a FLAG tag and GFP encodes the amino acid sequence of SEQ ID NO: 684 or a sequence that shares at least about 80%, at least about 85%, at least about 90%, at least about 94%, at least about 95%, at least about 96%, at least about 97%, at least about 98%, or at least about 99%, sequence identity, homology and/or functional equivalence with SEQ ID NO: 684. In additional embodiments, the polynucleotide encoding the chimeric UL18-B2M construct encodes the amino acid sequence of SEQ ID NO: 285 or a sequence that shares at least about 80%, at least about 85%, at least about 90%, at least about 94%, at least about 95%, at least about 96%, at least about 97%, at least about 98%, or at least about 99%, sequence identity, homology and/or functional equivalence with SEQ ID NO: 285. In several embodiments, the polynucleotide comprises SEQ ID NO: 281 or shares at least about 80%, at least about 85%, at least about 90%, at least about 94%, at least about 95%, at least about 96%, at least about 97%, at least about 98%, or at least about 99%, sequence identity with SEQ ID NO: 285.

In several embodiments, the engineered cells provided for herein comprise a chimeric receptor that targets NKG2D ligands, wherein the CAR comprises an amino acid of SEQ ID NO: 174 or 899, or comprises an amino acid sequence with at least about 80%, at least about 85%, at least about 90%, at least about 94%, at least about 95%, at least about 96%, at least about 97%, at least about 98%, or at least about 99%, sequence identity, homology and/or functional equivalence to SEQ ID NO: 174 or 899 and one or more copies of one or more of the following membrane-bound immunosuppressive constructs: SEQ ID NO: 273, 276, 279, 280, or 283.

In several embodiments, the engineered cells provided for herein comprise a CAR that targets CD19, wherein the CAR comprises an amino acid of SEQ ID NO: 178 or 901, or comprises an amino acid sequence with at least about 80%, at least about 85%, at least about 90%, at least about 94%, at least about 95%, at least about 96%, at least about 97%, at least about 98%, or at least about 99%, sequence identity, homology and/or functional equivalence to SEQ ID NO: 178 or 901 and one or more copies of one or more of the following membrane-bound immunosuppressive constructs: SEQ ID NO: 273, 276, 279, 280, or 283.

In several embodiments, the engineered cells provided for herein comprise a CAR that targets CD19, wherein the CAR is encoded by SEQ ID NO: 466, or comprises an amino acid sequence with at least about 80%, at least about 85%, at least about 90%, at least about 94%, at least about 95%, at least about 96%, at least about 97%, at least about 98%, or at least about 99%, sequence identity, homology and/or functional equivalence to the sequence encoded by SEQ ID NO: 466 and one or more copies of one or more of the following membrane-bound immunosuppressive constructs: SEQ ID NO: 273, 276, 279, 280, or 283.

In several embodiments, the engineered cells provided for herein comprise a chimeric receptor that targets NKG2D ligands, wherein the CAR comprises an amino acid of SEQ ID NO: 1024 or 1025, or comprises an amino acid sequence with at least about 80%, at least about 85%, at least about 90%, at least about 94%, at least about 95%, at least about 96%, at least about 97%, at least about 98%, or at least about 99%, sequence identity, homology and/or functional equivalence to SEQ ID NO: 1024 or 1025 and one or more copies of one or more of the following membrane-bound immunosuppressive constructs: SEQ ID NO: 273, 1094, 1095, 1042, or 1096.

In several embodiments, the engineered cells provided for herein comprise a CAR that targets CD19, wherein the CAR comprises an amino acid of SEQ ID NO: 1026 or 1027, or comprises an amino acid sequence with at least about 80%, at least about 85%, at least about 90%, at least about 94%, at least about 95%, at least about 96%, at least about 97%, at least about 98%, or at least about 99%, sequence identity, homology and/or functional equivalence to SEQ ID NO: 1026 or 1027 and one or more copies of one or more of the following membrane-bound immunosuppressive constructs: SEQ ID NO: 273, 1094, 1095, 1042, or 1096.

In several embodiments, the engineered cells provided for herein comprise a CAR that targets CD19, wherein the CAR is encoded by SEQ ID NO: 1097, or comprises an amino acid sequence with at least about 80%, at least about 85%, at least about 90%, at least about 94%, at least about 95%, at least about 96%, at least about 97%, at least about 98%, or at least about 99%, sequence identity, homology and/or functional equivalence to the sequence encoded by SEQ ID NO: 466 and one or more copies of one or more of the following membrane-bound immunosuppressive constructs: SEQ ID NO: 273, 1094, 1095, 1042, or 1096. In several embodiments, the engineered cells provided for herein comprise a CAR that targets CD19, wherein the CAR comprises a combination of one or more of SEQ ID NOs: 6, 8, 10, 12, 14, 16, 22, 118, 120, 895, 897, or 1009.

In several embodiments, the engineered cells provided for herein comprise a CAR that targets CD70, wherein the CAR comprises an amino acid of any of SEQ ID NOs: 383-465 or 912-994, or comprises an amino acid sequence with at least about 80%, at least about 85%, at least about 90%, at least about 94%, at least about 95%, at least about 96%, at least about 97%, at least about 98%, or at least about 99%, sequence identity, homology and/or functional equivalence to any of SEQ ID NO: 383-465 or 912-994 and one or more copies of one or more of the following membrane-bound immunosuppressive constructs: SEQ ID NO: 273, 276, 279, 280, or 283.

As discussed herein, in several embodiments, knockout of B2M expression results in loss of MHC expression on the edited cell. In several embodiments, the loss of MHC expression, such as on an edited T cell, renders that cell susceptible to attack from other cells, such as NK cells, that no longer recognize the edited cell as a “self” cell. However, in several embodiments, HLA can be re-expressed, for example HLA-E or HLA-G. In several embodiments, the re-expression of the HLA is accomplished using a disulfide trap single chain trimer (dtSCT) is used to express HLA-E and/or HLA-G and, optionally, an immunosuppressive peptide, as well as B2M (HLA-E_STE20, also referred to as HLA-E (PBL20). Provided for herein, in several embodiments, is a polynucleotide encoding a chimeric immunosuppressive construct comprising an HLA-G peptide, mature B2M and mature HLA-E. In several embodiments, such a construct comprises one or more linkers. In several embodiments, the immunosuppressive construct comprises a B2M signal peptide (at least about 85%, at least about 90%, at least about 94%, at least about 95%, at least about 96%, at least about 97%, at least about 98%, or at least about 99%, identical to SEQ ID NO:1018 (SRSVALAVLALLSLSGLEA), (e.g. SEQ ID NO: 1003) an HLA-G peptide (amino acids 3-11 of HLA-G; at least about 85%, at least about 90%, at least about 94%, at least about 95%, at least about 96%, at least about 97%, at least about 98%, or at least about 99%, identical to SEQ ID NO: 1005), a disulfide-bridge containing linker (e.g., a GS linker comprising at least two cysteine residues; at least about 85%, at least about 90%, at least about 94%, at least about 95%, at least about 96%, at least about 97%, at least about 98%, or at least about 99%, identical to SEQ ID NO: 1009), a mature B2M domain (at least about 85%, at least about 90%, at least about 94%, at least about 95%, at least about 96%, at least about 97%, at least about 98%, or at least about 99%, identical to SEQ ID NO: 1011), an additional linker (e.g., a GS linker), and a mature HLA-E domain (at least about 85%, at least about 90%, at least about 94%, at least about 95%, at least about 96%, at least about 97%, at least about 98%, or at least about 99%, identical to SEQ ID NO: 1013). In several embodiments, the HLA-E_STE20 is encoded by a polynucleotide that encodes an amino acid sequence comprising SEQ ID NO: 829. In several embodiments, the amino acid sequence shares at least about 90%, at least about 94%, at least about 95%, at least about 96%, at least about 97%, at least about 98%, or at least about 99%, sequence identity, homology and/or functional equivalence with SEQ ID NO: 829. In several embodiments, the B2M signal peptide is encoded by a nucleic acid that is at least about 85%, at least about 90%, at least about 94%, at least about 95%, at least about 96%, at least about 97%, at least about 98%, or at least about 99%, identical to SEQ ID NO: 1001. In several embodiments, the HLA-G peptide is encoded by a nucleic acid that is at least about 85%, at least about 90%, at least about 94%, at least about 95%, at least about 96%, at least about 97%, at least about 98%, or at least about 99%, identical to SEQ ID NO: 1004. In several embodiments, the disulfide-bridge containing linker is encoded by a nucleic acid that is at least about 85%, at least about 90%, at least about 94%, at least about 95%, at least about 96%, at least about 97%, at least about 98%, or at least about 99%, identical to SEQ ID NO: 1008. In several embodiments, the mature B2M is encoded by a nucleic acid that is at least about 85%, at least about 90%, at least about 94%, at least about 95%, at least about 96%, at least about 97%, at least about 98%, or at least about 99%, identical to SEQ ID NO: 1010. In several embodiments, an additional copy of the disulfide-bridge containing linker is use after the B2M and links the B2M with a mature HLA-E domain. In several embodiments, the mature HLA-e domain is encoded by a nucleic acid that is at least about 85%, at least about 90%, at least about 94%, at least about 95%, at least about 96%, at least about 97%, at least about 98%, or at least about 99%, identical to SEQ ID NO: 1012. In several embodiments, the polynucleotide encoding the immunosuppressive construct comprises SEQ ID NO: 830 or shares at least about 80%, at least about 85%, at least about 90%, at least about 94%, at least about 95%, at least about 96%, at least about 97%, at least about 98%, or at least about 99%, sequence identity with SEQ ID NO: 830. Optionally, the polynucleotide encodes a detectable tag (e.g., a FLAG tag) and/or can comprise an internal ribosome entry site (IRES) that allows for expression of an additional protein, such as a detectable tag (e.g., GFP). In several embodiments, a linker (e.g., a GS linker) is positioned between the HLA-E_STE20 and the tag. In several embodiments, the polynucleotide encoding HLA-E_STE20 and GFP comprises SEQ ID NO: 828 or shares at least about 80%, at least about 85%, at least about 90%, at least about 94%, at least about 95%, at least about 96%, at least about 97%, at least about 98%, or at least about 99%, sequence identity with SEQ ID NO: 828. In several embodiments, the HLA-E_STE20-GFP construct comprises the amino acid sequence of SEQ ID NO: 827 or an amino acid that shares at least about 80%, at least about 85%, at least about 90%, at least about 94%, at least about 95%, at least about 96%, at least about 97%, at least about 98%, or at least about 99%, sequence identity with SEQ ID NO: 827.

In additional embodiments, there is provided a chimeric immunosuppressive construct comprising an HLA-G peptide, mature B2M and mature HLA-E, and comprising an alternative B2M signal peptide (referred to as HLA-E (PBL20) (E1-5-sgRNA resistant)). In several embodiments, the nucleotide encoding the B2M signal peptide is different from, for example, that of the construct described above, but encodes the same amino acid sequence. In several embodiments, the alternative signal peptide confers enhanced resistance to degradation. In several embodiments, the B2M signal peptide is at least about 85%, at least about 90%, at least about 94%, at least about 95%, at least about 96%, at least about 97%, at least about 98%, or at least about 99%, identical to the amino acid sequence of SEQ ID NO: 1003, but is encoded by a polynucleotide at least about 85%, at least about 90%, at least about 94%, at least about 95%, at least about 96%, at least about 97%, at least about 98%, or at least about 99%, identical to SEQ ID NO: 1002. In several embodiments, the remainder of the construct remains the same as described above, e.g., an HLA-G peptide (amino acids 3-11 of HLA-G; at least about 85%, at least about 90%, at least about 94%, at least about 95%, at least about 96%, at least about 97%, at least about 98%, or at least about 99%, identical to SEQ ID NO: 1005), a disulfide-bridge containing linker (e.g., a GS linker comprising at least two cysteine residues; at least about 85%, at least about 90%, at least about 94%, at least about 95%, at least about 96%, at least about 97%, at least about 98%, or at least about 99%, identical to SEQ ID NO: 1009), a mature B2M domain (at least about 85%, at least about 90%, at least about 94%, at least about 95%, at least about 96%, at least about 97%, at least about 98%, or at least about 99%, identical to SEQ ID NO: 1011), an additional linker (e.g., another copy of the GS linker), and a mature HLA-E domain (at least about 85%, at least about 90%, at least about 94%, at least about 95%, at least about 96%, at least about 97%, at least about 98%, or at least about 99%, identical to SEQ ID NO: 1013). In several embodiments, the HLA-E (PBL20) (E1-5-sgRNA resistant) is encoded by a polynucleotide that encodes an amino acid sequence comprising SEQ ID NO: 997. In several embodiments, the amino acid sequence shares at least about 90%, at least about 94%, at least about 95%, at least about 96%, at least about 97%, at least about 98%, or at least about 99%, sequence identity, homology and/or functional equivalence with SEQ ID NO: 997. In several embodiments, the B2M signal peptide is encoded by a nucleic acid that is at least about 85%, at least about 90%, at least about 94%, at least about 95%, at least about 96%, at least about 97%, at least about 98%, or at least about 99%, identical to SEQ ID NO: 1002. In several embodiments, the remainder of the immunosuppressive construct is encoded by the polynucleotides as described above, e.g., the HLA-G peptide is encoded by a nucleic acid that is at least about 85%, at least about 90%, at least about 94%, at least about 95%, at least about 96%, at least about 97%, at least about 98%, or at least about 99%, identical to SEQ ID NO: 1004, the disulfide-bridge continaing linkers (one between the HLA-G and mature B2M domains, and another between the mature B2M and HLA-E domains) are encoded by a nucleic acid that is at least about 85%, at least about 90%, at least about 94%, at least about 95%, at least about 96%, at least about 97%, at least about 98%, or at least about 99%, identical to SEQ ID NO: 1008, the mature B2M is encoded by a nucleic acid that is at least about 85%, at least about 90%, at least about 94%, at least about 95%, at least about 96%, at least about 97%, at least about 98%, or at least about 99%, identical to SEQ ID NO: 1010, and a mature HLA-e domain is encoded by a nucleic acid that is at least about 85%, at least about 90%, at least about 94%, at least about 95%, at least about 96%, at least about 97%, at least about 98%, or at least about 99%, identical to SEQ ID NO: 1012. In several embodiments, the polynucleotide encoding the immunosuppressive construct comprises SEQ ID NO: 998 or shares at least about 80%, at least about 85%, at least about 90%, at least about 94%, at least about 95%, at least about 96%, at least about 97%, at least about 98%, or at least about 99%, sequence identity with SEQ ID NO: 998. Optionally, the polynucleotide encodes a detectable tag (e.g., a FLAG tag) and/or can comprise an internal ribosome entry site (IRES) that allows for expression of an additional protein, such as a detectable tag (e.g., GFP).

In additional embodiments, there is provided a chimeric immunosuppressive construct comprising an HLA-G peptide, mature B2M and mature HLA-E, and comprising an alternative linker structure (referred to as HLA-E (PBL15)). As above, in several embodiments, the B2M signal peptide is at least about 85%, at least about 90%, at least about 94%, at least about 95%, at least about 96%, at least about 97%, at least about 98%, or at least about 99%, identical to the amino acid sequence of SEQ ID NO: 1003. In several embodiments, the B2M signal peptide is at least about 85%, at least about 90%, at least about 94%, at least about 95%, at least about 96%, at least about 97%, at least about 98%, or at least about 99%, identical to the amino acid sequence of SEQ ID NO: 1018. In several embodiments, the disulfide-bridge containing linker between the HLA-G and mature B2M domains is at least about 85%, at least about 90%, at least about 94%, at least about 95%, at least about 96%, at least about 97%, at least about 98%, or at least about 99%, identical to the amino acid sequence of SEQ ID NO: 1007. In several embodiments, the remainder of the construct remains the same as described above, e.g., an HLA-G peptide (amino acids 3-11 of HLA-G; at least about 85%, at least about 90%, at least about 94%, at least about 95%, at least about 96%, at least about 97%, at least about 98%, or at least about 99%, identical to SEQ ID NO: 1005), a mature B2M domain (at least about 85%, at least about 90%, at least about 94%, at least about 95%, at least about 96%, at least about 97%, at least about 98%, or at least about 99%, identical to SEQ ID NO: 1011), a disulfide-bridge containing linker (e.g., a GS linker comprising at least two cysteine residues; at least about 85%, at least about 90%, at least about 94%, at least about 95%, at least about 96%, at least about 97%, at least about 98%, or at least about 99%, identical to SEQ ID NO: 1009), and a mature HLA-E domain (at least about 85%, at least about 90%, at least about 94%, at least about 95%, at least about 96%, at least about 97%, at least about 98%, or at least about 99%, identical to SEQ ID NO: 1013). In several embodiments, the HLA-E (PBL15) is encoded by a polynucleotide that encodes an amino acid sequence comprising SEQ ID NO: 1016, (SRSVALAVLALLSLSGLEAVMAPRTLFLGCGASGGGGSGGGGSIQRTPKIQVYSRHPAENGKSNFLNCYVS GFHPSDIEVDLLKNGERIEKVEHSDLSFSKDWSFYLLYYTEFTPTEKDEYACRVNHVTLSQPKIVKWDRDM GGGGSGGGGSGGGGSGGGGSGSHSLKYFHTSVSRPGRGEPRFISVGYVDDTQFVRFDNDAASPRMVPRAP WMEQEGSEYWDRETRSARDTAQIFRVNLRTLRGCYNQSEAGSHTLQWMHGCELGPDGRFLRGYEQFAYD GKDYLTLNEDLRSWTAVDTAAQISEQKSNDASEAEHQRAYLEDTCVEWLHKYLEKGKETLLHLEPPKTHV THHPISDHEATLRCWALGFYPAEITLTWQQDGEGHTQDTELVETRPAGDGTFQKWAAVVVPSGEEQRYTC HVQHEGLPEPVTLRWKPASQPTIPIVGIIAGLVLLGSVVSGAVVAAVIWRKKSSGGKGGSYSKAEWSDSAQ GSESHSL), (e.g. SEQ ID NO: 999). In several embodiments, the amino acid sequence shares at least about 90%, at least about 94%, at least about 95%, at least about 96%, at least about 97%, at least about 98%, or at least about 99%, sequence identity, homology and/or functional equivalence with SEQ ID NO: 999.

In several embodiments, the PBL15(G2C) linker between the HLA-G and mature B2M domains is encoded by a nucleic acid that is at least about 85%, at least about 90%, at least about 94%, at least about 95%, at least about 96%, at least about 97%, at least about 98%, or at least about 99%, identical to SEQ ID NO: 1006. In several embodiments, the remainder of the immunosuppressive construct is encoded by the polynucleotides as described above, e.g., the HLA-G peptide is encoded by a nucleic acid that is at least about 85%, at least about 90%, at least about 94%, at least about 95%, at least about 96%, at least about 97%, at least about 98%, or at least about 99%, identical to SEQ ID NO: 1004, the mature B2M is encoded by a nucleic acid that is at least about 85%, at least about 90%, at least about 94%, at least about 95%, at least about 96%, at least about 97%, at least about 98%, or at least about 99%, identical to SEQ ID NO: 1010, the disulfide-bridge continaing linker (between the mature B2M and HLA-E domains) is encoded by a nucleic acid that is at least about 85%, at least about 90%, at least about 94%, at least about 95%, at least about 96%, at least about 97%, at least about 98%, or at least about 99%, identical to SEQ ID NO: 1008, and a mature HLA-e domain is encoded by a nucleic acid that is at least about 85%, at least about 90%, at least about 94%, at least about 95%, at least about 96%, at least about 97%, at least about 98%, or at least about 99%, identical to SEQ ID NO: 1012. In several embodiments, the polynucleotide encoding the HLA-E (PBL15) immunosuppressive construct comprises SEQ ID NO: 1000 or shares at least about 80%, at least about 85%, at least about 90%, at least about 94%, at least about 95%, at least about 96%, at least about 97%, at least about 98%, or at least about 99%, sequence identity with SEQ ID NO: 1000. Optionally, the polynucleotide encodes a detectable tag (e.g., a FLAG tag) and/or can comprise an internal ribosome entry site (IRES) that allows for expression of an additional protein, such as a detectable tag (e.g., GFP).

In several embodiments, immunosuppressive constructs wherein HLA is re-expressed allow for inhibition of, for example, NK cell-based elimination of B2M deficient T-cells in a mixed NK+T cell population. See, for example, International Patent Application No. PCT/US2021/072715, filed Dec. 2, 2021, the entirety of which is incorporated by reference herein.

### Chimeric Receptors

### Extracellular Domains (Tumor Binder)

Some embodiments of the compositions and methods described herein relate to a chimeric antigen receptor that includes an extracellular domain that comprises a tumor-binding domain (also referred to as an antigen-binding protein or antigen-binding domain) as described herein. The tumor binding domain, depending on the embodiment, targets, for example CD19, CD123, CD70, Her2, mesothelin, Claudin 6, BCMA, EGFR, among others. Several embodiments of the compositions and methods described herein relate to a chimeric receptor that includes an extracellular domain that comprises a ligand binding domain that binds a ligand expressed by a tumor cell (also referred to as an activating chimeric receptor) as described herein. In some embodiments, the ligand binding domain binds to a ligand of NKG2D. The ligand binding domain, depending on the embodiment, targets for example MICA, MICB, ULBP1, ULBP2, ULBP3, ULBP4, ULBP5, and ULBP6 (among others).

In some embodiments, the antigen-binding domain is derived from or comprises wild-type or non-wild-type sequence of an antibody, an antibody fragment, an scFv, a Fv, a Fab, a (Fab′)₂, a single domain antibody (sdAb), a vH or vL domain, a camelid VHH domain, or a non-immunoglobulin scaffold such as a DARPIN, an affibody, an affilin, an adnectin, an affitin, a repebody, a fynomer, an alphabody, an avimer, an atrimer, a centyrin, a pronectin, an anticalin, a kunitz domain, an Armadillo repeat protein, an autoantigen, a receptor or a ligand. In some embodiments, the tumor-binding domain contains more than one antigen binding domain. In embodiments, the antigen-binding domain is operably linked directly or via an optional linker to the NH2-terminal end of a TCR domain (e.g., constant chains of TCR-alpha, TCR-beta1, TCR-beta2, preTCR-alpha, pre-TCR-alpha-Del48, TCR-gamma, or TCR-delta).

a. Antigen-Binding Proteins

There are provided, in several embodiments, antigen-binding proteins. As used herein, the term “antigen-binding protein” shall be given its ordinary meaning, and shall also refer to a protein comprising an antigen-binding fragment that binds to an antigen and, optionally, a scaffold or framework portion that allows the antigen-binding fragment to adopt a conformation that promotes binding of the antigen-binding protein to the antigen. In some embodiments, the antigen is a cancer antigen (e.g., CD19) or a fragment thereof. In some embodiments, the antigen-binding fragment comprises at least one CDR from an antibody that binds to the antigen. In some embodiments, the antigen-binding fragment comprises all three CDRs from the heavy chain of an antibody that binds to the antigen or from the light chain of an antibody that binds to the antigen. In still some embodiments, the antigen-binding fragment comprises all six CDRs from an antibody that binds to the antigen (three from the heavy chain and three from the light chain). In several embodiments, the antigen-binding fragment comprises one, two, three, four, five, or six CDRs from an antibody that binds to the antigen, and in several embodiments, the CDRs can be any combination of heavy and/or light chain CDRs. The antigen-binding fragment in some embodiments is an antibody fragment.

Nonlimiting examples of antigen-binding proteins include antibodies, antibody fragments (e.g., an antigen-binding fragment of an antibody), antibody derivatives, and antibody analogs. Further specific examples include, but are not limited to, a single-chain variable fragment (scFv), a nanobody (e.g., VH domain of camelid heavy chain antibodies; VHH fragment), a Fab fragment, a Fab′ fragment, a F(ab′)₂ fragment, a Fv fragment, a Fd fragment, and a complementarity determining region (CDR) fragment. These molecules can be derived from any mammalian source, such as human, mouse, rat, rabbit, or pig, dog, or camelid. Antibody fragments may compete for binding of a target antigen with an intact (e.g., native) antibody and the fragments may be produced by the modification of intact antibodies (e.g., enzymatic or chemical cleavage) or synthesized de novo using recombinant DNA technologies or peptide synthesis. The antigen-binding protein can comprise, for example, an alternative protein scaffold or artificial scaffold with grafted CDRs or CDR derivatives. Such scaffolds include, but are not limited to, antibody-derived scaffolds comprising mutations introduced to, for example, stabilize the three-dimensional structure of the antigen-binding protein as well as wholly synthetic scaffolds comprising, for example, a biocompatible polymer. In addition, peptide antibody mimetics (“PAMs”) can be used, as well as scaffolds based on antibody mimetics utilizing fibronectin components as a scaffold.

In some embodiments, the antigen-binding protein comprises one or more antibody fragments incorporated into a single polypeptide chain or into multiple polypeptide chains. For instance, antigen-binding proteins can include, but are not limited to, a diabody; an intrabody; a domain antibody (single VL or VH domain or two or more VH domains joined by a peptide linker;); a maxibody (2 scFvs fused to Fc region); a triabody; a tetrabody; a minibody (scFv fused to CH3 domain); a peptibody (one or more peptides attached to an Fc region); a linear antibody (a pair of tandem Fd segments (VH-CH1-VH-CH1) which, together with complementary light chain polypeptides, form a pair of antigen binding regions); a small modular immunopharmaceutical; and immunoglobulin fusion proteins (e.g. IgG-scFv, IgG-Fab, 2scFv-IgG, 4scFv-IgG, VH-IgG, IgG-VH, and Fab-scFv-Fc).

In some embodiments, the antigen-binding protein has the structure of an immunoglobulin. As used herein, the term “immunoglobulin” shall be given its ordinary meaning, and shall also refer to a tetrameric molecule, with each tetramer comprising two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function.

Within light and heavy chains, the variable (V) and constant regions (C) are joined by a “J” region of about 12 or more amino acids, with the heavy chain also including a “D” region of about 10 more amino acids. The variable regions of each light/heavy chain pair form the antibody binding site such that an intact immunoglobulin has two binding sites.

Immunoglobulin chains exhibit the same general structure of relatively conserved framework regions (FR) joined by three hypervariable regions, also called complementarity determining regions or CDRs. From N-terminus to C-terminus, both light and heavy chains comprise the domains FR1, CDR1, FR2, CDR2, FR3, CDR3 and FR4.

Human light chains are classified as kappa and lambda light chains. An antibody “light chain”, refers to the smaller of the two types of polypeptide chains present in antibody molecules in their naturally occurring conformations. Kappa (K) and lambda (k) light chains refer to the two major antibody light chain isotypes. A light chain may include a polypeptide comprising, from amino terminus to carboxyl terminus, a single immunoglobulin light chain variable region (VL) and a single immunoglobulin light chain constant domain (CL).

Heavy chains are classified as mu (μ), delta (Δ), gamma (γ), alpha (α), and epsilon (ε), and define the antibody's isotype as IgM, IgD, IgG, IgA, and IgE, respectively. An antibody “heavy chain” refers to the larger of the two types of polypeptide chains present in antibody molecules in their naturally occurring conformations, and which normally determines the class to which the antibody belongs. A heavy chain may include a polypeptide comprising, from amino terminus to carboxyl terminus, a single immunoglobulin heavy chain variable region (VH), an immunoglobulin heavy chain constant domain 1 (CH1), an immunoglobulin hinge region, an immunoglobulin heavy chain constant domain 2 (CH2), an immunoglobulin heavy chain constant domain 3 (CH3), and optionally an immunoglobulin heavy chain constant domain 4 (CH4). In some embodiments, the CAR/scFv comprises a VH domain comprising the CDR-H1, CDR-H2, and CDR-H3 of any VH domain sequence provided herein, and a VL domain comprising the CDR-L1, CRD-L2, and CDR-L3 of any VL domain sequence provided herein.

The IgG-class is further divided into subclasses, namely, IgG1, IgG2, IgG3, and IgG4. The IgA-class is further divided into subclasses, namely IgA1 and IgA2. The IgM has subclasses including, but not limited to, IgM1 and IgM2. The heavy chains in IgG, IgA, and IgD antibodies have three domains (CH1, CH2, and CH3), whereas the heavy chains in IgM and IgE antibodies have four domains (CH1, CH2, CH3, and CH4). The immunoglobulin heavy chain constant domains can be from any immunoglobulin isotype, including subtypes. The antibody chains are linked together via inter-polypeptide disulfide bonds between the CL domain and the CH1 domain (e.g., between the light and heavy chain) and between the hinge regions of the antibody heavy chains.

In some embodiments, the antigen-binding protein is an antibody. The term “antibody”, as used herein, refers to a protein, or polypeptide sequence derived from an immunoglobulin molecule which specifically binds with an antigen. Antibodies can be monoclonal, or polyclonal, multiple or single chain, or intact immunoglobulins, and may be derived from natural sources or from recombinant sources. Antibodies can be tetramers of immunoglobulin molecules. The antibody may be “humanized”, “chimeric” or non-human. An antibody may include an intact immunoglobulin of any isotype, and includes, for instance, chimeric, humanized, human, and bispecific antibodies. An intact antibody will generally comprise at least two full-length heavy chains and two full-length light chains. Antibody sequences can be derived solely from a single species, or can be “chimeric,” that is, different portions of the antibody can be derived from two different species as described further below. Unless otherwise indicated, the term “antibody” also includes antibodies comprising two substantially full-length heavy chains and two substantially full-length light chains provided the antibodies retain the same or similar binding and/or function as the antibody comprised of two full length light and heavy chains. For example, antibodies having 1, 2, 3, 4, or 5 amino acid residue substitutions, insertions or deletions at the N-terminus and/or C-terminus of the heavy and/or light chains are included in the definition provided that the antibodies retain the same or similar binding and/or function as the antibodies comprising two full length heavy chains and two full length light chains. Examples of antibodies include monoclonal antibodies, polyclonal antibodies, chimeric antibodies, humanized antibodies, human antibodies, bispecific antibodies, and synthetic antibodies. There is provided, in some embodiments, monoclonal and polyclonal antibodies. As used herein, the term “polyclonal antibody” shall be given its ordinary meaning, and shall also refer to a population of antibodies that are typically widely varied in composition and binding specificity. As used herein, the term “monoclonal antibody” (“mAb”) shall be given its ordinary meaning, and shall also refer to one or more of a population of antibodies having identical sequences. Monoclonal antibodies bind to the antigen at a particular epitope on the antigen.

In some embodiments, the antigen-binding protein is a fragment or antigen-binding fragment of an antibody. The term “antibody fragment” refers to at least one portion of an antibody, that retains the ability to specifically interact with (e.g., by binding, steric hindrance, stabilizing/destabilizing, spatial distribution) an epitope of an antigen. Examples of antibody fragments include, but are not limited to, Fab, Fab′, F(ab′)2, Fv fragments, scFv antibody fragments, disulfide-linked Fvs (sdFv), a Fd fragment consisting of the VH and CH1 domains, linear antibodies, single domain antibodies such as sdAb (either vL or vH), camelid vHH domains, multi-specific antibodies formed from antibody fragments such as a bivalent fragment comprising two Fab fragments linked by a disulfide bridge at the hinge region, and an isolated CDR or other epitope binding fragments of an antibody. An antigen binding fragment can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23: 1126-1136, 2005). Antigen binding fragments can also be grafted into scaffolds based on polypeptides such as a fibronectin type III (Fn3)(see U.S. Pat. No. 6,703,199, which describes fibronectin polypeptide mini bodies). An antibody fragment may include a Fab, Fab′, F(ab′)₂, and/or Fv fragment that contains at least one CDR of an immunoglobulin that is sufficient to confer specific antigen binding to a cancer antigen (e.g., CD19). Antibody fragments may be produced by recombinant DNA techniques or by enzymatic or chemical cleavage of intact antibodies.

In some embodiments, Fab fragments are provided. A Fab fragment is a monovalent fragment having the VL, VH, CL and CH1 domains; a F(ab′)2 fragment is a bivalent fragment having two Fab fragments linked by a disulfide bridge at the hinge region; a Fd fragment has the VH and CH1 domains; an Fv fragment has the VL and VH domains of a single arm of an antibody; and a dAb fragment has a VH domain, a VL domain, or an antigen-binding fragment of a VH or VL domain. In some embodiments, these antibody fragments can be incorporated into single domain antibodies, single-chain antibodies, maxibodies, minibodies, intrabodies, diabodies, triabodies, tetrabodies, v-NAR and bis-scFv. In some embodiments, the antibodies comprise at least one CDR as described herein.

There is also provided for herein, in several embodiments, single-chain variable fragments. As used herein, the term “single-chain variable fragment” (“scFv”) shall be given its ordinary meaning, and shall also refer to a fusion protein in which a VL and a VH region are joined via a linker (e.g., a synthetic sequence of amino acid residues) to form a continuous protein chain wherein the linker is long enough to allow the protein chain to fold back on itself and form a monovalent antigen binding site). For the sake of clarity, unless otherwise indicated as such, a “single-chain variable fragment” is not an antibody or an antibody fragment as defined herein. Diabodies are bivalent antibodies comprising two polypeptide chains, wherein each polypeptide chain comprises VH and VL domains joined by a linker that is configured to reduce or not allow for pairing between two domains on the same chain, thus allowing each domain to pair with a complementary domain on another polypeptide chain. According to several embodiments, if the two polypeptide chains of a diabody are identical, then a diabody resulting from their pairing will have two identical antigen binding sites. Polypeptide chains having different sequences can be used to make a diabody with two different antigen binding sites. Similarly, tribodies and tetrabodies are antibodies comprising three and four polypeptide chains, respectively, and forming three and four antigen binding sites, respectively, which can be the same or different.

In several embodiments, the antigen-binding protein comprises one or more CDRs. As used herein, the term “CDR” shall be given its ordinary meaning, and shall also refer to the complementarity determining region (also termed “minimal recognition units” or “hypervariable region”) within antibody variable sequences. The CDRs permit the antigen-binding protein to specifically bind to a particular antigen of interest. There are three heavy chain variable region CDRs (CDRH1, CDRH2 and CDRH3) and three light chain variable region CDRs (CDRL1, CDRL2 and CDRL3). The CDRs in each of the two chains typically are aligned by the framework regions to form a structure that binds specifically to a specific epitope or domain on the target protein. From N-terminus to C-terminus, naturally-occurring light and heavy chain variable regions both typically conform to the following order of these elements: FR1, CDR1, FR2, CDR2, FR3, CDR3 and FR4. A numbering system has been devised for assigning numbers to amino acids that occupy positions in each of these domains. This numbering system is defined in Kabat Sequences of Proteins of Immunological Interest (1987 and 1991, NIH, Bethesda, MD), or Chothia & Lesk, 1987, J. Mol. Biol. 196:901-917; Chothia et al., 1989, Nature 342: 878-883. Complementarity determining regions (CDRs) and framework regions (FR) of a given antibody may be identified using this system. Other numbering systems for the amino acids in immunoglobulin chains include IMGT® (the international ImMunoGeneTics information system; Lefranc et al, Dev. Comp. Immunol. 29: 185-203; 2005) and AHo (Honegger and Pluckthun, J. Mol. Biol. 309(3): 657-670; 2001). One or more CDRs may be incorporated into a molecule either covalently or noncovalently to make it an antigen-binding protein.

In some embodiments, the antigen-binding proteins provided herein comprise one or more CDR(s) as part of a larger polypeptide chain. In some embodiments, the antigen-binding proteins covalently link the one or more CDR(s) to another polypeptide chain. In some embodiments, the antigen-binding proteins incorporate the one or more CDR(s) noncovalently. In some embodiments, the antigen-binding proteins may comprise at least one of the CDRs described herein incorporated into a biocompatible framework structure. In some embodiments, the biocompatible framework structure comprises a polypeptide or portion thereof that is sufficient to form a conformationally stable structural support, or framework, or scaffold, which is able to display one or more sequences of amino acids that bind to an antigen (e.g., CDRs, a variable region, etc.) in a localized surface region. Such structures can be a naturally occurring polypeptide or polypeptide “fold” (a structural motif), or can have one or more modifications, such as additions, deletions and/or substitutions of amino acids, relative to a naturally occurring polypeptide or fold. Depending on the embodiment, the scaffolds can be derived from a polypeptide of a variety of different species (or of more than one species), such as a human, a non-human primate or other mammal, other vertebrate, invertebrate, plant, bacteria or virus.

Depending on the embodiment, the biocompatible framework structures are based on protein scaffolds or skeletons other than immunoglobulin domains. In some such embodiments, those framework structures are based on fibronectin, ankyrin, lipocalin, neocarzinostain, cytochrome b, CP1 zinc finger, PST1, coiled coil, LACI-D1, Z domain and/or tendamistat domains.

There is also provided, in some embodiments, antigen-binding proteins with more than one binding site. In several embodiments, the binding sites are identical to one another while in some embodiments the binding sites are different from one another. For example, an antibody typically has two identical binding sites, while a “bispecific” or “bifunctional” antibody has two different binding sites. The two binding sites of a bispecific antigen-binding protein or antibody will bind to two different epitopes, which can reside on the same or different protein targets. In several embodiments, this is particularly advantageous, as a bispecific chimeric antigen receptor can impart to an engineered cell the ability to target multiple tumor markers. For example, CD19 and an additional tumor marker, such as CD123, CD70, Her2, mesothelin, Claudin 6, BCMA, EGFR, MICA, MICB, ULBP1, ULBP2, ULBP3, ULBP4, ULBP5, and ULBP6, among others, or any other marker disclosed herein or appreciated in the art as a tumor specific antigen or tumor associated antigen can be bound by a bispecific antibody.

As used herein, the term “chimeric antibody” shall be given its ordinary meaning, and shall also refer to an antibody that contains one or more regions from one antibody and one or more regions from one or more other antibodies. In some embodiments, one or more of the CDRs are derived from an anti-cancer antigen (e.g., CD19, CD123, CD70, Her2, mesothelin, PD-L1, Claudin 6, BCMA, EGFR, etc.) antibody. In several embodiments, all of the CDRs are derived from an anti-cancer antigen antibody (such as an anti-CD19 antibody). In some embodiments, the CDRs from more than one anti-cancer antigen antibodies are mixed and matched in a chimeric antibody. For instance, a chimeric antibody may comprise a CDR1 from the light chain of a first anti-cancer antigen antibody, a CDR2 and a CDR3 from the light chain of a second anti-cancer antigen antibody, and the CDRs from the heavy chain from a third anti-cancer antigen antibody. Further, the framework regions of antigen-binding proteins disclosed herein may be derived from one of the same anti-cancer antigen (e.g., CD19, CD123, CD70, Her2, mesothelin, Claudin 6, BCMA, EGFR, etc.) antibodies, from one or more different antibodies, such as a human antibody, or from a humanized antibody. In one example of a chimeric antibody, a portion of the heavy and/or light chain is identical with, homologous to, or derived from an antibody from a particular species or belonging to a particular antibody class or subclass, while the remainder of the chain(s) is/are identical with, homologous to, or derived from an antibody or antibodies from another species or belonging to another antibody class or subclass. In some embodiments, an antigen-binding domain comprises a heavy chain variable region (VH) and a light chain variable region (VL), wherein the VH comprises the CDR-H1, the CDR-H2, and the CDR-H3 as comprised within any of the VH regions provided herein, and the VL comprises the CDR-L1, the CDR-L2, and the CDR-L3 comprised within any of the VL regions provided herein.

Also provided herein are fragments of such antibodies that exhibit the desired biological activity.

### CD38

In several embodiments, an antigen binding protein is directed against CD38 (also known as ADP-ribosyl cyclase 1, cADPr hydrolase 1, Cyclic ADP-ribose hydrolase 1, or T10).

According to one embodiment, the CD38 antigen binding protein is an antigen binding domain of a CAR which comprises a transmembrane domain, a signaling domain, and optionally a co-stimulatory domain as disclosed herein. In several embodiments, the antigen binding protein binds to an epitope of the human CD38, and in particular to an epitope of the extracellular domain of the human CD38.

In several embodiments, the CD38 binding protein comprises an scFv comprising a light chain variable region (vL domain) and a heavy chain variable region (vH domain). In several embodiments, the vH domain comprises a complementarity-determining region 1 (CDR-H1), a CDR-H2, and a CDR-H3. In several embodiments, the vL domain comprises a complementarity-determining region 1 (CDR-L1), a CDR-L2, and a CDR-L3. In several embodiments, the anti-CD38 binding protein comprises at least one CDR from SEQ ID NO: 526-531 or a CDR having at least about 95%, at least about 96%, at least about 97%, at least about 98%, or at least about 99%, sequence identity, homology and/or functional equivalence to any of SEQ ID NO: 526-531. In several embodiments, the anti-CD38 vH domain comprises the sequence of SEQ ID NO: 524, or an amino acid sequence with at least about 80%, at least about 85%, at least about 90%, at least about 94%, at least about 95%, at least about 96%, at least about 97%, at least about 98%, or at least about 99%, sequence identity, homology and/or functional equivalence to SEQ ID NO: 524. In several embodiments, the anti-CD38 vH domain comprises the sequence of SEQ ID NO: 524. In some embodiments, the vH comprises a CDR-H1, a CDR-H2, and a CDR-H3 as comprised in the sequence set forth in SEQ ID NO:524. In several embodiments, the anti-CD38 vL domain comprises the sequence of SEQ ID NO: 523, or an amino acid sequence with at least about 80%, at least about 85%, at least about 90%, at least about 94%, at least about 95%, at least about 96%, at least about 97%, at least about 98%, or at least about 99%, sequence identity, homology and/or functional equivalence to SEQ ID NO: 523. In several embodiments, the anti-CD38 VL domain comprises the sequence of SEQ ID NO: 523. In some embodiments, the vL comprises a CDR-L1, a CDR-L2, and a CDR-L3 as comprised in the sequence set forth in SEQ ID NO:523. In several embodiments, the anti-CD38 binding protein is an scFv that comprises the sequence of SEQ ID NO: 532, or an amino acid sequence with at least about 80%, at least about 85%, at least about 90%, at least about 94%, at least about 95%, at least about 96%, at least about 97%, at least about 98%, or at least about 99%, sequence identity, homology and/or functional equivalence to SEQ ID NO: 532. In several embodiments, the scFv comprises the sequence of SEQ ID NO: 532. In several embodiments, the anti-CD38 CAR comprises the sequence of SEQ ID NO: 525, or an amino acid sequence with at least about 80%, at least about 85%, at least about 90%, at least about 94%, at least about 95%, at least about 96%, at least about 97%, at least about 98%, or at least about 99%, sequence identity, homology and/or functional equivalence to SEQ ID NO: 525. In several embodiments, the CAR comprises the sequence of SEQ ID NO: 532. In several embodiments, the antigen binding protein is affinity matured to enhance binding to CD38. In several embodiments, the nucleotide sequence encoding the antigen binding protein is codon-optimized to enhance expression and/or stability of the protein.

### GPRC5D

In several embodiments, an antigen binding protein is directed against GPRC5D. According to one embodiment, the GPRC5D antigen binding protein is an antigen binding domain of a CAR which comprises a transmembrane domain, a signaling domain, and optionally a co-stimulatory domain as disclosed herein. In several embodiments, the antigen binding protein binds to an epitope of the human GPRC5D. In several embodiments, the GPRC5D antigen binding domain is an scFv comprising the amino acid sequence of any one of SEQ ID NOs: 621-630, or an amino acid sequence with at least about 80%, at least about 85%, at least about 90%, at least about 94%, at least about 95%, at least about 96%, at least about 97%, at least about 98%, or at least about 99%, sequence identity, homology and/or functional equivalence to any of SEQ ID NOs:621-630. In several embodiments, the GPRCSD antigen binding domain is an scFv comprising the amino acid sequence of any one of SEQ ID NOs: 621-630. In several embodiments, the GPRCSD antigen binding domain comprises the amino acid sequence of SEQ ID NO:621. In several embodiments, the GPRCSD antigen binding domain comprises the amino acid sequence of SEQ ID NO:622. In several embodiments, the GPRCSD antigen binding domain comprises the amino acid sequence of SEQ ID NO:623. In several embodiments, the GPRCSD antigen binding domain comprises the amino acid sequence of SEQ ID NO:624. In several embodiments, the GPRCSD antigen binding domain comprises the amino acid sequence of SEQ ID NO:625. In several embodiments, the GPRCSD antigen binding domain comprises the amino acid sequence of SEQ ID NO:626. In several embodiments, the GPRCSD antigen binding domain comprises the amino acid sequence of SEQ ID NO:627. In several embodiments, the GPRCSD antigen binding domain comprises the amino acid sequence of SEQ ID NO:628. In several embodiments, the GPRCSD antigen binding domain comprises the amino acid sequence of SEQ ID NO:629. In several embodiments, the GPRCSD antigen binding domain comprises the amino acid sequence of SEQ ID NO:630. In several embodiments, the GPRCSD antigen binding domain comprises the amino acid sequence of SEQ ID NO:631. In several embodiments, the antigen binding protein is affinity matured to enhance binding to GPRCSD. In several embodiments, the nucleotide sequence encoding the antigen binding protein is codon-optimized to enhance expression and/or stability of the protein.

### CD138

In several embodiments, an antigen binding protein is directed against CD138. In several embodiments, the anti-CD138 binding protein comprises a vL and/or vH chain. In several embodiments, the anti-CD138 binding protein comprises a vL and a vH. In several embodiments, the vH domain comprises a complementarity-determining region 1 (CDR-H1), a CDR-H2, and a CDR-H3. In several embodiments, the vL domain comprises a complementarity-determining region 1 (CDR-L1), a CDR-L2, and a CDR-L3. In several embodiments, the anti-CD138 binding protein comprises at least one CDR from SEQ ID NO: 536-541 or a CDR having at least about 95%, at least about 96%, at least about 97%, at least about 98%, or at least about 99%, sequence identity, homology and/or functional equivalence to any of SEQ ID NO: 536-541. In several embodiments, the vH chain comprises the amino acid sequence of SEQ ID NO: 534, or an amino acid sequence with at least about 80%, at least about 85%, at least about 90%, at least about 94%, at least about 95%, at least about 96%, at least about 97%, at least about 98%, or at least about 99%, sequence identity, homology and/or functional equivalence to SEQ ID NO: 534. In several embodiments, the vH comprises the amino acid sequence of SEQ ID NO: 534. In some embodiments, the VH comprises a CDR-H1, a CDR-H2, and a CDR-H3 as comprised in SEQ ID NO:534. In several embodiments, the vL chain comprises the amino acid sequence of SEQ ID NO: 533, or an amino acid sequence with at least about 80%, at least about 85%, at least about 90%, at least about 94%, at least about 95%, at least about 96%, at least about 97%, at least about 98%, or at least about 99%, sequence identity, homology and/or functional equivalence to SEQ ID NO: 533. In several embodiments, the vL chain comprises the amino acid sequence of SEQ ID NO: 533. In some embodiments, the VL comprises a CDR-L1, a CDR-L2, and a CDR-L 3 as comprised in SEQ ID NO:533. In several embodiments, the anti-CD138 binding domain comprises a single-chain variable fragment (scFv). In several embodiments, the scFv comprises a linker between the vH and vL domains. In several embodiments, the anti-CD138 binding protein is an scFv comprising the amino acid sequence of SEQ ID NO: 542, or an amino acid sequence with at least about 80%, at least about 85%, at least about 90%, at least about 94%, at least about 95%, at least about 96%, at least about 97%, at least about 98%, or at least about 99%, sequence identity, homology and/or functional equivalence to SEQ ID NO: 542. In several embodiments, the scFv comprises the amino acid sequence of SEQ ID NO: 542. In several embodiments, the anti-CD138 binding protein is integrated into a CAR which comprises a transmembrane domain, a signaling domain, and optionally a co-stimulatory domain as disclosed herein comprising the amino acid sequence of SEQ ID NO: 535 or 543, or an amino acid sequence with at least about 80%, at least about 85%, at least about 90%, at least about 94%, at least about 95%, at least about 96%, at least about 97%, at least about 98%, or at least about 99%, sequence identity, homology and/or functional equivalence to SEQ ID NO: 535 or 543. In several embodiments, the CAR comprises the amino acid sequence of SEQ ID NO: 535. In several embodiments, the CAR comprises the amino acid sequence of SEQ ID NO:543. In several embodiments, the antigen binding protein is affinity matured to enhance binding to CD138. In several embodiments, the nucleotide sequence encoding the antigen binding protein is codon-optimized to enhance expression and/or stability of the protein.

### DLL3

In several embodiments, an antigen binding protein is directed against DLL3. In several embodiments, the anti-DLL3 binding protein is an antigen binding domain of a CAR which comprises a transmembrane domain, a signaling domain, and optionally a co-stimulatory domain as disclosed herein. In several embodiments, the anti-CD138 binding protein comprises a vL and a vH. In several embodiments, the vH domain comprises a complementarity-determining region 1 (CDR-H1), a CDR-H2, and a CDR-H3. In several embodiments, the vL domain comprises a complementarity-determining region 1 (CDR-L1), a CDR-L2, and a CDR-L3. In several embodiments, the anti-DLL3 antigen binding protein comprises a vH chain comprising the amino acid sequence of any of SEQ ID NOs: 570-581, or a sequence having at least about 95%, at least about 96%, at least about 97%, at least about 98%, or at least about 99%, sequence identity, homology and/or functional equivalence to any of SEQ ID NO: 570-581. In several embodiments, the anti-DLL3 antigen binding protein comprises a vH chain comprising the amino acid sequence of any of SEQ ID NOs: 570-581. In several embodiments, the anti-DLL3 antigen binding protein comprises a vL chain comprising the amino acid sequence of any of SEQ ID NOs: 582-593, or a sequence having at least about 95%, at least about 96%, at least about 97%, at least about 98%, or at least about 99%, sequence identity, homology and/or functional equivalence to any of SEQ ID NO: 582-593. In several embodiments, the anti-DLL3 antigen binding protein comprises a vL chain comprising the amino acid sequence of any of SEQ ID NOs: 582-593. In several embodiments, the anti-DLL3 binding protein comprises a polypeptide that targets DLL3 and comprises the amino acid sequence of any of SEQ ID NO: 594-595, or a sequence having at least about 95%, at least about 96%, at least about 97%, at least about 98%, or at least about 99%, sequence identity, homology and/or functional equivalence to any of SEQ ID NO: 594-595. In some embodiments, the anti-DLL3 binding protein comprises a polypeptide that targets DLL3 and comprises the amino acid sequence of SEQ ID NO:594. In some embodiments, the anti-DLL3 binding protein comprises a polypeptide that targets DLL3 and comprises the amino acid sequence of SEQ ID NO:595. In several embodiments, the anti-DLL3 binding protein comprises an scFv. In several embodiments, the anti-DLL3 binding protein comprises an scFv comprising the sequence of any of SEQ ID NO: 596-599, or a sequence having at least about 95%, at least about 96%, at least about 97%, at least about 98%, or at least about 99%, sequence identity, homology and/or functional equivalence to any of SEQ ID NO: 596-599. In several embodiments, the scFv comprises the sequence of any of SEQ ID NO: 596-599. In several embodiments, the scFv comprises the sequence of SEQ ID NO:596. In several embodiments, the scFv comprises the sequence of SEQ ID NO:597. In several embodiments, the scFv comprises the sequence of SEQ ID NO:598. In several embodiments, the scFv comprises the sequence of SEQ ID NO:599. In several embodiments, the antigen binding protein is affinity matured to enhance binding to DLL3. In several embodiments, the nucleotide sequence encoding the antigen binding protein is codon-optimized to enhance expression and/or stability of the protein.

### EGFR

In several embodiments, an antigen binding protein is directed against the epidermal growth factor receptor EGFR. In several embodiments, the anti-EGFR binding protein is an antigen binding domain of a CAR which comprises a transmembrane domain, a signaling domain, and optionally a co-stimulatory domain as disclosed herein. In several embodiments, the anti-EGFR binding protein comprises a vH chain comprising the amino acid sequence of any of SEQ ID NO: 600, 606-607, or a sequence having at least about 95%, at least about 96%, at least about 97%, at least about 98%, or at least about 99%, sequence identity, homology and/or functional equivalence to any of SEQ ID NO: 600, 606-607. In several embodiments, the vH comprises the amino acid sequence SEQ ID NO:600, 606, or 607. In several embodiments, the vH comprises the amino acid sequence SEQ ID NO:600. In several embodiments, the vH comprises the amino acid sequence SEQ ID NO:606. In several embodiments, the vH comprises the amino acid sequence SEQ ID NO: 607. In several embodiments, the anti-EGFR binding protein comprises a vL chain comprising the amino acid sequence of any of SEQ ID NO: 601, 608-609, or a sequence having at least about 95%, at least about 96%, at least about 97%, at least about 98%, or at least about 99%, sequence identity, homology and/or functional equivalence to any of SEQ ID NO: 601, 608-609. In several embodiments, the vL comprises the amino acid sequence of SEQ ID NO: 601, 608, or 609. In several embodiments, the vL comprises the amino acid sequence of SEQ ID NO: 601. In several embodiments, the vL comprises the amino acid sequence of SEQ ID NO: 608. In several embodiments, the vL comprises the amino acid sequence of SEQ ID NO: 609. In several embodiments, the anti-EGFR binding protein is an scFv comprising the amino acid sequence of any of SEQ ID NOs: 610-620, or a sequence having at least about 95%, at least about 96%, at least about 97%, at least about 98%, or at least about 99%, sequence identity, homology and/or functional equivalence to any of SEQ ID NO: 610-620. In several embodiments, the scFv comprises the amino acid sequence of any of SEQ ID NOs: 610-620. In several embodiments, the anti-EGFR binding protein is incorporated into a CAR having the sequence of any of SEQ ID NOs: 602-605, or a sequence having at least about 95%, at least about 96%, at least about 97%, at least about 98%, or at least about 99%, sequence identity, homology and/or functional equivalence to any of SEQ ID NO: 602-605. In some embodiments, the CAR comprises the sequence of any one of SEQ ID NO:602-605. In some embodiments, the CAR comprises the sequence of SEQ ID NO:602. In some embodiments, the CAR comprises the sequence of SEQ ID NO:603. In some embodiments, the CAR comprises the sequence of SEQ ID NO:604. In some embodiments, the CAR comprises the sequence of SEQ ID NO:605. In several embodiments, the antigen binding protein is affinity matured to enhance binding to the EGFR. In several embodiments, the nucleotide sequence encoding the antigen binding protein is codon-optimized to enhance expression and/or stability of the protein.

### PSMA

In several embodiments, an antigen binding protein is directed against PSMA. In several embodiments, the anti-PSMA binding protein is an antigen binding domain of a CAR which comprises a transmembrane domain, a signaling domain, and optionally a co-stimulatory domain as disclosed herein. In several embodiments, the anti-PSMA binding protein comprises a vL chain comprising the amino acid sequence of SEQ ID NO: 634, or a sequence having at least about 95%, at least about 96%, at least about 97%, at least about 98%, or at least about 99%, sequence identity, homology and/or functional equivalence to SEQ ID NO: 634. In some embodiments, the vL comprises the amino acid sequence of SEQ ID NO: 634. In several embodiments, the anti-PSMA binding protein comprises a vH chain comprising the amino acid sequence of SEQ ID NO: 635, or a sequence having at least about 95%, at least about 96%, at least about 97%, at least about 98%, or at least about 99%, sequence identity, homology and/or functional equivalence to SEQ ID NO: 635. In some embodiments, the vH comprises the amino acid sequence of SEQ ID NO: 635. In several embodiments, the anti-PSMA binding protein comprises an scFv comprising the amino acid sequence of SEQ ID NO: 632 or 633, or a sequence having at least about 95%, at least about 96%, at least about 97%, at least about 98%, or at least about 99%, sequence identity, homology and/or functional equivalence to SEQ ID NO: 632 or 633. In some embodiments, the scFv comprises the amino acid sequence of SEQ ID NO:632. In some embodiments, the scFc comprises the amino acid sequence of SEQ ID NO:633. In several embodiments, the anti-PSMA binding protein comprises an antibody comprising the amino acid sequence of SEQ ID NO: 631, or a sequence having at least about 95%, at least about 96%, at least about 97%, at least about 98%, or at least about 99%, sequence identity, homology and/or functional equivalence to SEQ ID NO: 631. In some embodiments, the anti-PSMA binding protein comprises the amino acid sequence set forth in SEQ ID NO:631. In several embodiments, the antigen binding protein is affinity matured to enhance binding to PSMA. In several embodiments, the nucleotide sequence encoding the antigen binding protein is codon-optimized to enhance expression and/or stability of the protein.

### FLT3

In several embodiments, an antigen binding protein is directed against FLT3. In several embodiments, the anti-FLT3 binding protein is an antigen binding domain of a CAR which comprises a transmembrane domain, a signaling domain, and optionally a co-stimulatory domain as disclosed herein. In several embodiments, the anti-FLT3 binding protein comprises one or more CDRs from the vL and/or vH chain selected from SEQ ID NOs: 636-644, or a CDR having at least about 95%, at least about 96%, at least about 97%, at least about 98%, or at least about 99%, sequence identity, homology and/or functional equivalence to any of SEQ ID NO: 636-644. In several embodiments, the anti-FLT3 binding protein comprises a vL chain comprising the amino acid sequence of SEQ ID NO: 645, or a sequence having at least about 95%, at least about 96%, at least about 97%, at least about 98%, or at least about 99%, sequence identity, homology and/or functional equivalence to SEQ ID NO: 645. In some embodiments, the vL comprises the amino acid sequence set forth in SEQ ID NO:645. In several embodiments, the anti-FLT3 binding protein comprises a vH chain comprising the amino acid sequence of SEQ ID NO: 646, or a sequence having at least about 95%, at least about 96%, at least about 97%, at least about 98%, or at least about 99%, sequence identity, homology and/or functional equivalence to SEQ ID NO: 646. In some embodiments, the vH comprises the amino acid sequence set forth in SEQ ID NO:646. In some embodiments, the anti-KREMEN binding protein comprises a scFv. In several embodiments, the antigen binding protein is affinity matured to enhance binding to FLT3. In several embodiments, the nucleotide sequence encoding the antigen binding protein is codon-optimized to enhance expression and/or stability of the protein.

### KREMEN2

In several embodiments, an antigen binding protein is directed against KREMEN2. In several embodiments, the anti-KREMEN2 binding protein is an antigen binding domain of a CAR which comprises a transmembrane domain, a signaling domain, and optionally a co-stimulatory domain as disclosed herein. In several embodiments, the anti-KREMEN2 binding protein comprises a vL chain comprising the amino acid sequence of any of SEQ ID NOs: 647-651, or a sequence having at least about 95%, at least about 96%, at least about 97%, at least about 98%, or at least about 99%, sequence identity, homology and/or functional equivalence to any of SEQ ID NOs: 647-651. In several embodiments, the vL comprises the amino acid sequence of any of SEQ ID NOs: 647-651. In several embodiments, the anti-KREMEN2 binding protein comprises a vH chain comprising the amino acid sequence of any of SEQ ID NOs: 652-656, or a sequence having at least about 95%, at least about 96%, at least about 97%, at least about 98%, or at least about 99%, sequence identity, homology and/or functional equivalence to any of SEQ ID NOs: 652-656. In several embodiments, the vH comprises the amino acid sequence of any of SEQ ID NOs: 652-656. In some embodiments, the anti-KREMEN binding protein comprises a scFv. In several embodiments, the antigen binding protein is affinity matured to enhance binding to KREMEN2. In several embodiments, the nucleotide sequence encoding the antigen binding protein is codon-optimized to enhance expression and/or stability of the protein.

### ALPPL2

In several embodiments, an antigen binding protein is directed against ALPPL2. In several embodiments, the anti-ALPPL2 binding protein is an antigen binding domain of a CAR which comprises a transmembrane domain, a signaling domain, and optionally a co-stimulatory domain as disclosed herein. In several embodiments, the anti-ALPPL2 binding protein comprises a vL chain comprising the amino acid sequence of any of SEQ ID NOs: 657-659, or a sequence having at least about 95%, at least about 96%, at least about 97%, at least about 98%, or at least about 99%, sequence identity, homology and/or functional equivalence to any of SEQ ID NOs: 657-659. In some embodiments, the vL comprises the amino acid sequence set forth in any of SEQ ID NO:657-659. In several embodiments, the anti-ALPPL2 binding protein comprises a vH chain comprising the amino acid sequence of any of SEQ ID NOs: 660-662, or a sequence having at least about 95%, at least about 96%, at least about 97%, at least about 98%, or at least about 99%, sequence identity, homology and/or functional equivalence to any of SEQ ID NOs: 660-662. In some embodiments, the vH comprises the amino acid sequence set forth in any of SEQ ID NO:660-662. In several embodiments, the anti-ALPPL2 binding protein is an antibody, or scFv, containing one or more combinations of the vL and vH domains comprising the amino acid sequence of any of SEQ ID NOs: 657-662, or a sequence having at least about 95%, at least about 96%, at least about 97%, at least about 98%, or at least about 99%, sequence identity, homology and/or functional equivalence to any of SEQ ID NO: 657-662. In several embodiments, the anti-ALPPL2 binding protein is a scFv. In several embodiments, the antigen binding protein is affinity matured to enhance binding to ALPPL2. In several embodiments, the nucleotide sequence encoding the antigen binding protein is codon-optimized to enhance expression and/or stability of the protein.

### CLDN4

In several embodiments, an antigen binding protein is directed against CLDN4. In several embodiments, the anti-CLDN4 binding protein is an antigen binding domain of a CAR which comprises a transmembrane domain, a signaling domain, and optionally a co-stimulatory domain as disclosed herein. In several embodiments, the anti-CLDN4 binding protein comprises a vL chain comprising the amino acid sequence of any of SEQ ID NOs: 663, 664, or 667, or a sequence having at least about 95%, at least about 96%, at least about 97%, at least about 98%, or at least about 99%, sequence identity, homology and/or functional equivalence to any of SEQ ID NOs: 663, 664, or 667. In some embodiments, the vL comprises the amino acid sequence set forth in any of SEQ ID NOS:663, 664, and 667. In several embodiments, the anti-CLDN4 binding protein comprises a vH chain comprising the amino acid sequence of any of SEQ ID NOs: 665, 666, or 668, or a sequence having at least about 95%, at least about 96%, at least about 97%, at least about 98%, or at least about 99%, sequence identity, homology and/or functional equivalence to any of SEQ ID NOs: 665, 666, or 668. In some embodiments, the vH comprises the amino acid sequence set forth in any of SEQ ID NOS:665, 666, and 668. In several embodiments, the anti-CLDN4 binding protein is an antibody, or scFv, containing one or more combinations of the vL and vH domains comprising the amino acid sequence of any of SEQ ID NOs: 663-668, or a sequence having at least about 95%, at least about 96%, at least about 97%, at least about 98%, or at least about 99%, sequence identity, homology and/or functional equivalence to any of SEQ ID NOs: 663-668. In several embodiments, the anti-CLDN4 binding protein comprises an scFv. In several embodiments, the antigen binding protein is affinity matured to enhance binding to CLDN4. In several embodiments, the nucleotide sequence encoding the antigen binding protein is codon-optimized to enhance expression and/or stability of the protein. In several embodiments, the antigen binding protein binds to CLDN4, but not to other claudins.

### CLDN6

In several embodiments, an antigen binding protein is directed against CLDN6. In several embodiments, the anti-CLDN6 binding protein is an antigen binding domain of a CAR which comprises a transmembrane domain, a signaling domain, and optionally a co-stimulatory domain as disclosed herein. In several embodiments, the anti-CLDN6 binding protein comprises a vL chain comprising the amino acid sequence of any of SEQ ID NOs: 669-678, or a sequence having at least about 95%, at least about 96%, at least about 97%, at least about 98%, or at least about 99%, sequence identity, homology and/or functional equivalence to any of SEQ ID NOs: 669-678. In some embodiments, the vL comprises the amino acid sequence of any of SEQ ID NOS:669-678. In several embodiments, the anti-CLDN6 binding protein comprises a vH chain comprising the amino acid sequence of any of SEQ ID NOs: 679-682, or a sequence having at least about 95%, at least about 96%, at least about 97%, at least about 98%, or at least about 99%, sequence identity, homology and/or functional equivalence to any of SEQ ID NOs: 679-682. In some embodiments, the vH comprises the amino acid sequence of any of SEQ ID NOS:679-682. In several embodiments, the anti-CLDN6 binding protein is an antibody, or scFv, containing one or more combinations of the vL and vH domains comprising the amino acid sequence of any of SEQ ID NOs: 669-682, or a sequence having at least about 95%, at least about 96%, at least about 97%, at least about 98%, or at least about 99%, sequence identity, homology and/or functional equivalence to any of SEQ ID NOs: 669-682. In several embodiments, the anti-CLDN6 binding protein is a scFv. In several embodiments, the antigen binding protein is affinity matured to enhance binding to CLDN6. In several embodiments, the nucleotide sequence encoding the antigen binding protein is codon-optimized to enhance expression and/or stability of the protein. In several embodiments, the antigen binding protein binds to CLDN6, but not to other claudins.

Several embodiments relate to CARs that are directed to Claudin 6, and show little or no binding to Claudin 3, 4, or 9 (or other Claudins). In some embodiments, the antigen-binding protein comprises a heavy chain variable region (VH). In some embodiments, the VH comprises a CDR-H1 comprising the amino acid sequence set forth in SEQ ID NO:92, a CDR-H2 comprising the amino acid sequence set forth in SEQ ID NO:93, and a CDR-H3 comprising the amino acid sequence set forth in SEQ ID NO:94. In some embodiments, the antigen-binding protein comprises a heavy chain variable having at least 90% identity to the VH amino acid sequence set forth in SEQ ID NO: 88. In some embodiments, the antigen-binding protein comprises a heavy chain variable having at least 95% identity to the VH amino acid sequence set forth in SEQ ID NO: 88. In some embodiments, the antigen-binding protein comprises a heavy chain variable having at least 96, 97, 98, or 99% identity to the VH amino acid sequence set forth in SEQ ID NO: 88. In several embodiments, the heavy chain variable may have one or more additional mutations (e.g., for purposes of humanization) in the VH amino acid sequence set forth in SEQ ID NO: 88, but retains specific binding to a cancer antigen (e.g., CLDN6). In several embodiments, the heavy chain variable may have one or more additional mutations in the VH amino acid sequence set forth in SEQ ID NO: 88, but has improved specific binding to a cancer antigen (e.g., CLDN6). In some embodiments, the antigen-binding protein comprises a heavy chain variable comprising the amino acid sequence of SEQ ID NO: 88.

In some embodiments, the antigen-binding protein comprises a light chain variable region having at least 90% identity to the VL amino acid sequence set forth in SEQ ID NO: 89, 90 or 91. In some embodiments, the antigen-binding protein comprises a light chain variable having at least 95% identity to the VL amino acid sequence set forth in SEQ ID NO: 89, 90 or 91. In some embodiments, the antigen-binding protein comprises a light chain variable having at least 96, 97, 98, or 99% identity to the VL amino acid sequence set forth in SEQ ID NO: 89, 90 or 91. In several embodiments, the light chain variable may have one or more additional mutations (e.g., for purposes of humanization) in the VL amino acid sequence set forth in SEQ ID NO: 89, 90 or 91, but retains specific binding to a cancer antigen (e.g., CLDN6). In several embodiments, the light chain variable may have one or more additional mutations in the VL amino acid sequence set forth in SEQ ID NO: 89, 90 or 91, but has improved specific binding to a cancer antigen (e.g., CLDN6). In some embodiments, the antigen-binding protein comprises a light chain variable comprising the amino acid sequence of SEQ ID NO: 89, 90 or 91. In some embodiments, the VL comprises the amino acid sequence of SEQ ID NO: 89. In some embodiments, the VL comprises the amino acid sequence of SEQ ID NO: 90. In some embodiments, the VL comprises the amino acid sequence of SEQ ID NO: 91.

### CD19

In some embodiments, the antigen-binding protein binds to CD19. In some embodiments, an antigen-binding protein is provided comprising a heavy chain variable domain having at least 90% identity to the VH domain amino acid sequence set forth in SEQ ID NO: 33. In some embodiments, the antigen-binding protein comprises a heavy chain variable domain having at least 95% identity to the VH domain amino acid sequence set forth in SEQ ID NO: 33. In some embodiments, the antigen-binding protein comprises a heavy chain variable domain having at least 96, 97, 98, or 99% identity to the VH domain amino acid sequence set forth in SEQ ID NO: 33. In several embodiments, the heavy chain variable domain may have one or more additional mutations (e.g., for purposes of humanization) in the VH domain amino acid sequence set forth in SEQ ID NO: 33, but retains specific binding to a cancer antigen (e.g., CD19). In several embodiments, the heavy chain variable domain may have one or more additional mutations in the VH domain amino acid sequence set forth in SEQ ID NO: 33, but has improved specific binding to a cancer antigen (e.g., CD19). In some embodiments, the VH comprises the amino acid sequence set forth in SEQ ID NO:33.

In some embodiments, the antigen-binding protein comprises a light chain variable domain having at least 90% identity to the VL domain amino acid sequence set forth in SEQ ID NO: 32. In some embodiments, the antigen-binding protein comprises a light chain variable domain having at least 95% identity to the VL domain amino acid sequence set forth in SEQ ID NO: 32. In some embodiments, the antigen-binding protein comprises a light chain variable domain having at least 96, 97, 98, or 99% identity to the VL domain amino acid sequence set forth in SEQ ID NO: 32. In several embodiments, the light chain variable domain may have one or more additional mutations (e.g., for purposes of humanization) in the VL domain amino acid sequence set forth in SEQ ID NO: 32, but retains specific binding to a cancer antigen (e.g., CD19). In several embodiments, the light chain variable domain may have one or more additional mutations in the VL domain amino acid sequence set forth in SEQ ID NO: 32, but has improved specific binding to a cancer antigen (e.g., CD19). In some embodiments, the VH comprises the amino acid sequence set forth in SEQ ID NO:32.

In some embodiments, the antigen-binding protein comprises a heavy chain variable domain having at least 90% identity to the VH domain amino acid sequence set forth in SEQ ID NO: 33, and a light chain variable domain having at least 90% identity to the VL domain amino acid sequence set forth in SEQ ID NO: 32. In some embodiments, the antigen-binding protein comprises a heavy chain variable domain having at least 95% identity to the VH domain amino acid sequence set forth in SEQ ID NO: 33, and a light chain variable domain having at least 95% identity to the VL domain amino acid sequence set forth in SEQ ID NO: 32. In some embodiments, the antigen-binding protein comprises a heavy chain variable domain having at least 96, 97, 98, or 99% identity to the VH domain amino acid sequence set forth in SEQ ID NO: 33, and a light chain variable domain having at least 96, 97, 98, or 99% identity to the VL domain amino acid sequence set forth in SEQ ID NO: 32.

In some embodiments, the antigen-binding protein comprises a heavy chain variable domain having the VH domain amino acid sequence set forth in SEQ ID NO: 33, and a light chain variable domain having the VL domain amino acid sequence set forth in SEQ ID NO: 32. In some embodiments, the light-chain variable domain comprises a sequence of amino acids that is at least 70%, 75%, 80%, 85%, 90%, 91%, 92%, 93%, 94%, 95%, 96%, 97%, 98%, 99%, or more, identical to the sequence of a light chain variable domain of SEQ ID NO: 32. In some embodiments, the light-chain variable domain comprises a sequence of amino acids that is at least 70%, 75%, 80%, 85%, 90%, 91%, 92%, 93%, 94%, 95%, 96%, 97%, 98%, 99%, or more, identical to the sequence of a heavy chain variable domain in accordance with SEQ ID NO: 33.

In some embodiments, the light chain variable domain comprises a sequence of amino acids that is encoded by a nucleotide sequence that is at least 70%, 75%, 80%, 85%, 90%, 91%, 92%, 93%, 94%, 95%, 96%, 97%, 98%, 99%, or more, identical to the polynucleotide sequence SEQ ID NO: 32. In some embodiments, the light chain variable domain comprises a sequence of amino acids that is encoded by a polynucleotide that hybridizes under moderately stringent conditions to the complement of a polynucleotide that encodes a light chain variable domain in accordance with the sequence in SEQ ID NO: 32. In some embodiments, the light chain variable domain comprises a sequence of amino acids that is encoded by a polynucleotide that hybridizes under stringent conditions to the complement of a polynucleotide that encodes a light chain variable domain in accordance with the sequence in SEQ ID NO: 32.

In some embodiments, the heavy chain variable domain comprises a sequence of amino acids that is at least 70%, 75%, 80%, 85%, 90%, 91%, 92%, 93%, 94%, 95%, 96%, 97%, 98%, 99%, or more, identical to the sequence of a heavy chain variable domain in accordance with the sequence of SEQ ID NO: 33. In some embodiments, the heavy chain variable domain comprises a sequence of amino acids that is encoded by a polynucleotide that hybridizes under moderately stringent conditions to the complement of a polynucleotide that encodes a heavy chain variable domain in accordance with the sequence of SEQ ID NO: 33. In some embodiments, the heavy chain variable domain comprises a sequence of amino acids that is encoded by a polynucleotide that hybridizes under stringent conditions to the complement of a polynucleotide that encodes a heavy chain variable domain in accordance with the sequence of SEQ ID NO: 33.

In some embodiments, the anti-CD19 binding protein is an scFv comprising a VH and a VL. In several embodiments, additional anti-CD19 binding constructs are provided. For example, in several embodiments, there is provided an scFv that targets CD19 wherein the scFv comprises a heavy chain variable region comprising the sequence of SEQ ID NO. 35. In some embodiments, the antigen-binding protein comprises a heavy chain variable domain having at least 95% identity to the HCV domain amino acid sequence set forth in SEQ ID NO: 35. In some embodiments, the antigen-binding protein comprises a heavy chain variable domain having at least 96, 97, 98, or 99% identity to the HCV domain amino acid sequence set forth in SEQ ID NO: 35. In several embodiments, the heavy chain variable domain may have one or more additional mutations (e.g., for purposes of humanization) in the HCV domain amino acid sequence set forth in SEQ ID NO: 35, but retains specific binding to a cancer antigen (e.g., CD19). In several embodiments, the heavy chain variable domain may have one or more additional mutations in the HCV domain amino acid sequence set forth in SEQ ID NO: 35, but has improved specific binding to a cancer antigen (e.g., CD19).

Additionally, in several embodiments, an scFv that targets CD19 comprises a light chain variable region comprising the sequence of SEQ ID NO. 36. In some embodiments, the antigen-binding protein comprises a light chain variable domain having at least 95% identity to the LCV domain amino acid sequence set forth in SEQ ID NO: 36. In some embodiments, the antigen-binding protein comprises a light chain variable domain having at least 96, 97, 98, or 99% identity to the LCV domain amino acid sequence set forth in SEQ ID NO: 36. In several embodiments, the light chain variable domain may have one or more additional mutations (e.g., for purposes of humanization) in the LCV domain amino acid sequence set forth in SEQ ID NO: 36, but retains specific binding to a cancer antigen (e.g., CD19). In several embodiments, the light chain variable domain may have one or more additional mutations in the LCV domain amino acid sequence set forth in SEQ ID NO: 36, but has improved specific binding to a cancer antigen (e.g., CD19). In some embodiments, the scFv comprises a VH comprising the amino acid sequence of SEQ ID NO:35 and a VL comprising the amino acid sequence of SEQ ID NO:36.

In several embodiments, there is also provided an anti-CD19 binding moiety that comprises a light chain CDR comprising a first, second and third complementarity determining region (LC CDR1, LC CDR2, and LC CDR3, respectively. In several embodiments, the anti-CD19 binding moiety further comprises a heavy chain CDR comprising a first, second and third complementarity determining region (HC CDR1, HC CDR2, and HC CDR3, respectively. In several embodiments, the LC CDR1 comprises the sequence of SEQ ID NO. 37. In several embodiments, the LC CDR1 comprises an amino acid sequence with at least about 85%, about 90%, about 95%, or about 98% sequence identity to the sequence of SEQ NO. 37. In several embodiments, the LC CDR2 comprises the sequence of SEQ ID NO. 38. In several embodiments, the LC CDR2 comprises an amino acid sequence with at least about 85%, about 90%, about 95%, or about 98% sequence identity to the sequence of SEQ NO. 38. In several embodiments, the LC CDR3 comprises the sequence of SEQ ID NO. 39. In several embodiments, the LC CDR3 comprises an amino acid sequence with at least about 85%, about 90%, about 95%, or about 98% sequence identity to the sequence of SEQ NO. 39. In several embodiments, the HC CDR1 comprises the sequence of SEQ ID NO. 40. In several embodiments, the HC CDR1 comprises an amino acid sequence with at least about 85%, about 90%, about 95%, or about 98% sequence identity to the sequence of SEQ NO. 40. In several embodiments, the HC CDR2 comprises the sequence of SEQ ID NO. 41, 42, or 43. In several embodiments, the HC CDR2 comprises an amino acid sequence with at least about 85%, about 90%, about 95%, or about 98% sequence identity to the sequence of SEQ NO. 41, 42, or 43. In several embodiments, the HC CDR3 comprises the sequence of SEQ ID NO. 44. In several embodiments, the HC CDR3 comprises an amino acid sequence with at least about 85%, about 90%, about 95%, or about 98% sequence identity to the sequence of SEQ NO. 44.

In several embodiments, there is also provided an anti-CD19 binding moiety that comprises a light chain variable region (VL) and a heavy chain variable region (HL), the VL region comprising a first, second and third complementarity determining region (VL CDR1, VL CDR2, and VL CDR3, respectively and the VH region comprising a first, second and third complementarity determining region (VH CDR1, VH CDR2, and VH CDR3, respectively. In several embodiments, the VL region comprises the sequence of SEQ ID NO. 45, 46, 47, or 48. In several embodiments, the VL region comprises an amino acid sequence with at least about 85%, about 90%, about 95%, or about 98% sequence identity to the sequence of SEQ NO. 45, 46, 47, or 48. In several embodiments, the VH region comprises the sequence of SEQ ID NO. 49, 50, 51 or 52. In several embodiments, the VH region comprises an amino acid sequence with at least about 85%, about 90%, about 95%, or about 98% sequence identity to the sequence of SEQ NO. 49, 50, 51 or 52.

In several embodiments, there is also provided an anti-CD19 binding moiety that comprises a light chain CDR comprising a first, second and third complementarity determining region (LC CDR1, LC CDR2, and LC CDR3, respectively. In several embodiments, the anti-CD19 binding moiety further comprises a heavy chain CDR comprising a first, second and third complementarity determining region (HC CDR1, HC CDR2, and HC CDR3, respectively. In several embodiments, the LC CDR1 comprises the sequence of SEQ ID NO. 53. In several embodiments, the LC CDR1 comprises an amino acid sequence with at least about 85%, about 90%, about 95%, or about 98% sequence identity to the sequence of SEQ NO. 53. In several embodiments, the LC CDR2 comprises the sequence of SEQ ID NO. 54. In several embodiments, the LC CDR2 comprises an amino acid sequence with at least about 85%, about 90%, about 95%, or about 98% sequence identity to the sequence of SEQ NO. 54. In several embodiments, the LC CDR3 comprises the sequence of SEQ ID NO. 55. In several embodiments, the LC CDR3 comprises an amino acid sequence with at least about 85%, about 90%, about 95%, or about 98% sequence identity to the sequence of SEQ NO. 55. In several embodiments, the HC CDR1 comprises the sequence of SEQ ID NO. 56. In several embodiments, the HC CDR1 comprises an amino acid sequence with at least about 85%, about 90%, about 95%, or about 98% sequence identity to the sequence of SEQ NO. 56. In several embodiments, the HC CDR2 comprises the sequence of SEQ ID NO. 57. In several embodiments, the HC CDR2 comprises an amino acid sequence with at least about 85%, about 90%, about 95%, or about 98% sequence identity to the sequence of SEQ NO. 57. In several embodiments, the HC CDR3 comprises the sequence of SEQ ID NO. 58. In several embodiments, the HC CDR3 comprises an amino acid sequence with at least about 85%, about 90%, about 95%, or about 98% sequence identity to the sequence of SEQ NO. 58.

In some embodiments, the antigen-binding protein comprises a heavy chain variable region comprising the amino acid sequence of SEQ ID NO: 104. In some embodiments, the antigen-binding protein comprises a heavy chain variable region having at least 90% identity to the VH domain amino acid sequence set forth in SEQ ID NO: 104. In some embodiments, the antigen-binding protein comprises a heavy chain variable domain having at least 95% sequence identity to the VH domain amino acid sequence set forth in SEQ ID NO: 104. In some embodiments, the antigen-binding protein comprises a heavy chain variable domain having at least 96, 97, 98, or 99% sequence identity to the VH domain amino acid sequence set forth in SEQ ID NO: 104. In several embodiments, the heavy chain variable domain may have one or more additional mutations (e.g., for purposes of humanization) in the VH domain amino acid sequence set forth in SEQ ID NO: 104, but retains specific binding to a cancer antigen (e.g., CD19). In several embodiments, the heavy chain variable domain may have one or more additional mutations in the VH domain amino acid sequence set forth in SEQ ID NO: 104, but has improved specific binding to a cancer antigen (e.g., CD19).

In some embodiments, the antigen-binding protein comprises a light chain variable region comprising the amino acid sequence of SEQ ID NO: 105. In some embodiments, the antigen-binding protein comprises a light chain variable region having at least 90% sequence identity to the VL domain amino acid sequence set forth in SEQ ID NO: 105. In some embodiments, the antigen-binding protein comprises a light chain variable domain having at least 95% sequence identity to the VL domain amino acid sequence set forth in SEQ ID NO: 105. In some embodiments, the antigen-binding protein comprises a light chain variable domain having at least 96, 97, 98, or 99% sequence identity to the VL domain amino acid sequence set forth in SEQ ID NO: 105. In several embodiments, the light chain variable domain may have one or more additional mutations (e.g., for purposes of humanization) in the VL domain amino acid sequence set forth in SEQ ID NO: 105, but retains specific binding to a cancer antigen (e.g., CD19). In several embodiments, the light chain variable domain may have one or more additional mutations in the VL domain amino acid sequence set forth in SEQ ID NO: 105, but has improved specific binding to a cancer antigen (e.g., CD19).

In some embodiments, the antigen-binding protein comprises a heavy chain variable domain having the VH domain amino acid sequence set forth in SEQ ID NO: 104, and a light chain variable domain having the VL domain amino acid sequence set forth in SEQ ID NO: 105. In some embodiments, the light-chain variable domain comprises a sequence of amino acids that is at least 70%, 75%, 80%, 85%, 90%, 91%, 92%, 93%, 94%, 95%, 96%, 97%, 98%, 99%, or more, identical to the sequence of a light chain variable domain of SEQ ID NO: 105. In some embodiments, the heavy-chain variable domain comprises a sequence of amino acids that is at least 70%, 75%, 80%, 85%, 90%, 91%, 92%, 93%, 94%, 95%, 96%, 97%, 98%, 99%, or more, identical to the sequence of a heavy chain variable domain in accordance with SEQ ID NO: 104.

In some embodiments, the antigen-binding protein comprises a heavy chain variable comprising the amino acid sequence of SEQ ID NO: 106. In some embodiments, the antigen-binding protein comprises a heavy chain variable having at least 90% sequence identity to the VH amino acid sequence set forth in SEQ ID NO: 106. In some embodiments, the antigen-binding protein comprises a heavy chain variable having at least 95% sequence identity to the VH amino acid sequence set forth in SEQ ID NO: 106. In some embodiments, the antigen-binding protein comprises a heavy chain variable having at least 96, 97, 98, or 99% identity to the VH amino acid sequence set forth in SEQ ID NO: 106. In several embodiments, the heavy chain variable may have one or more additional mutations (e.g., for purposes of humanization) in the VH amino acid sequence set forth in SEQ ID NO: 106, but retains specific binding to a cancer antigen (e.g., CD19). In several embodiments, the heavy chain variable may have one or more additional mutations in the VH amino acid sequence set forth in SEQ ID NO: 106, but has improved specific binding to a cancer antigen (e.g., CD19).

In some embodiments, the antigen-binding protein comprises a light chain variable comprising the amino acid sequence of SEQ ID NO: 107. In some embodiments, the antigen-binding protein comprises a light chain variable region having at least 90% sequence identity to the VL amino acid sequence set forth in SEQ ID NO: 107. In some embodiments, the antigen-binding protein comprises a light chain variable having at least 95% sequence identity to the VL amino acid sequence set forth in SEQ ID NO: 107. In some embodiments, the antigen-binding protein comprises a light chain variable having at least 96, 97, 98, or 99% identity to the VL amino acid sequence set forth in SEQ ID NO: 107. In several embodiments, the light chain variable may have one or more additional mutations (e.g., for purposes of humanization) in the VL amino acid sequence set forth in SEQ ID NO: 107, but retains specific binding to a cancer antigen (e.g., CD19). In several embodiments, the light chain variable may have one or more additional mutations in the VL amino acid sequence set forth in SEQ ID NO: 107, but has improved specific binding to a cancer antigen (e.g., CD19).

In several embodiments, there is also provided an anti-CD19 binding moiety that comprises a light chain CDR comprising a first, second and third complementarity determining region (LC CDR1, LC CDR2, and LC CDR3, respectively. In several embodiments, the anti-CD19 binding moiety further comprises a heavy chain CDR comprising a first, second and third complementarity determining region (HC CDR1, HC CDR2, and HC CDR3, respectively. In several embodiments, the LC CDR1 comprises the sequence of SEQ ID NO. 108. In several embodiments, the LC CDR1 comprises an amino acid sequence with at least about 85%, about 90%, about 95%, or about 98% sequence identity to the sequence of SEQ NO. 108. In several embodiments, the LC CDR2 comprises the sequence of SEQ ID NO. 109. In several embodiments, the LC CDR2 comprises an amino acid sequence with at least about 85%, about 90%, about 95%, or about 98% sequence identity to the sequence of SEQ NO. 109. In several embodiments, the LC CDR3 comprises the sequence of SEQ ID NO. 110. In several embodiments, the LC CDR3 comprises an amino acid sequence with at least about 85%, about 90%, about 95%, or about 98% sequence identity to the sequence of SEQ NO. 110. In several embodiments, the HC CDR1 comprises the sequence of SEQ ID NO. 111. In several embodiments, the HC CDR1 comprises an amino acid sequence with at least about 85%, about 90%, about 95%, or about 98% sequence identity to the sequence of SEQ NO. 111. In several embodiments, the HC CDR2 comprises the sequence of SEQ ID NO. 112, 113, or 114. In several embodiments, the HC CDR2 comprises an amino acid sequence with at least about 85%, about 90%, about 95%, or about 98% sequence identity to the sequence of SEQ NO. 112, 113, or 114. In several embodiments, the HC CDR3 comprises the sequence of SEQ ID NO. 115. In several embodiments, the HC CDR3 comprises an amino acid sequence with at least about 85%, about 90%, about 95%, or about 98% sequence identity to the sequence of SEQ NO. 115. In several embodiments, the anti-CD19 binding moiety comprises SEQ ID NO: 116, or is sequence with at least about 85%, about 90%, about 95%, or about 98% sequence identity to the sequence of SEQ NO. 116.

In some embodiments, the antigen-binding protein comprises a light chain variable comprising the amino acid sequence of SEQ ID NO: 117, 118, or 119. In some embodiments, the antigen-binding protein comprises a light chain variable region having at least 90% identity to the VL amino acid sequence set forth in SEQ ID NO: 117, 118, or 119. In some embodiments, the antigen-binding protein comprises a light chain variable having at least 95% identity to the VL amino acid sequence set forth in SEQ ID NO: 117, 118, or 119. In some embodiments, the antigen-binding protein comprises a light chain variable having at least 96, 97, 98, or 99% identity to the VL amino acid sequence set forth in SEQ ID NO: 117, 118, or 119. In several embodiments, the light chain variable may have one or more additional mutations (e.g., for purposes of humanization) in the VL amino acid sequence set forth in SEQ ID NO: 117, 118, or 119, but retains specific binding to a cancer antigen (e.g., CD19). In several embodiments, the light chain variable may have one or more additional mutations in the VL amino acid sequence set forth in SEQ ID NO: 117, 118, or 119, but has improved specific binding to a cancer antigen (e.g., CD19).

In some embodiments, the antigen-binding protein comprises a heavy chain variable comprising the amino acid sequence of SEQ ID NO: 120, 121, 122, or 123. In some embodiments, the antigen-binding protein comprises a heavy chain variable having at least 90% identity to the VH amino acid sequence set forth in SEQ ID NO: 120, 121, 122, or 123. In some embodiments, the antigen-binding protein comprises a heavy chain variable having at least 95% identity to the VH amino acid sequence set forth in SEQ ID NO: 120, 121, 122, or 123. In some embodiments, the antigen-binding protein comprises a heavy chain variable having at least 96, 97, 98, or 99% identity to the VH amino acid sequence set forth in SEQ ID NO: 120, 121, 122, or 123. In several embodiments, the heavy chain variable may have one or more additional mutations (e.g., for purposes of humanization) in the VH amino acid sequence set forth in SEQ ID NO: 120, 121, 122, or 123, but retains specific binding to a cancer antigen (e.g., CD19). In several embodiments, the heavy chain variable may have one or more additional mutations in the VH amino acid sequence set forth in SEQ ID NO: 120, 121, 122, or 123, but has improved specific binding to a cancer antigen (e.g., CD19).

In several embodiments, there is also provided an anti-CD19 binding moiety that comprises a light chain CDR comprising a first, second and third complementarity determining region (LC CDR1, LC CDR2, and LC CDR3, respectively. In several embodiments, the anti-CD19 binding moiety further comprises a heavy chain CDR comprising a first, second and third complementarity determining region (HC CDR1, HC CDR2, and HC CDR3, respectively. In several embodiments, the LC CDR1 comprises the sequence of SEQ ID NO. 124, 127, or 130. In several embodiments, the LC CDR1 comprises an amino acid sequence with at least about 85%, about 90%, about 95%, or about 98% sequence identity to the sequence of SEQ NO. 124, 127, or 130. In several embodiments, the LC CDR2 comprises the sequence of SEQ ID NO. 125, 128, or 131. In several embodiments, the LC CDR2 comprises an amino acid sequence with at least about 85%, about 90%, about 95%, or about 98% sequence identity to the sequence of SEQ NO. 125, 128, or 131. In several embodiments, the LC CDR3 comprises the sequence of SEQ ID NO. 126, 129, or 132. In several embodiments, the LC CDR3 comprises an amino acid sequence with at least about 85%, about 90%, about 95%, or about 98% sequence identity to the sequence of SEQ NO. 126, 129, or 132. In several embodiments, the HC CDR1 comprises the sequence of SEQ ID NO. 133, 136, 139, or 142. In several embodiments, the HC CDR1 comprises an amino acid sequence with at least about 85%, about 90%, about 95%, or about 98% sequence identity to the sequence of SEQ NO. 133, 136, 139, or 142. In several embodiments, the HC CDR2 comprises the sequence of SEQ ID NO. 134, 137, 140, or 143. In several embodiments, the HC CDR2 comprises an amino acid sequence with at least about 85%, about 90%, about 95%, or about 98% sequence identity to the sequence of SEQ NO. 134, 137, 140, or 143. In several embodiments, the HC CDR3 comprises the sequence of SEQ ID NO. 135, 138, 141, or 144. In several embodiments, the HC CDR3 comprises an amino acid sequence with at least about 85%, about 90%, about 95%, or about 98% sequence identity to the sequence of SEQ NO. 135, 138, 141, or 144.

Additional anti-CD19 binding moieties are known in the art, such as those disclosed in, for example, U.S. Pat. No. 8,399,645, US Patent Publication No. 2018/0153977, US Patent Publication No. 2014/0271635, US Patent Publication No. 2018/0251514, US Patent Publication No. 2018/0312588, and WO 2020/180882, the entirety of each of which is incorporated by reference herein.

In several embodiments, there is also provided an anti-CLDN6 binding moiety that comprises a light chain CDR comprising a first, second and third complementarity determining region (LC CDR1, LC CDR2, and LC CDR3, respectively. In several embodiments, the anti-CD19 binding moiety further comprises a heavy chain CDR comprising a first, second and third complementarity determining region (HC CDR1, HC CDR2, and HC CDR3, respectively. In several embodiments, the LC CDR1 comprises the sequence of SEQ ID NO. 95, 98, or 101. In several embodiments, the LC CDR1 comprises an amino acid sequence with at least about 85%, about 90%, about 95%, or about 98% sequence identity to the sequence of SEQ NO. 95, 98, or 101. In several embodiments, the LC CDR2 comprises the sequence of SEQ ID NO. 96, 99, or 102. In several embodiments, the LC CDR2 comprises an amino acid sequence with at least about 85%, about 90%, about 95%, or about 98% sequence identity to the sequence of SEQ NO. 96, 99, or 102. In several embodiments, the LC CDR3 comprises the sequence of SEQ ID NO. 97, 100, or 103. In several embodiments, the LC CDR3 comprises an amino acid sequence with at least about 85%, about 90%, about 95%, or about 98% sequence identity to the sequence of SEQ NO. 97, 100, or 103. In several embodiments, the HC CDR1 comprises the sequence of SEQ ID NO. 92. In several embodiments, the HC CDR1 comprises an amino acid sequence with at least about 85%, about 90%, about 95%, or about 98% sequence identity to the sequence of SEQ NO. 92. In several embodiments, the HC CDR2 comprises the sequence of SEQ ID NO. 93. In several embodiments, the HC CDR2 comprises an amino acid sequence with at least about 85%, about 90%, about 95%, or about 98% sequence identity to the sequence of SEQ NO. 93. In several embodiments, the HC CDR3 comprises the sequence of SEQ ID NO. 94. In several embodiments, the HC CDR3 comprises an amino acid sequence with at least about 85%, about 90%, about 95%, or about 98% sequence identity to the sequence of SEQ NO. 94. In several embodiments, the antigen-binding protein does not bind claudins other than CLDN6.

b. Natural Killer Group Domains that Bind Tumor Ligands

In several embodiments, engineered immune cells such as NK cells are leveraged for their ability to recognize and destroy tumor cells. For example, an engineered NK cell may include a CD19-directed chimeric antigen receptor or a nucleic acid encoding said chimeric antigen receptor (or a CAR directed against, for example, one or more of CD123, CD70, Her2, mesothelin, Claudin 6, BCMA, EGFR, etc.). NK cells express both inhibitory and activating receptors on the cell surface. Inhibitory receptors bind self-molecules expressed on the surface of healthy cells (thus preventing immune responses against “self” cells), while the activating receptors bind ligands expressed on abnormal cells, such as tumor cells. When the balance between inhibitory and activating receptor activation is in favor of activating receptors, NK cell activation occurs and target (e.g., tumor) cells are lysed.

Natural killer Group 2 member D (NKG2D) is an NK cell activating receptor that recognizes a variety of ligands expressed on cells. The surface expression of various NKG2D ligands is generally low in healthy cells but is upregulated upon, for example, malignant transformation. Non-limiting examples of ligands recognized by NKG2D include, but are not limited to, MICA, MICB, ULBP1, ULBP2, ULBP3, ULBP4, ULBP5, and ULBP6, as well as other molecules expressed on target cells that control the cytolytic or cytotoxic function of NK cells. In several embodiments, T cells are engineered to express an extracellular domain to binds to one or more tumor ligands and activate the T cell. For example, in several embodiments, T cells are engineered to express an NKG2D receptor as the binder/activation moiety. In several embodiments, engineered cells as disclosed herein are engineered to express another member of the NKG2 family, e.g., NKG2A, NKG2C, and/or NKG2E. Combinations of such receptors are engineered in some embodiments. Moreover, in several embodiments, other receptors are expressed, such as the Killer-cell immunoglobulin-like receptors (KIRs).

In several embodiments, cells are engineered to express a cytotoxic receptor complex comprising a full length NKG2D as an extracellular component to recognize ligands on the surface of tumor cells (e.g., liver cells). In one embodiment, full length NKG2D has the nucleic acid sequence of SEQ ID NO: 27. In several embodiments, the full length NKG2D, or functional fragment thereof is human NKG2D. Additional information about chimeric receptors for use in the presently disclosed methods and compositions can be found in PCT Patent Publication No. WO/2018/183385, which is incorporated in its entirety by reference herein.

In several embodiments, cells are engineered to express a cytotoxic receptor complex comprising a functional fragment of NKG2D as an extracellular component to recognize ligands on the surface of tumor cells or other diseased cells. In one embodiment, the functional fragment of NKG2D has the nucleic acid sequence of SEQ ID NO: 25. In several embodiments, the fragment of NKG2D is at least 70%, at least 75%, at least 80%, at least 85%, at least 90%, or at least 95% homologous with full-length wild-type NKG2D. In several embodiments, the fragment may have one or more additional mutations from SEQ ID NO: 25, but retains, or in some embodiments, has enhanced, ligand-binding function. In several embodiments, the functional fragment of NKG2D comprises the amino acid sequence of SEQ ID NO: 26. In several embodiments, the NKG2D fragment is provided as a dimer, trimer, or other concatameric format, such embodiments providing enhanced ligand-binding activity. In several embodiments, the sequence encoding the NKG2D fragment is optionally fully or partially codon optimized. In one embodiment, a sequence encoding a codon optimized NKG2D fragment comprises the sequence of SEQ ID NO: 28. Advantageously, according to several embodiments, the functional fragment lacks its native transmembrane or intracellular domains but retains its ability to bind ligands of NKG2D as well as transduce activation signals upon ligand binding. A further advantage of such fragments is that expression of DAP10 to localize NKG2D to the cell membrane is not required. Thus, in several embodiments, the cytotoxic receptor complex encoded by the polypeptides disclosed herein does not comprise DAP10. In several embodiments, immune cells, such as NK or T cells (e.g., non-alloreactive T cells engineered according to embodiments disclosed herein), are engineered to express one or more chimeric receptors that target, for example CD19, CD123, CD70, Her2, mesothelin, Claudin 6, BCMA, EGFR, and an NKG2D ligand, such as MICA, MICB, ULBP1, ULBP2, ULBP3, ULBP4, ULBP5, and/or ULBP6. Such cells, in several embodiments, also co-express mbIL15.

In several embodiments, the cytotoxic receptor complexes are configured to dimerize. Dimerization may comprise homodimers or heterodimers, depending on the embodiment. In several embodiments, dimerization results in improved ligand recognition by the cytotoxic receptor complexes (and hence the NK cells expressing the receptor), resulting in a reduction in (or lack) of adverse toxic effects. In several embodiments, the cytotoxic receptor complexes employ internal dimers, or repeats of one or more component subunits. For example, in several embodiments, the cytotoxic receptor complexes may optionally comprise a first NKG2D extracellular domain coupled to a second NKG2D extracellular domain, and a transmembrane/signaling region (or a separate transmembrane region along with a separate signaling region).

In several embodiments, the various domains/subdomains are separated by a linker such as, a GS3 linker (SEQ ID NO: 15 and 16, nucleotide and protein, respectively) is used (or a GSn linker). Other linkers used according to various embodiments disclosed herein include, but are not limited to those encoded by SEQ ID NO: 17, 19, 21 or 23. This provides the potential to separate the various component parts of the receptor complex along the polynucleotide, which can enhance expression, stability, and/or functionality of the receptor complex.

### Cytotoxic Signaling Complex

Some embodiments of the compositions and methods described herein relate to a chimeric receptor, such as a chimeric antigen receptor (e.g., a CAR directed to CD19, CD123, CD70, Her2, mesothelin, Claudin 6, BCMA, or EGFR (among others), or a chimeric receptor directed against an NKG2D ligand, such as MICA, MICB, ULBP1, ULBP2, ULBP3, ULBP4, ULBP5, and/or ULBP6) that includes a cytotoxic signaling complex. As disclosed herein, according to several embodiments, the provided cytotoxic receptor complexes comprise one or more transmembrane and/or intracellular domains that initiate cytotoxic signaling cascades upon the extracellular domain(s) binding to ligands on the surface of target cells.

Thus, in some embodiments, the chimeric receptor comprises an extracellular binding domain, a transmembrane domain, and an intracellular signaling domain. In several embodiments, the cytotoxic signaling complex comprises at least one transmembrane domain, at least one co-stimulatory domain, and/or at least one signaling domain. In some embodiments, more than one component part makes up a given domain—e.g., a co-stimulatory domain may comprise two subdomains. In some embodiments, the intracellular signaling domain comprises a primary signaling domain (e.g., CD3zeta) and a co-stimulatory signaling domain. Moreover, in some embodiments, a domain may serve multiple functions, for example, a transmembrane domain may also serve to provide signaling function.

a. Transmembrane Domains

Some embodiments of the compositions and methods described herein relate to chimeric receptors (e.g., tumor antigen-directed CARs and/or ligand-directed chimeric receptors) that comprise a transmembrane domain. Some embodiments include a transmembrane domain from NKG2D or another transmembrane protein. In several embodiments in which a transmembrane domain is employed, the portion of the transmembrane protein employed retains at least a portion of its normal transmembrane domain.

The antigen-binding protein generally is linked to one or more intracellular signaling components, such as signaling components that mimic activation through an antigen receptor complex, such as a TCR complex, in the case of a CAR, and/or signal via another cell surface receptor. Thus, in some embodiments, the antigen-binding component (e.g., antibody) is linked to one or more transmembrane and intracellular signaling domains. In some embodiments, the transmembrane domain is fused to the extracellular domain. In one embodiment, a transmembrane domain that naturally is associated with one of the domains in the receptor, e.g., CAR, is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane regions include those derived from (i.e., comprise at least the transmembrane region(s) of) the alpha, beta or zeta chain of the T-cell receptor, CD28, CD3 epsilon, CD45, CD4, CD5, CDS, CD9, CD 16, CD22, CD33, CD37, CD64, CD80, CD86, CD 134, CD137, CD 154 and/or transmembrane regions containing functional variants thereof such as those retaining a substantial portion of the structural, e.g., transmembrane, properties thereof. In some embodiments, the transmembrane domain is a transmembrane domain derived from CD4, CD28, or CD8, e.g., CD8alpha, or functional variant thereof. Alternatively, the transmembrane domain in some embodiments is synthetic.

In several embodiments, however, the transmembrane domain comprises at least a portion of CD8, a transmembrane glycoprotein normally expressed on both T cells and NK cells. In several embodiments, the transmembrane domain comprises CD8α. In several embodiments, the transmembrane domain comprises a CD8 (e.g., CD8 a) hinge and a CD8 (e.g., CD8 a) transmembrane region. In several embodiments, the transmembrane domain comprises a “hinge”, e.g., a CD8α hinge. In several embodiments, the “hinge” of CD8α has the nucleic acid sequence of SEQ ID NO: 1. In several embodiments, the CD8α hinge is truncated or modified and is at least 70%, at least 75%, at least 80%, at least 85%, at least 90%, at least 95% homologous with the CD8α having the sequence of SEQ ID NO: 1. In several embodiments, the “hinge” of CD8α comprises the amino acid sequence of SEQ ID NO: 2. In several embodiments, the CD8α can be truncated or modified, such that it is at least 70%, at least 75%, at least 80%, at least 85%, at least 90%, at least 95% homologous with the sequence of SEQ ID NO: 2.

In several embodiments, the transmembrane domain comprises a CD8α transmembrane region. In several embodiments, the CD8α transmembrane region is encoded by a nucleic acid sequence of SEQ ID NO: 3. In several embodiments, the CD8α transmembrane region is truncated or modified and is encoded by a nucleic acid sequence that is at least 70%, at least 75%, at least 80%, at least 85%, at least 90%, at least 95% homologous with the CD8α having the sequence of SEQ ID NO: 3. In several embodiments, the CD8α transmembrane region comprises the amino acid sequence of SEQ ID NO: 4. In several embodiments, the CD8α transmembrane region is truncated or modified and is at least 70%, at least 75%, at least 80%, at least 85%, at least 90%, at least 95% homologous with the CD8α having the sequence of SEQ ID NO: 4.

Taken together in several embodiments, the CD8 transmembrane domain is encoded by the nucleic acid sequence of SEQ ID NO: 13. In several embodiments, the CD8 transmembrane domain is truncated or modified and is at least 70%, at least 75%, at least 80%, at least 85%, at least 90%, at least 95% homologous with the CD8 transmembrane domain having the sequence of SEQ ID NO: 13. In several embodiments, the CD8 transmembrane domain comprises the amino acid sequence of SEQ ID NO: 14. In several embodiments, the CD8 transmembrane domain is truncated or modified and is at least 70%, at least 75%, at least 80%, at least 85%, at least 90%, at least 95% homologous with the CD8 transmembrane domain having the sequence of SEQ ID NO: 14.

In some embodiments, the transmembrane domain comprises a CD28 transmembrane domain or a fragment thereof. In several embodiments, the CD28 transmembrane domain comprises the amino acid sequence of SEQ ID NO: 30. In several embodiments, the CD28 transmembrane domain is truncated or modified and is at least 70%, at least 75%, at least 80%, at least 85%, at least 90%, at least 95% homologous with the CD28 transmembrane domain having the sequence of SEQ ID NO: 30.

b. Intracellular Signaling Domains

Some embodiments of the compositions and methods described herein relate to a chimeric receptor (e.g., tumor antigen-directed CARs and/or tumor ligand-directed chimeric receptors) that includes a signaling domain. Among the intracellular signaling domains are those that mimic or approximate a signal through a natural antigen receptor, a signal through such a receptor in combination with a costimulatory receptor, and/or a signal through a costimulatory receptor alone.

The receptor, e.g., the CAR, generally includes at least one intracellular signaling component or components. In some embodiments, the receptor includes an intracellular component of a TCR complex, such as a TCR CD3 chain that mediates T-cell activation and cytotoxicity, e.g., CD3 zeta chain. Thus, in some aspects, the antigen-binding portion is linked to one or more cell signaling modules. In some embodiments, upon ligation of the CAR or other chimeric receptor, the cytoplasmic domain or intracellular signaling domain of the receptor activates at least one of the normal effector functions or responses of the immune cell, e.g., T cell engineered to express the CAR. For example, in some contexts, the CAR induces a function of a T cell such as cytolytic activity or T-helper activity, such as secretion of cytokines or other factors. In some embodiments, a truncated portion of an intracellular signaling domain of an antigen receptor component or costimulatory molecule is used in place of an intact immuno stimulatory chain, for example, if it transduces the effector function signal. In some embodiments, the intracellular signaling domain or domains include the cytoplasmic sequences of the T cell receptor (TCR), and in some aspects also those of co-receptors that in the natural context act in concert with such receptors to initiate signal transduction following antigen receptor engagement.

In the context of a natural TCR, full activation generally requires not only signaling through the TCR, but also a costimulatory signal. Thus, in some embodiments, to promote full activation, a component for generating secondary or co-stimulatory signal is also included in the receptor.

T cell activation is in some aspects described as being mediated by two classes of cytoplasmic signaling sequences: those that initiate antigen-dependent primary activation through the TCR (primary cytoplasmic signaling sequences), and those that act in an antigen-independent manner to provide a secondary or co-stimulatory signal (secondary cytoplasmic signaling sequences). In some aspects, the receptor includes one or both of such signaling components.

In some aspects, the receptor includes a primary cytoplasmic signaling sequence that regulates primary activation of the TCR complex. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences include those derived from TCR zeta, FcR gamma, FcR beta, CD3 gamma, CD3 delta, CD3 epsilon, CDS, CD22, CD79a, CD79b, and CD66d. In some embodiments, cytoplasmic signaling molecule(s) in the CAR contain(s) a cytoplasmic signaling domain, portion thereof, or sequence derived from CD3 zeta.

For example, immune cells engineered according to several embodiments disclosed herein may comprise at least one subunit of the CD3 T cell receptor complex (or a fragment thereof). In several embodiments, the signaling domain comprises the CD3 zeta subunit. In several embodiments, the CD3 zeta is encoded by the nucleic acid sequence of SEQ ID NO: 7. In several embodiments, the CD3 zeta can be truncated or modified, such that it is at least 70%, at least 75%, at least 80%, at least 85%, at least 90%, at least 95% homologous with the CD3 zeta having the sequence of SEQ ID NO: 7. In several embodiments, the CD3 zeta domain comprises the amino acid sequence of SEQ ID NO: 8. In several embodiments, the CD3 zeta domain is truncated or modified and is at least 70%, at least 75%, at least 80%, at least 85%, at least 90%, at least 95% homologous with the CD3 zeta domain having the sequence of SEQ ID NO: 8.

In some embodiments, the receptor includes a signaling domain and/or transmembrane portion of a costimulatory receptor, such as CD28, 4-1BB, OX40, DAP10, and ICOS. In some aspects, the same receptor includes both the activating and costimulatory components. In some embodiments, the intracellular signaling component of the recombinant receptor, such as CAR, comprises a CD3 zeta intracellular domain and a costimulatory signaling region. In some embodiments, the intracellular signaling domain comprises a chimeric OX40 co-stimulatory domain linked to a CD3 zeta intracellular domain.

In several embodiments, unexpectedly enhanced signaling is achieved through the use of multiple signaling domains whose activities act synergistically. For example, in several embodiments, the signaling domain further comprises an OX40 domain. In several embodiments, the OX40 domain is an intracellular signaling domain. In several embodiments, the OX40 intracellular signaling domain has the nucleic acid sequence of SEQ ID NO: 5. In several embodiments, the OX40 intracellular signaling domain can be truncated or modified, such that it is at least 70%, at least 75%, at least 80%, at least 85%, at least 90%, at least 95% homologous with the OX40 having the sequence of SEQ ID NO: 5. In several embodiments, the OX40 intracellular signaling domain comprises the amino acid sequence of SEQ ID NO: 6. In several embodiments, the OX40 intracellular signaling domain is truncated or modified and is at least 70%, at least 75%, at least 80%, at least 85%, at least 90%, at least 95% homologous with the OX40 intracellular signaling domain having the sequence of SEQ ID NO: 6. In several embodiments, OX40 is used as the sole transmembrane/signaling domain in the construct, however, in several embodiments, OX40 can be used with one or more other domains. For example, combinations of OX40 and CD3zeta are used in some embodiments. By way of further example, combinations of CD28, OX40, 4-1BB, and/or CD3zeta are used in some embodiments.

In several embodiments, the signaling domain comprises a 4-1BB domain. In several embodiments, the 4-1BB domain is an intracellular signaling domain. In several embodiments, the 4-1BB intracellular signaling domain is encoded by the nucleic acid sequence of SEQ ID NO: 29. In several embodiments, the 4-1BB intracellular signaling domain is truncated or modified and is at least 70%, at least 75%, at least 80%, at least 85%, at least 90%, at least 95% homologous with the 4-1BB intracellular signaling domain having the sequence of SEQ ID NO: 29. In several embodiments, 4-1BB is used as the sole transmembrane/signaling domain in the construct, however, in several embodiments, 4-1BB can be used with one or more other domains. For example, combinations of 4-1BB and CD3zeta are used in some embodiments. By way of further example, combinations of CD28, OX40, 4-1BB, and/or CD3zeta are used in some embodiments.

In several embodiments, the signaling domain comprises a CD28 domain. In several embodiments the CD28 domain is an intracellular signaling domain. In several embodiments, the CD28 intracellular signaling domain comprises the amino acid sequence of SEQ ID NO: 31. In several embodiments, the CD28 intracellular signaling domain is truncated or modified and is at least 70%, at least 75%, at least 80%, at least 85%, at least 90%, at least 95% homologous with the CD28 intracellular signaling domain having the sequence of SEQ ID NO: 31. In several embodiments, CD28 is used as the sole transmembrane/signaling domain in the construct, however, in several embodiments, CD28 can be used with one or more other domains. For example, combinations of CD28 and CD3zeta are used in some embodiments. By way of further example, combinations of CD28, OX40, 4-1BB, and/or CD3zeta are used in some embodiments.

In any of the provided embodiments, the nucleic acid encoding the chimeric receptor, or a portion thereof, is codon-optimized. In some embodiments, the polynucleotides are optimized, or contain certain features designed for optimization, such as for codon usage, to reduce RNA heterogeneity and/or to modify, e.g., increase or render more consistent among cell product lots, expression, such as surface expression, of the encoded receptor. In some embodiments, polynucleotides, encoding chimeric receptors, are modified as compared to a reference polynucleotide, such as to remove cryptic or hidden splice sites, to reduce RNA heterogeneity. In some embodiments, polynucleotides, encoding chimeric receptors, are codon optimized, such as for expression in a mammalian, e.g., human, cell such as in a human T cell. In some aspects, the modified polynucleotides result in in improved, e.g., increased or more uniform or more consistent level of, expression, e.g., surface expression, when expressed in a cell.

### Stimulatory Molecules

In some embodiments, the intracellular signaling domain of a chimeric receptor provided herein comprises a co-stimulatory signaling domain, including any of those as described in the preceding section. Thus, some embodiments of the compositions and methods described herein relate to chimeric receptors (e.g., tumor antigen-directed CARs and/or tumor ligand-directed chimeric receptors) that comprise a co-stimulatory domain. In addition to the various transmembrane domains and signaling domain (and the combination transmembrane/signaling domains), additional co-activating molecules (“stimulatory molecules”) can be provided, in several embodiments. These can be certain molecules that, for example, further enhance activity of the immune cells. Cytokines may be used in some embodiments. For example, certain interleukins, such as IL-2 and/or IL-15 as non-limiting examples, are used. In some embodiments, the immune cells for therapy are engineered to express such molecules as a secreted form. In additional embodiments, such stimulatory molecules are engineered to be membrane bound, acting as autocrine stimulatory molecules (or even as paracrine stimulators to neighboring cells).

In several embodiments, NK cells are engineered to express membrane-bound interleukin 15 (mbIL15). In such embodiments, mbIL15 expression on the NK enhances the cytotoxic effects of the engineered NK cell by enhancing the proliferation and/or longevity of the NK cells. In some embodiments, the IL15 is expressed from a separate cassette on the construct comprising any one of the CARs disclosed herein. In some embodiments, the IL15 is expressed from the same cassette as any one of the CARs disclosed herein. In some embodiments, the chimeric receptor and IL15 are separated by a nucleic acid sequence encoding a cleavage site, for example, a proteolytic cleavage site or a T2A, β2A, E2A, or F2A self-cleaving peptide cleavage site. In some embodiments, the chimeric receptor and IL15 are separated by a T2A sequence. In some embodiments, the IL15 is a membrane-bound IL15 (mbIL15). In several embodiments, T cells, such as the genetically engineered non-alloreactive T cells disclosed herein are engineered to express membrane-bound interleukin 15 (mbIL15). In such embodiments, mbIL15 expression on the T cell enhances the cytotoxic effects of the engineered T cell by enhancing the activity and/or propagation (e.g., longevity) of the engineered T cells. In some embodiments, the mbIL15 comprises a native IL15 sequence, such as a human native IL15 sequence, and at least one transmembrane domain. In several embodiments, IL15 is encoded by the nucleic acid sequence of SEQ ID NO: 11. In several embodiments, IL15 can be truncated or modified, such that it encoded by a nucleic acid sequence that is at least 70%, at least 75%, at least 80%, at least 85%, at least 90%, at least 95% homologous with the sequence of SEQ ID NO: 11. In several embodiments, the IL15 comprises the amino acid sequence of SEQ ID NO: 12. In several embodiments, the IL15 is truncated or modified and is at least 70%, at least 75%, at least 80%, at least 85%, at least 90%, at least 95% homologous with the IL15 having the sequence of SEQ ID NO: 12.

In some embodiments, the mbIL15 is membrane-bound by virtue of the fusion of IL15 to a transmembrane domain. Thus, in some embodiments, mbIL15 comprises a transmembrane domain. In some embodiments, the transmembrane domain comprises a CD8 transmembrane domain. In some embodiments, the mbIL15 may comprise additional components, such as a leader sequence and/or a hinge sequence. In some embodiments, the leader sequence is a CD8 leader sequence. In some embodiments, the hinge sequence is a CD8 hinge sequence. In some embodiments, the mbIL15 comprises a native IL15 sequence, such as a human native IL15 sequence, and at least one transmembrane domain. In several embodiments, mbIL15 is encoded by the nucleic acid sequence of SEQ ID NO: 489. In several embodiments, mbIL15 can be truncated or modified, such that it encoded by a nucleic acid sequence that is at least 70%, at least 75%, at least 80%, at least 85%, at least 90%, at least 95% homologous with the sequence of SEQ ID NO: 489. In several embodiments, the mbIL15 comprises the amino acid sequence of SEQ ID NO: 490. In several embodiments, the mbIL15 is truncated or modified and is at least 70%, at least 75%, at least 80%, at least 85%, at least 90%, at least 95% homologous with the mbIL15 having the sequence of SEQ ID NO: 490. It is contemplated that any of the amino acid sequences provided herein may be provided with or without a signal sequence (e.g., a CD8α signal sequence, such as MALPVTALLLPLALLLHAARP).

Membrane-bound IL15 sequences are described in PCT publications WO 2018/183385 and WO 2020/056045, each of which is hereby expressly incorporated by reference in its entirety.

In some embodiments, the tumor antigen-directed CARs and/or tumor ligand-directed chimeric receptors are encoded by a polynucleotide that includes one or more cytosolic protease cleavage sites, for example a T2A cleavage site, a P2A cleavage site, an E2A cleavage site, and/or a F2A cleavage site. Such sites are recognized and cleaved by a cytosolic protease, which can result in separation (and separate expression) of the various component parts of the receptor encoded by the polynucleotide. As a result, depending on the embodiment, the various constituent parts of an engineered cytotoxic receptor complex can be delivered to an NK cell or T cell in a single vector or by multiple vectors. Thus, as shown schematically, in the Figures, a construct can be encoded by a single polynucleotide, but also include a cleavage site, such that downstream elements of the constructs are expressed by the cells as a separate protein (as is the case in some embodiments with IL-15). In several embodiments, a T2A cleavage site is used. In several embodiments, a T2A cleavage site is encoded by the nucleic acid sequence of SEQ ID NO: 9. In several embodiments, T2A cleavage site can be truncated or modified, such that it is encoded by a nucleic acid sequence that is at least 70%, at least 75%, at least 80%, at least 85%, at least 90%, at least 95% homologous with the sequence of SEQ ID NO: 9. In several embodiments, the T2A cleavage site comprises the amino acid sequence of SEQ ID NO: 10. In several embodiments, the T2A cleavage site is truncated or modified. In several embodiments, the T2A cleavage site is truncated or modified and is at least 70%, at least 75%, at least 80%, at least 85%, at least 90%, at least 95% homologous with the T2A cleavage site having the sequence of SEQ ID NO: 10.

### Cytotoxic Receptor Complex Constructs

Some embodiments of the compositions and methods described herein relate to chimeric antigen receptors, such as a CD19-directed chimeric receptor, as well as chimeric receptors, such as an activating chimeric receptor (ACR) that targets a ligand of NKG2D. Unless otherwise indicated, a cytotoxic receptor complex shall refer either to a CAR or an ACR (an engineered complex as opposed to an endogenous (e.g., naturally occurring) cytotoxic receptor). The expression of these cytotoxic receptor complexes in immune cells, such as genetically modified non-alloreactive T cells and/or NK cells, allows the targeting and destruction of particular target cells, such as cancerous cells. Non-limiting examples of such cytotoxic receptor complexes are discussed in more detail below.

### Chimeric Antigen Receptor Cytotoxic Receptor Complex Constructs

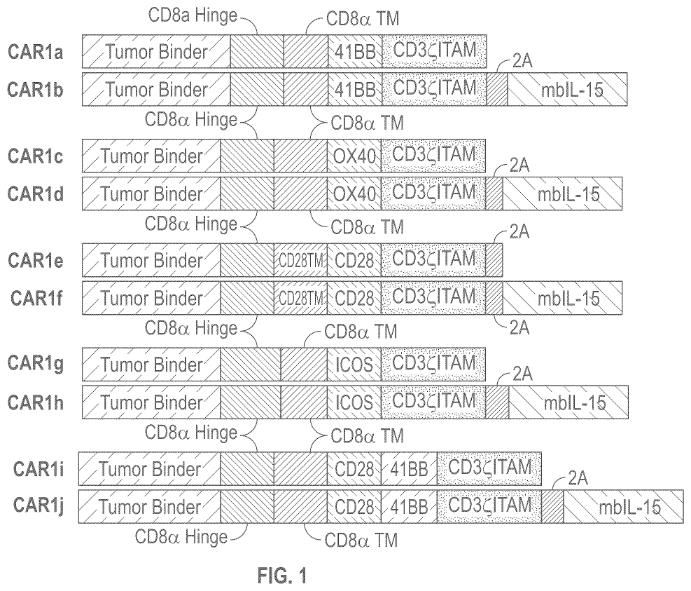

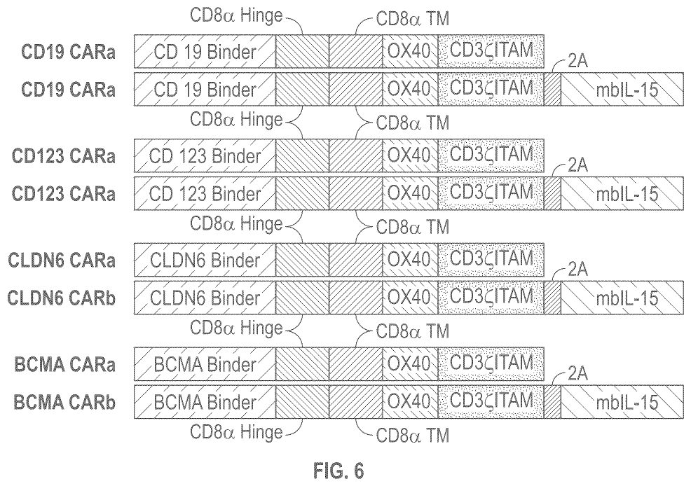

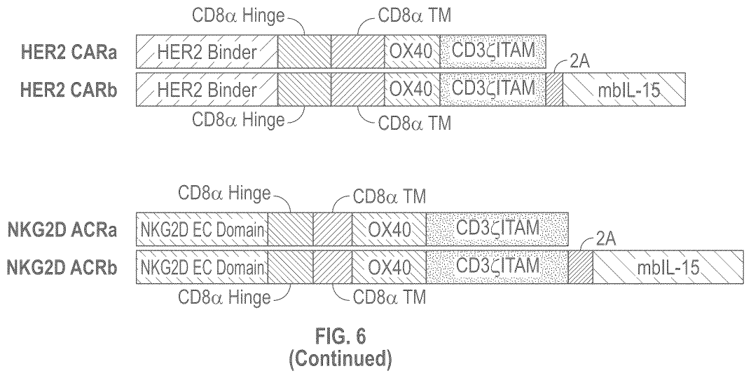

In several embodiments, there are provided for herein a variety of cytotoxic receptor complexes (also referred to as cytotoxic receptors) are provided for herein with the general structure of a chimeric antigen receptor. FIGS. 1-7 schematically depict non-limiting schematics of constructs that include a tumor binding moiety that binds to a tumor antigen or tumor-associated antigen expressed on the surface of cancer cells and activates the engineered cell expressing the chimeric antigen receptor. FIG. 6 shows a schematic of a chimeric receptor complex, with an NKG2D activating chimeric receptor as a non-limiting example (see NKG2D ACRa and ACRb). FIG. 6 shows a schematic of a bispecific CAR/chimeric receptor complex, with an NKG2D activating chimeric receptor as a non-limiting example (see Bi-spec CAR/ACRa and CAR/ACRb).

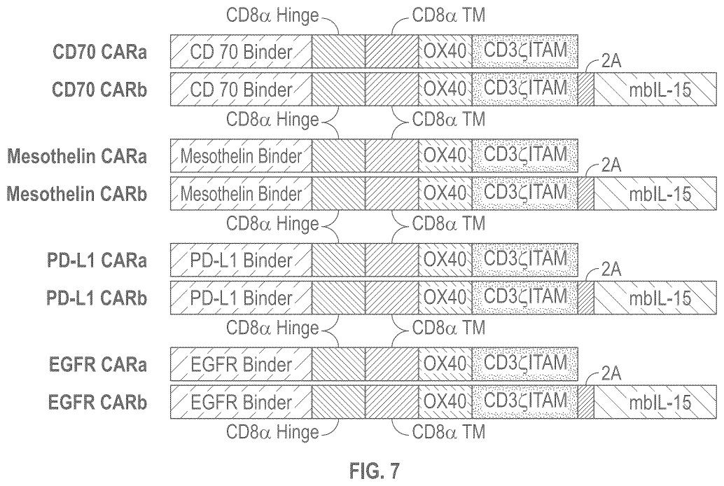

As shown in the figures, several embodiments of the chimeric receptor include an anti-tumor binder, a CD8α hinge domain, an Ig4 SH domain (or hinge), a CD8α transmembrane domain, a CD28 transmembrane domain, an OX40 domain, a 4-1BB domain, a CD28 domain, a CD3ζ ITAM domain or subdomain, a CD3zeta domain, an NKp80 domain, a CD16 IC domain, a 2A cleavage site, and a membrane-bound IL-15 domain (though, as above, in several embodiments soluble IL-15 is used). In several embodiments, the binding and activation functions are engineered to be performed by separate domains. Several embodiments relate to complexes with more than one tumor binder moiety or other binder/activation moiety. In some embodiments, the binder/activation moiety targets other markers besides CD19, such as a cancer target described herein. For example, FIGS. 6 and 7 depict schematics of non-limiting examples of CAR constructs that target different antigens, such as CD123, CLDN6, BCMA, HER2, CD70, Mesothelia, PD-L1, and EGFR. In several embodiments, the general structure of the chimeric antigen receptor construct includes a hinge and/or transmembrane domain. These may, in some embodiments, be fulfilled by a single domain, or a plurality of subdomains may be used, in several embodiments. The receptor complex further comprises a signaling domain, which transduces signals after binding of the homing moiety to the target cell, ultimately leading to the cytotoxic effects on the target cell. In several embodiments, the complex further comprises a co-stimulatory domain, which operates, synergistically, in several embodiments, to enhance the function of the signaling domain. Expression of these complexes in immune cells, such as T cells and/or NK cells, allows the targeting and destruction of particular target cells, such as cancerous cells that express a given tumor marker. Some such receptor complexes comprise an extracellular domain comprising an anti-CD19 moiety, or CD19-binding moiety, that binds CD19 on the surface of target cells and activates the engineered cell. The CD3zeta ITAM subdomain may act in concert as a signaling domain. The IL-15 domain, e.g., mbIL-15 domain, may act as a co-stimulatory domain. The IL-15 domain, e.g. mbIL-15 domain, may render immune cells (e.g., NK or T cells) expressing it particularly efficacious against target tumor cells. It shall be appreciated that the IL-15 domain, such as an mbIL-15 domain, can, in accordance with several embodiments, be encoded on a separate construct. Additionally, each of the components may be encoded in one or more separate constructs. In some embodiments, the cytotoxic receptor or CD19-directed receptor comprises a sequence of amino acids that is at least 70%, 75%, 80%, 85%, 90%, 91%, 92%, 93%, 94%, 95%, 96%, 97%, 98%, 99%, or more, or a range defined by any two of the aforementioned percentages, identical to the sequence of SEQ ID NO: 34. In some embodiments, the cytotoxic receptor or CD19-directed receptor comprises the amino acid sequence set forth in SEQ ID NO:34.

Depending on the embodiment, various binders can be used to target CD19. In several embodiments, peptide binders are used, while in some embodiments antibodies, or fragments thereof are used. In several embodiments employing antibodies, antibody sequences are optimized, humanized or otherwise manipulated or mutated from their native form in order to increase one or more of stability, affinity, avidity or other characteristic of the antibody or fragment. In several embodiments, an antibody is provided that is specific for CD19. In several embodiments, an scFv is provided that is specific for CD19. In several embodiments, the antibody or scFv specific for CD19 comprises a heavy chain variable comprising the amino acid sequence of SEQ ID NO: 104 or 106. In some embodiments, the heavy chain variable comprises a sequence of amino acids that is at least 70%, 75%, 80%, 85%, 90%, 91%, 92%, 93%, 94%, 95%, 96%, 97%, 98%, 99%, or more, identical to the sequence of SEQ ID NO. 104 or 106. In some embodiments, the heavy chain variable comprises a sequence of amino acids that is encoded by a polynucleotide that hybridizes under moderately stringent conditions to the complement of a polynucleotide that encodes a heavy chain variable of SEQ ID NO. 104 or 106. In some embodiments, the heavy chain variable domain a sequence of amino acids that is encoded by a polynucleotide that hybridizes under stringent conditions to the complement of a polynucleotide that encodes a heavy chain variable encodes a heavy chain variable of SEQ ID NO. 104 or 106. In several embodiments, the heavy chain variable region comprises the CDR-H1, the CDRH-2, and the CDRH-3 as comprised within the amino acid sequence set forth in SEQ ID NO:104. In several embodiments, the heavy chain variable region comprises the CDR-H1, the CDRH-2, and the CDRH-3 as comprised within the amino acid sequence set forth in SEQ ID NO:106.

In several embodiments, the antibody or scFv specific for CD19 comprises a light chain variable comprising the amino acid sequence of any of SEQ ID NO. 105 or 107. In several embodiments, the light chain variable comprises a sequence of amino acids that is encoded by a nucleotide sequence that is at least 70%, 75%, 80%, 85%, 90%, 91%, 92%, 93%, 94%, 95%, 96%, 97%, 98%, 99%, or more, identical to the identical to the sequence of SEQ ID NO. 105 or 107. In some embodiments, the light chain variable comprises a sequence of amino acids that is encoded by a polynucleotide that hybridizes under moderately stringent conditions to the complement of a polynucleotide that encodes a light chain variable of SEQ ID NO. 105 or 107. In some embodiments, the light chain variable domain comprises a sequence of amino acids that is encoded by a polynucleotide that hybridizes under stringent conditions to the complement of a polynucleotide that encodes a light chain variable domain of SEQ ID NO. 105 or 107. In several embodiments, the light chain variable region comprises the CDR-L1, the CDRL-2, and the CDRL-3 as comprised within the amino acid sequence set forth in SEQ ID NO:105. In several embodiments, the lightchain variable region comprises the CDR-L1, the CDRL-2, and the CDRL-3 as comprised within the amino acid sequence set forth in SEQ ID NO:107.

In several embodiments, the anti-CD19 antibody or scFv comprises one, two, or three heavy chain complementarity determining region (CDR) and one, two, or three light chain CDRs. In several embodiments, a first heavy chain CDR has the amino acid sequence of SEQ ID NO: 111. In some embodiments, the first heavy chain CDR comprises a sequence of amino acids that is at least 70%, 75%, 80%, 85%, 90%, 91%, 92%, 93%, 94%, 95%, 96%, 97%, 98%, 99%, or more, identical to the sequence of SEQ ID NO. 111. In several embodiments, a second heavy chain CDR has the amino acid sequence of SEQ ID NO: 112, 113, or 114. In some embodiments, the second heavy chain CDR comprises a sequence of amino acids that is at least 70%, 75%, 80%, 85%, 90%, 91%, 92%, 93%, 94%, 95%, 96%, 97%, 98%, 99%, or more, identical to the sequence of SEQ ID NO. 112, 113, or 114. In several embodiments, a third heavy chain CDR has the amino acid sequence of SEQ ID NO: 115. In some embodiments, the third heavy chain CDR comprises a sequence of amino acids that is at least 70%, 75%, 80%, 85%, 90%, 91%, 92%, 93%, 94%, 95%, 96%, 97%, 98%, 99%, or more, identical to the sequence of SEQ ID NO. 115.

In several embodiments, a first light chain CDR has the amino acid sequence of SEQ ID NO: 108. In some embodiments, the first light chain CDR comprises a sequence of amino acids that is at least 70%, 75%, 80%, 85%, 90%, 91%, 92%, 93%, 94%, 95%, 96%, 97%, 98%, 99%, or more, identical to the sequence of SEQ ID NO. 108. In several embodiments, a second light chain CDR has the amino acid sequence of SEQ ID NO: 109. In some embodiments, the second light chain CDR comprises a sequence of amino acids that is at least 70%, 75%, 80%, 85%, 90%, 91%, 92%, 93%, 94%, 95%, 96%, 97%, 98%, 99%, or more, identical to the sequence of SEQ ID NO. 109. In several embodiments, a third light chain CDR has the amino acid sequence of SEQ ID NO: 110. In some embodiments, the third light chain CDR comprises a sequence of amino acids that is at least 70%, 75%, 80%, 85%, 90%, 91%, 92%, 93%, 94%, 95%, 96%, 97%, 98%, 99% or more, identical to the sequence of SEQ ID NO. 110.

In several embodiments, there is provided an anti-CD19 CAR comprising the amino acid sequence of SEQ ID NO. 116. In some embodiments, there is provided an anti-CD19 CAR comprising a sequence of amino acids that is at least 70%, 75%, 80%, 85%, 90%, 91%, 92%, 93%, 94%, 95%, 96%, 97%, 98%, 99% or more, identical to the sequence of SEQ ID NO. 116.

In one embodiment, there is provided a polynucleotide encoding a Tumor Binder/CD8hinge-CD8TM/OX40/CD3zeta chimeric antigen receptor complex (see FIG. 1, CAR1c). The polynucleotide comprises or is composed of tumor binder, a CD8α hinge, a CD8α transmembrane domain, an OX40 domain, and a CD3zeta domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a tumor binder/CD8hinge-CD8TM/OX40/CD3zeta/2A/mIL-15 chimeric antigen receptor complex (see FIG. 1, CAR Id). The polynucleotide comprises or is composed of a Tumor Binder, a CD8α hinge, a CD8α transmembrane domain, an OX40 domain, a CD3zeta domain, a 2A cleavage site, and an mIL-15 domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site).

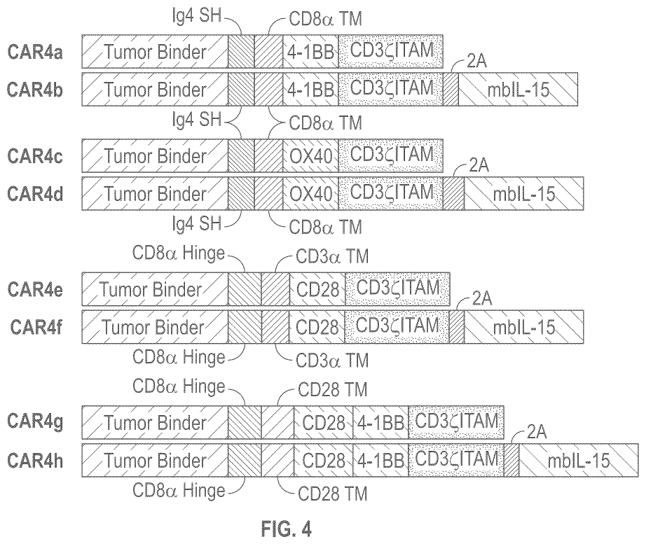

In several embodiments, there is provided a polynucleotide encoding a Tumor Binder/Ig4SH-CD8TM/4-1BB/CD3zeta chimeric antigen receptor complex (see FIG. 4, CAR4a). The polynucleotide comprises or is composed of a Tumor Binder, an Ig4 SH domain, a CD8α transmembrane domain, a 4-1BB domain, and a CD3zeta domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/Ig4SH-CD8TM/4-1BB/CD3zeta/2A/mIL-15 chimeric antigen receptor complex (see FIG. 4, CAR4b). The polynucleotide comprises or is composed of a Tumor Binder, a Ig4 SH domain, a CD8a transmembrane domain, a 4-1BB domain, a CD3zeta domain, a 2A cleavage site, and an mIL-15 domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/CD8hinge-CD28TM/CD28/CD3zeta chimeric antigen receptor complex (see FIG. 1, CAR1e). The polynucleotide comprises or is composed of a Tumor Binder, a CD8a hinge, a CD28 transmembrane domain, a CD28 domain, and a CD3zeta domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/CD8hinge-CD28TM/CD28/CD3zeta/2A/mIL-15 chimeric antigen receptor complex (see FIG. 1, CAR1f). The polynucleotide comprises or is composed of a Tumor Binder, a CD8a hinge, a CD28 transmembrane domain, a CD28 domain, a CD3zeta domain, a 2A cleavage site, and an mIL-15 domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site).

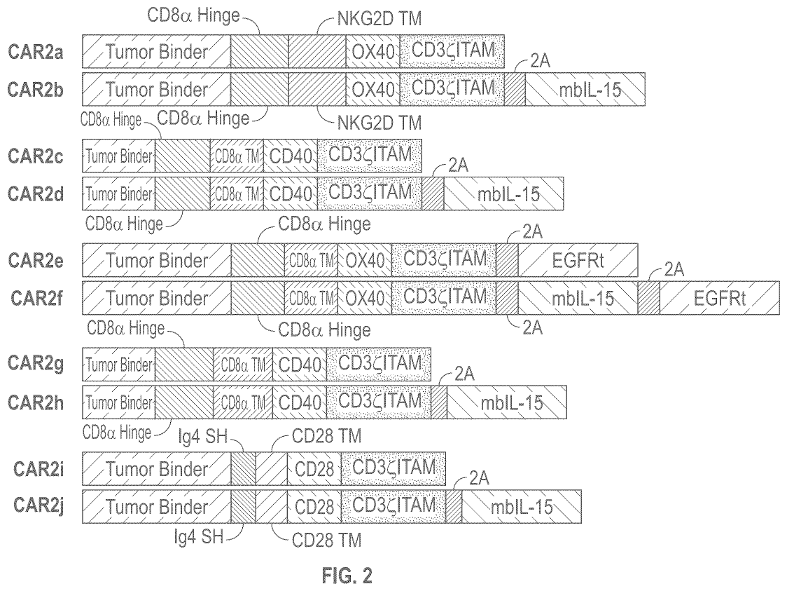

In several embodiments, there is provided a polynucleotide encoding a Tumor Binder/Ig4SH-CD28TM/CD28/CD3zeta chimeric antigen receptor complex (see FIG. 2, CAR2i). The polynucleotide comprises or is composed of a Tumor Binder, an Ig4 SH domain, a CD28 transmembrane domain, a CD28 domain, and a CD3zeta domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/Ig4SH-CD28TM/CD28/CD3zeta/2A/mIL-15 chimeric antigen receptor complex (see FIG. 2, CAR2j). The polynucleotide comprises or is composed of a Tumor Binder, an Ig4 SH domain, a CD28 transmembrane domain, a CD28 domain, a CD3zeta domain, a 2A cleavage site, and an mIL-15 domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/Ig4SH-CD8TM/OX40/CD3zeta chimeric antigen receptor complex (see FIG. 4, CAR4c). The polynucleotide comprises or is composed of a Tumor Binder, a Ig4 SH domain, a CD8a transmembrane domain, an OX40 domain, and a CD3zeta domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/Ig4SH-CD8TM/OX40/CD3zeta/2A/mIL-15 chimeric antigen receptor complex (see FIG. 4, CAR4d). The polynucleotide comprises or is composed of a Tumor Binder, a Ig4 SH domain, a CD8a transmembrane domain, an OX40 domain, a CD3zeta domain, a 2A cleavage site, and an mIL-15 domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/CD8hinge-CD3αTM/CD28/CD3zeta chimeric antigen receptor complex (see FIG. 4, CAR4e). The polynucleotide comprises or is composed of a Tumor Binder, a CD8a hinge, a CD3α transmembrane domain, a CD28 domain, and a CD3zeta domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/CD8hinge-CD3αTM/CD28/CD3zeta/2A/mIL-15 chimeric antigen receptor complex (see FIG. 4, CAR4f). The polynucleotide comprises or is composed of a Tumor Binder, a CD8a hinge, a CD3α transmembrane domain, a CD28 domain, a CD3zeta domain, a 2A cleavage site, and an mIL-15 domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/CD8hinge-CD28TM/CD28/4-1BB/CD3zeta chimeric antigen receptor complex (see FIG. 4, CAR 4g). The polynucleotide comprises or is composed of a Tumor Binder, a CD8a hinge, a CD28 transmembrane domain, a CD28 domain, a 4-1BB domain, and a CD3zeta domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/CD8hinge-CD28TM/CD28/4-1BB/CD3zeta/2A/mIL-15 chimeric antigen receptor complex (see FIG. 4, CAR 4h). The polynucleotide comprises or is composed of a Tumor Binder, a CD8a hinge, a CD28 transmembrane domain, a CD28 domain, a 4-1BB domain, a CD3zeta domain, a 2A cleavage site, and an mIL-15 domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site).

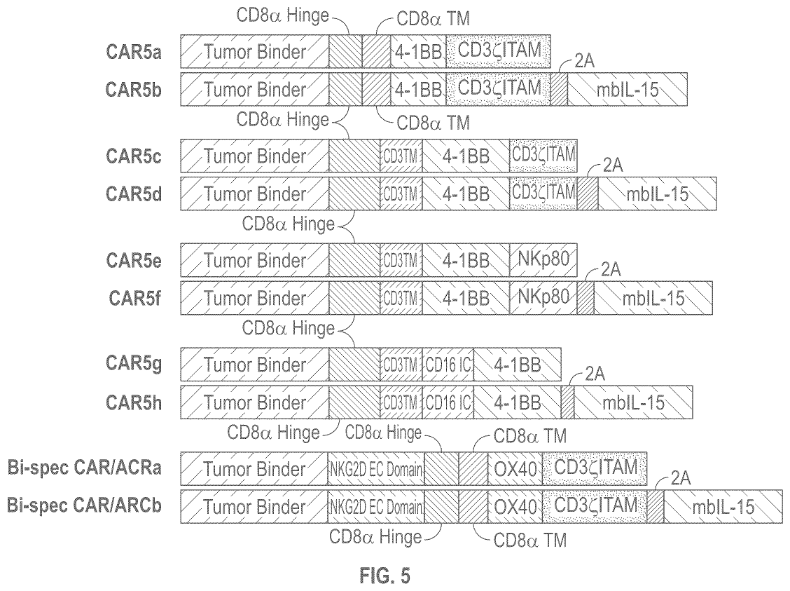

In several embodiments, there is provided a polynucleotide encoding a Tumor Binder/CD8 alpha hinge/CD8 alpha TM/4-1BB/CD3zeta chimeric antigen receptor complex (see FIG. 5, CAR5a). The polynucleotide comprises or is composed of an anti-CD19 moiety, a CD8a hinge, a CD8a transmembrane domain, a 4-1BB domain, and a CD3zeta domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/CD8 alpha hinge/CD8 alpha TM/4-1BB/CD3zeta/2A/mIL-15 chimeric antigen receptor complex (see FIG. 5, CAR 5b). The polynucleotide comprises or is composed of a Tumor Binder, a CD8a hinge, a CD8a transmembrane domain, a 4-1BB domain, a CD3zeta domain, a 2A cleavage site, and an mIL-15 domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/CD8 alpha hinge/CD3 TM/4-1BB/CD3zeta chimeric antigen receptor complex (see FIG. 5, CAR5c). The polynucleotide comprises or is composed of a Tumor Binder, a CD8a hinge, a CD3 transmembrane domain, a 4-1BB domain, and a CD3zeta domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/CD8 alpha hinge/CD3 TM/4-1BB/CD3zeta/2A/mIL-15 chimeric antigen receptor complex (see FIG. 5, CAR5d). The polynucleotide comprises or is composed of a Tumor Binder, a CD8a hinge, a CD8a transmembrane domain, a 4-1BB domain, a CD3zeta domain, a 2A cleavage site, and an mIL-15 domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/CD8 alpha hinge/CD3 TM/4-1BB/NKp80 chimeric antigen receptor complex (see FIG. 5, CAR5e). The polynucleotide comprises or is composed of a Tumor Binder, a CD8a hinge, a CD3 transmembrane domain, a 4-1BB domain, and an NKp80 domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/CD8 alpha hinge/CD3 TM/4-1BB/NKp80/2A/mIL-15 chimeric antigen receptor complex (see FIG. 5, CAR5f). The polynucleotide comprises or is composed of a Tumor Binder, a CD8a hinge, a CD8a transmembrane domain, a 4-1BB domain, an NKp80 domain, a 2A cleavage site, and an mIL-15 domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/CD8 alpha hinge/CD3 TM/CD16 intracellular domain/4-1BB chimeric antigen receptor complex (see FIG. 5, CAR5g). The polynucleotide comprises or is composed of a Tumor Binder, a CD8a hinge, a CD3 transmembrane domain, CD16 intracellular domain, and a 4-1BB domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/CD8 alpha hinge/CD3 TM/CD16/4-1BB/2A/mIL-15 chimeric antigen receptor complex (see FIG. 5, CAR5h). The polynucleotide comprises or is composed of a Tumor Binder, a CD8a hinge, a CD8a transmembrane domain, a CD16 intracellular domain, a 4-1BB domain, a 2A cleavage site, and an mIL-15 domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/NKG2D Extracellular Domain/CD8hinge-CD8TM/OX40/CD3zeta chimeric antigen receptor complex (see FIG. 5, Bi-spec CAR/ACRa). The polynucleotide comprises or is composed of a Tumor Binder, an NKG2D extracellular domain (either full length or a fragment), a CD8a hinge, a CD8a transmembrane domain, an OX40 domain, and a CD3zeta domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/NKG2D EC Domain/CD8hinge-CD8TM/OX40/CD3zeta/2A/mIL-15 chimeric antigen receptor complex (see FIG. 5, Bi-spec CAR/ACRb). The polynucleotide comprises or is composed of a Tumor Binder, an NKG2D extracellular domain (either full length or a fragment), a CD8a hinge, a CD8a transmembrane domain, an OX40 domain, a CD3zeta domain, a 2A cleavage site, and an mIL-15 domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/CD8hinge/CD8TM/4-1BB/CD3zeta chimeric antigen receptor complex (see FIG. 1, CAR1a). The polynucleotide comprises or is composed of a Tumor Binder, a CD8a hinge, a CD8a transmembrane domain, a 4-1BB domain, and a CD3zeta domain. By way of non-limiting embodiment, there is provided herein an anti-CD19/CD8hinge/CD8TM/4-1BB/CD3zeta chimeric antigen receptor complex. In several embodiments, this receptor complex is encoded by a nucleic acid molecule having the sequence of SEQ ID NO: 85. In several embodiments, a nucleic acid sequence encoding an CAR1a chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 85. In several embodiments, the chimeric receptor comprises the amino acid sequence of SEQ ID NO: 86. In several embodiments, a CAR1a chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 86. It shall be appreciated that certain sequence variability, extensions, and/or truncations of the disclosed sequences may result when combining sequences, as a result of, for example, ease or efficiency in cloning (e.g., for creation of a restriction site). In several embodiments, there is provided an CAR1a construct that further comprises mbIL15, as disclosed herein (see e.g., FIG. 1 CAR1b).

In several embodiments, there is provided a polynucleotide encoding a Tumor Binder/CD8hinge/CD8TM/OX40/CD3zeta chimeric antigen receptor complex (see FIG. 1, CAR1c). The polynucleotide comprises or is composed of a Tumor Binder, a CD8a hinge, a CD8a transmembrane domain, an OX40 domain, and a CD3zeta domain. In several embodiments, the chimeric antigen receptor further comprises mbIL15 (see FIG. 1, CAR1d). By way of non-limiting embodiment, there is provided herein an anti CD19/CD8hinge/CD8TM/OX40/CD3zeta/2A/mIL-15 chimeric antigen receptor. In such embodiments, the polynucleotide comprises or is composed of an anti-CD19 scFv, a CD8a hinge, a CD8a transmembrane domain, an OX40 domain, a CD3zeta domain, a 2A cleavage site, and an mbIL-15 domain as described herein. In several embodiments, this receptor complex is encoded by a nucleic acid molecule having the sequence of SEQ ID NO: 59. In several embodiments, a nucleic acid sequence encoding an CAR1d chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 59. In several embodiments, the chimeric receptor comprises the amino acid sequence of SEQ ID NO: 60. In several embodiments, a NK19 chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 60. In several embodiments, the CD19 scFv does not comprise a Flag tag. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/CD8hinge/CD28TM/CD28/CD3zeta chimeric antigen receptor complex (see FIG. 1, CAR1e). The polynucleotide comprises or is composed of a Tumor Binder, a CD8a hinge, a CD28 transmembrane domain, CD28 signaling domain, and a CD3zeta domain. In several embodiments, the chimeric antigen receptor further comprises mbIL15 (see FIG. 1, CAR1d). By way of non-limiting embodiment, there is provided herein an anti-CD19moiety/CD8hinge/CD28TM/CD28/CD3zeta/2A/mIL15 chimeric antigen receptor complex. In such embodiments, the polynucleotide comprises or is composed of an anti-CD19 scFv, a CD8a hinge, a CD28 transmembrane domain, CD28 signaling domain, a CD3zeta domain a 2A cleavage site, and an mbIL-15 domain as described herein. In several embodiments, this receptor complex is encoded by a nucleic acid molecule having the sequence of SEQ ID NO: 61. In several embodiments, a nucleic acid sequence encoding an CAR1d chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 61. In several embodiments, the chimeric receptor comprises the amino acid sequence of SEQ ID NO: 62. In several embodiments, a CAR1d chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 62. In several embodiments, the CD19 scFv does not comprise a Flag tag. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/CD8hinge/CD8aTM/ICOS/CD3zeta chimeric antigen receptor complex (see FIG. 1, CAR1g). The polynucleotide comprises or is composed of a Tumor Binder, a CD8a hinge, a CD8a transmembrane domain, inducible costimulator (ICOS) signaling domain, and a CD3zeta domain. In several embodiments, the chimeric antigen receptor further comprises mbIL15 (see 1, CAR1h). By way of non-limiting embodiment, there is provided herein an anti-CD19moiety/CD8hinge/CD8aTM/ICOS/CD3zeta/2A/mIL15 chimeric antigen receptor complex. In such embodiments, the polynucleotide comprises or is composed of an anti-CD19 scFv, a CD8a hinge, a CD8a transmembrane domain, inducible costimulator (ICOS) signaling domain, a CD3zeta domain, a 2A cleavage site, and an mbIL-15 domain as described herein. In several embodiments, this receptor complex is encoded by a nucleic acid molecule having the sequence of SEQ ID NO: 63. In several embodiments, a nucleic acid sequence encoding an CAR1h chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 63. In several embodiments, the chimeric receptor comprises the amino acid sequence of SEQ ID NO: 64. In several embodiments, a CAR1h chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 64. In several embodiments, the CAR1h scFv does not comprise a Flag tag. It shall be appreciated that certain sequence variability, extensions, and/or truncations of the disclosed sequences may result when combining sequences, as a result of, for example, ease or efficiency in cloning (e.g., for creation of a restriction site).

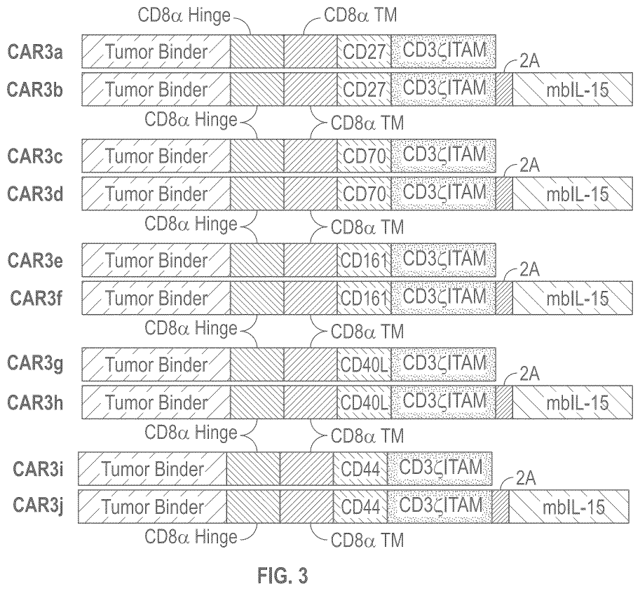

In several embodiments, there is provided a polynucleotide encoding a Tumor Binder/CD8hinge/CD8aTM/CD28/4-1BB/CD3zeta chimeric antigen receptor complex (see FIG. 1, CAR1i). The polynucleotide comprises or is composed of a Tumor Binder, a CD8a hinge, a CD8a transmembrane domain, a CD28 signaling domain, a 4-1BB signaling domain, and a CD3zeta domain. In several embodiments, the chimeric antigen receptor further comprises mbIL15 (see FIG. 3A, NK19-4b). By way of non-limiting embodiment, there is provided herein an anti-CD19moiety/CD8hinge/CD8aTM/CD28/4-1BB/CD3zeta/2A/mIL-15. In such embodiments, the polynucleotide comprises or is composed of an anti-CD19 scFv, a CD8a hinge, a CD8a transmembrane domain, a CD28 signaling domain, a 4-1BB signaling domain, a CD3zeta domain, a 2A cleavage site, and an mbIL-15 domain as described herein. In several embodiments, this receptor complex is encoded by a nucleic acid molecule having the sequence of SEQ ID NO: 65. In several embodiments, a nucleic acid sequence encoding an CAR1h chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 65. In several embodiments, the chimeric receptor comprises the amino acid sequence of SEQ ID NO: 66. In several embodiments, a CAR1h chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 66. In several embodiments, the CAR1h scFv does not comprise a Flag tag. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/CD8hinge/NKG2DTM/OX40/CD3zeta chimeric antigen receptor complex (see FIG. 2, CAR2a). The polynucleotide comprises or is composed of a Tumor Binder, a CD8a hinge, a NKG2D transmembrane domain, an OX40 signaling domain, and a CD3zeta domain. In several embodiments, the chimeric antigen receptor further comprises mbIL15 (see FIG. 2, CAR2b). By way of non-limiting embodiment, there is provided herein an anti-CD19moiety/CD8hinge/NKG2DTM/OX40/CD3zeta/2A/mIL-15 chimeric antigen receptor complex. In such embodiments, the polynucleotide comprises or is composed of an anti-CD19 scFv, a CD8a hinge, a NKG2D transmembrane domain, an OX40 signaling domain, a CD3zeta domain, a 2A cleavage site, and an mbIL-15 domain as described herein. In several embodiments, this receptor complex is encoded by a nucleic acid molecule having the sequence of SEQ ID NO: 67. In several embodiments, a nucleic acid sequence encoding an CAR2b chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 67. In several embodiments, the chimeric receptor comprises the amino acid sequence of SEQ ID NO: 68. In several embodiments, a CAR2b chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 68. In several embodiments, the CD19 scFv does not comprise a Flag tag. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/CD8hinge/CD8aTM/CD40/CD3zeta chimeric antigen receptor complex (see Figure CAR2c). The polynucleotide comprises or is composed of Tumor Binder, a CD8a hinge, a CD8a transmembrane domain, a CD40 signaling domain, and a CD3zeta domain. In several embodiments, the chimeric antigen receptor further comprises mbIL15 (see FIG. 1, CAR2d). By way of non-limiting embodiment, there is provided herein an anti-CD19moiety/CD8hinge/CD8aTM/CD40/CD3zeta/2A/mIL-15 chimeric antigen receptor complex. In such embodiments, the polynucleotide comprises or is composed of an anti-CD19 scFv variable heavy chain, a CD8a hinge, a CD8a transmembrane domain, a CD40 signaling domain, a CD3zeta domain, a 2A cleavage site, and an mbIL-15 domain as described herein. In several embodiments, this receptor complex is encoded by a nucleic acid molecule having the sequence of SEQ ID NO: 69. In several embodiments, a nucleic acid sequence encoding an CAR2d chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 69. In several embodiments, the chimeric receptor comprises the amino acid sequence of SEQ ID NO: 70. In several embodiments, a CAR2d chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 70. In several embodiments, the CD19 scFv does not comprise a Flag tag. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/CD8hinge/CD8aTM/OX40/CD3zeta/2A/EGFRt chimeric antigen receptor complex (see FIG. 2, CAR2e). The polynucleotide comprises or is composed of a Tumor Binder, a CD8a hinge, a CD8a transmembrane domain, an OX40 signaling domain, a CD3zeta domain, a 2A cleavage side, and a truncated version of the epidermal growth factor receptor (EGFRt). In several embodiments, the chimeric antigen receptor further comprises mbIL15 (see FIG. 2, CAR2f). By way of non-limiting embodiment, there is provided herein an anti-CD19moiety/CD8hinge/CD8aTM/OX40/CD3zeta/2A/mIL-15/2A/EGFRt chimeric antigen receptor complex. In such embodiments, the polynucleotide comprises or is composed of an anti-CD19 scFv, a CD8a hinge, a CD8a transmembrane domain, an OX40 signaling domain, a CD3zeta domain, a 2A cleavage side, a truncated version of the epidermal growth factor receptor (EGFRt), an additional 2A cleavage site, and an mbIL-15 domain as described herein. In several embodiments, this receptor complex is encoded by a nucleic acid molecule having the sequence of SEQ ID NO: 71. In several embodiments, a nucleic acid sequence encoding an CAR2f chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 71. In several embodiments, the chimeric receptor comprises the amino acid sequence of SEQ ID NO: 72. In several embodiments, a CAR2f chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 72. In several embodiments, the CD19 scFv does not comprise a Flag tag. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/CD8hinge/CD8aTM/CD40/CD3zeta chimeric antigen receptor complex (see FIG. 2, CAR2g). The polynucleotide comprises or is composed of a Tumor Binder, a CD8a hinge, a CD8a transmembrane domain, a CD40 signaling domain, and a CD3zeta domain. In several embodiments, the chimeric antigen receptor further comprises mbIL15 (see FIG. 2, CAR2h). By way of non-limiting embodiment, there is provided herein an anti-CD19moiety/CD8hinge/CD8aTM/CD40/CD3zeta/2A/mIL-15 chimeric antigen receptor complex. In such embodiments, the polynucleotide comprises or is composed of an anti-CD19 scFv variable light chain, a CD8a hinge, a CD8a transmembrane domain, a CD40 signaling domain, a CD3zeta domain, a 2A cleavage site, and an mbIL-15 domain as described herein. In several embodiments, this receptor complex is encoded by a nucleic acid molecule having the sequence of SEQ ID NO: 73. In several embodiments, a nucleic acid sequence encoding an CAR2h chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 73. In several embodiments, the chimeric receptor comprises the amino acid sequence of SEQ ID NO: 74. In several embodiments, a CAR2h chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 74. In several embodiments, the CD19 scFv does not comprise a Flag tag. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/CD8hinge/CD8aTM/CD27/CD3zeta chimeric antigen receptor complex (see FIG. 3, CAR3a). The polynucleotide comprises or is composed of a Tumor Binder, a CD8α hinge, a CD8α transmembrane domain, a CD27 signaling domain, and a CD3zeta domain. In several embodiments, the chimeric antigen receptor further comprises mbIL15 (see FIG. 3, CAR3b). By way of non-limiting embodiment, there is provided herein an anti-CD19moiety/CD8hinge/CD8aTM/CD27/CD3zeta/2A/mIL-15 chimeric antigen receptor complex. In such embodiments, the polynucleotide comprises or is composed of an anti-CD19 scFv, a CD8a hinge, a CD8a transmembrane domain, a CD27 signaling domain, a CD3zeta domain, a 2A cleavage site, and an mbIL-15 domain as described herein. In several embodiments, this receptor complex is encoded by a nucleic acid molecule having the sequence of SEQ ID NO: 75. In several embodiments, a nucleic acid sequence encoding an CAR3b chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 75. In several embodiments, the chimeric receptor comprises the amino acid sequence of SEQ ID NO: 76. In several embodiments, a CAR3b chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 76. In several embodiments, the CD19 scFv does not comprise a Flag tag. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/CD8hinge/CD8aTM/CD70/CD3zeta chimeric antigen receptor complex (see FIG. 3, CAR3c). The polynucleotide comprises or is composed of a Tumor Binder, a CD8a hinge, a CD8a transmembrane domain, a CD70 signaling domain, and a CD3zeta domain. In several embodiments, the chimeric antigen receptor further comprises mbIL15 (see FIG. 3, CAR3d). By way of non-limiting embodiment, there is provided herein an anti-CD19moiety/CD8hinge/CD8aTM/CD70/CD3zeta/2A/mIL-15 chimeric antigen receptor complex. In such embodiments, the polynucleotide comprises or is composed of an anti-CD19 scFv, a CD8a hinge, a CD8a transmembrane domain, a CD70 signaling domain, a CD3zeta domain, a 2A cleavage site, and an mbIL-15 domain as described herein. In several embodiments, this receptor complex is encoded by a nucleic acid molecule having the sequence of SEQ ID NO: 77. In several embodiments, a nucleic acid sequence encoding an CAR3d chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 77. In several embodiments, the chimeric receptor comprises the amino acid sequence of SEQ ID NO: 78. In several embodiments, a CAR3d chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 78. In several embodiments, the CD19 scFv does not comprise a Flag tag. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/CD8hinge/CD8aTM/CD161/CD3zeta chimeric antigen receptor complex (see FIG. 3, CAR3e). The polynucleotide comprises or is composed of a Tumor Binder, a CD8a hinge, a CD8a transmembrane domain, a CD161 signaling domain, and a CD3zeta domain. In several embodiments, the chimeric antigen receptor further comprises mbIL15 (see FIG. 3, CAR3f). By way of non-limiting embodiment, there is provided herein an anti-CD19moiety/CD8hinge/CD8aTM/CD161/CD3zeta/2A/mIL-15 chimeric antigen receptor complex. In such embodiments, the polynucleotide comprises or is composed of an anti-CD19 scFv, a CD8a hinge, a CD8a transmembrane domain, a CD161 signaling domain, a CD3zeta domain, a 2A cleavage site, and an mbIL-15 domain as described herein. In several embodiments, this receptor complex is encoded by a nucleic acid molecule having the sequence of SEQ ID NO: 79. In several embodiments, a nucleic acid sequence encoding an CAR3f chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 79. In several embodiments, the chimeric receptor comprises the amino acid sequence of SEQ ID NO: 80. In several embodiments, a CAR3f chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 80. In several embodiments, the CD19 scFv does not comprise a Flag tag. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/CD8hinge/CD8aTM/CD40L/CD3zeta chimeric antigen receptor complex (see FIG. 3, CAR3g). The polynucleotide comprises or is composed of a Tumor Binder, a CD8a hinge, a CD8a transmembrane domain, a CD40L signaling domain, and a CD3zeta domain. In several embodiments, the chimeric antigen receptor further comprises mbIL15 (see FIG. 3, CAR3h). By way of non-limiting embodiment, there is provided herein an anti-CD19moiety/CD8hinge/CD8aTM/CD40L/CD3zeta/2A/mIL-15 chimeric antigen receptor complex. In such embodiments, the polynucleotide comprises or is composed of an anti-CD19 scFv, a CD8a hinge, a CD8a transmembrane domain, a CD40L signaling domain, a CD3zeta domain, a 2A cleavage site, and an mbIL-15 domain as described herein. In several embodiments, this receptor complex is encoded by a nucleic acid molecule having the sequence of SEQ ID NO: 81. In several embodiments, a nucleic acid sequence encoding an CAR3h chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 81. In several embodiments, the chimeric receptor comprises the amino acid sequence of SEQ ID NO: 82. In several embodiments, a CAR3h chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 82. In several embodiments, the CD19 scFv does not comprise a Flag tag. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding a Tumor Binder/CD8hinge/CD8aTM/CD44/CD3zeta chimeric antigen receptor complex (see FIG. 3, CAR3i). The polynucleotide comprises or is composed of a Tumor Binder, a CD8a hinge, a CD8a transmembrane domain, a CD44 signaling domain, and a CD3zeta domain. In several embodiments, the chimeric antigen receptor further comprises mbIL15 (see FIG. 3, CAR3j). By way of non-limiting embodiment, there is provided herein an anti-CD19moiety/CD8hinge/CD8aTM/CD44/CD3zeta/2A/mIL-15 chimeric antigen receptor complex. In such embodiments, the polynucleotide comprises or is composed of an anti-CD19 scFv, a CD8a hinge, a CD8a transmembrane domain, a CD44 signaling domain, a CD3zeta domain, a 2A cleavage site, and an mbIL-15 domain as described herein. In several embodiments, this receptor complex is encoded by a nucleic acid molecule having the sequence of SEQ ID NO: 83. In several embodiments, a nucleic acid sequence encoding an CAR3j chimeric antigen receptor comprises a sequence that shares at least about 90%, at least about 94%, at least about 95%, at least about 96%, at least about 97%, at least about 98%, or at least about 99%, sequence identity, homology and/or functional equivalence with SEQ ID NO: 83. In several embodiments, the chimeric receptor comprises the amino acid sequence of SEQ ID NO: 84. In several embodiments, a CAR3j chimeric antigen receptor comprises an amino acid sequence that shares at least about 90%, at least about 94%, at least about 95%, at least about 96%, at least about 97%, at least about 98%, or at least about 99%, sequence identity, homology and/or functional equivalence with SEQ ID NO: 84. In several embodiments, the CD19 scFv does not comprise a Flag tag. It shall be appreciated that certain sequence variability, extensions, and/or truncations of the disclosed sequences may result when combining sequences, as a result of, for example, ease or efficiency in cloning (e.g., for creation of a restriction site).

In several embodiments, there is provided a polynucleotide encoding an anti CD123/CD8a hinge/CD8a transmembrane domain/OX40/CD3zeta chimeric antigen receptor complex (see FIG. 6, CD123 CARa). The polynucleotide comprises or is composed of an anti CD123 moiety, a CD8alpha hinge, a CD8a transmembrane domain, an OX40 domain, and a CD3zeta domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site). In several embodiments, there is provided an CD123 CAR construct that further comprises mbIL15, as disclosed herein (see e.g., FIG. 6, CD123 CARb).

In several embodiments, there is provided a polynucleotide encoding an anti CLDN6/CD8a hinge/CD8a transmembrane domain/OX40/CD3zeta chimeric antigen receptor complex (see FIG. 6, CLDN6 CARa). The polynucleotide comprises or is composed of an anti CLDN6 binding moiety, a CD8alpha hinge, a CD8a transmembrane domain, an OX40 domain, and a CD3zeta domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site). In several embodiments, there is provided a CLDN6 CAR construct that further comprises mbIL15, as disclosed herein (see e.g., FIG. 6, CLDN6 CARb).

Depending on the embodiment, various binders can be used to target CLDN6. In several embodiments, peptide binders are used, while in some embodiments antibodies, or fragments thereof are used. In several embodiments employing antibodies, antibody sequences are optimized, humanized or otherwise manipulated or mutated from their native form in order to increase one or more of stability, affinity, avidity or other characteristic of the antibody or fragment. In several embodiments, an antibody is provided that is specific for CLDN6. In several embodiments, an scFv is provided that is specific for CLDN6. In several embodiments, the antibody or scFv specific for CLDN6 comprises a heavy chain variable comprising the amino acid sequence of SEQ ID NO. 88. In some embodiments, the heavy chain variable comprises a sequence of amino acids that is at least 70%, 75%, 80%, 85%, 90%, 91%, 92%, 93%, 94%, 95%, 96%, 97%, 98%, 99%, or more, identical to the sequence of SEQ ID NO. 88. In some embodiments, the heavy chain variable comprises a sequence of amino acids that is encoded by a polynucleotide that hybridizes under moderately stringent conditions to the complement of a polynucleotide that encodes a heavy chain variable of SEQ ID NO. 88. In some embodiments, the heavy chain variable domain a sequence of amino acids that is encoded by a polynucleotide that hybridizes under stringent conditions to the complement of a polynucleotide that encodes a heavy chain variable encodes a heavy chain variable of SEQ ID NO. 88.

In several embodiments, the antibody or scFv specific for CLDN6 comprises a light chain variable comprising the amino acid sequence of any of SEQ ID NO. 89, 90, or 91. In several embodiments, the light chain variable comprises a sequence of amino acids that is encoded by a nucleotide sequence that is at least 70%, 75%, 80%, 85%, 90%, 91%, 92%, 93%, 94%, 95%, 96%, 97%, 98%, 99%, or more, identical to the identical to the sequence of SEQ ID NO. 89, 90, or 91. In some embodiments, the light chain variable comprises a sequence of amino acids that is encoded by a polynucleotide that hybridizes under moderately stringent conditions to the complement of a polynucleotide that encodes a light chain variable of SEQ ID NO. 89, 90, or 91. In some embodiments, the light chain variable domain comprises a sequence of amino acids that is encoded by a polynucleotide that hybridizes under stringent conditions to the complement of a polynucleotide that encodes a light chain variable domain of SEQ ID NO. 89, 90, or 91.

In several embodiments, the anti-CLDN6 antibody or scFv comprises one, two, or three heavy chain complementarity determining region (CDR) and one, two, or three light chain CDRs. In several embodiments, a first heavy chain CDR has the amino acid sequence of SEQ ID NO: 92. In some embodiments, the first heavy chain CDR comprises a sequence of amino acids that is at least 70%, 75%, 80%, 85%, 90%, 91%, 92%, 93%, 94%, 95%, 96%, 97%, 98%, 99%, or more, identical to the sequence of SEQ ID NO. 92. In several embodiments, a second heavy chain CDR has the amino acid sequence of SEQ ID NO: 93. In some embodiments, the second heavy chain CDR comprises a sequence of amino acids that is at least 70%, 75%, 80%, 85%, 90%, 91%, 92%, 93%, 94%, 95%, 96%, 97%, 98%, 99%, or more, identical to the sequence of SEQ ID NO. 93. In several embodiments, a third heavy chain CDR has the amino acid sequence of SEQ ID NO: 94. In some embodiments, the third heavy chain CDR comprises a sequence of amino acids that is at least 70%, 75%, 80%, 85%, 90%, 91%, 92%, 93%, 94%, 95%, 96%, 97%, 98%, 99%, or more, identical to the sequence of SEQ ID NO. 94.

In several embodiments, a first light chain CDR has the amino acid sequence of SEQ ID NO: 95, 98, or 101. In some embodiments, the first light chain CDR comprises a sequence of amino acids that is at least 70%, 75%, 80%, 85%, 90%, 91%, 92%, 93%, 94%, 95%, 96%, 97%, 98%, 99%, or more, identical to the sequence of SEQ ID NO. 95, 98, or 101. In several embodiments, a second light chain CDR has the amino acid sequence of SEQ ID NO: 96, 99, or 102. In some embodiments, the second light chain CDR comprises a sequence of amino acids that is at least 70%, 75%, 80%, 85%, 90%, 91%, 92%, 93%, 94%, 95%, 96%, 97%, 98%, 99%, or more, identical to the sequence of SEQ ID NO. 96, 99, or 102. In several embodiments, a third light chain CDR has the amino acid sequence of SEQ ID NO: 97, 100, or 103. In some embodiments, the third light chain CDR comprises a sequence of amino acids that is at least 70%, 75%, 80%, 85%, 90%, 91%, 92%, 93%, 94%, 95%, 96%, 97%, 98%, 99%, or more, identical to the sequence of SEQ ID NO. 97, 100, or 103.

Advantageously, in several embodiments, the CLDN6 CARs are highly specific to CLDN6 and do not substantially bind to any of CLDN3, 4, or 9.

In several embodiments, there is provided a polynucleotide encoding an anti BCMA/CD8a hinge/CD8a transmembrane domain/OX40/CD3zeta chimeric antigen receptor complex (see FIG. 6, BCMA CARa). The polynucleotide comprises or is composed of an anti BCMA binding moiety, a CD8alpha hinge, a CD8a transmembrane domain, an OX40 domain, and a CD3zeta domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site). In several embodiments, there is provided a BCMA CAR construct that further comprises mbIL15, as disclosed herein (see e.g., FIG. 6, BCMA CARb). Additional information about anti-BCMA CARs for use in the presently disclosed methods and compositions can be found in U.S. Provisional Patent Application No. 62/960,285, which is incorporated in its entirety by reference herein.

In several embodiments, there is provided a polynucleotide encoding an anti HER2/CD8a hinge/CD8a transmembrane domain/OX40/CD3zeta chimeric antigen receptor complex (see FIG. 6, HER2 CARa). The polynucleotide comprises or is composed of an anti HER2 binding moiety, a CD8alpha hinge, a CD8a transmembrane domain, an OX40 domain, and a CD3zeta domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site). In several embodiments, there is provided a HER2 CAR construct that further comprises mbIL15, as disclosed herein (see e.g., FIG. 6, HER2 CARb).

In several embodiments, there is provided a polynucleotide encoding an NKG2D/CD8a hinge/CD8a transmembrane domain/OX40/CD3zeta activating chimeric receptor complex (see FIG. 6, NKG2D ACRa). The polynucleotide comprises or is composed of a fragment of the NKG2D receptor capable of binding a ligand of the NKG2D receptor, a CD8alpha hinge, a CD8α transmembrane domain, an OX40 domain, and a CD3zeta domain as described herein. In several embodiments, this receptor complex is encoded by a nucleic acid molecule comprising the nucleic acid sequence of SEQ ID NO: 145. In yet another embodiment, this chimeric receptor is encoded by the amino acid sequence of SEQ ID NO: 174. In some embodiments, the sequence of the chimeric receptor may vary from SEQ ID NO. 145, but remains, depending on the embodiment, at least 70%, at least 75%, at least 80%, at least 85%, at least 90%, or at least 95% homologous with SEQ ID NO. 145. In several embodiments, while the chimeric receptor may vary from SEQ ID NO. 145, the chimeric receptor retains, or in some embodiments, has enhanced, NK cell activating and/or cytotoxic function. Additionally, in several embodiments, this construct can optionally be co-expressed with mbIL15 (FIG. 7, NKG2D ACRb). Additional information about chimeric receptors for use in the presently disclosed methods and compositions can be found in PCT Patent Publication No. WO/2018/183385, which is incorporated in its entirety by reference herein.

In several embodiments, there is provided a polynucleotide encoding an anti CD70/CD8a hinge/CD8a transmembrane domain/OX40/CD3zeta chimeric antigen receptor complex (see FIG. 7, CD70 CARa). The polynucleotide comprises or is composed of an anti CD70 binding moiety, a CD8alpha hinge, a CD8a transmembrane domain, an OX40 domain, and a CD3zeta domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site). In several embodiments, there is provided a CD70 CAR construct that further comprises mbIL15, as disclosed herein (see e.g., FIG. 7, CD70 CARb). Additional information about anti-CD70 CAR for use in the presently disclosed methods and compositions can be found in U.S. Provisional Patent Application Nos. 63/038,645 and 63/090,041, which are each incorporated in their entirety by reference herein.

In several embodiments, there is provided a polynucleotide encoding an anti mesothelin/CD8a hinge/CD8a transmembrane domain/OX40/CD3zeta chimeric antigen receptor complex (see FIG. 7, Mesothelin CARa). The polynucleotide comprises or is composed of an anti mesothelin binding moiety, a CD8alpha hinge, a CD8a transmembrane domain, an OX40 domain, and a CD3zeta domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site). In several embodiments, there is provided a Mesothelin CAR construct that further comprises mbIL15, as disclosed herein (see e.g., FIG. 7, Mesothelin CARb).

In several embodiments, there is provided a polynucleotide encoding an anti PD-L1/CD8a hinge/CD8a transmembrane domain/OX40/CD3zeta chimeric antigen receptor complex (see FIG. 7, PD-L1 CARa). The polynucleotide comprises or is composed of an anti PD-L1 binding moiety, a CD8alpha hinge, a CD8a transmembrane domain, an OX40 domain, and a CD3zeta domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site). In several embodiments, there is provided a PD-L1 CAR construct that further comprises mbIL15, as disclosed herein (see e.g., FIG. 7, PD-L1 CARb).

In several embodiments, there is provided a polynucleotide encoding an anti EGFR/CD8a hinge/CD8a transmembrane domain/OX40/CD3zeta chimeric antigen receptor complex (see FIG. 7, EGFR CARa). The polynucleotide comprises or is composed of an anti EGFR binding moiety, a CD8alpha hinge, a CD8a transmembrane domain, an OX40 domain, and a CD3zeta domain as described herein. In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site). In several embodiments, there is provided a EGFR CAR construct that further comprises mbIL15, as disclosed herein (see e.g., FIG. 7, EGFR CARb).

In several embodiments, an expression vector, such as a MSCV-IRES-GFP plasmid, a non-limiting example of which is provided in SEQ ID NO: 87, is used to express any of the chimeric antigen receptors provided for herein.

### Methods of Treatment

Provided herein are methods of treatment, e.g., comprising administering any of the engineered immune cells described herein or a composition containing engineered immune cells. In some aspects, also provided are methods of administering any of the engineered immune cells described herein or a composition containing engineered immune cells to a subject (e.g., a subject having a disease or disorder). In some aspects, there is also provided a use of any of the engineered immune cells described herein or a composition containing the engineered immune cells for treating a disease or disorder. In some aspects, there is also provided a use of any of the engineered immune cells described herein or a composition containing the engineered immune cells for the manufacture of a medicament to treat a disease or disorder. In some aspects, also provided is any of the engineered immune cells described herein or a composition containing the engineered immune cells for use in treating a disease or disorder, or for administration to a subject having a disease or disorder.

Diseases and disorders include tumors, including solid tumors, hematologic malignancies, and melanoma, and include local and metastatic tumors; infectious diseases, such as infection by a virus or other pathogen, e.g., HIV, HCV, HBV, CMV, HPV and parasitic diseases; and autoimmune and inflammatory diseases. In some embodiments, the disease or disorder is a tumor, cancer, malignancy, neoplasm, or other proliferative disease or disorder. In some embodiments, the disease or disorder is an infectious disease or disorder, such as, but not limited to, viral, retroviral, bacterial and protozoal infections, immunodeficiency, Cytomegalovirus (CMV), epstein-barr virus (EBV), adenovirus, BK polyoma virus. In some embodiments, the disease or disorder is an autoimmune or inflammatory disease or disorder, such as arthritis (e.g., Rheumatoid Arthritis (RA)), type I diabetes, Systemic Lupus Erythematosus (SLE), inflammatory bowel disease, psoriasis, scleroderma, autoimmune thyroid disease, Graces' disease, Crohn's disease, multiple sclerosis, asthma, and/or a disease or disorder associated with transplantation.

In some embodiments, the disease or disorder is cancer. Some embodiments relate to a method of treating, ameliorating, inhibiting, or preventing cancer with a cell or immune cell comprising a chimeric antigen receptor and/or an activating chimeric receptor, as disclosed herein. In some embodiments, the method includes treating or preventing cancer. In some embodiments, the method includes administering a therapeutically effective amount of immune cells expressing a tumor-directed chimeric antigen receptor and/or tumor-directed chimeric receptor as described herein. Examples of types of cancer that may be treated as such are described herein.

In certain embodiments, treatment of a subject with a genetically engineered cell(s) described herein achieves one, two, three, four, or more of the following effects, including, for example: (i) reduction or amelioration the severity of disease or symptom associated therewith; (ii) reduction in the duration of a symptom associated with a disease; (iii) protection against the progression of a disease or symptom associated therewith; (iv) regression of a disease or symptom associated therewith; (v) protection against the development or onset of a symptom associated with a disease; (vi) protection against the recurrence of a symptom associated with a disease; (vii) reduction in the hospitalization of a subject; (viii) reduction in the hospitalization length; (ix) an increase in the survival of a subject with a disease; (x) a reduction in the number of symptoms associated with a disease; (xi) an enhancement, improvement, supplementation, complementation, or augmentation of the prophylactic or therapeutic effect(s) of another therapy. Advantageously, the non-alloreactive engineered T cells disclosed herein further enhance one or more of the above. Administration can be by a variety of routes, including, without limitation, intravenous, intra-arterial, subcutaneous, intramuscular, intrahepatic, intraperitoneal and/or local delivery to an affected tissue.

### Cancer Types

Some embodiments of the compositions and methods described herein relate to administering immune cells comprising a tumor-directed chimeric antigen receptor and/or tumor-directed chimeric receptor to a subject with cancer. Various embodiments provided for herein include treatment or prevention of the following non-limiting examples of cancers. Examples of cancer include, but are not limited to, acute lymphoblastic leukemia (ALL), acute myeloid leukemia (AML), adrenocortical carcinoma, Kaposi sarcoma, lymphoma, gastrointestinal cancer, appendix cancer, central nervous system cancer, basal cell carcinoma, bile duct cancer, bladder cancer, bone cancer, brain tumors (including but not limited to astrocytomas, spinal cord tumors, brain stem glioma, craniopharyngioma, ependymoblastoma, ependymoma, medulloblastoma, medulloepithelioma), breast cancer, bronchial tumors, Burkitt lymphoma, cervical cancer, colon cancer, chronic lymphocytic leukemia (CLL), chronic myelogenous leukemia (CML), chronic myeloproliferative disorders, ductal carcinoma, endometrial cancer, esophageal cancer, gastric cancer, Hodgkin lymphoma, non-Hodgkin lymphoma, hairy cell leukemia, renal cell cancer, leukemia, oral cancer, nasopharyngeal cancer, liver cancer, lung cancer (including but not limited to, non-small cell lung cancer, (NSCLC) and small cell lung cancer), pancreatic cancer, bowel cancer, lymphoma, melanoma, ocular cancer, ovarian cancer, pancreatic cancer, prostate cancer, pituitary cancer, uterine cancer, and vaginal cancer.

In some embodiments, the cancer comprises a tumor. In some embodiments, the cancer comprises a solid tumor. In some embodiments, the cancer is a hematologic malignancy.

In some embodiments, the cancer is multiple myeloma. In some embodiments, the cancer is a leukemia or a lymphoma. In some embodiments, the cancer is a leukemia. In some embodiments, the leukemia is ALL, AML, CLL, or CML. In some embodiments, the cancer is a lymphoma. In some embodiments, the lymphoma is Hodgkin lymphoma or non-Hodgkin lymphoma (NHL). In some embodiments, the cancer is NHL. In some embodiments, the NHL is large B-cell lymphoma (LBCL). In some embodiments, the NHL is aggressive NHL. In some embodiments the NHL is a diffuse large B-cell lymphoma (DLBCL), Burkitt lymphoma, follicular lymphoma (FL, including grade 1, 2, 3a, and 3b), small lymphocytic lymphoma (SLL), mantle cell lymphoma, or marginal zone lymphoma. In some embodiments, the NHL is DLBCL. In some embodiments, the NHL is FL. In some embodiments, the NHL is mantle cell lymphoma. In some embodiments, the NHL is marginal zone lymphoma.

In some embodiments, the cancer is relapsed/refractory. In some embodiments, the cancer is relapsed refractory to a prior line of therapy. In some embodiments, the prior line of therapy comprises one prior line of therapy. In some embodiments, the prior line of therapy comprises two prior lines of therapy. In some embodiments, the prior line of therapy comprises three prior lines of therapy.

### Cancer Targets

The disease or disorder to be treated can be any disease or condition in which expression of an antigen is associated with and/or involved in the etiology of the disease or disorder, e.g., causing, exacerbating or otherwise participating in such disease or disorder. Non-limiting examples of diseases and disorders can include diseases or disorders associated with malignancies or cellular transformation (e.g., cancer), autoimmune or inflammatory diseases or infectious diseases caused by, for example, bacteria, viruses, or other pathogens. Additionally, non-limiting examples of antigens are described herein, including antigens associated with various diseases and disorder that can be treated. In particular embodiments, the chimeric receptor specifically binds to an antigen associated with the disease or disorder.

Some embodiments of the compositions and methods described herein relate to immune cells comprising a chimeric receptor that targets a cancer antigen. Non-limiting examples of target antigens include: CD19; TNF receptor family member B cell maturation (BCMA); CD38; DLL3; G protein coupled receptor class C group 5, member D (GPRC5D); epidermal growth factor receptor (EGFR) CD138; prostate-specific membrane antigen (PSMA); Fms Like Tyrosine Kinase 3 (FLT3); KREMEN2 (Kringle Containing Transmembrane Protein 2); ALPPL2 (Alkaline phosphatase, placental-like 2); CLDN4 (Claudin 4); CLDN6 (Claudin 6); CD123; CD22; CD30; CD171; CS1 (also referred to as CD2 subset 1, CRACC, SLAMF7, CD319, and 19A24); CD5, C-type lectin-like molecule-1 (CLL-1 or CLECL1); CD33; epidermal growth factor receptor variant III (EGFRviii); ganglioside G2 (GD2); ganglioside GD3 (aNeu5Ac(2-8)aNeu5Ac(2-3)bDGalp(1-4)bDGlcp(1-1)Cer); Tn antigen ((Tn Ag) or (GalNAca-Ser/Thr)); Receptor tyrosine kinase-like orphan receptor 1 (ROR1); Tumor-associated glycoprotein 72 (TAG72); CD44v6; a glycosylated CD43 epitope expressed on acute leukemia or lymphoma but not on hematopoietic progenitors, a glycosylated CD43 epitope expressed on non-hematopoietic cancers, Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha (FRa or FR1); Folate receptor beta (FRb); Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp1OO); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); sialyl Lewis adhesion molecule (sLe); ganglioside GM3 (aNeu5Ac(2-3)bDClalp(1-4)bDGlcp(1-1)Cer); transglutaminase 5 (TGS5); high molecular weight-melanoma associated antigen (HMWMAA); o-acetyl-GD2 ganglioside (OAcGD2); tumor endothelial marker 1 (TEM1/CD248); tumor endothelial marker 7-related (TEM7R); claudin 6 (CLDN6); thyroid stimulating hormone receptor (TSHR); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; survivin; telomerase; prostate carcinoma tumor antigen-1 (PCT A-1 or Galectin 8), melanoma antigen recognized by T cells 1 (MelanA or MARTI); Rat sarcoma (Ras) mutant; human Telomerase; reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 IB 1 (CYPIB 1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Gly cation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLL1), MPL, Biotin, c-MYC epitope Tag, CD34, LAMP1 TROP2, GFRalpha4, CDH17, CDH6, NYBR1, CDH19, CD200R, Slea (CA19.9; Sialyl Lewis Antigen); Fucosyl-GM1, PTK7, gpNMB, CDH1-CD324, CD276/B7H3, IL1 1Ra, IL13Ra2, CD179b-IGL11, TCRgamma-delta, NKG2D, CD32 (FCGR2A), Tn ag, Tim1-/HVCR1, CSF2RA (GM-CSFR-alpha), TGFbetaR2, Lews Ag, TCR-beta1 chain, TCR-beta2 chain, TCR-gamma chain, TCR-delta chain, FITC, Leutenizing hormone receptor (LHR), Follicle stimulating hormone receptor (FSHR), Gonadotropin Hormone receptor (CGHR or GR), CCR4, GD3, SLAMF6, SLAMF4, HIV1 envelope glycoprotein, HTLV1-Tax, CMV pp65, EBV-EBNA3c, KSHV K8.1, KSHV-gH, influenza A hemagglutinin (HA), GAD, PDL1, Guanylyl cyclase C (GCC), auto antibody to desmoglein 3 (Dsg3), auto antibody to desmoglein 1 (Dsg1), HLA, HLA-A, HLA-A2, HLA-B, HLA-C, HLA-DP, HLA-DM, HLA-DOA, HLA-DOB, HLA-DQ, HLA-DR, HLA-G, IgE, CD99, Ras G12V, Tissue Factor 1 (TF1), AFP, Claudin1 8.2 (CLD18A2 or CLDN18A.2)), P-glycoprotein, STEAP1, Liv1, Nectin-4, Cripto, gpA33, BST1/CD157, low conductance chloride channel, and the antigen recognized by TNT antibody. In some embodiments, the antigen is CD19. In some embodiments, the antigen is a ligand of NKG2D. In some embodiments, the antigen is CD70. In some embodiments, the antigen is BCMA.

### Administration and Dosing

Further provided herein are methods of treating a subject having cancer, comprising administering to the subject a composition comprising immune cells (such as NK and/or T cells) engineered to express a cytotoxic receptor complex as disclosed herein. For example, some embodiments of the compositions and methods described herein relate to use of a tumor-directed chimeric antigen receptor and/or tumor-directed chimeric receptor, or use of cells expressing a tumor-directed chimeric antigen receptor and/or tumor-directed chimeric receptor, for treating a cancer patient. Uses of such engineered immune cells for treating cancer are also provided.

In certain embodiments, treatment of a subject with a genetically engineered cell(s) described herein achieves one, two, three, four, or more of the following effects, including, for example: (i) reduction or amelioration the severity of disease or symptom associated therewith; (ii) reduction in the duration of a symptom associated with a disease; (iii) protection against the progression of a disease or symptom associated therewith; (iv) regression of a disease or symptom associated therewith; (v) protection against the development or onset of a symptom associated with a disease; (vi) protection against the recurrence of a symptom associated with a disease; (vii) reduction in the hospitalization of a subject; (viii) reduction in the hospitalization length; (ix) an increase in the survival of a subject with a disease; (x) a reduction in the number of symptoms associated with a disease; (xi) an enhancement, improvement, supplementation, complementation, or augmentation of the prophylactic or therapeutic effect(s) of another therapy. Each of these comparisons are versus, for example, a different therapy for a disease, which includes a cell-based immunotherapy for a disease using cells that do not express the constructs disclosed herein.

In some embodiments, the immune cells are obtained from a subject (e.g., a first subject) other than the subject that will receive or ultimately receives the cell therapy. In such embodiments, the cells are then administered to a different subject of the same species, e.g., a second subject. In some embodiments, the first and second subjects are genetically similar. In some embodiments, the second subject expresses the same HLA class or supertype as the first subject.

Advantageously, the non-alloreactive engineered T cells disclosed herein further enhance one or more of the above.

Administration can be by a variety of routes, including, without limitation, intravenous, intra-arterial, subcutaneous, intramuscular, intrahepatic, intraperitoneal, and/or local delivery to an affected tissue. In some embodiments, a given dose is administered by a single infusion of cells. In some embodiments, a given dose is administered by multiple infusions of cells, or by continuous infusion of cells. In some embodiments, administration of the cell dose or any other therapy (e.g., lymphodepletion therapy and/or combination therapy) is by outpatient delivery.

Doses of immune cells such as NK and/or T cells can be readily determined for a given subject based on their body mass, disease type and state, and desired aggressiveness of treatment, but range, depending on the embodiments, from about 10⁵ cells per kg to about 10¹² cells per kg (e.g., 10⁵-10⁷, 10⁷-10¹⁰, 10¹⁰-10¹² and overlapping ranges therein). In one embodiment, a dose escalation regimen is used. In several embodiments, a range of immune cells such as NK and/or T cells is administered, for example between about 1×10⁶ cells/kg to about 1×10⁸ cells/kg. In several embodiments, a range of NK cells is administered, for example between about 1×10⁶ cells/kg to about 1×10⁸ cells/kg. In several embodiments, a range of T cells is administered, for example between about 1×10⁶ cells/kg to about 1×10⁸ cells/kg.

In some embodiments, a dose of engineered cells comprises between about 300×10⁶ and 1.5×10⁹ NK cells. In some embodiments, a dose of engineered cells comprises about 300×10⁶ NK cells. In some embodiments, a dose of engineered cells comprises about 1×10⁹ NK cells. In some embodiments, a dose of engineered cells comprises about 1.5×10⁹ NK cells. In some embodiments, a dose of engineered cells comprises between about 300×10⁶ and 1.5×10⁹ T cells. In some embodiments, a dose of engineered cells comprises about 300×10⁶ T cells. In some embodiments, a dose of engineered cells comprises about 1×10⁹T cells. In some embodiments, a dose of engineered cells comprises about 1.5×10⁹ T cells.

In some embodiments, a dose of engineered cells comprises both NK and T cells. In some embodiments, a dose of engineered cells comprises between about 300×10⁶ and 1.5×10⁹ NK cells and between about 300×10⁶ and 1.5×10⁹ T cells. In some embodiments, a dose comprises about an equal number of NK cells and T cells. In some embodiments, a dose comprises more NK cells than T cell. In some embodiments, a dose comprises more T cells than NK cells.

Depending on the embodiment, various types of cancer can be treated. In several embodiments, hepatocellular carcinoma is treated. Additional embodiments provided for herein include treatment or prevention of the following non-limiting examples of cancers including, but not limited to, acute lymphoblastic leukemia (ALL), acute myeloid leukemia (AML), adrenocortical carcinoma, Kaposi sarcoma, lymphoma, gastrointestinal cancer, appendix cancer, central nervous system cancer, basal cell carcinoma, bile duct cancer, bladder cancer, bone cancer, brain tumors (including but not limited to astrocytomas, spinal cord tumors, brain stem glioma, glioblastoma, craniopharyngioma, ependymoblastoma, ependymoma, medulloblastoma, medulloepithelioma), breast cancer, bronchial tumors, Burkitt lymphoma, cervical cancer, colon cancer, chronic lymphocytic leukemia (CLL), chronic myelogenous leukemia (CML), chronic myeloproliferative disorders, ductal carcinoma, endometrial cancer, esophageal cancer, gastric cancer, Hodgkin lymphoma, non-Hodgkin lymphoma, hairy cell leukemia, renal cell cancer, leukemia, oral cancer, nasopharyngeal cancer, liver cancer, lung cancer (including but not limited to, non-small cell lung cancer, (NSCLC) and small cell lung cancer), pancreatic cancer, bowel cancer, lymphoma, melanoma, ocular cancer, ovarian cancer, pancreatic cancer, prostate cancer, pituitary cancer, uterine cancer, and vaginal cancer.

In some embodiments, also provided herein are nucleic acid and amino acid sequences that have sequence identity and/or homology of at least 80%, 85%, 90%, 95%, 96%, 97%, 98%, 99% (and ranges therein) as compared with the respective nucleic acid or amino acid sequences of SEQ ID NOS. 1-174 (or combinations of two or more of SEQ ID NOS: 1-174) and that also exhibit one or more of the functions as compared with the respective SEQ ID NOS. 1-174 (or combinations of two or more of SEQ ID NOS: 1-174) including but not limited to, (i) enhanced proliferation, (ii) enhanced activation, (iii) enhanced cytotoxic activity against cells presenting ligands to which NK cells harboring receptors encoded by the nucleic acid and amino acid sequences bind, (iv) enhanced homing to tumor or infected sites, (v) reduced off target cytotoxic effects, (vi) enhanced secretion of immunostimulatory cytokines and chemokines (including, but not limited to IFNg, TNFa, IL-22, CCL3, CCL4, and CCL5), (vii) enhanced ability to stimulate further innate and adaptive immune responses, and (viii) combinations thereof.

Additionally, in several embodiments, there are provided amino acid sequences that correspond to any of the nucleic acids disclosed herein, while accounting for degeneracy of the nucleic acid code. Furthermore, those sequences (whether nucleic acid or amino acid) that vary from those expressly disclosed herein, but have functional similarity or equivalency are also contemplated within the scope of the present disclosure. The foregoing includes mutants, truncations, substitutions, or other types of modifications.

In several embodiments, polynucleotides encoding the disclosed cytotoxic receptor complexes are mRNA. In some embodiments, the polynucleotide is DNA. In some embodiments, the polynucleotide is operably linked to at least one regulatory element for the expression of the cytotoxic receptor complex.

Additionally provided, according to several embodiments, is a vector comprising the polynucleotide encoding any of the polynucleotides provided for herein, wherein the polynucleotides are optionally operatively linked to at least one regulatory element for expression of a cytotoxic receptor complex. In several embodiments, the vector is a retrovirus.

Further provided herein are engineered immune cells (such as NK and/or T cells) comprising the polynucleotide, vector, or cytotoxic receptor complexes as disclosed herein. Further provided herein are compositions comprising a mixture of engineered immune cells (such as NK cells and/or engineered T cells), each population comprising the polynucleotide, vector, or cytotoxic receptor complexes as disclosed herein. Additionally, there are provided herein compositions comprising a mixture of engineered immune cells (such as NK cells and/or engineered T cells), each population comprising the polynucleotide, vector, or cytotoxic receptor complexes as disclosed herein and the T cell population having been genetically modified to reduce/eliminate GvHD and/or HvD. In some embodiments, the NK cells and the T cells are from the same donor. In some embodiments, the NK cells and the T cells are from different donors.

Doses of immune cells such as NK cells or T cells can be readily determined for a given subject based on their body mass, disease type and state, and desired aggressiveness of treatment, but range, depending on the embodiments, from about 10⁵ cells per kg to about 10¹² cells per kg (e.g., 10-10⁷, 10⁷-10¹⁰, 10¹⁰-10¹² and overlapping ranges therein). In one embodiment, a dose escalation regimen is used. In some embodiments, a dose of immune cells comprises NK cells. In some embodiments, a dose of immune cells comprises T cells. In some embodiments, a dose of immune cells comprises NK cells and T cells. In several embodiments, a range of NK cells is administered, for example between about 1×10⁶ cells/kg to about 1×10⁸ cells/kg. In several embodiments, a range of T cells is administered, for example between about 1×10⁶ cells/kg to about 1×10⁸ cells/kg. Depending on the embodiment, various types of cancer or infection disease can be treated.

For the prevention or treatment of cancer, the appropriate dosage may depend on the type of cancer to be treated, the type of cells or recombinant receptors, the severity and course of the cancer, whether the cells are administered for preventive or therapeutic purposes, previous therapy, the subject's clinical history and response to the cells, and the discretion of the attending physician. The compositions and cells are in some embodiments suitably administered to the subject at one time or over a series of treatments.

In some embodiments, the engineered immune cells are administered as part of a combination treatment, such as simultaneously with or sequentially with, in any order, another or additional therapeutic intervention, such as an antibody or engineered cell or receptor or agent, such as a cytotoxic or therapeutic agent. The engineered immune cells in some embodiments are co-administered with one or more additional therapeutic agents or in connection with another therapeutic intervention, either simultaneously or sequentially in any order. In some embodiments, the additional therapeutic agent is any interventions or agents described herein, such as any interventions or agents descried that can ameliorate symptoms of toxicity. In some contexts, the engineered immune cells are co-administered with another therapy sufficiently close in time such that the engineered immune cell populations enhance the effect of one or more additional therapeutic agents, or vice versa. In some embodiments, the engineered immune cells are administered prior to the one or more additional therapeutic agents. In some embodiments, the engineered immune cells are administered after the one or more additional therapeutic agents. In some embodiments, the one or more additional agents include a cytokine, such as IL-2, for example, to enhance persistence. In some embodiments, the methods comprise administration of a chemotherapeutic agent. In some embodiments, the methods comprise administration of a chemotherapeutic agent, e.g., a conditioning chemotherapeutic agent, for example, to reduce tumor burden prior to the administration, including as described in the subsequent section.

The dose of the additional agent can be any therapeutically effective amount, e.g. any dose amount described herein, and the appropriate dosage of the additional agent may depend on the type of disease to be treated, the type, dose and/or frequency of the binding molecule, recombinant receptor, cell and/or composition administered, the severity and course of the disease, previous therapy, the patient's clinical history and response to cell therapy, and the discretion of the attending physician.

### Lymphodepleting Therapy

Preconditioning subjects with immunodepleting (e.g., lymphodepleting) therapies in some aspects can improve the effects of an adoptive cell therapy, including any of those provided herein. Thus, in some embodiments, the methods include administering a preconditioning agent, such as a lymphodepleting or chemotherapeutic agent, such as cyclophosphamide, fludarabine, or combinations thereof, to a subject prior to administration of the engineered immune cells. For example, the subject may be administered a preconditioning agent at least 2 days prior, such as at least 3, 4, 5, 6, or 7 days prior, to administration of the engineered immune cells. In some embodiments, the subject is administered a preconditioning agent no more than 7 days prior, such as no more than 6, 5, 4, 3, or 2 days prior, to administration of the engineered immune cells.

In some embodiments, where the lymphodepleting agent comprises cyclophosphamide, the subject is administered cyclophosphamide at a dose between or between about 100 mg/m² and 500 mg/m², such as between or between about 200 mg/m² and 400 mg/m², or 250 mg/m² and 350 mg/m², inclusive. In some instances, the subject is administered about 300 mg/m² of cyclophosphamide. In some instances, the subject is administered about 500 mg/m² of cyclophosphamide. In some embodiments, the cyclophosphamide can be administered in a single dose or can be administered in a plurality of doses, such as given daily, every other day or every three days. In some embodiments, cyclophosphamide is administered daily, such as for 1-5 days, for example, for 3 to 5 days. In some instances, the subject is administered about 300 mg/m² of cyclophosphamide, daily for 3 days, prior to initiation of the cell therapy. In some instances, the subject is administered about 500 mg/m² of cyclophosphamide, daily for 3 days, prior to initiation of the cell therapy.

In some embodiments, where the lymphodepleting agent comprises fludarabine, the subject is administered fludarabine at a dose between or between about 1 mg/m² and 100 mg/m², such as between or between about 10 mg/m² and 75 mg/m², 15 mg/m² and 50 mg/m², 20 mg/m² and 40 mg/m², or 24 mg/m² and 35 mg/m², inclusive. In some instances, the subject is administered about 30 mg/m² of fludarabine. In some embodiments, the fludarabine can be administered in a single dose or can be administered in a plurality of doses, such as given daily, every other day or every three days. In some embodiments, fludarabine is administered daily, such as for 1-5 days, for example, for 3 to 5 days. In some instances, the subject is administered about 30 mg/m² of fludarabine, daily for 3 days, prior to initiation of the cell therapy.

In some embodiments, the lymphodepleting agent comprises a combination of agents, such as a combination of cyclophosphamide and fludarabine. Thus, the combination of agents may include cyclophosphamide at any dose or administration schedule, such as those described above, and fludarabine at any dose or administration schedule, such as those described above. For example, in some aspects, the subject is administered between 3-5 doses each of 300 mg/m² of cyclophosphamide and 50 mg/m² fludarabine prior to administration of the engineered immune cells. For example, in some aspects, the subject is administered between 3-5 doses each of 500 mg/m² of cyclophosphamide and 50 mg/m² fludarabine prior to administration of the engineered immune cells.

### Compositions and Formulations

In some embodiments, a cell dose comprising cells engineered with a chimeric receptor (e.g., a CAR) is provided as a composition or formulation, such as a pharmaceutical composition or formulation. Such compositions can be used in accordance with and/or with provided articles or compositions, such as for the prevention or treatment of diseases, conditions, and disorders, or in detection, diagnosis, and prognosis methods.

The term “pharmaceutical formulation” refers to a formulation in a form that allows the biological activity of the active ingredient contained therein to be effective and that is free of additional components having unacceptable toxicity to the subject to which the formulation will be applied.

By “pharmaceutically acceptable carrier” is meant an ingredient of a pharmaceutical formulation that is non-toxic to a subject, except for the active ingredient. Pharmaceutically acceptable carriers include, but are not limited to, buffers, excipients, stabilizers, or preservatives.

In some aspects, the choice of carrier depends in part on the particular cell or agent and/or the method of administration. Thus, there are a variety of suitable formulations. For example, the pharmaceutical composition may contain a preservative. Suitable preservatives may include, for example, methyl paraben, propyl paraben, sodium benzoate and benzalkonium chloride. In some aspects, a mixture of two or more preservatives is used. Preservatives or mixtures thereof are typically present in amounts of about 0.0001% to about 2% by weight of the total composition Pharmaceutically acceptable carriers are generally non-toxic to recipients at the dosages and concentrations used, and include, but are not limited to: buffers such as phosphate, citrate and other organic acids; antioxidants, including ascorbic acid and methionine; preservatives (such as octadecyl dimethyl benzyl ammonium chloride; hexamethonium chloride; benzalkonium chloride; benzethonium chloride; phenol, butanol or benzyl alcohol; alkyl parabens, such as methyl or propyl paraben, catechol; resorcinol; cyclohexanol; 3-pentanol; and m-cresol); low molecular weight (less than about 10 residues) polypeptides; proteins, such as serum albumin, gelatin or immunoglobulins; hydrophilic polymers such as polyvinylpyrrolidone; amino acids such as glycine, glutamine, asparagine, histidine, arginine or lysine; monosaccharides, disaccharides, and other carbohydrates including glucose, mannose, or dextrins; chelating agents, such as EDTA; sugars such as sucrose, mannitol, trehalose or sorbitol; salt-forming counterions, such as sodium; metal complexes (e.g., zinc-protein complexes); and/or a non-ionic surfactant, such as polyethylene glycol (PEG).

In some aspects, a buffer is included in the composition. Suitable buffering agents include, for example, citric acid, sodium citrate, phosphoric acid, potassium phosphate, and various other acids and salts. In some aspects, a mixture of two or more buffers is used. The buffering agent or mixtures thereof are typically present in an amount of from about 0.001% to about 4% by weight of the total composition. Methods for preparing administrable pharmaceutical compositions are known.

The formulation or composition may also contain more than one active ingredient that may be used for a particular indication, disease or condition that is prevented or treated with the cell or agent, where the respective activities do not adversely affect each other. Such active ingredients are present in combination in an amount effective for the intended purpose in a suitable manner. Thus, in some embodiments, the pharmaceutical composition further comprises other pharmaceutically active agents or drugs, such as chemotherapeutic agents, e.g., asparaginase, busulfan, carboplatin, cisplatin, daunomycin, doxorubicin, fluorouracil, gemcitabine, hydroxyurea, methotrexate, paclitaxel, rituximab, vinblastine, vincristine, and the like. In some embodiments, the agent or cell is administered in the form of a salt (e.g., a pharmaceutically acceptable salt). Suitable pharmaceutically acceptable acid addition salts include those derived from inorganic acids (such as hydrochloric, hydrobromic, phosphoric, metaphosphoric, nitric and sulfuric) and organic acids (such as tartaric, acetic, citric, malic, lactic, fumaric, benzoic, glycolic, gluconic, succinic and arylsulfonic, e.g., p-toluenesulfonic acid).

In some embodiments, the pharmaceutical composition contains the agent or cell in an amount effective to treat or prevent the disease or disorder (e.g., a therapeutically effective amount or a prophylactically effective amount). In some embodiments, treatment or prevention efficacy is monitored by periodic assessment of the treated subject. For repeated administrations over several days or longer, depending on the condition, the treatment is repeated until the desired suppression of disease symptoms occurs. However, other dosage regimens may be useful and may be determined. The desired dose may be delivered by administering the composition as a single bolus, by administering the composition as multiple boluses, or by administering the composition as a continuous infusion.

Formulations include those for oral, intravenous, intraperitoneal, subcutaneous, pulmonary, transdermal, intramuscular, intranasal, buccal, sublingual, or suppository administration. In some embodiments, the agent or cell population is administered to the subject by intravenous, intraperitoneal, or subcutaneous injection using peripheral systemic delivery.

In some embodiments, the compositions are provided as sterile liquid formulations (e.g., isotonic aqueous solutions, suspensions, emulsions, dispersions, or viscous compositions), which in some aspects may be buffered to a selected pH. Liquid formulations are generally easier to prepare than gels, other viscous compositions, and solid compositions. In addition, liquid compositions are somewhat more convenient to administer, particularly by injection. The liquid composition can comprise a carrier, which can be a solvent or dispersion medium containing, for example, water, saline, phosphate buffered saline, polyols (e.g., glycerol, propylene glycol, liquid polyethylene glycol), and suitable mixtures thereof.

Sterile injectable solutions can be prepared by incorporating the agent or cell into a solvent, such as an admixture with a suitable carrier, diluent, or excipient (e.g., sterile water, saline, glucose, dextrose, and the like).

Formulations for in vivo administration are typically sterile. Sterility can be readily achieved, for example, by filtration through sterile filtration membranes. In some embodiments, the dose of engineered cells administered is in a cryopreserved composition. In some aspects, the composition is administered after thawing the cryopreserved composition.

### Articles and Kits

Also provided are articles of manufacture, systems, devices, and kits for performing the provided methods. Also provided are articles of manufacture comprising (i) any of the compositions described herein; and (ii) instructions for administering the composition to a subject.

In some embodiments, an article of manufacture or kit comprises one or more containers (typically a plurality of containers), packaging material, and a label or package insert located on or associated with the one or more containers and/or packages, the label or package insert typically comprising instructions for performing any of the methods provided herein, e.g., for administering engineered immune cells to a subject. In some embodiments, the instructions provide guidance or assignment methods for assessing whether a subject is likely or suspected to be likely to respond prior to receiving the engineered immune and/or the extent or level of response after administration of the engineered immune cells. In some aspects, the article of manufacture may contain a dose or composition of engineered immune cells.

The articles provided herein contain packaging materials. Packaging materials for packaging provided materials are well known to those skilled in the art. Examples of packaging materials include, but are not limited to, blister packs, bottles, tubes, inhalers, pumps, bags, vials, containers, syringes, disposable laboratory items (e.g., pipette tips and/or plastic sheets), or bottles. The article or kit may include means to facilitate dispensing of materials or to facilitate use in a high throughput or large scale manner, for example to facilitate use in a robotic device. Typically, the package does not react with the composition contained therein.

In some embodiments, the reagents and/or cell compositions are packaged separately. In some embodiments, each vessel may have a single compartment. In some embodiments, the other components of the article of manufacture or kit are packaged separately, or together in a single compartment. For example, in some embodiments, engineered NK cells are provided separately from engineered T cells.

### Definitions

The terms “polypeptide” and “protein” are used interchangeably to refer to a polymer of amino acid residues, and are not limited to a minimum length. Polypeptides, including the provided receptors and other polypeptides, e.g., linkers or peptides, may include amino acid residues including natural and/or non-natural amino acid residues. The terms also include post-expression modifications of the polypeptide, for example, glycosylation, sialylation, acetylation, and phosphorylation. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

As used herein, a “subject” is a mammal, such as a human or other animal, and typically is human. In some embodiments, the subject, e.g., patient, to whom the agent or agents, cells, cell populations, or compositions are administered, is a mammal, typically a primate, such as a human. In some embodiments, the primate is a monkey or an ape. The subject can be male or female and can be any suitable age, including infant, juvenile, adolescent, adult, and geriatric subjects. In some embodiments, the subject is a non-primate mammal, such as a rodent.

As used herein, “treatment” (and grammatical variations thereof such as “treat” or “treating”) refers to complete or partial amelioration or reduction of a disease or condition or disorder, or a symptom, adverse effect or outcome, or phenotype associated therewith. Desirable effects of treatment include, but are not limited to, alleviation of symptoms, diminishment of any direct or indirect pathological consequences of the disease, preventing metastasis, decreasing the rate of disease progression, amelioration or palliation of the disease state, and remission or improved prognosis. The terms do not imply complete curing of a disease or complete elimination of any symptom or effect(s) on all symptoms or outcomes.

“Preventing” (and grammatical variations thereof such as “prevent” or “prevention”) as used herein, includes providing prophylaxis with respect to the occurrence or recurrence of a disease in a subject that may be predisposed to the disease but has not yet been diagnosed with the disease. In some embodiments, the provided cells and compositions are used to delay development of a disease or to slow the progression of a disease.

A “therapeutically effective amount” of an agent, e.g., a pharmaceutical formulation or cells, refers to an amount effective, at dosages and for periods of time necessary, to achieve a desired therapeutic result, such as for treatment of a disease, condition, or disorder, and/or pharmacokinetic or pharmacodynamic effect of the treatment. The therapeutically effective amount may vary according to factors such as the disease state, age, sex, and weight of the subject, and the populations of cells administered. In some embodiments, the provided methods involve administering the cells and/or compositions at effective amounts, e.g., therapeutically effective amounts.

The term “about” as used herein refers to the usual error range for the respective value readily known to the skilled person in this technical field. Reference to “about” a value or parameter herein includes (and describes) embodiments that are directed to that value or parameter per se.

As used herein, the singular forms “a,” “an,” and “the” include plural referents unless the context clearly dictates otherwise. For example, “a” or “an” means “at least one” or “one or more.”

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. Language such as “up to,” “at least,” “greater than,” “less than,” “between,” and the like includes the number recited. Numbers preceded by a term such as “about” or “approximately” include the recited numbers. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

In addition, when a sequence is disclosed as “comprising” a nucleotide or amino acid sequence, such a reference shall also include, unless otherwise indicated, that the sequence “comprises”, “consists of” or “consists essentially of” the recited sequence.

As used herein, a composition refers to any mixture of two or more products, substances, or compounds, including cells. It may be a solution, a suspension, liquid, powder, a paste, aqueous, non-aqueous or any combination thereof.

Unless defined otherwise, all terms of art, notations and other technical and scientific terms or terminology used herein are intended to have the same meaning as is commonly understood to which the claimed subject matter pertains. In some cases, terms with commonly understood meanings are defined herein for clarity and/or for ready reference, and the inclusion of such definitions herein should not necessarily be construed to represent a substantial difference over what is generally understood.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### Non-Limiting Embodiments

1. A population of genetically engineered immune cells for cancer immunotherapy, comprising:
genetically engineered immune cells that express a cytotoxic receptor comprising an extracellular ligand binding domain, a transmembrane domain, and a cytotoxic signaling complex,
wherein the immune cells are genetically edited at one or more target sites in the genome of the immune cell to yield reduced levels of expression of a protein which is encoded by a gene which comprises an edited target site as compared to a non-edited immune cell,
wherein the edits are made using a Crispr/Cas system,
wherein the edits result in a decrease in the frequency of cell surface expression of major histocompatibility complex class I (MHC I) molecules within the population of genetically engineered immune cells, and
wherein the genetically engineered immune cells are further engineered to express at least one immunosuppressive effector,
wherein the at least one immunosuppressive effector exerts suppressive effects on the cytotoxic activity of natural killer cells and/or T cells, and
wherein the genetically engineered and edited immune cells exhibit one or more of enhanced expansion capability, enhanced cytotoxicity against target cells, and enhanced persistence, as compared to cells that do not comprise said immunosuppressive effector and said edited gene.

2. A population of genetically engineered immune cells for cancer immunotherapy, comprising:
one or more of genetically engineered NK cells and genetically engineered T cells;
wherein the genetically engineered immune cells are engineered to express a cytotoxic receptor,
wherein the cytotoxic receptor comprises an extracellular ligand binding domain, a transmembrane domain, and a cytotoxic signaling complex,
wherein the immune cells are genetically edited at one or more target sites in the genome of the immune cell to yield reduced levels of expression of a protein which is encoded by a gene which comprises an edited target site as compared to a non-edited immune cell,
wherein the edits are made using an RNA-guided endonuclease,
wherein the edits result in a decrease in the frequency of cell surface expression of MHC I molecules within the population of genetically engineered immune cells, and
wherein the genetically engineered immune cells are further genetically engineered to express at least one immunosuppressive effector,
wherein the at least one immunosuppressive effector exerts suppressive effects on the cytotoxic activity of undesired cells,
wherein the undesired cells comprise one or more of non-engineered natural killer cells, non-engineered T cells, or undesired engineered cells, and
wherein the genetically engineered and edited immune cells exhibit one or more of enhanced expansion capability, enhanced cytotoxicity against target cells, and enhanced persistence, as compared to cells that do not comprise said immunosuppressive effector and said edited gene.

3. A population of genetically engineered immune cells for cancer immunotherapy, comprising:
one or more of genetically engineered NK cells and genetically engineered T cells;
wherein the plurality of genetically engineered immune cells are engineered to express a cytotoxic receptor,
wherein the cytotoxic receptor comprises an extracellular ligand binding domain, a transmembrane domain, and a cytotoxic signaling complex,
wherein the immune cells are genetically edited at one or more target sites in the genome of the immune cell to yield reduced levels of expression of a protein which is encoded by a gene which comprises an edited target site as compared to a non-edited immune cell,
wherein the edits result in a decrease in the frequency of cell surface expression of MHC I molecules within the population of genetically engineered immune cells, and
wherein the genetically engineered immune cells are further genetically engineered to express at least one immunosuppressive effector,
wherein the at least one immunosuppressive effector exerts suppressive effects on undesired cytotoxic activity of suppressive cells,
wherein the genetically engineered immune cells exhibit one or more of enhanced expansion capability, enhanced cytotoxicity against target cells, and enhanced persistence, as compared to cells that do not comprise said immunosuppressive effector.

4. The genetically engineered immune cells of any one of Embodiments 1 to 3, wherein the genetic edit is to a gene involved in antigen processing and/or MHC I complex assembly.

5. The genetically engineered immune cells of any one of Embodiments 1 to 4, wherein the suppressive cells comprise host cells.

6. The genetically engineered immune cells of any one of Embodiments 1 to 4, wherein the suppressive cells comprise one or more of non-engineered natural killer cells, non-engineered T cells, or suppressive engineered cells.

7. The genetically engineered immune cells of Embodiment 6, wherein suppressive engineered cells comprise the genetically engineered immune cells.

8. The genetically engineered immune cells of any one of the preceding Embodiments, wherein the cells that do not comprise said immunosuppressive effector are either non-engineered or engineered cells.

9. The genetically engineered immune cells of any one of the preceding Embodiments, wherein the genetic edit is to one or more of UGT-1 (UGTA-1), TAPBPL (TAPBPR), TAPBP (Tapasin), TAP-1, TAP-2; ERp57; Calreticulin (CRT); Endoplasmic reticulum aminopeptidases ERAP1 and/or ERAP2, and/or one or more immunoproteasome components selected from the standard proteasome catalytic subunits β1, β2, and/or β5, and the inducible proteasome catalytic subunits LMP2, MECL-1, and/or LMP7.

10. The genetically engineered immune cells of Embodiment 9, wherein the genetic edit is to TAPBP and/or TAP2 and reduced expression of TAPBP and/or TAP2 enables the immune cells to be used in allogeneic cancer immunotherapy with reduced host versus graft rejection as compared to immune cells expressing endogenous levels of TAPBP and/or TAP2.

11. The genetically engineered immune cells of any one of the preceding Embodiments, wherein the cytotoxic receptor targets one or more of NKG2D ligands, CD19, BCMA, CD70, and CD38 expressed by target tumor cells.

12. The genetically engineered immune cells of any one of the preceding Embodiments, wherein the cytotoxic signaling complex comprises an OX40 subdomain or a 4-1BB domain, and a CD3zeta subdomain.

13. The genetically engineered immune cells of any one of Embodiments 1 to 12, wherein the at least one immunosuppressive effector comprises a virally-derived peptide.

14. The genetically engineered immune cells of any one of Embodiments 1 to 13, wherein the at least one immunosuppressive effector comprises a peptide derived from a retrovirus.

15. The genetically engineered immune cells of any one of Embodiments 1 to 14, wherein the at least one immunosuppressive effector comprises a peptide derived from an envelope protein of a retrovirus.

16. The genetically engineered immune cells of any one of Embodiments 1 to 12, wherein the at least one immunosuppressive effector comprises at least a portion of a human protein and/or at least a portion of a human protein complex.

17. The genetically engineered immune cells of any one of Embodiments 1 to 12, wherein the at least one immunosuppressive effector comprises at least a portion of human protein.

18. The genetically engineered immune cells of any one of any one of the preceding Embodiments, wherein the at least one immunosuppressive effector comprises a chimeric construct comprises at least one virally-derived peptide and at least a portion of a human protein and/or at least a portion of a human protein complex.

19. The genetically engineered immune cells of any one of Embodiments 1 to 18, wherein the at least one immunosuppressive effector is integrated into the cytotoxic receptor.

20. The genetically engineered immune cells of Embodiment 19, wherein the at least one immunosuppressive effector is integrated into the cytotoxic receptor between the transmembrane domain and the extracellular ligand-binding domain.

21. The genetically engineered immune cells of Embodiment 19, wherein the at least one immunosuppressive effector is integrated into the cytotoxic receptor within the extracellular ligand-binding domain.

22. The genetically engineered immune cells of Embodiment 21 wherein the extracellular ligand-binding domain comprises an scFv and the at least one immunosuppressive effector is integrated into a linker region of the scFv.

23. The genetically engineered immune cells of Embodiment 19, wherein the at least one immunosuppressive effector is integrated into the cytotoxic receptor within an N-terminal region of the cytotoxic receptor distally positioned from the extracellular ligand-binding domain.

24. The genetically engineered immune cells of any one of Embodiments 1 to 23, wherein the at least one immunosuppressive effector is integrated into the cytotoxic receptor at a plurality of locations within an extracellular region of the cytotoxic receptor.

25. The genetically engineered immune cells of any one of Embodiments 1 to 18, wherein the at least one immunosuppressive effector is bound to an extracellular membrane of the immune cells.

26. The genetically engineered immune cells of any one of Embodiments 1 to 18, wherein the at least one immunosuppressive effector comprises a transmembrane protein.

27. The genetically engineered immune cells of Embodiment 26, wherein the transmembrane protein is selected from CD8α, CD4, CD3ε, CD3γ, CD3δ, CD3ζ, CD28, CD137, glycophorin A, glycophorin D, nicotinic acetylcholine receptor, a GABA receptor, FcεRIγ, and a T-cell receptor.

28. The genetically engineered immune cells of Embodiment 26 or 27, wherein the transmembrane protein comprises a CD8α transmembrane protein.

29. The genetically engineered immune cells of Embodiment 26, 27, or 28, wherein immunosuppressive effector is expressed on the immune cells by a disulfide trap single chain trimer (dtSCT).

30. The genetically engineered immune cells of any one of Embodiments 1 to 29, wherein the immunosuppressive effector is encoded by a nucleic acid or comprises a peptide having at least 85% sequence identity to one or more of the nucleotide or amino acid sequences of SEQ ID NOs: 683-894 or 997-1000.

31. The genetically engineered immune cells of any one of Embodiments 1 to 30, wherein the immunosuppressive effector comprises a peptide having at least 95% sequence identity to SEQ ID NO: 830, 997, 999, or 689.

32. The genetically engineered immune cells of any one of Embodiments 1 to 31, wherein the immunosuppressive effector is encoded by a nucleic acid having at least 95% sequence identity to SEQ ID NO: 829, 998, 1000, or 690.

33. The genetically engineered immune cells of any one of the preceding Embodiments, wherein the immune cells comprise genetically engineered Natural Killer (NK) cells, genetically engineered T cells, or combinations thereof.

34. The genetically engineered immune cells of any one of the preceding Embodiments, wherein the genetically engineered immune cells are suitable for use in allogeneic cancer cell therapy with reduced risk of graft versus host disease.

35. The genetically engineered immune cells of any one of the preceding Embodiments, wherein the genetically engineered immune cells are suitable for use in allogeneic cancer cell therapy with reduced risk of cytotoxic activity between the genetically engineered immune cells.

36. The genetically engineered immune cells of any one of the preceding Embodiments, wherein at least a portion of the genetically engineered immune cells are engineered to express membrane bound IL-15.

37. A method for the treatment of cancer in a subject comprising administering to the subject genetically engineered immune cells according to any of the preceding Embodiments.

38. Use of genetically engineered immune cells according to any of Embodiments 1 to 37 for the treatment of cancer.

39. Use of genetically engineered immune cells according to any of Embodiments 1 to 37 for the preparation of a medicament for the treatment of cancer.

40. A method of manufacturing a population of genetically engineered immune cells for cancer immunotherapy, comprising:
contacting a population of immune cells with an RNA-guided endonuclease to genetically edit one or more target sites in the genome of the immune cell to yield reduced levels of expression of a protein which is encoded by a gene which comprises an edited target site as compared to a non-edited immune cell,
wherein the edit results in a decrease in the frequency of cell surface expression of major MHC I molecules within the population of genetically engineered immune cells, and;
contacting the population of immune cells with a polynucleotide encoding a cytotoxic receptor comprising an extracellular ligand binding domain, a transmembrane domain, and a cytotoxic signaling complex;
contacting the population of immune cells with an additional polynucleotide encoding at least one immunosuppressive effector,
wherein the immunosuppressive effector is encoded by a nucleic acid or comprises a peptide having at least 85% sequence identity to one or more of the nucleotide or amino acid sequences of SEQ ID NOs: 683-894 or 997-1000;
wherein the at least one immunosuppressive effector exerts suppressive effects on the cytotoxic activity of suppressive cells,
and wherein the genetically engineered immune cells exhibit one or more of enhanced expansion capability, enhanced cytotoxicity against target cells, and enhanced persistence, as compared to cells that do not comprise said at least one immunosuppressive effector.

41. The method of Embodiment 40, wherein the genetic edit is to one or more of UGT-1 (UGTA-1), TAPBPL (TAPBPR), TAPBP (Tapasin), TAP-1, TAP-2; ERp57; Calreticulin (CRT); Endoplasmic reticulum aminopeptidases ERAP1 and/or ERAP2, and/or one or more immunoproteasome components selected from the standard proteasome catalytic subunits β1, β2, and/or β5, and the inducible proteasome catalytic subunits LMP2, MECL-1, and/or LMP7.

42. The method of Embodiment 40 or 41, wherein an additional genetic edit is made to one or more of a CISH gene, a CBLB gene, a B2M gene, a CD70 gene, an adenosine receptor gene, an NKG2A gene, a CIITA gene, a TGFBR gene, or any combination thereof.

43. A population of genetically engineered immune cells for cancer immunotherapy, comprising:
genetically engineered immune cells that express a cytotoxic receptor comprising an extracellular ligand binding domain, a transmembrane domain, and a cytotoxic signaling complex,
wherein the immune cells are genetically edited at one or more target sites in the genome of the immune cell to yield reduced levels of expression of a protein which is encoded by a gene which comprises an edited target site as compared to a non-edited immune cell,
wherein the edits are made using a Crispr/Cas system, and
wherein the edits result in a decrease in the frequency of cell surface expression of MHC I molecules within the population of genetically engineered immune cells, and
wherein the genetically engineered and edited immune cells exhibit one or more of enhanced expansion capability, enhanced cytotoxicity against target cells, and enhanced persistence, as compared to cells that do not comprise said edited gene.

44. A population of genetically engineered immune cells for cancer immunotherapy, comprising:
one or more of genetically engineered NK cells and genetically engineered T cells;
wherein the genetically engineered immune cells are engineered to express a cytotoxic receptor,
wherein the cytotoxic receptor comprises an extracellular ligand binding domain, a transmembrane domain, and a cytotoxic signaling complex,
wherein the immune cells are genetically edited at one or more target sites in the genome of the immune cell to yield reduced levels of expression of a protein which is encoded by a gene which comprises an edited target site as compared to a non-edited immune cell,
wherein the edits are made using an RNA-guided endonuclease, and
wherein the edits result in a decrease in the frequency of cell surface expression of MHC I molecules within the population of genetically engineered immune cells, and
wherein the genetically engineered and edited immune cells exhibit one or more of enhanced expansion capability, enhanced cytotoxicity against target cells, an enhanced persistence, as compared to cells that do not comprise said edited gene.

45. The genetically engineered immune cells of Embodiment 43 or 44, wherein the genetic edit is to one or more of UGT-1 (UGTA-1), TAPBPL (TAPBPR), TAPBP (Tapasin), TAP-1, TAP-2; ERp57; Calreticulin (CRT); Endoplasmic reticulum aminopeptidases ERAP1 and/or ERAP2, and/or one or more immunoproteasome components selected from the standard proteasome catalytic subunits β1, β2, and/or β5, and the inducible proteasome catalytic subunits LMP2, MECL-1, and/or LMP7.

46. The genetically engineered immune cells of Embodiment 45, wherein the genetic edit is to TAPBP and/or TAP2 and reduced expression of TAPBP and/or TAP2 enables the immune cells to be used in allogeneic cancer immunotherapy with reduced host versus graft rejection as compared to immune cells expressing endogenous levels of TAPBP and/or TAP2.

47. The genetically engineered immune cells of any one of Embodiments 43 to 46, further comprising at least one immunosuppressive effector exerts suppressive effects on undesired cytotoxic activity of suppressive cells.

48. The genetically engineered immune cells of Embodiment 47, wherein the suppressive cells comprise host cells.

49. The genetically engineered immune cells of Embodiment 47, wherein the suppressive cells comprise one or more of non-engineered natural killer cells, non-engineered T cells, or suppressive engineered cells.

50. The genetically engineered immune cells of Embodiment 49, wherein suppressive engineered cells comprise the genetically engineered immune cells.

51. The genetically engineered immune cells of any one of Embodiments 43 to 50, wherein the cells that do not comprise said immunosuppressive effector are either non-engineered or engineered cells.

52. The genetically engineered immune cells of any one of Embodiments 47 to 51, wherein the immunosuppressive effector is encoded by a nucleic acid or comprises a peptide having at least 85% sequence identity to one or more of the nucleotide or amino acid sequences of SEQ ID NOs: 683-894 or 997-1000, wherein the immunosuppressive effector comprises at least a portion of an HLA-E molecule, and/or wherein the immunosuppressive effector comprises at least a portion of CD47.

53. A population of genetically engineered immune cells for cancer immunotherapy, comprising:
a subpopulation of genetically engineered NK cells and a subpopulation of genetically engineered T cells,
wherein each of the subpopulations comprise an engineered cytotoxic receptor,
wherein each of the subpopulations comprises a genetic edit to a gene encoding TAP-2 and/or TAPBP, resulting in a decrease in the frequency of MHC I molecules within each subpopulation, and
wherein the genetically engineered and edited immune cells exhibit one or more of enhanced expansion capability, enhanced cytotoxicity against target cells, and enhanced persistence, as compared to cells that do not comprise said edited genetic edits.

54. A population of genetically engineered immune cells for cancer immunotherapy, comprising:
a subpopulation of genetically engineered NK cells and a subpopulation of genetically engineered T cells,
wherein each of the subpopulations comprise an engineered cytotoxic receptor,
wherein each of the subpopulations comprises a genetic edit to a gene involved in antigen processing and/or MHC I complex assembly, resulting in a decrease in the frequency of MHC I molecules within each subpopulation, and
wherein the genetically engineered and edited immune cells exhibit one or more of enhanced expansion capability, enhanced cytotoxicity against target cells, and enhanced persistence, as compared to cells that do not comprise said edited genetic edits.

55. A method for the treatment of cancer in a subject comprising administering to the subject genetically engineered immune cells according to any of Embodiments 43 to 54.

56. Use of genetically engineered immune cells according to any of Embodiments 43 to 54 for the treatment of cancer.

57. Use of genetically engineered immune cells according to any of Embodiments 43 to 54 for the preparation of a medicament for the treatment of cancer.

58. A method of manufacturing a population of genetically engineered immune cells for cancer immunotherapy, comprising:
contacting a population of immune cells with an RNA-guided endonuclease to genetically edit one or more target sites in the genome of the immune cell to yield reduced levels of expression of a protein which is encoded by a gene which comprises an edited target site as compared to a non-edited immune cell,
wherein the edit results in a decrease in the frequency of cell surface expression of MHC I molecules within the population of genetically engineered immune cells, and;
contacting the population of immune cells with an additional polynucleotide encoding at least one immunosuppressive effector,
wherein the immunosuppressive effector is encoded by a nucleic acid or comprises a peptide having at least 85% sequence identity to one or more of the nucleotide or amino acid sequences of SEQ ID NOs: 683-894 or 997-1000;
wherein the at least one immunosuppressive effector exerts suppressive effects on the cytotoxic activity of suppressive cells,
and wherein the genetically engineered immune cells exhibit one or more of enhanced expansion capability, enhanced cytotoxicity against target cells, and enhanced persistence, as compared to cells that do not comprise said at least one immunosuppressive effector.

59. A method of enhancing the persistence of immune cells for use in allogeneic therapy, comprising:
contacting a population of immune cells with an RNA-guided endonuclease to genetically edit one or more target sites in the genome of the immune cell to yield reduced levels of expression of a protein which is encoded by a gene which comprises an edited target site as compared to a non-edited immune cell,
wherein the edit results in a decrease in the frequency of cell surface expression of MHC I molecules within the population of genetically engineered immune cells, and;
and wherein the genetically edited immune cells are less likely to trigger cytotoxic effects from one or more of host NK or T cells, thereby exhibiting enhanced persistence, as compared to cells that do not comprise said gene edit.

60. A method of enhancing the persistence of immune cells for use in allogeneic therapy, comprising:
contacting a population of immune cells with an RNA-guided endonuclease to genetically edit one or more target sites in the genome of the immune cell to yield reduced levels of expression of a protein which is encoded by a gene which comprises an edited target site as compared to a non-edited immune cell,
wherein the edit is to a gene involved in antigen processing and/or MHC I complex assembly; and
wherein the genetically edited immune cells are less likely to trigger cytotoxic effects from one or more of host NK or T cells, thereby exhibiting enhanced persistence, as compared to cells that do not comprise said gene edit.

61. The method of any one of Embodiments 58 to 60, wherein the genetic edit is to one or more of UGT-1 (UGTA-1), TAPBPL (TAPBPR), TAPBP (Tapasin), TAP-1, TAP-2; ERp57; Calreticulin (CRT); Endoplasmic reticulum aminopeptidases ERAP1 and/or ERAP2, and/or one or more immunoproteasome components selected from the standard proteasome catalytic subunits β1, β2, and/or β5, and the inducible proteasome catalytic subunits LMP2, MECL-1, and/or LMP7.

62. The method of any one of Embodiments 58 to 61, wherein an additional genetic edit is made to one or more of a CISH gene, a CBLB gene, a B2M gene, a CD70 gene, an adenosine receptor gene, an NKG2A gene, a CIITA gene, a TGFBR gene, or any combination thereof.

63. The method of any one of Embodiments 58 to 62, further comprising contacting the population of immune cells with a polynucleotide encoding a cytotoxic receptor comprising an extracellular ligand binding domain, a transmembrane domain, and a cytotoxic signaling complex.

64. The method of any one of Embodiments 58 to 63, wherein the population of immune cells comprises NK cells, T cells and/or NK and T cells.

### EXAMPLES

The following are non-limiting descriptions of experimental methods and materials that were used in examples disclosed below.

### Example 1—Disruption of MHC I Presentation Pathway Genes to Confer Resistance to Cytotoxicity

As discussed herein, allogeneic cell populations for immunotherapy are subject to potential reduced lifespan when administered due to host versus graft rejection. This is in addition to potential graft versus host issues that can arise, as well as fratricide among the cells to be administered. The major histocompatibility complex (MHC), which in humans is referred to as human leukocyte antigen (HLA), relates to a group of genes encoding a variety of cell surface markers, antigen-presenting molecules, and other proteins involved in immune function, which mediate such host versus graft and graft versus host rejections. In order to reduce host versus graft rejection, therapeutic cells can, in several embodiments, be edited, for example by knocking out B2M expression, which eliminates expression of HLA class I molecules from the surface of the cells (see, for example FIG. 8H). However, complete HLA class I removal can result in in vulnerability of the allogeneic product to fratricide (e.g., from a co-administered NK cell bearing a CAR) and/or elimination by host NK cells (since the HLA-negative cells are missing a normally expressed self signal). As disclosed herein and in PCT Patent Application No. PCT/US2021/072715 (which is incorporated in its entirety by reference herein), engineered expression of HLA-E, CD47, and/or viral immunosuppressive peptides can restore inhibition of the cytotoxic activity of NK cells (host or co-administered) and increase the persistence of the allogeneic cell product (including mixtures of NK cells and T cells). Experiments were undertaken to evaluate alternative approaches to complete B2M knockout (and resulting lack of MHC I surface expression). Rather, in several embodiments, the frequency of expression of B2M is reduced (not eliminated) methods are used to reduce MHC I surface expression (to avoid the cells being targeted by host T cells, or co-administered T cells), but still maintain sufficient levels of MHC I expression to inhibit the cytotoxic activities of host NK cells (or co-administered NK cells) against the edited cells.

As discussed above, various approaches can be used to reduce, but not eliminate, MHC I expression. In several embodiments, B2M expression is reduced temporarily using RNA interference. In some embodiments, viral protein expression can reduce MHC expression. In some embodiments gene editing to knockout expression of genes that function in the MHC I antigen expression pathway is used. Combinations of these approaches are used in several embodiments.

The present examples explore gene edits made to disrupt the secretory pathway by which peptide-loaded MHC I molecules are transported to the plasma membrane.

ATP-Binding Cassette (ABC) transporters comprise membrane proteins that translocate a wide variety of substrates across extra- and intracellular membranes. One such family (ABCB, also known as MDR/TAP) plays a crucial role in the processing and presentation of MHC I antigens. As depicted schematically in FIG. 25 (adapted from Pfisterer et al., Brazilian Journal of Allergy and Immunology (BJAI) 2014), TAP1 and TAP2 function to translocate the peptides into the endoplasmic reticulum.

Calreticulin (CRT) is a calcium-binding chaperone that, in the ER, facilitates folding of MHC I molecules along with the related MHC I recruitment/assembly factor, Tapasin. The thiol oxidoreductase ERp57 has been found to interact with Tapasin, and in its absence, the recruitment of MHC class I molecules into this complex by Tapasin significantly reduced. In addition to Tapasin, an additional intracellular peptide editor TABPR (also called TABPL; not shown in FIG. 25) functions to select/exchange peptides for attachment to MHC I. Acting as a quality control protein, UDP-glucose:glycoprotein glucosyltransferase 1 (UGT1, also known as UGTA-1) recognizes misfolded proteins/peptides due to lack of glycosylation and re-glycosylates them, allowing proper folding. It has been determined that while peptide formation still occurs in the absence of UGT-1 (there are redundancies in the folding process), surface level of MHC I is reduced, maturation and assembly are delayed, and peptide selection is impaired (Zhang et al., PNAS (2011) 108(12): 4956-61).

FIG. 16 shows a non-limiting experimental schematic to evaluate the effect of reduction of MHC I expression on the persistence of T cells, in a mixed NK+T cell population. At Day 0, T cells are purified and electroporated with a guide RNA targeting a gene of interested in the MHC I pathway, such as UGT-1 (UGTA-1); TAPBPL (TAPBPR); TAPBP (Tapasin); TAP-1; TAP-2; ERp57; Calreticulin (CRT); Endoplasmic reticulum aminopeptidases (ERAP1, ERAP2) and/or immunoproteasome components. Gene edits were made using a Crispr/Cas9 complex and a guide RNA in this study, though as described herein, other methods are used in additional embodiments. T cells are then expanded using CD3/CD28 antibody-containing stimulatory beads according to art-established methods. The stimulatory beads are removed at Day 2 and the expression of MHC I is evaluated at one or more time points during continued culture of the cells, for example Day 5 and/or Day 9. In a parallel system, on Day 2, NK cells are expanded in culture. On Day 8 the NK cells are transduced with a vector encoding an anti-CD19 CAR. In additional embodiments, other CARs are used. At Day 12, the NK and T cells are co-cultured at a desired ratio, with an additional phenotype assessment and/or survival assessment of the T and/or NK cells one day (Day 13) and two days (Day 15) after inception of co-culture.

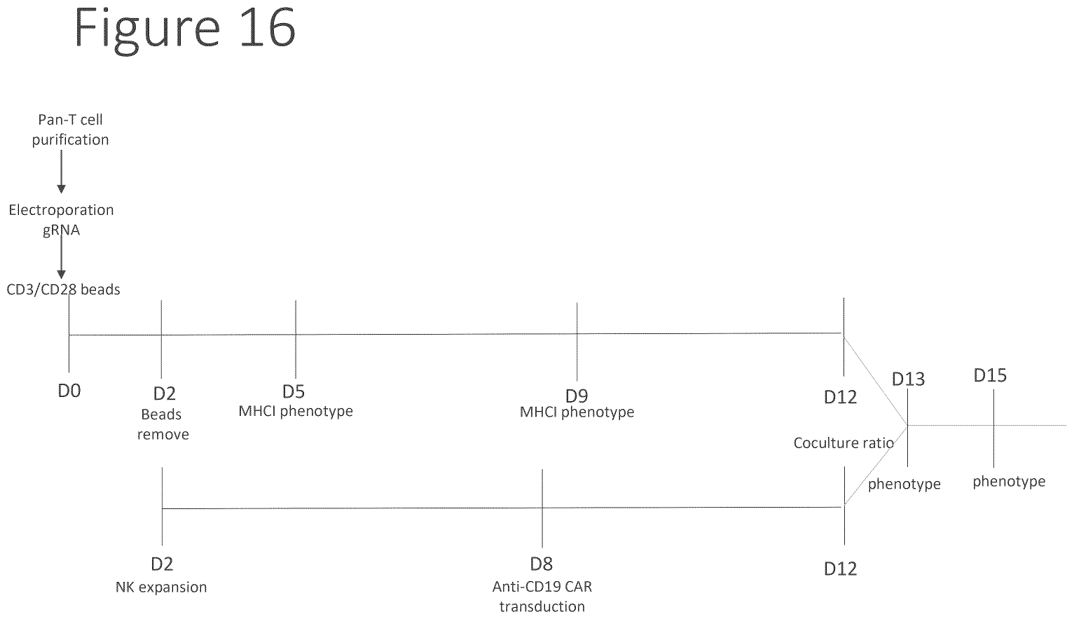

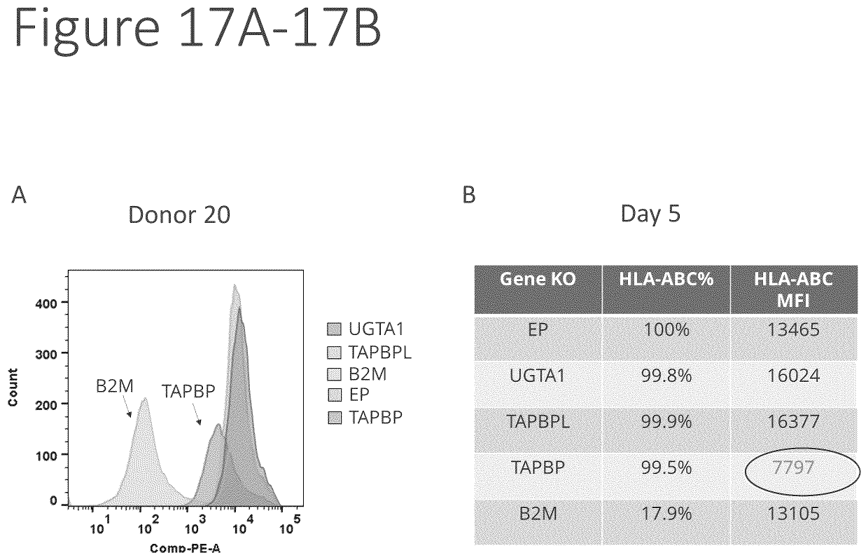

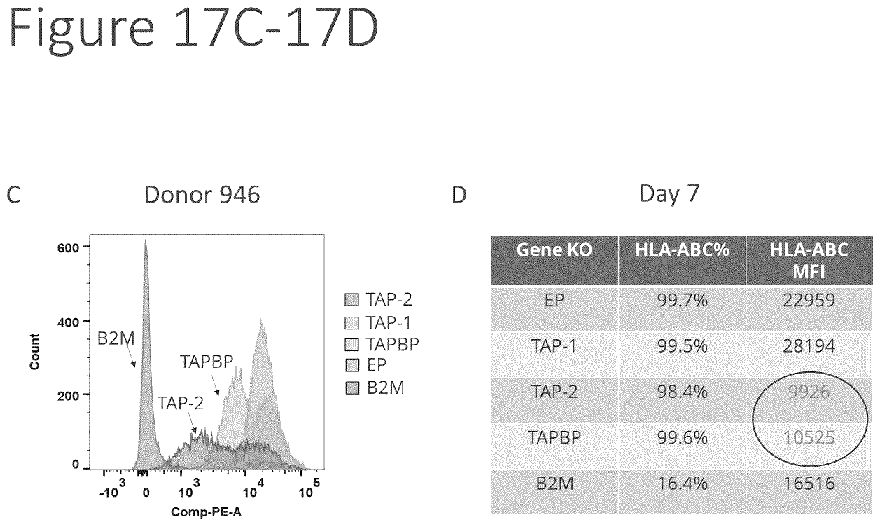

FIGS. 17A-17D show data from the initial MHC I phenotype assessment that takes place during expansion of the T cells after gene knockdown. FIG. 17A shows flow cytometry data assessing the expression of MHC I (using an anti-HLA-ABC antibody) after gene editing the gene encoding UGTA-1, TAPBPL, B2M, TAPBP (or an electroporation only control). This assessment was for cells obtained from a first donor and was performed 5 days post-editing. As would be expected, the gene editing of B2M resulted in substantial decrease in the amount of HLA detected, with TAPBP also showing a reduction. FIG. 17B shows data for each group in two formats. The central column shows the percentage of T cells evaluated that express HLA-ABC (MHC I). Consistent with the image of FIG. 17A, the most marked decrease was in the B2M edited group. The right column of FIG. 17B shows the mean fluorescence intensity (MFI) of the T cells evaluated, which reflects the degree (intensity) to which the population expresses MHC I. Thus, for example, fewer cells in the B2M-edited group actually express MHC I, but those that do express it, express it at levels similar to electroporated controls. In contrast, with the TAPBP group, while 99.5% of the population express MHC I, on average, each cell in the population expresses a lower amount of MHC I (circled value). FIGS. 17C-17D show similar data for cells from a second donor, with the evaluation being performed 7 days post-editing. A similar pattern is seen with the edits, in that B2M editing reduces the number of cells expressing MHC I, but not the mean amount expressed by each of the cells in the population. In contrast, cells edited to knockout either TAP-2 or TAPBP showed a reduced MFI (circled values), indicating that the amount of MHC I expressed by each of the cells is reduced, despite nearly 100% of the population still expressing some amount of MHC I.

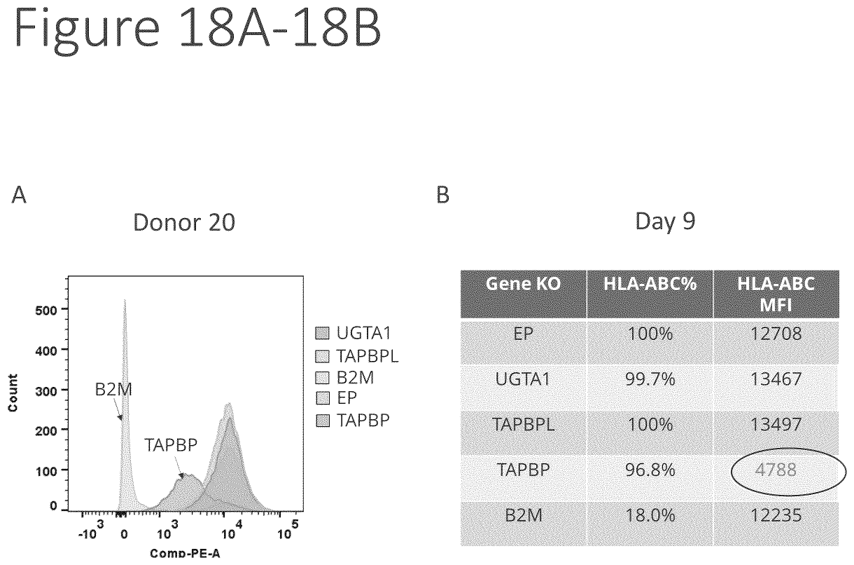

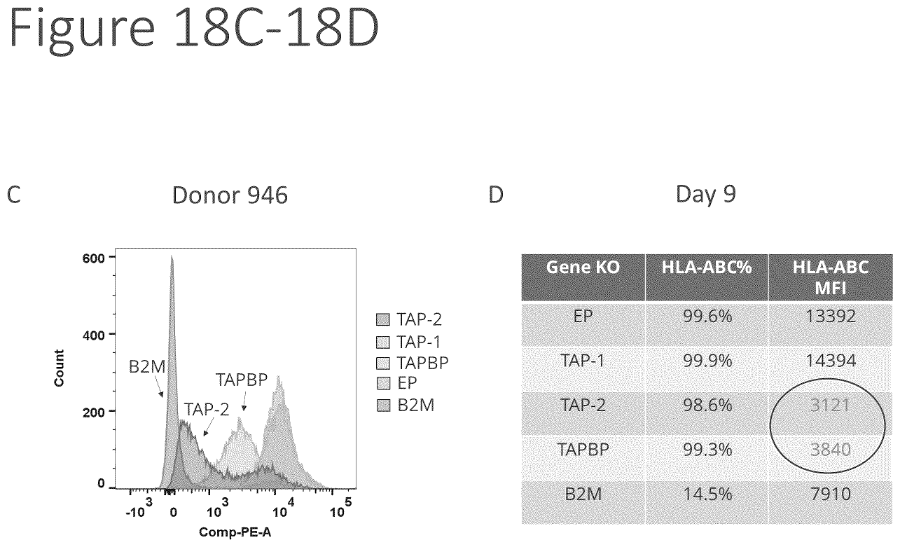

FIGS. 18A-18B show corresponding data for the first donor, at 9 days post-edit. FIGS. 18C-18D show corresponding data for the second donor, also at 9 days post-edit. Consistent with the data above from the earlier timepoint, TAPBP gene edits in the first donor resulted in reduced MHC I MFI (circled value). In the second donor, edits to both TAP-2 and TAPBP resulted in reduced MHC I expression ((circled values).

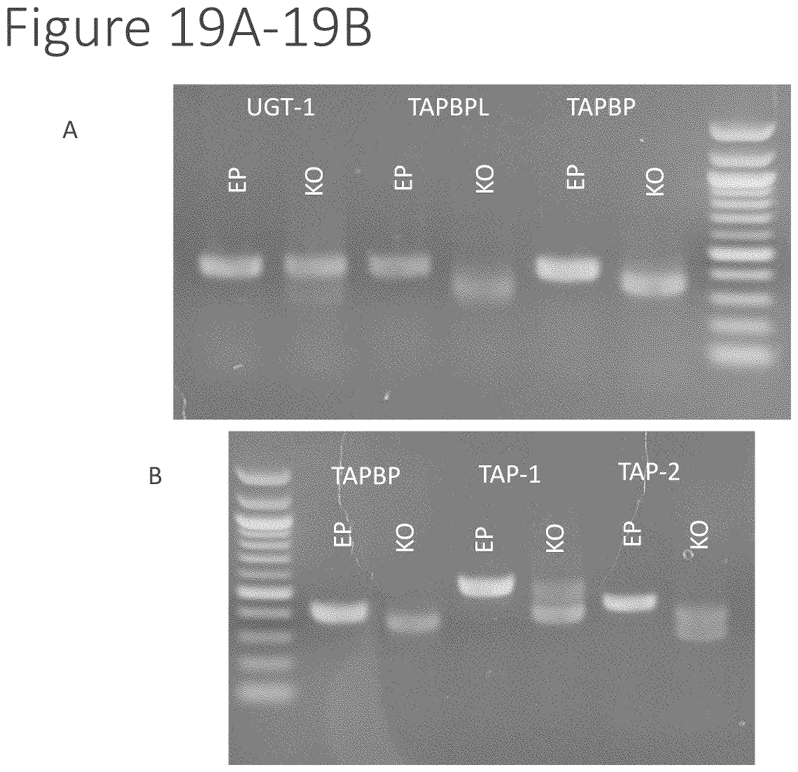

FIGS. 19A and 19B show agarose gel electrophoresis separation of DNA amplicons for the indicated gene (and condition, either EP control or edited) for the first donor (19A) and the second donor (19B). As can be seen, many of the gene edited samples show a size difference (smaller) than the corresponding control, which is consistent with a deletion of a region of the amplicon between the two corresponding PCR primers, which yields the shorter amplicon. These data show that the CRISPR editing is effective and, according to some embodiments, can be used to edit certain genes in the MHC I processing/peptide loading pathway to reduce the degree of MHC I expression by an edited cell.

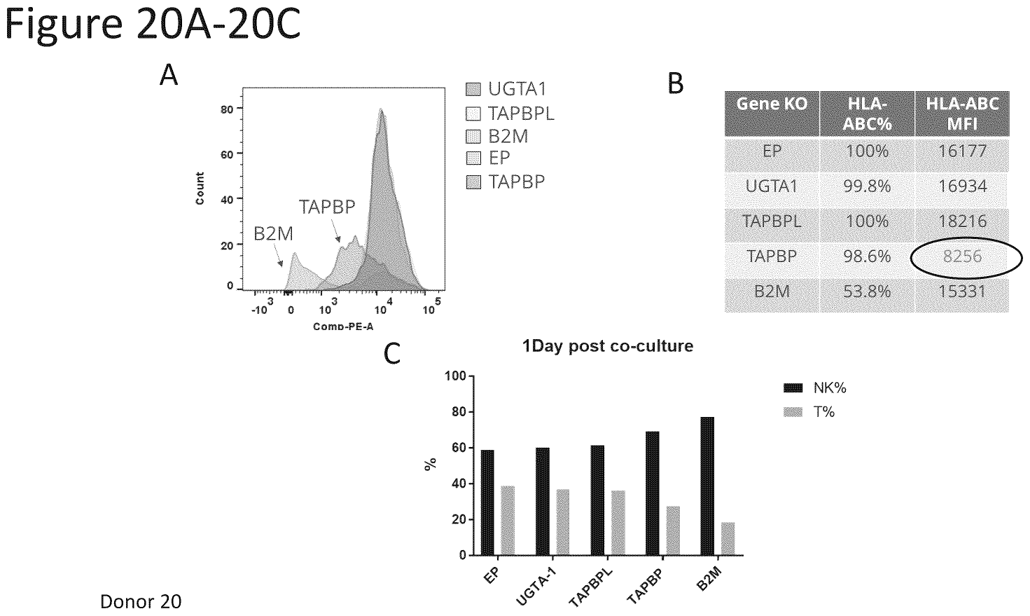

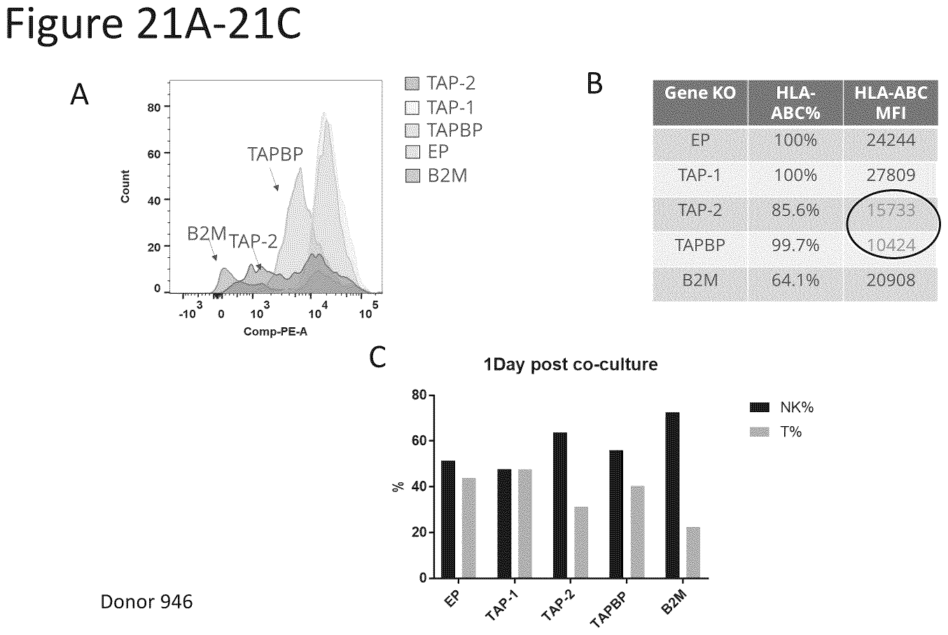

FIGS. 20A-20C show data from the first donor from the gene edit/expression, phenotype and expression degree of expression of edited T cells and CAR-expressing NK cells after 1 day of co-culture. Consistent with the data discussed above, edits to TAPBP yielded reduced mean MHC expression (20A-20B) and a greater T cell percentage as compared to those T cells edited at B2M. Those cells edited at B2M, due to that portion of the population lacking MHC expression (but with the MHC-expressing cells expressing at approximately normal levels) are subject to NK cell-based cytotoxicity. In contrast, the T cells edited at TAPBP (or other genes disclosed herein, in several embodiments) are maintained at a higher percentage in the co-culture, because the maintenance of some degree of expression of MHC I by nearly all the edited cells helps reduce the NK cell-based cytotoxic activity against the edited T cells. Similar data is shown for the second donor in FIGS. 21A-21C. Again, the reduced mean expression of MHC I after edits to TAP-2 or TAPBP conferred a greater degree of persistence to those populations of T cells, as compared to B2M-edited T cells (see FIG. 21C).

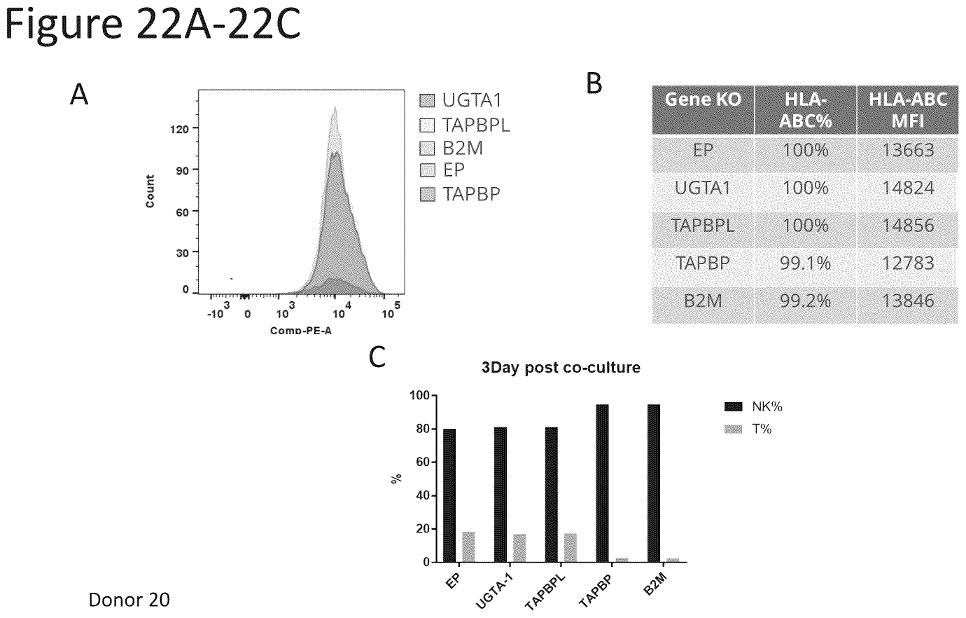

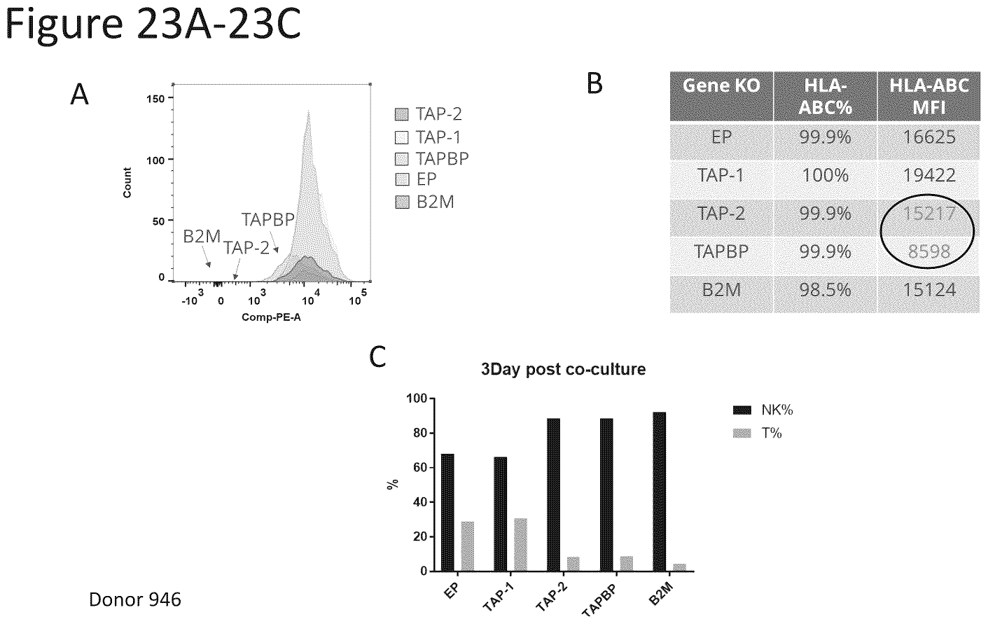

FIGS. 22A-22C show corresponding data from the first donor at three days post-coculture. In this particular experiment there was not a significant reduction in the MHC I for any of the groups and as such, the T cells were nearly absent from the co-culture at three days. Shown in FIGS. 23A-23C, the edited cells from the second donor did exhibit reduced mean MHC I expression when edited at TAP-2 and TAPBP (circled values in 23B). While much of the T cells were eliminated after three days, the percentage of T cells did appear to be greater for these groups compared to B2M edited cells (23C). Accordingly, in some embodiments, additional manipulation or edits to the cells are made to confer enhanced persistence to the T cells (or NK cells, depending on the embodiment). For example, in several embodiments, use of the viral immunosuppressive peptides disclosed herein in conjunction with reducing mean MHC I expression across the population functions to strike a balance between avoiding cytotoxicity from NK cells and host T cells.

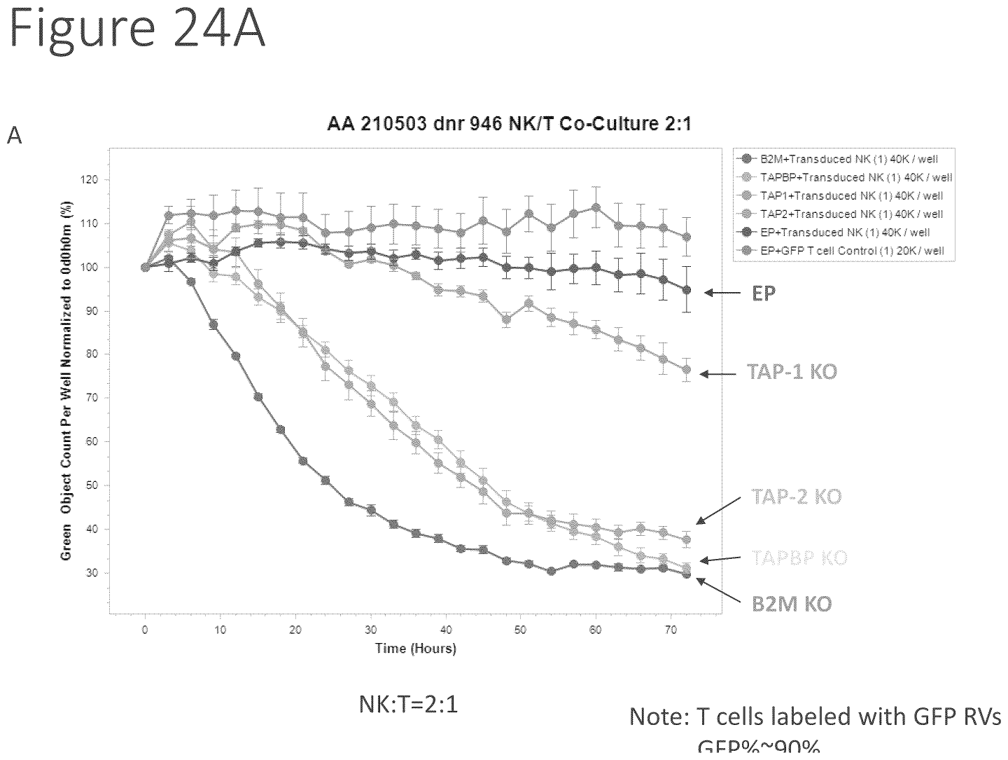

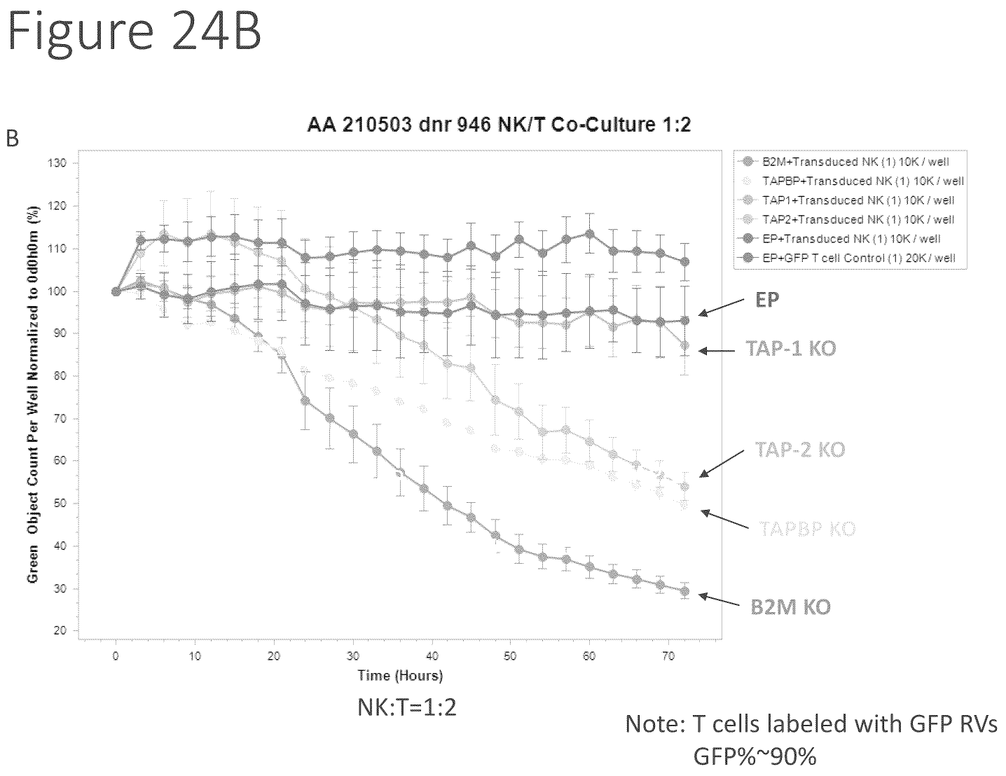

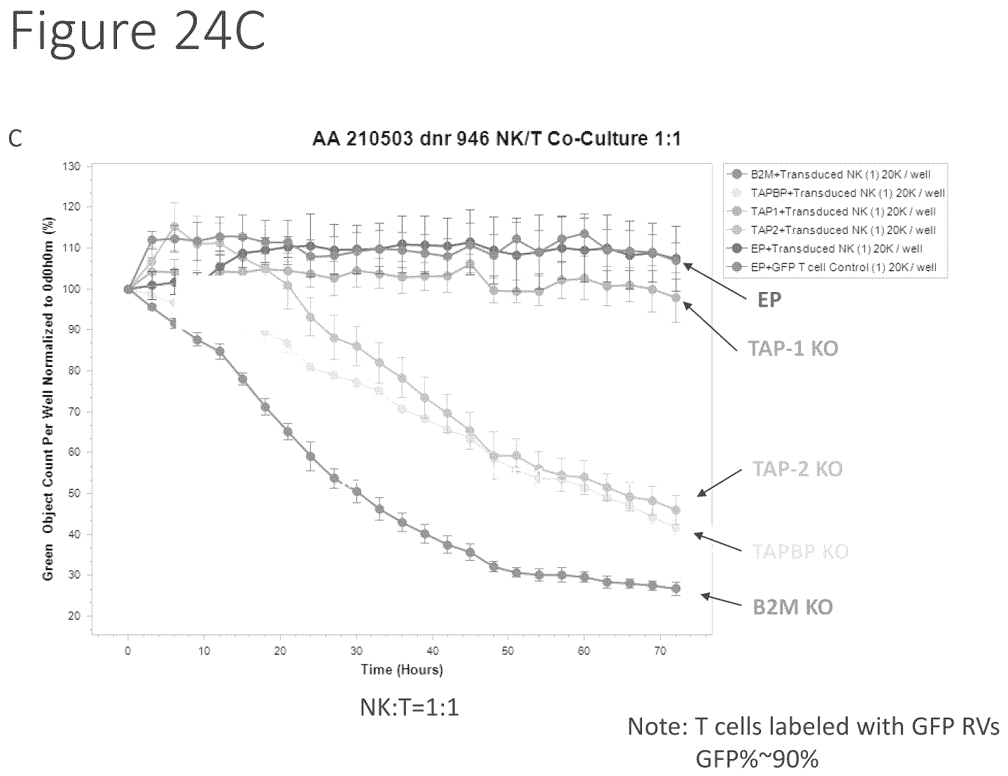

A more detailed assessment of T cell survival during NK+T co-culture using varied NK:T ratios with gene edited cells from the second donor is shown in FIGS. 24A-24C. In FIG. 24A, a 2:1 NK:T ratio was used, with T cells being labeled through retroviral GFP labeling/detection. As can be seen from the traces, the EP control T cells were maintained at approximately starting culture levels over the 70 hours co-culture period. Each of the TAP-2, TAPBP and B2M knockouts showed reduced T cell numbers over time. Despite not reducing MHC I mean expression, TAP-1 knockout cells persisted reasonably well, though perhaps for other reasons unrelated to the MHC I mode of action. That said, TAP-2 and TAPBP-edited cells did appear to remain at levels moderately above the B2M-edited group. Moving to a 1:2 NK:T cell ratio in FIG. 24B, the TAP-2 and TAPBP knockouts show substantially better maintenance of T cells within the mixed population. It also appears that the decline in T cells occurs after some delay as compared to the 2:1 ratio in FIG. 24A. Moving to a 1:1 NK:T ratio in FIG. 24C, even at matched cell numbers, the TAP-2 and TAPBP knockouts still exhibit greater persistence as compared to B2M-edited cells, representing their enhanced resistance to cytotoxic effects of NK cells. Thus, according to some embodiments, the edits of genes involved in the processing/presentation of peptides by MHC I can advantageously reduce average MHC I expression by edited T cells, without eliminating its expression, which allows the balance between avoiding cytotoxic effects from NK cells and host T cells. In several embodiments, synergy can be found between two or more of such genes being edited and/or optionally expressing another protein (or protein complex) to reduce the cytotoxic effects of NK cells (whether host or co-administered cells).

It is contemplated that various combinations or subcombinations of the specific features and aspects of the embodiments disclosed above may be made and still fall within one or more of the inventions. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above. Moreover, while the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication. In addition, where features or aspects of the disclosure are described in terms of Markush groups, those skilled in the art will recognize that the disclosure is also thereby described in terms of any individual member or subgroup of members of the Markush group.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers. For example, “about 90%” includes “90%.” In some embodiments, at least 95% sequence identity or homology includes 96%, 97%, 98%, 99%, and 100% sequence identity or homology to the reference sequence. In addition, when a sequence is disclosed as “comprising” a nucleotide or amino acid sequence, such a reference shall also include, unless otherwise indicated, that the sequence “comprises”, “consists of” or “consists essentially of” the recited sequence. Any titles or subheadings used herein are for organization purposes and should not be used to limit the scope of embodiments disclosed herein.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

### Sequences

In several embodiments, there are provided amino acid sequences that correspond to any of the nucleic acids disclosed herein (and/or included in the accompanying sequence listing), while accounting for degeneracy of the nucleic acid code. Furthermore, those sequences (whether nucleic acid or amino acid) that vary from those expressly disclosed herein (and/or included in the accompanying sequence listing), but have functional similarity or equivalency are also contemplated within the scope of the present disclosure. The foregoing includes mutants, truncations, substitutions, or other types of modifications. Additionally, it shall be appreciated that certain nucleic acid sequences, even if not expressly included within the sequence, comprise one of the three standard stop codons (TAA, TAG, or TGA) known in the standard genetic code. In accordance with some embodiments described herein, any of the sequences may be used, or a truncated or mutated form of any of the sequences disclosed herein (and/or included in the accompanying sequence listing) may be used and in any combination. A Sequence Listing in electronic format is submitted herewith. Some of the sequences provided in the Sequence Listing may be designated as Artificial Sequences by virtue of being non-naturally occurring fragments or portions of other sequences, including naturally occurring sequences. Some of the sequences provided in the Sequence Listing may be designated as Artificial Sequences by virtue of being combinations of sequences from different origins, such as humanized antibody sequences.

1-84. (canceled)

85. A population of genetically engineered immune cells comprising natural killer (NK) cells and T cells, wherein:
the T cells are genetically edited to comprise reduced expression of TAPBP protein; and
the T cells are engineered to express a chimeric antigen receptor (CAR) comprising an extracellular ligand-binding domain, a transmembrane domain, and a cytotoxic signaling complex.

86. The population of genetically engineered immune cells of claim 85, wherein at least about 80%, at least about 90%, at least about 95%, or at least about 99% of the T cells express major histocompatibility complex class I (MHC I) molecules.

87. The population of genetically engineered immune cells of claim 85, wherein the NK cells express a CAR comprising an extracellular ligand-binding domain, a transmembrane domain, and a cytotoxic signaling complex.

88. A method for the treating a subject having a disease or disorder comprising administering to the subject the population of genetically engineered immune cells according to claim 85.

89. A population of genetically engineered immune cells comprising natural killer (NK) cells and T cells, wherein:
the T cells are genetically edited to comprise reduced expression of TAP-1 protein; and
the T cells are engineered to express a chimeric antigen receptor (CAR) comprising an extracellular ligand-binding domain, a transmembrane domain, and a cytotoxic signaling complex.

90. The population of genetically engineered immune cells of claim 89, wherein at least about 80%, at least about 90%, at least about 95%, or at least about 99% of the T cells express major histocompatibility complex class I (MHC I) molecules.

91. The population of genetically engineered immune cells of claim 89, wherein the NK cells express a CAR comprising an extracellular ligand-binding domain, a transmembrane domain, and a cytotoxic signaling complex.

92. A method for the treating a subject having a disease or disorder comprising administering to the subject the population of genetically engineered immune cells according to claim 89.

93. A population of genetically engineered immune cells comprising natural killer (NK) cells and T cells, wherein:
the T cells are genetically edited to comprise reduced expression of TAP-2 protein; and
the T cells are engineered to express a chimeric antigen receptor (CAR) comprising an extracellular ligand-binding domain, a transmembrane domain, and a cytotoxic signaling complex.

94. The population of genetically engineered immune cells of claim 93, wherein at least about 80%, at least about 90%, at least about 95%, or at least about 99% of the T cells express major histocompatibility complex class I (MHC I) molecules.

95. The population of genetically engineered immune cells of claim 93, wherein the NK cells express a CAR comprising an extracellular ligand-binding domain, a transmembrane domain, and a cytotoxic signaling complex.

96. A method for the treating a subject having a disease or disorder comprising administering to the subject the population of genetically engineered immune cells according to claim 93.

97. A method of enhancing the in vivo persistence of genetically engineered immune cells, the method comprising:
(a) genetically editing a population of T cells to reduce expression of TAPBP, TAP-1, or TAP-2 protein;
(b) contacting the population of T cells with a polynucleotide encoding a chimeric antigen receptor (CAR) comprising an extracellular ligand binding domain, a transmembrane domain, and a cytotoxic signaling complex; and
(c) combining the population of T cells with a population of natural killer (NK) cells.

98. The method of claim 97, wherein the combining in (c) is in vivo.

99. The method of claim 97, wherein at least about 80%, at least about 90%, at least about 95%, or at least about 99% of the T cells express major histocompatibility complex class I (MHC I) molecules.

100. The method of claim 97, wherein the combining in (c) is done in vitro.

101. The method of claim 100, wherein the NK cells express a CAR comprising an extracellular ligand binding domain, a transmembrane domain, and a cytotoxic signaling complex.

102. The method of claim 100, wherein at least about 80%, at least about 90%, at least about 95%, or at least about 99% of the T cells express major histocompatibility complex class I (MHC I) molecules.

103. The method of claim 97, further comprising administering the combined population of T cells and NK cells to a subject having a disease or condition.

104. The method of claim 103, wherein at least about 80%, at least about 90%, at least about 95%, or at least about 99% of the T cells express major histocompatibility complex class I (MHC I) molecules.
